(19) United States
(12) Patent Application Publication
(10) Pub. No.: US 2020/0270232 A1
(43) Pub. Date: Aug. 27, 2020

(54) INHIBITORS OF INTEGRATED STRESS RESPONSE PATHWAY

(71) Applicant: Praxis Biotech LLC, San Francisco, CA (US)

(72) Inventors: Sebastian BERNALES, Piedmont, CA (US); Luz Marina DELGADO OYARZO, Santiago (CL); Gonzalo Esteban NÙÑEZ VASQUEZ, Santiago (CL); Gonzalo Andrés URETA DÍAZ, Santiago (CL); Brahmam PUJALA, Greater Noida (IN); Dayanand PANPATIL, Noida (IN)

(21) Appl. No.: 16/799,765

(22) Filed: Feb. 24, 2020

Related U.S. Application Data
(60) Provisional application No. 62/943,643, filed on Dec. 4, 2019.
(60) Provisional application No. 62/810,324, filed on Feb. 25, 2019.

(51) Int. Cl. C07D 405/12; A61P 25/28; A61P 35/00; A61P 21/00; C07C 233/52; C07D 307/85; C07D 215/48; C07D 401/06; C07D 405/06; C07D 307/14; C07D 401/12; C12P 21/00

(52) U.S. Cl. CPC C07D 405/12; A61P 25/28; A61P 35/00; A61P 21/00; C07C 233/52; C12P 21/00; C07D 215/48; C07D 401/06; C07D 405/06; C07D 307/14; C07D 401/12; C07D 307/85

(57) ABSTRACT

The present disclosure relates generally to therapeutic agents that may be useful as inhibitors of Integrated Stress Response (ISR) pathway.

### CROSS REFERENCE TO RELATED APPLICATIONS

This application claims the priority benefit of U.S. Provisional Patent Application Nos. 62/810,324, filed Feb. 25, 2019, and 62/943,643, filed Dec. 4, 2019, the disclosures of which are hereby incorporated herein by reference in their entireties.

### FIELD

The present disclosure relates generally to therapeutic agents that may be useful as inhibitors of Integrated Stress Response (ISR) pathway.

### BACKGROUND

Diverse cellular conditions and stresses activate a widely conserved signaling pathway termed the Integrated Stress Response (ISR) pathway. The ISR pathway is activated in response to intrinsic and extrinsic stresses, such as viral infections, hypoxia, glucose and amino acid deprivation, oncogene activation, UV radiation, and endoplasmic reticulum stress. Upon activation of ISR by one or more of these factors, the eukaryotic initiation factor 2 (eIF2, which is comprised of three subunits, α, β and γ) becomes phosphorylated in its α-subunit and rapidly reduces overall protein translation by binding to the eIF2B complex. This phosphorylation inhibits the eIF2B-mediated exchange of GDP for GTP (i.e., a guanine nucleotide exchange factor (GEF) activity), sequestering eIF2B in a complex with eIF2 and reducing general protein translation of most mRNA in the cell. Paradoxically, eIF2a phosphorylation also increases translation of a subset of mRNAs that contain one or more upstream open reading frames (uORFs) in their 5′ untranslated region (UTR). These transcripts include the transcriptional modulator activating transcription factor 4 (ATF4), the transcription factor CHOP, the growth arrest and DNA damage-inducible protein GADD34 and the β-secretase BACE-1.

In animals, the ISR modulates a broad translational and transcriptional program involved in diverse processes such as learning memory, immunity, intermediary metabolism, insulin production and resistance to unfolded protein stress in the endoplasmic reticulum, among others. Activation of the ISR pathway has also been associated with numerous pathological conditions including cancer, neurodegenerative diseases, metabolic diseases (metabolic syndrome), autoimmune diseases, inflammatory diseases, musculoskeletal diseases (such as myopathy), vascular diseases, ocular diseases, and genetic disorders. Aberrant protein synthesis through eIF2α phosphorylation is also characteristic of several other human genetic disorders, cystic fibrosis, amyotrophic lateral sclerosis, Huntington disease and prion disease.

### BRIEF SUMMARY

Inhibitors of the Integrated Stress Response (ISR) pathway are described, as are methods of making and using the compounds, or salts thereof.

### BRIEF DESCRIPTION OF THE DRAWINGS

FIG. 1 shows relative fluorescence intensity (RFU) of GFP resulting from a cell-free protein expression system treated with or without compound 90 and compound 94.

### DETAILED DESCRIPTION

Described herein are compounds, including therapeutic agents, that can inhibit the ISR pathway. These compounds could be used in the prevention and/or treatment of certain pathological conditions as described herein, and/or in biotechnology applications that would benefit from increased protein translation.

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkylene” as used herein refers to the same residues as alkyl, but having bivalency. Particular alkylene groups are those having 1 to 20 carbon atoms (a “C₁-C₂₀ alkylene”), having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

“Alkenyl” as used herein refers to and includes, unless otherwise stated, an unsaturated linear (i.e., unbranched) or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). An alkenyl group may have “cis” or “trans” configurations, or alternatively have “E” or “Z” configurations. Particular alkenyl groups are those having 2 to 20 carbon atoms (a “C₂-C₂₀ alkenyl”), having 6 to 10 carbon atoms (a “C₆-C₁₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl group include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, pent-1-enyl, pent-2-enyl, hex-1-enyl, hex-2-enyl, hex-3-enyl, and the like.

“Alkenylene” as used herein refers to the same residues as alkenyl, but having bivalency. Particular alkenylene groups are those having 2 to 20 carbon atoms (a “C₂-C₂₀ alkenylene”), having 2 to 10 carbon atoms (a “C₂-C₁₀ alkenylene”), having 6 to 10 carbon atoms (a “C₆-C₁₀ alkenylene”), having 2 to 6 carbon atoms (a “C₂-C₆ alkenylene”), 2 to 4 carbon atoms (a “C₂-C₄ alkenylene”) or 2 to 3 carbon atoms (a “C₂-C₃ alkenylene”). Examples of alkenylene include, but are not limited to, groups such as ethenylene (or vinylene) (—CH═CH—), propenylene (—CH═CHCH₂—), 1,4-but-1-enylene (—CH═CH—CH₂CH₂—), 1,4-but-2-enylene (—CH₂CH═CHCH₂—), 1,6-hex-1-enylene (—CH═CH—(CH₂)₃CH₂—), and the like.

“Alkynyl” as used herein refers to and includes, unless otherwise stated, an unsaturated linear (i.e., unbranched) or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). Particular alkynyl groups are those having 2 to 20 carbon atoms (a “C₂-C₂₀ alkynyl”), having 6 to 10 carbon atoms (a “C₆-C₁₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl group include, but are not limited to, groups such as ethynyl (or acetylenyl), prop-1-ynyl, prop-2-ynyl (or propargyl), but-1-ynyl, but-2-ynyl, but-3-ynyl, and the like.

“Alkynylene” as used herein refers to the same residues as alkynyl, but having bivalency. Particular alkynylene groups are those having 2 to 20 carbon atoms (a “C₂-C₂₀ alkynylene”), having 2 to 10 carbon atoms (a “C₂-C₁₀ alkynylene”), having 6 to 10 carbon atoms (a “C₆-C₁₀ alkynylene”), having 2 to 6 carbon atoms (a “C₂-C₆ alkynylene”), 2 to 4 carbon atoms (a “C₂-C₄ alkynylene”) or 2 to 3 carbon atoms (a “C₂-C₃ alkynylene”). Examples of alkynylene include, but are not limited to, groups such as ethynylene (or acetylenylene) (—C≡C—), propynylene (—C≡CCH₂—), and the like.

“Cycloalkyl” as used herein refers to and includes, unless otherwise stated, saturated cyclic univalent hydrocarbon structures, having the number of carbon atoms designated (i.e., C₃-C₁₀ means three to ten carbon atoms). Cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃-C₈ cycloalkyl”), having 3 to 6 carbon atoms (a “C₃-C₆ cycloalkyl”), or having from 3 to 4 annular carbon atoms (a “C₃-C₄ cycloalkyl”). Examples of cycloalkyl include, but are not limited to, cyclopropyl, cyclobutyl, cyclopentyl, cyclohexyl, cycloheptyl, norbornyl, and the like.

“Cycloalkylene” as used herein refers to the same residues as cycloalkyl, but having bivalency. Cycloalkylene can consist of one ring or multiple rings which may be fused, spiro or bridged, or combinations thereof. Particular cycloalkylene groups are those having from 3 to 12 annular carbon atoms. A preferred cycloalkylene is a cyclic hydrocarbon having from 3 to 8 annular carbon atoms (a “C₃-C₈ cycloalkylene”), having 3 to 6 carbon atoms (a “C₃-C₆ cycloalkylene”), or having from 3 to 4 annular carbon atoms (a “C₃-C₄ cycloalkylene”). Examples of cycloalkylene include, but are not limited to, cyclopropylene, cyclobutylene, cyclopentylene, cyclohexylene, cycloheptylene, norbornylene, and the like. A cycloalkylene may attach to the remaining structures via the same ring carbon atom or different ring carbon atoms. When a cycloalkylene attaches to the remaining structures via two different ring carbon atoms, the connecting bonds may be cis- or trans- to each other. For example, cyclopropylene may include 1,1-cyclopropylene and 1,2-cyclopropylene (e.g., cis-1,2-cyclopropylene or trans-1,2-cyclopropylene), or a mixture thereof.

“Cycloalkenyl” refers to and includes, unless otherwise stated, an unsaturated cyclic non-aromatic univalent hydrocarbon structure, having at least one site of olefinic unsaturation (i.e., having at least one moiety of the formula C═C) and having the number of carbon atoms designated (i.e., C₂-C₁₀ means two to ten carbon atoms). Cycloalkenyl can consist of one ring, such as cyclohexenyl, or multiple rings, such as norbornenyl. A preferred cycloalkenyl is an unsaturated cyclic hydrocarbon having from 3 to 8 annular carbon atoms (a “C₃-C₈ cycloalkenyl”). Examples of cycloalkenyl groups include, but are not limited to, cyclopropenyl, cyclobutenyl, cyclopentenyl, cyclohexenyl, norbornenyl, and the like.

“Cycloalkenylene” as used herein refers to the same residues as cycloalkenyl, but having bivalency.

“Aryl” or “Ar” as used herein refers to an unsaturated aromatic carbocyclic group having a single ring (e.g., phenyl) or multiple condensed rings (e.g., naphthyl or anthryl) which condensed rings may or may not be aromatic. Particular aryl groups are those having from 6 to 14 annular carbon atoms (a “C₆-C₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Arylene” as used herein refers to the same residues as aryl, but having bivalency. Particular arylene groups are those having from 6 to 14 annular carbon atoms (a “C₆-C₁₄ arylene”).

“Heteroaryl” as used herein refers to an unsaturated aromatic cyclic group having from 1 to 14 annular carbon atoms and at least one annular heteroatom, including but not limited to heteroatoms such as nitrogen, oxygen, and sulfur. A heteroaryl group may have a single ring (e.g., pyridyl, furyl) or multiple condensed rings (e.g., indolizinyl, benzothienyl) which condensed rings may or may not be aromatic. Particular heteroaryl groups are 5 to 14-membered rings having 1 to 12 annular carbon atoms and 1 to 6 annular heteroatoms independently selected from nitrogen, oxygen, and sulfur, 5 to 10-membered rings having 1 to 8 annular carbon atoms and 1 to 4 annular heteroatoms independently selected from nitrogen, oxygen, and sulfur, or 5, 6 or 7-membered rings having 1 to 5 annular carbon atoms and 1 to 4 annular heteroatoms independently selected from nitrogen, oxygen, and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen, and sulfur. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position. A heteroaryl group may be connected to the parent structure at a ring carbon atom or a ring heteroatom.

“Heteroarylene” as used herein refers to the same residues as heteroaryl, but having bivalency.

“Heterocycle”, “heterocyclic”, or “heterocyclyl” as used herein refers to a saturated or an unsaturated non-aromatic cyclic group having a single ring or multiple condensed rings, and having from 1 to 14 annular carbon atoms and from 1 to 6 annular heteroatoms, such as nitrogen, sulfur or oxygen, and the like. A heterocycle comprising more than one ring may be fused, bridged or spiro, or any combination thereof, but excludes heteroaryl. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur.

“Heterocyclylene” as used herein refers to the same residues as heterocyclyl, but having bivalency.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Thiocarbonyl” refers to the group C═S.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. The methods of the present disclosure contemplate any one or more of these aspects of treatment.

As used herein, the term “effective amount” intends such amount of a compound of the invention which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

A “therapeutically effective amount” refers to an amount of a compound or salt thereof sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the present disclosure in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound of the present disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

It is understood that aspects and embodiments described herein as “comprising” include “consisting of” and “consisting essentially of” embodiments.

When a composition is described as “consisting essentially of” the listed components, the composition contains the components expressly listed, and may contain other components which do not substantially affect the disease or condition being treated such as trace impurities. However, the composition either does not contain any other components which do substantially affect the disease or condition being treated other than those components expressly listed; or, if the composition does contain extra components other than those listed which substantially affect the disease or condition being treated, the composition does not contain a sufficient concentration or amount of those extra components to substantially affect the disease or condition being treated. When a method is described as “consisting essentially of” the listed steps, the method contains the steps listed, and may contain other steps that do not substantially affect the disease or condition being treated, but the method does not contain any other steps which substantially affect the disease or condition being treated other than those steps expressly listed.

When a moiety is indicated as substituted by “at least one” substituent, this also encompasses the disclosure of exactly one substituent.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

### Compounds

In one aspect, provided is a compound of formula (I):

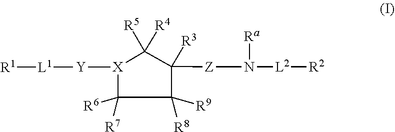

or a pharmaceutically acceptable salt thereof,
wherein:
X is N or CR¹²;
Y is a bond, NR^{a}, or NR^{a}NR^{a}; provided that:
   (a) when X is N, then Y is a bond or NR^{a}; and
   (b) when X is CR¹², then Y is NR^{a} or NR^{a}NR^{a};
Z is a bond, C(═O), CR¹⁰R¹¹, or NR^{a};
L¹ is selected from the group consisting of *1-C(═O)-#1, *1-CH₂-#1, *1-CH₂CH₂-#1, *1-CH₂CH₂CH₂-#1, *1-OCH₂C(═O)-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1;
   wherein *1 represents the attachment point to R¹ and #1 represents the attachment point to the remainder of the molecule;
L² is selected from the group consisting of #2-C(═O)-*2, #2-CH₂-*2, #2-CH₂CH₂-*2, #2-CH₂CH₂CH₂-*2, #2-C(═O)CH₂O—*2, #2-C(═O)CH₂CH₂O—*2, #2-C(═O)CH₂CH₂CH₂O-*2, #2-CH₂CH(OH)CH₂O—*2, #2-CH₂O-*2, #2-CH₂CH₂O-*2, and #2-CH₂CH₂CH₂O-*2;
   wherein *2 represents the attachment point to R² and #2 represents the attachment point to the remainder of the molecule;
R¹ is selected from the group consisting of:
   C₆-C₁₄ aryl substituted with one or more halo groups and optionally substituted with one or more R^{b}; and
   5-14 membered heteroaryl substituted with one or more halo groups and optionally substituted with one or more R^{b};
R² is selected from the group consisting of:
   C₆-C₁₄ aryl substituted with one or more halo groups and optionally substituted one or more R^{b}; and
   5-14 membered heteroaryl substituted with one or more halo groups and optionally substituted one or more R^{b};
R³ is hydrogen, halogen, or C₁-C₆ alkyl; or R³ and R¹² are taken together to form a CR¹³R¹⁴ group;
R⁴, R⁵, R⁶, R⁷, R⁸, and R⁹, independently of each other, are selected from the group consisting of hydrogen, halogen, and C₁-C₆ alkyl;
R¹⁰ and R¹¹, independently of each other, are selected from the group consisting of hydrogen, halogen, and C₁-C₆ alkyl;
R¹² is hydrogen, halogen, or C₁-C₆ alkyl; or R³ and R¹² are taken together to form a CR¹³R¹⁴ group;
R¹³ and R¹⁴, independently of each other, are selected from the group consisting of hydrogen, halogen, and C₁-C₆ alkyl;
R^{a}, independently at each occurrence, is hydrogen or C₁-C₆ alkyl;
R^{b}, independently at each occurrence, is selected from the group consisting of NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{c}R^{d}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{14-a}R^{14-b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{c}R^{d}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl),
   wherein R^{c} and R^{d} are taken together with the nitrogen atom to which they are attached to form a 3-10 membered heterocycle; and
   provided that:
   (i) when X is CR¹², Y is NR^{a}, Z is a bond, L¹ is *1-CH₂-#1, and L² is #2-CH₂-*2; then either:
      (i-1) at least one of R³, R⁴, and R⁵ is hydrogen or halogen; or
      (i-2) R³ and R¹² are taken together to form a CR¹³R¹⁴ group;
   (ii) when X is CR¹², Y is NR^{a}, Z is a bond, and R³ and R¹² are taken together to form CR¹³R¹⁴; then either:
      (ii-1) L¹ is selected from the group consisting of *1-C(═O)-#1, *1-CH₂-#1, *1-CH₂CH₂-#1, *1-CH₂CH₂CH₂-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1; or
      (ii-2) L² is selected from the group consisting of #2-C(═O)-*2, #2-CH₂-*2, #2-CH₂CH₂-*2, #2-CH₂CH₂CH₂-*2, #2-C(═O)CH₂CH₂O—*2, #2-C(═O)CH₂CH₂CH₂O-*2, #2-CH₂CH(OH)CH₂O—*2, #2-CH₂O-*2, #2-CH₂CH₂O-*2, and #2-CH₂CH₂CH₂O-*2; and
   (iii) when X is N and Y is a bond; then:
      L¹ is selected from the group consisting of *1-OCH₂CH(OH)CH₂-#1, *1-OCH₂-#1, *1-OCH₂CH₂-#1, and *1-OCH₂CH₂CH₂-#1; and
         further provided that, when Z is CR¹⁰R¹¹, then at least one of R¹ and R² is substituted by two or more halo groups.

In some embodiments of the compound of formula (I), X is N. In some embodiments, X is CR¹².

In some embodiments, Y is a bond. In some embodiments, Y is NR^{a}. In some embodiments, Y is NR^{a}, wherein R^{a} is hydrogen. In some embodiments, Y is NR^{a}, wherein R^{a} is C₁-C₆ alkyl, such as methyl, ethyl, n-propyl, isopropyl, n-butyl, t-butyl, isobutyl, or sec-butyl. In some embodiments, Y is NR^{a}NR^{a}. In some embodiments, Y is N(C₁-C₆ alkyl)NH. In some embodiments, Y is N(H)N(C₁-C₆ alkyl). In some embodiments, Y is N(C₁-C₆ alkyl)N(C₁-C₆ alkyl). In some embodiments, Y is NHNH.

In some embodiments, Z is a bond. In some embodiments, Z is CR¹⁰R¹¹. In some embodiments, Z is NR^{a}. In some embodiments, Z is NR^{a}, wherein R^{a} is hydrogen. In some embodiments, Z is NR^{a}, wherein R^{a} is C₁-C₆ alkyl, such as methyl, ethyl, n-propyl, isopropyl, n-butyl, t-butyl, isobutyl, or sec-butyl. In some embodiments, Z is C(═O).

In some embodiments, the compound of formula (I), or a pharmaceutically acceptable salt thereof, is a compound of formula (II):

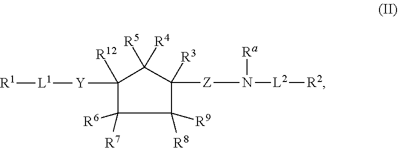

or a pharmaceutically acceptable salt thereof,
wherein R¹, R², R³, R⁴, R⁵, R⁶, R⁷, R⁸, R⁹, R¹², R^{a}, L¹, L², Y, and Z are as defined in compounds of formula (I),
provided that:
(i) when Y is NR^{a}, Z is a bond, L¹ is *1-CH₂-#1, and L² is #2-CH₂-*2; then either:
   (i-1) at least one of R³, R⁴, and R⁵ is hydrogen or halogen; or
   (i-2) R³ and R¹² are taken together to form a CR¹³R¹⁴ group; and
(ii) when Y is NR^{a}, Z is a bond, and R³ and R¹² are taken together to form CR¹³R¹⁴; then either:
   (ii-1) L¹ is selected from the group consisting of *1-C(═O)-#1, *1-CH₂-#1, *1-CH₂CH₂-#1, *1-CH₂CH₂CH₂-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1; or
   (ii-2) L2 is selected from the group consisting of #2-C(═O)-*2, #2-CH2-*2, #2-CH2CH2-*2, #2-CH2CH2CH2-*2, #2-C(═O)CH2CH2O-*2, #2-C(═O)CH2CH2CH2O-*2, #2-CH2CH(OH)CH2O-*2, #2-CH2O-*2, #2-CH2CH2O-*2, and #2-CH2CH2CH2O-*2.

In some embodiments, the compound of formula (I), or a pharmaceutically acceptable salt thereof, is a compound of formula (IV):

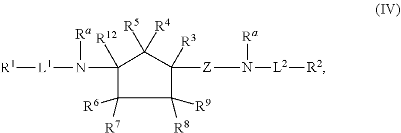

or a pharmaceutically acceptable salt thereof,
wherein R¹, R², R³, R⁴, R⁵, R⁶, R⁷, R⁸, R⁹, R¹², R^{a}, L¹, L², and Z are as defined in compounds of formula (I),
provided that:
(i) when Z is a bond, L¹ is *1-CH₂-#1, and L² is #2-CH₂-*2; then either:
   (i-1) at least one of R³, R⁴, and R⁵ is hydrogen or halogen; or
   (i-2) R³ and R¹² are taken together to form a CR¹³R¹⁴ group; and
(ii) when Z is a bond, and R³ and R¹² are taken together to form CR¹³R¹⁴; then either:
   (ii-1) L¹ is selected from the group consisting of *1-C(═O)-#1, *1-CH₂-#1, *1-CH₂CH₂-#1, *1-CH₂CH₂CH₂-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1; or
   (ii-2) L² is selected from the group consisting of #2-C(═O)-*2, #2-CH₂-*2, #2-CH₂CH₂-*2, #2-CH₂CH₂CH₂-*2, #2-C(═O)CH₂CH₂O—*2, #2-C(═O)CH₂CH₂CH₂O-*2, #2-CH₂CH(OH)CH₂O—*2, #2-CH₂O-*2, #2-CH₂CH₂O-*2, and #2-CH₂CH₂CH₂O-*2.

In some embodiments, the compound of formula (I), or a pharmaceutically acceptable salt thereof, is a compound of formula (IV-a):

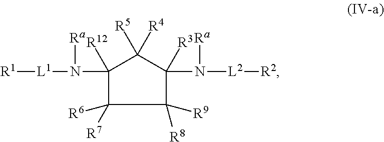

or a pharmaceutically acceptable salt thereof,
wherein:
R³ is hydrogen, halogen, or C₁-C₆ alkyl;
R¹² is hydrogen, halogen, or C₁-C₆ alkyl; and
provided that when L¹ is *1-CH₂-#1, and L² is #2-CH₂-*2; then at least one of R³, R⁴, and R⁵ is hydrogen or halogen; and
R¹, R², R⁴, R⁵, R⁶, R⁷, R⁸, R⁹, R^{a}, L¹, and L² are as defined in compounds of formula (I).

In some embodiments, the compound of formula (I), or a pharmaceutically acceptable salt thereof, is a compound of formula (IV-b)

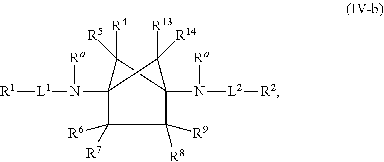

or a pharmaceutically acceptable salt thereof,
wherein R¹, R², R⁴, R⁵, R⁶, R⁷, R⁸, R⁹, R¹³, R¹⁴, R^{a}, L¹, and L² are as defined in compounds of formula (I).

In some embodiments, the compound of formula (I), or a pharmaceutically acceptable salt thereof, is a compound of formula (IV-c)

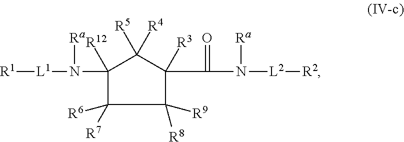

or a pharmaceutically acceptable salt thereof,
wherein:
R³ is hydrogen, halogen, or C₁-C₆ alkyl;
R¹² is hydrogen, halogen, or C₁-C₆ alkyl; and
R¹, R², R⁴, R⁵, R⁶, R⁷, R⁸, R⁹, R^{a}, L¹, and L² are as defined in compounds of formula (I).

In some embodiments, the compound of formula (I), or a pharmaceutically acceptable salt thereof, is a compound of formula (IV-d)

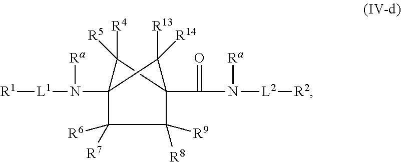

or a pharmaceutically acceptable salt thereof,
wherein R¹, R², R⁴, R⁵, R⁶, R⁷, R⁸, R⁹, R¹³, R¹⁴, R^{a}, L¹, and L² are as defined in compounds of formula (I).

In some embodiments, the compound of formula (I), or a pharmaceutically acceptable salt thereof, is a compound of formula (IV-e)

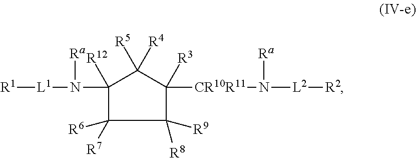

or a pharmaceutically acceptable salt thereof,
wherein:
R³ is hydrogen, halogen, or C₁-C₆ alkyl;
R¹² is hydrogen, halogen, or C₁-C₆ alkyl; and
R¹, R², R⁴, R⁵, R⁶, R⁷, R⁸, R⁹, R¹⁰, R¹¹, R^{a}, L¹, and L² are as defined in compounds of formula (I).

In some embodiments, the compound of formula (I), or a pharmaceutically acceptable salt thereof, is a compound of formula (IV-f)

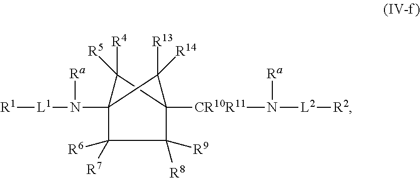

or a pharmaceutically acceptable salt thereof,
wherein R¹, R², R⁴, R⁵, R⁶, R⁷, R⁸, R, R¹⁰, R¹¹, R¹³, R¹⁴, R^{a}, L¹, and L² are as defined in compounds of formula (I).

In some embodiments, the compound of formula (I), or a pharmaceutically acceptable salt thereof, is a compound of formula (IV-g)

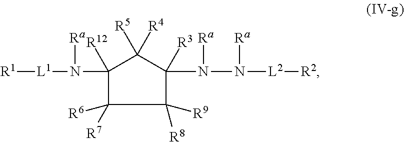

or a pharmaceutically acceptable salt thereof,
wherein:
R³ is hydrogen, halogen, or C₁-C₆ alkyl;
R¹² is hydrogen, halogen, or C₁-C₆ alkyl; and
R¹, R², R⁴, R⁵, R⁶, R⁷, R⁸, R⁹, R^{a}, L¹, and L² are as defined in compounds of formula (I).

In some embodiments, the compound of formula (I), or a pharmaceutically acceptable salt thereof, is a compound of formula (IV-h)

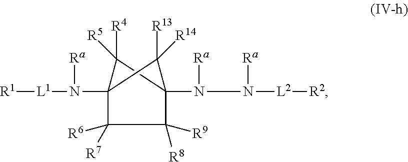

or a pharmaceutically acceptable salt thereof,
wherein R¹, R², R⁴, R⁵, R⁶, R⁷, R⁸, R⁹, R¹³, R¹⁴, R^{a}, L¹, and L² are as defined in compounds of formula (I).

In some embodiments, the compound of formula (I), or a pharmaceutically acceptable salt thereof, is a compound of formula (V)

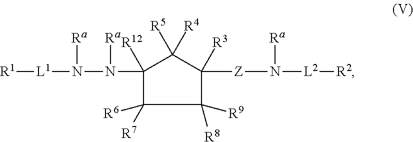

or a pharmaceutically acceptable salt thereof,
wherein R¹, R², R³, R⁴, R⁵, R⁶, R⁷, R⁸, R⁹, R¹², R^{a}, Z, L¹, and L² are as defined in compounds of formula (I).

In some embodiments, the compound of formula (I), or a pharmaceutically acceptable salt thereof, is a compound of formula (V-a)

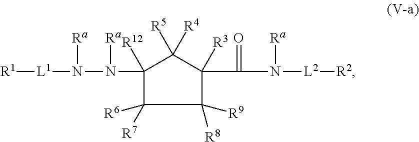

or a pharmaceutically acceptable salt thereof,
wherein:
R³ is hydrogen, halogen, or C₁-C₆ alkyl;
R¹² is hydrogen, halogen, or C₁-C₆ alkyl; and
R¹, R², R⁴, R⁵, R⁶, R⁷, R⁸, R⁹, R^{a}, L¹, and L² are as defined in compounds of formula (I).

In some embodiments, the compound of formula (I), or a pharmaceutically acceptable salt thereof, is a compound of formula (V-b)

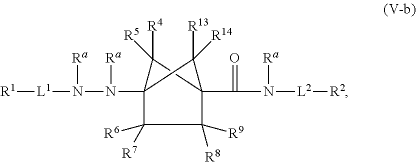

or a pharmaceutically acceptable salt thereof,
wherein R¹, R², R⁴, R⁵, R⁶, R⁷, R⁸, R⁹, R¹³, R¹⁴, R^{a}, L¹, and L² are as defined in compounds of formula (I).

In some embodiments, the compound of formula (I), or a pharmaceutically acceptable salt thereof, is a compound of formula (V-c)

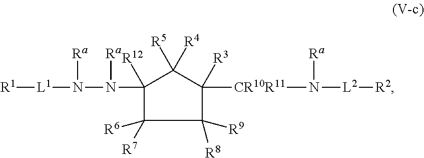

or a pharmaceutically acceptable salt thereof,
wherein:
R³ is hydrogen, halogen, or C₁-C₆ alkyl;
R¹² is hydrogen, halogen, or C₁-C₆ alkyl; and
R¹, R², R⁴, R⁵, R⁶, R⁷, R⁸, R⁹, R¹⁰, R¹¹, R^{a}, L¹, and L² are as defined in compounds of formula (I).

In some embodiments, the compound of formula (I), or a pharmaceutically acceptable salt thereof, is a compound of formula (V-d)

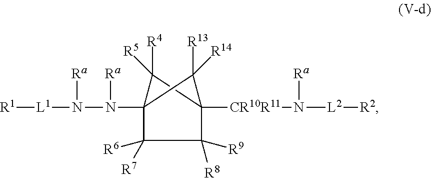

or a pharmaceutically acceptable salt thereof,
wherein R¹, R², R⁴, R⁵, R⁶, R⁷, R⁸, R⁹, R¹⁰, R¹¹, R¹³, R¹⁴, R^{a}, L¹, and L² are as defined in compounds of formula (I).

In some embodiments, the compound of formula (I), or a pharmaceutically acceptable salt thereof, is a compound of formula (V-e)

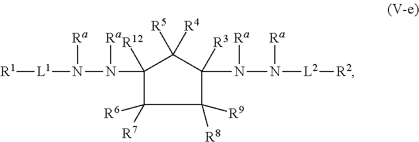

or a pharmaceutically acceptable salt thereof,
wherein:
R³ is hydrogen, halogen, or C₁-C₆ alkyl;
R¹² is hydrogen, halogen, or C₁-C₆ alkyl; and
R¹, R², R⁴, R⁵, R⁶, R⁷, R⁸, R⁹, R^{a}, L¹, and L² are as defined in compounds of formula (I).

In some embodiments, the compound of formula (I), or a pharmaceutically acceptable salt thereof, is a compound of formula (V-f)

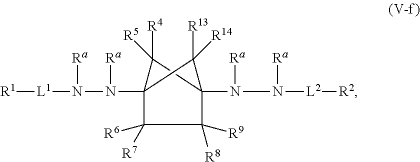

or a pharmaceutically acceptable salt thereof,
wherein R¹, R², R⁴, R⁵, R⁶, R⁷, R⁸, R⁹, R¹³, R¹⁴, R^{a}, L¹, and L² are as defined in compounds of formula (I).

In some embodiments, the compound of formula (I), or a pharmaceutically acceptable salt thereof, is a compound of formula (III)

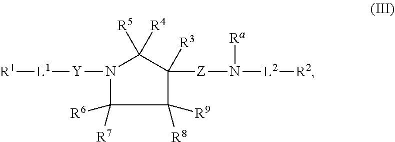

or a pharmaceutically acceptable salt thereof,
wherein R¹, R², R³, R⁴, R⁵, R⁶, R⁷, R⁸, R⁹, R^{a}, Y, Z, L¹, and L² are as defined in compounds of formula (I),
provided that when Y is a bond; then:
   L¹ is selected from the group consisting of *1-OCH₂CH(OH)CH₂-#1, *1-OCH₂-#1, *1-OCH₂CH₂-#1, and *1-OCH₂CH₂CH₂-#1; and
      further provided that, when Z is CR¹⁰R¹¹, then at least one of R¹ and R² is substituted by two or more halo groups.

In some embodiments, the compound of formula (I), or a pharmaceutically acceptable salt thereof, is a compound of formula (VI)

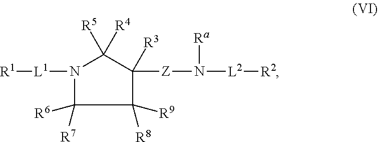

or a pharmaceutically acceptable salt thereof,
wherein R¹, R², R³, R⁴, R⁵, R⁶, R⁷, R⁸, R⁹, R^{a}, Z, L¹, and L² are as defined in compounds of formula (I),
provided that L¹ is selected from the group consisting of *1-OCH₂CH(OH)CH₂-#1, *1-OCH₂-#1, *1-OCH₂CH₂-#1, and *1-OCH₂CH₂CH₂-#1; and
   further provided that, when Z is CR¹⁰R¹¹, then at least one of R¹ and R² is substituted by two or more halo groups.

In some embodiments, the compound of formula (I), or a pharmaceutically acceptable salt thereof, is a compound of formula (VI-a)

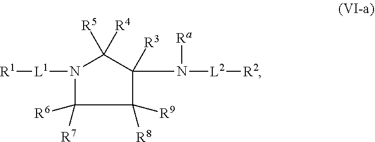

or a pharmaceutically acceptable salt thereof,
wherein R¹, R², R³, R⁴, R⁵, R⁶, R⁷, R⁸, R⁹, R^{a}, L¹, and L² are as defined in compounds of formula (I),
provided that L¹ is selected from the group consisting of *1-OCH₂CH(OH)CH₂-#1, *1-OCH₂-#1, *1-OCH₂CH₂-#1, and *1-OCH₂CH₂CH₂-#1.

In some embodiments, the compound of formula (I), or a pharmaceutically acceptable salt thereof, is a compound of formula (VI-b)

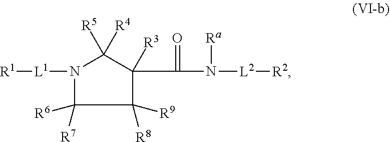

or a pharmaceutically acceptable salt thereof,
wherein R¹, R², R³, R⁴, R⁵, R⁶, R⁷, R⁸, R⁹, R^{a}, L¹, and L² are as defined in compounds of formula (I),
provided that L¹ is selected from the group consisting of *1-OCH₂CH(OH)CH₂-#1, *1-OCH₂-#1, *1-OCH₂CH₂-#1, and *1-OCH₂CH₂CH₂-#1.

In some embodiments, the compound of formula (I), or a pharmaceutically acceptable salt thereof, is a compound of formula (VI-c)

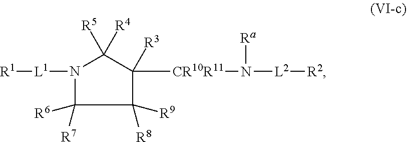

or a pharmaceutically acceptable salt thereof,
wherein R¹, R², R³, R⁴, R⁵, R⁶, R⁷, R⁸, R⁹, R¹⁰, R¹, R^{a}, L¹, and L² are as defined in compounds of formula (I),
provided that L¹ is selected from the group consisting of *1-OCH₂CH(OH)CH₂-#1, *1-OCH₂-#1, *1-OCH₂CH₂-#1, and *1-OCH₂CH₂CH₂-#1; and at least one of R¹ and R² is substituted by two or more halo groups.

In some embodiments, the compound of formula (I), or a pharmaceutically acceptable salt thereof, is a compound of formula (VI-d)

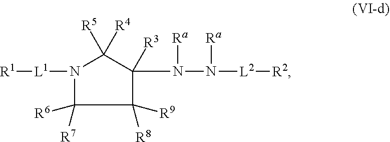

or a pharmaceutically acceptable salt thereof,
wherein R¹, R², R³, R⁴, R⁵, R⁶, R⁷, R⁸, R⁹, R^{a}, L¹, and L² are as defined in compounds of formula (I),
provided that L¹ is selected from the group consisting of *1-OCH₂CH(OH)CH₂-#1, *1-OCH₂-#1, *1-OCH₂CH₂-#1, and *1-OCH₂CH₂CH₂-#1.

In some embodiments, the compound of formula (I), or a pharmaceutically acceptable salt thereof, is a compound of formula (VII)

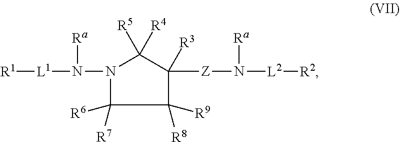

or a pharmaceutically acceptable salt thereof,
wherein R¹, R², R³, R⁴, R⁵, R⁶, R⁷, R⁸, R⁹, R^{a}, Z, L¹, and L² are as defined in compounds of formula (I).

In some embodiments, the compound of formula (I), or a pharmaceutically acceptable salt thereof, is a compound of formula (VII-a)

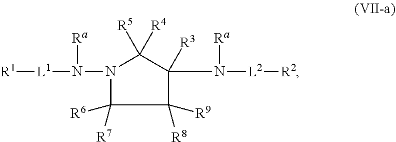

or a pharmaceutically acceptable salt thereof,
wherein R¹, R², R³, R⁴, R⁵, R⁶, R⁷, R⁸, R⁹, R^{a}, L¹, and L² are as defined in compounds of formula (I).

In some embodiments, the compound of formula (I), or a pharmaceutically acceptable salt thereof, is a compound of formula (VII-b)

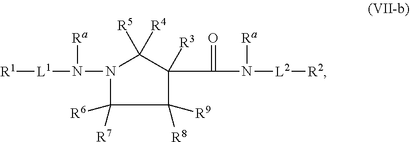

or a pharmaceutically acceptable salt thereof,
wherein R¹, R², R³, R⁴, R⁵, R⁶, R⁷, R⁸, R⁹, R^{a}, L¹, and L² are as defined in compounds of formula (I).

In some embodiments, the compound of formula (I), or a pharmaceutically acceptable salt thereof, is a compound of formula (VII-c)

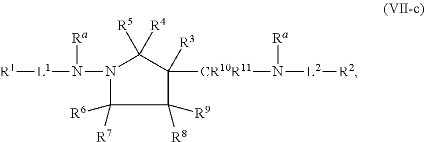

or a pharmaceutically acceptable salt thereof,
wherein R¹, R², R³, R⁴, R⁵, R⁶, R⁷, R⁸, R⁹, R¹⁰, R¹, R^{a}, L¹, and L² are as defined in compounds of formula (I).

In some embodiments, the compound of formula (I), or a pharmaceutically acceptable salt thereof, is a compound of formula (VII-d)

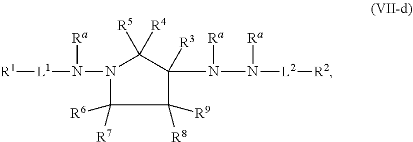

or a pharmaceutically acceptable salt thereof,
wherein R¹, R², R³, R⁴, R⁵, R⁶, R⁷, R⁸, R⁹, R^{a}, L¹, and L² are as defined in compounds of formula (I).

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R³ is hydrogen. In some embodiments, R³ is halogen such as fluoro, chloro, bromo, or iodo. In some embodiments, R³ is C₁-C₆ alkyl such as methyl, ethyl, n-propyl, isopropyl, n-butyl, t-butyl, isobutyl, or sec-butyl. In some embodiments, R³ is hydrogen, fluoro, chloro, bromo, iodo, methyl, ethyl, n-propyl, isopropyl, n-butyl, t-butyl, isobutyl, or sec-butyl. In some embodiments, R³ and R¹² are taken together to form a CR¹³R¹⁴ group.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R⁴ is hydrogen. In some embodiments, R⁴ is halogen such as fluoro, chloro, bromo, or iodo. In some embodiments, R⁴ is C₁-C₆ alkyl such as methyl, ethyl, n-propyl, isopropyl, n-butyl, t-butyl, isobutyl, or sec-butyl. In some embodiments, R⁴ is hydrogen, fluoro, chloro, bromo, iodo, methyl, ethyl, n-propyl, isopropyl, n-butyl, t-butyl, isobutyl, or sec-butyl.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R⁵ is hydrogen. In some embodiments, R⁵ is halogen such as fluoro, chloro, bromo, or iodo. In some embodiments, R⁵ is C₁-C₆ alkyl such as methyl, ethyl, n-propyl, isopropyl, n-butyl, t-butyl, isobutyl, or sec-butyl. In some embodiments, R⁵ is hydrogen, fluoro, chloro, bromo, iodo, methyl, ethyl, n-propyl, isopropyl, n-butyl, t-butyl, isobutyl, or sec-butyl.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R⁶ is hydrogen. In some embodiments, R⁶ is halogen such as fluoro, chloro, bromo, or iodo. In some embodiments, R⁶ is C₁-C₆ alkyl such as methyl, ethyl, n-propyl, isopropyl, n-butyl, t-butyl, isobutyl, or sec-butyl. In some embodiments, R⁶ is hydrogen, fluoro, chloro, bromo, iodo, methyl, ethyl, n-propyl, isopropyl, n-butyl, t-butyl, isobutyl, or sec-butyl.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R⁷ is hydrogen. In some embodiments, R⁷ is halogen such as fluoro, chloro, bromo, or iodo. In some embodiments, R⁷ is C₁-C₆ alkyl such as methyl, ethyl, n-propyl, isopropyl, n-butyl, t-butyl, isobutyl, or sec-butyl. In some embodiments, R⁷ is hydrogen, fluoro, chloro, bromo, iodo, methyl, ethyl, n-propyl, isopropyl, n-butyl, t-butyl, isobutyl, or sec-butyl.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R⁸ is hydrogen. In some embodiments, R⁸ is halogen such as fluoro, chloro, bromo, or iodo. In some embodiments, R⁸ is C₁-C₆ alkyl such as methyl, ethyl, n-propyl, isopropyl, n-butyl, t-butyl, isobutyl, or sec-butyl. In some embodiments, R⁸ is hydrogen, fluoro, chloro, bromo, iodo, methyl, ethyl, n-propyl, isopropyl, n-butyl, t-butyl, isobutyl, or sec-butyl.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R⁹ is hydrogen. In some embodiments, R⁹ is halogen such as fluoro, chloro, bromo, or iodo. In some embodiments, R⁹ is C₁-C₆ alkyl such as methyl, ethyl, n-propyl, isopropyl, n-butyl, t-butyl, isobutyl, or sec-butyl. In some embodiments, R⁹ is hydrogen, fluoro, chloro, bromo, iodo, methyl, ethyl, n-propyl, isopropyl, n-butyl, t-butyl, isobutyl, or sec-butyl.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R³, R⁴, R⁵, R⁶, R⁷, R⁸, and R⁹ are all hydrogen. In some embodiments, R³ and R¹² are taken together to form a CR¹³R¹⁴ group; and R⁴, R⁵, R⁶, R⁷, R⁸, and R⁹ are all hydrogen. In some embodiments, at least 1, 2, 3, 4, 5, or 6 of R³, R⁴, R⁵, R⁶, R⁷, R⁸, and R⁹ are hydrogen. In some embodiments, R³ and R¹² are taken together to form a CR¹³R¹⁴ group; and at least 1, 2, 3, 4, or 5 of R⁴, R⁵, R⁶, R⁷, R⁸, and R⁹ are all hydrogen.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R¹⁰ is hydrogen. In some embodiments, R¹⁰ is halogen such as fluoro, chloro, bromo, or iodo. In some embodiments, R¹⁰ is C₁-C₆ alkyl such as methyl, ethyl, n-propyl, isopropyl, n-butyl, t-butyl, isobutyl, or sec-butyl. In some embodiments, R¹⁰ is hydrogen, fluoro, chloro, bromo, iodo, methyl, ethyl, n-propyl, isopropyl, n-butyl, t-butyl, isobutyl, or sec-butyl.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R¹¹ is hydrogen. In some embodiments, R¹¹ is halogen such as fluoro, chloro, bromo, or iodo. In some embodiments, R¹¹ is C₁-C₆ alkyl such as methyl, ethyl, n-propyl, isopropyl, n-butyl, t-butyl, isobutyl, or sec-butyl. In some embodiments, R¹¹ is hydrogen, fluoro, chloro, bromo, iodo, methyl, ethyl, n-propyl, isopropyl, n-butyl, t-butyl, isobutyl, or sec-butyl. In some embodiments, R¹⁰ and R¹¹ are both hydrogen.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R¹² is hydrogen. In some embodiments, R¹² is halogen such as fluoro, chloro, bromo, or iodo. In some embodiments, R¹² is C₁-C₆ alkyl such as methyl, ethyl, n-propyl, isopropyl, n-butyl, t-butyl, isobutyl, or sec-butyl. In some embodiments, R¹² is hydrogen, fluoro, chloro, bromo, iodo, methyl, ethyl, n-propyl, isopropyl, n-butyl, t-butyl, isobutyl, or sec-butyl. In some embodiments, R³ and R¹² are taken together to form a CR¹³R¹⁴ group. In some embodiments of the compound of formula (I), R³, R⁴, R⁵, R⁶, R⁷, R⁸, R⁹, and R¹² are all hydrogen.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R¹³ is hydrogen. In some embodiments, R¹³ is halogen such as fluoro, chloro, bromo, or iodo. In some embodiments, R¹³ is C₁-C₆ alkyl such as methyl, ethyl, n-propyl, isopropyl, n-butyl, t-butyl, isobutyl, or sec-butyl. In some embodiments, R¹³ is hydrogen, fluoro, chloro, bromo, iodo, methyl, ethyl, n-propyl, isopropyl, n-butyl, t-butyl, isobutyl, or sec-butyl.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R¹⁴ is hydrogen. In some embodiments, R¹⁴ is halogen such as fluoro, chloro, bromo, or iodo. In some embodiments, R¹⁴ is C₁-C₆ alkyl such as methyl, ethyl, n-propyl, isopropyl, n-butyl, t-butyl, isobutyl, or sec-butyl. In some embodiments, R¹⁴ is hydrogen, fluoro, chloro, bromo, iodo, methyl, ethyl, n-propyl, isopropyl, n-butyl, t-butyl, isobutyl, or sec-butyl. In some embodiments, R¹³ and R¹⁴ are both hydrogen. In some embodiments, R³ and R¹² are taken together to form a CR¹³R¹⁴ group; and R⁴, R⁵, R⁶, R⁷, R⁸, R⁹, R¹³ and R¹⁴ are all hydrogen.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R^{a}, at each occurrence, is hydrogen. In some embodiments, at least 1, 2 or 3 R^{a} is hydrogen. In some embodiments, R^{a}, independently at each occurrence, is C₁-C₆ alkyl such as methyl, ethyl, n-propyl, isopropyl, n-butyl, t-butyl, isobutyl, or sec-butyl. In some embodiments, R^{a}, independently at each occurrence, is hydrogen, methyl, ethyl, n-propyl, isopropyl, n-butyl, t-butyl, isobutyl, or sec-butyl.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), L¹ is *1-C(═O)-#1. In some embodiments, L¹ is *1-CH₂-#1. In some embodiments, L¹ is *1-CH₂CH₂-#1. In some embodiments, L¹ is *1-CH₂CH₂CH₂-#1. In some embodiments, L¹ is *1-OCH₂C(═O)-#1. In some embodiments, L¹ is *1-OCH₂CH₂C(═O)-#1. In some embodiments, L¹ is *1-OCH₂CH₂CH₂C(═O)-#1. In some embodiments, L¹ is *1-OCH₂CH(OH)CH₂-#1. In some embodiments, L¹ is *1-OCH₂-#1. In some embodiments, L¹ is *1-OCH₂CH₂-#1. In some embodiments, L¹ is *1-OCH₂CH₂CH₂-#1. As provided herein, *1 represents the attachment point to R¹ and #1 represents the attachment point to the remainder of the molecule.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), L² is #2-C(═O)-*2. In some embodiments, L² is #2-CH₂-*2. In some embodiments, L² is #2-CH₂CH₂-*2. In some embodiments, L² is #2-CH₂CH₂CH₂-*2. In some embodiments, L² is #2-C(═O)CH₂O—*2. In some embodiments, L² is #2-C(═O)CH₂CH₂O—*2. In some embodiments, L² is #2-C(═O)CH₂CH₂CH₂O-*2. In some embodiments, L² is #2-CH₂CH(OH)CH₂O—*2. In some embodiments, L² is #2-CH₂O-*2. In some embodiments, L² is #2-CH₂CH₂O-*2. In some embodiments, L² is #2-CH₂CH₂CH₂O-*2. As provided herein, *2 represents the attachment point to R² and #2 represents the attachment point to the remainder of the molecule.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R¹ is C₆-C₁₄ aryl substituted with one or more halo groups and optionally substituted with one or more R^{b}. In some embodiments, the C₆-C₁₄ aryl of R¹ is phenyl. In some embodiments, the C₆-C₁₄ aryl of R¹ is bicyclic C₆-C₁₄ aryl. In some embodiments, R¹ is C₆-C₁₄ aryl substituted with one or more halo groups. In some embodiments, R¹ is C₆-C₁₄ aryl substituted with 1 halo group, such as fluoro, chloro, or bromo. In some embodiments, R¹ is C₆-C₁₄ aryl substituted with 2 halo groups, each of which is independently fluoro, chloro, or bromo. In some embodiments, R¹ is C₆-C₁₄ aryl substituted with 3 halo groups, each of which is independently fluoro, chloro, or bromo. In some embodiments, R¹ is phenyl substituted with one or more halo groups. In some embodiments, R¹ is phenyl substituted with one or more halo groups and optionally substituted with one or more R^{b}. In some embodiments, R¹ is phenyl substituted with 1 halo group, such as fluoro, chloro, or bromo. In some embodiments, R¹ is phenyl substituted with 2 halo groups, each of which is independently fluoro, chloro, or bromo. In some embodiments, R¹ is phenyl substituted with 3 halo groups, each of which is independently fluoro, chloro, or bromo. In some embodiments, R¹ is bicyclic C₆-C₁₄ aryl substituted with one or more halo groups. In some embodiments, R¹ is bicyclic C₆-C₁₄ aryl substituted with one or more halo groups and optionally substituted with one or more R^{b}. In some embodiments, R¹ is bicyclic C₆-C₁₄ aryl substituted with 1 halo group, such as fluoro, chloro, or bromo. In some embodiments, R¹ is bicyclic C₆-C₁₄ aryl substituted with 2 halo groups, each of which is independently fluoro, chloro, or bromo. In some embodiments, R¹ is bicyclic C₆-C₁₄ aryl substituted with 3 halo groups, each of which is independently fluoro, chloro, or bromo.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R¹ is 5-14 membered heteroaryl substituted with one or more halo groups and optionally substituted with one or more R^{b}. In some embodiments, the 5-14 membered heteroaryl of R¹ is monocyclic 5-14 membered heteroaryl. In some embodiments, the 5-14 membered heteroaryl of R¹ is bicyclic 5-14 membered heteroaryl. In some embodiments, R¹ is 5-14 membered heteroaryl substituted with one or more halo groups. In some embodiments, R¹ is 5-14 membered heteroaryl substituted with 1 halo group, such as fluoro, chloro, or bromo. In some embodiments, R¹ is 5-14 membered heteroaryl substituted with 2 halo groups, each of which is independently fluoro, chloro, or bromo. In some embodiments, R¹ is 5-14 membered heteroaryl substituted with 3 halo groups, each of which is independently fluoro, chloro, or bromo. In some embodiments, R¹ is monocyclic 5-14 membered heteroaryl substituted with one or more halo groups. In some embodiments, R¹ is monocyclic 5-14 membered heteroaryl substituted with one or more halo groups and optionally substituted with one or more R^{b}. In some embodiments, R¹ is monocyclic 5-14 membered heteroaryl substituted with 1 halo group, such as fluoro, chloro, or bromo. In some embodiments, R¹ is monocyclic 5-14 membered heteroaryl substituted with 2 halo groups, each of which is independently fluoro, chloro, or bromo. In some embodiments, R¹ is monocyclic 5-14 membered heteroaryl substituted with 3 halo groups, each of which is independently fluoro, chloro, or bromo. In some embodiments, R¹ is bicyclic 5-14 membered heteroaryl substituted with one or more halo groups. In some embodiments, R¹ is bicyclic 5-14 membered heteroaryl substituted with one or more halo groups and optionally substituted with one or more R^{b}. In some embodiments, R¹ is bicyclic 5-14 membered heteroaryl substituted with 1 halo group, such as fluoro, chloro, or bromo. In some embodiments, R¹ is bicyclic 5-14 membered heteroaryl substituted with 2 halo groups, each of which is independently fluoro, chloro, or bromo. In some embodiments, R¹ is bicyclic 5-14 membered heteroaryl substituted with 3 halo groups, each of which is independently fluoro, chloro, or bromo.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R¹ is a substituent selected from the group consisting of:

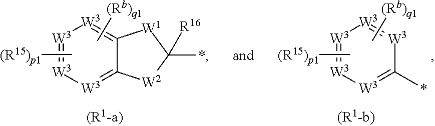

wherein:
* represents the attachment point to the remainder of the molecule;
W¹ is selected from the group consisting of —C(R^{W1-1}R^{W1-2})—, —N(R^{W1-2})—, —C(R^{W1-1}R^{W1-2})N(R^{W1-2})—, —N(R^{W1-2})C(R^{W1-1}R^{W1-2})—, —C(R^{W1-2})═N—, —N═C(R^{W1-2})—, —O—, —C(R^{W1-1}R^{W1-2})O—, —OC(R^{W1-1}R^{W1-2})—, —S—, —C(R^{W1-1}R^{W1-2})S—, —SC(R^{W1-1}R^{W1-2})—, and —CR^{W1-1}═CR^{W1-2}
wherein R^{W1-1} is H, R¹⁵, or R^{b}, and R^{W1-2} is H, R¹⁵, or R^{b};
   W² is selected from the group consisting of —C(R^{W2-1}R^{W2-2})—, —N(R^{W2-2})—, —C(R^{W2-1}R^{W2-2})N(R^{W2-2})—, —N(R^{W2-2})C(R^{W2-1}R^{W2-2})—, —C(R^{W2-2})═N—, —N═C(R^{W2-2})—, —O—, —C(R^{W2-1}R^{W2-2})O—, —OC(R^{W2-1}R^{W2-2})—, —S—, —C(R^{W2-1}R^{W2-2})S—, —SC(R^{W2-1}R^{W2-2})—, and —CR^{W2-1}═CR^{W2-2}
wherein R^{W2-1} is H, R¹⁵, or R^{b}, and R^{W2-2} is H, R¹⁵ or R^{b};
   W³, independently at each occurrence, is CR^{W3} or N, wherein R^{W3} is H, R¹⁵, or R^{b};
   R¹⁵ is halogen;
   p1 is 1, 2, 3, or 4;
   R^{b}, independently at each occurrence, is selected from the group consisting of NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{c}R^{d}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{14-a}R^{14-b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{c}R^{d}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl),
wherein R^{c} and R^{d} are taken together with the nitrogen atom to which they are attached to form a 3-10 membered heterocycle;
   q1 is 0, 1, 2, 3, or 4; and
   R¹⁶ is hydrogen, R¹⁵, or R^{b}, or R¹⁶ and R^{W1-2} are taken together to form a double bond between the carbon atom bearing R¹⁶ and W¹, or R¹⁶ and R^{W2-2} are taken together to form a double bond between the carbon atom bearing R¹⁶ and W².

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R¹ is a substituent selected from the group consisting of:

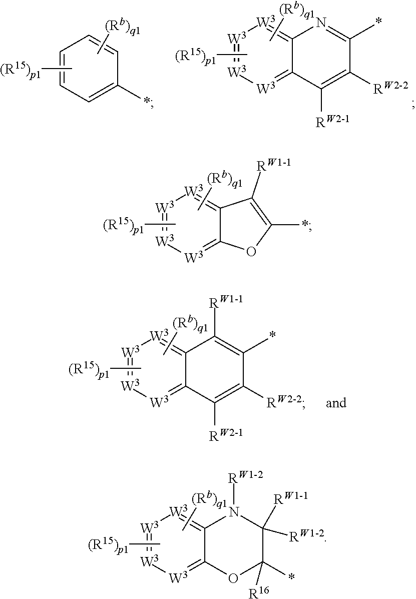

In some embodiments of the substituent of formula (R¹-a) or formula (R¹-b) or any related formula, R¹⁵ is fluoro or chloro. In some embodiments, p1 is 1. In some embodiments, p1 is 2. In some embodiments, p1 is 3. In some embodiments, p1 is 4. In some embodiments, q1 is 0. In some embodiments, q1 is 1. In some embodiments, q1 is 2. In some embodiments, q1 is 3. In some embodiments, q1 is 4. In some embodiments, p1 is 1 and q1 is 0. In some embodiments, p1 is 2 and q1 is 0.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R¹ is a substituent selected from the group consisting of:

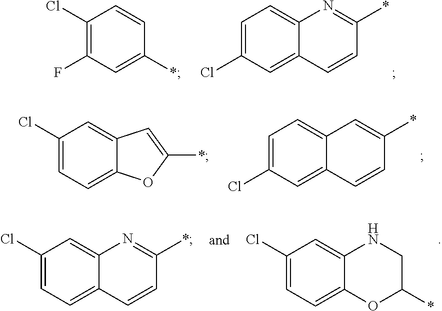

In some embodiments, R¹ is

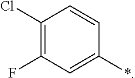

In some embodiments, R¹ is

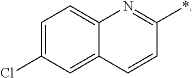

In some embodiments, R¹ is

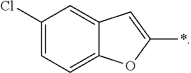

In some embodiments, R¹ is

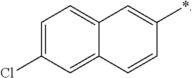

In some embodiments, R¹ is

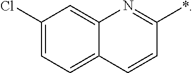

In some embodiments, R¹ is

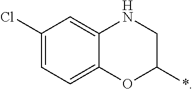

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R² is C₆-C₁₄ aryl substituted with one or more halo groups and optionally substituted with one or more R^{b}. In some embodiments, the C₆-C₁₄ aryl of R² is phenyl. In some embodiments, the C₆-C₁₄ aryl of R² is bicyclic C₆-C₁₄ aryl. In some embodiments, R² is C₆-C₁₄ aryl substituted with one or more halo groups. In some embodiments, R² is C₆-C₁₄ aryl substituted with 1 halo group, such as fluoro, chloro, or bromo. In some embodiments, R² is C₆-C₁₄ aryl substituted with 2 halo groups, each of which is independently fluoro, chloro, or bromo. In some embodiments, R² is C₆-C₁₄ aryl substituted with 3 halo groups, each of which is independently fluoro, chloro, or bromo. In some embodiments, R² is phenyl substituted with one or more halo groups. In some embodiments, R² is phenyl substituted with one or more halo groups and optionally substituted with one or more R^{b}. In some embodiments, R² is phenyl substituted with 1 halo group, such as fluoro, chloro, or bromo. In some embodiments, R² is phenyl substituted with 2 halo groups, each of which is independently fluoro, chloro, or bromo. In some embodiments, R² is phenyl substituted with 3 halo groups, each of which is independently fluoro, chloro, or bromo. In some embodiments, R² is bicyclic C₆-C₁₄ aryl substituted with one or more halo groups. In some embodiments, R² is bicyclic C₆-C₁₄ aryl substituted with one or more halo groups and optionally substituted with one or more R^{b}. In some embodiments, R² is bicyclic C₆-C₁₄ aryl substituted with 1 halo group, such as fluoro, chloro, or bromo. In some embodiments, R² is bicyclic C₆-C₁₄ aryl substituted with 2 halo groups, each of which is independently fluoro, chloro, or bromo. In some embodiments, R² is bicyclic C₆-C₁₄ aryl substituted with 3 halo groups, each of which is independently fluoro, chloro, or bromo.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R² is 5-14 membered heteroaryl substituted with one or more halo groups and optionally substituted with one or more R^{b}. In some embodiments, the 5-14 membered heteroaryl of R² is monocyclic 5-14 membered heteroaryl. In some embodiments, the 5-14 membered heteroaryl of R² is bicyclic 5-14 membered heteroaryl. In some embodiments, R² is 5-14 membered heteroaryl substituted with one or more halo groups. In some embodiments, R² is 5-14 membered heteroaryl substituted with 1 halo group, such as fluoro, chloro, or bromo. In some embodiments, R² is 5-14 membered heteroaryl substituted with 2 halo groups, each of which is independently fluoro, chloro, or bromo. In some embodiments, R² is 5-14 membered heteroaryl substituted with 3 halo groups, each of which is independently fluoro, chloro, or bromo. In some embodiments, R² is monocyclic 5-14 membered heteroaryl substituted with one or more halo groups. In some embodiments, R² is monocyclic 5-14 membered heteroaryl substituted with one or more halo groups and optionally substituted with one or more R^{b}. In some embodiments, R² is monocyclic 5-14 membered heteroaryl substituted with 1 halo group, such as fluoro, chloro, or bromo. In some embodiments, R² is monocyclic 5-14 membered heteroaryl substituted with 2 halo groups, each of which is independently fluoro, chloro, or bromo. In some embodiments, R² is monocyclic 5-14 membered heteroaryl substituted with 3 halo groups, each of which is independently fluoro, chloro, or bromo. In some embodiments, R² is bicyclic 5-14 membered heteroaryl substituted with one or more halo groups. In some embodiments, R² is bicyclic 5-14 membered heteroaryl substituted with one or more halo groups and optionally substituted with one or more R^{b}. In some embodiments, R² is bicyclic 5-14 membered heteroaryl substituted with 1 halo group, such as fluoro, chloro, or bromo. In some embodiments, R² is bicyclic 5-14 membered heteroaryl substituted with 2 halo groups, each of which is independently fluoro, chloro, or bromo. In some embodiments, R² is bicyclic 5-14 membered heteroaryl substituted with 3 halo groups, each of which is independently fluoro, chloro, or bromo.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R² is a substituent selected from the group consisting of:

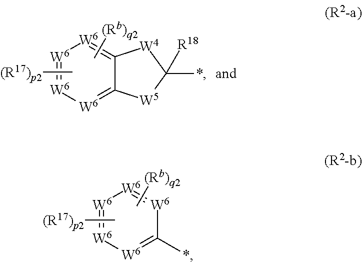

wherein:
* represents the attachment point to the remainder of the molecule;
W⁴ is selected from the group consisting of —C(R^{W4-1}R^{W4-2})—, —N(R^{W4-2})—, —C(R^{W4-1}R^{W4-2})N(R^{W4-2})—, —N(R^{W4-2})C(R^{W4-1}R^{W4-2})—, —C(R^{W4-2})═N—, —N═C(R^{W4-2})—, —O—, —C(R^{W4-1}R^{W4-2})O—, —OC(R^{W4-1}R^{W4-2})—, —S—, —C(R^{W4-1}R^{W4-2})S—, —SC(R^{W4-1}R^{W4-2})—, and —CR^{W4-1}═CR^{W4-2}—,
wherein R^{W4-1} is H, R¹⁷, or R^{b}, and R^{W4-2} is H, R¹⁷, or R^{b};
   W⁵ is selected from the group consisting of —C(R^{W5-1}R^{W5-2})—, —N(R^{W5-2})⁻, —C(R^{W5-1}R^{W5-2})N(R^{W5-2})—, —N(R^{W5-2})C(R^{W5-1}R^{W5-2})—, —C(R^{W5-2})═N—, —N═C(R^{W5-2})—, —O—, —C(R^{W5-1}R^{W5-2})O—, —OC(R^{W5-1}R^{W5-2})—, —S—, —C(R^{W5-1}R^{W5-2})S—, —SC(R^{W5-1}R^{W5-2})—, and —CR^{W5-1}═CR^{W5-2}—,
wherein R^{W5-1} is H, R¹⁷, or R^{b}, and R^{W5-2} is H, R¹⁷ or R^{b};
   W⁶, independently at each occurrence, is CR^{W6} or N, wherein R^{W6} is H, R¹⁷, or R^{b};
   R¹⁷ is halogen;
   p2 is 1, 2, 3, or 4;
   R^{b}, independently at each occurrence, is selected from the group consisting of NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{c}R^{d}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{14-a}R^{14-b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{c}R^{d}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl),
wherein R^{c} and R^{d} are taken together with the nitrogen atom to which they are attached to form a 3-10 membered heterocycle;
   q2 is 0, 1, 2, 3, or 4; and
   R¹⁸ is hydrogen, R¹⁷, or R^{b}, or R¹⁸ and R^{W4-2} are taken together to form a double bond between the carbon atom bearing R¹⁸ and W⁴, or R¹⁸ and R^{W5-2} are taken together to form a double bond between the carbon atom bearing R¹⁸ and W⁵.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R² is a substituent selected from the group consisting of:

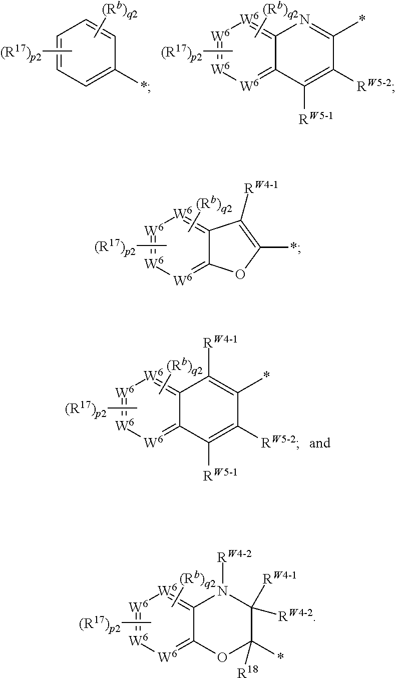

In some embodiments of the substituent of formula (R²-a) or formula (R²-b) or any related formula, R¹⁷ is fluoro or chloro. In some embodiments, p2 is 1. In some embodiments, p2 is 2. In some embodiments, p2 is 3. In some embodiments, p2 is 4. In some embodiments, q2 is 0. In some embodiments, q2 is 1. In some embodiments, q2 is 2. In some embodiments, q2 is 3. In some embodiments, q2 is 4. In some embodiments, p2 is 1 and q2 is 0. In some embodiments, p2 is 2 and q2 is 0.

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), R² is a substituent selected from the group consisting of:

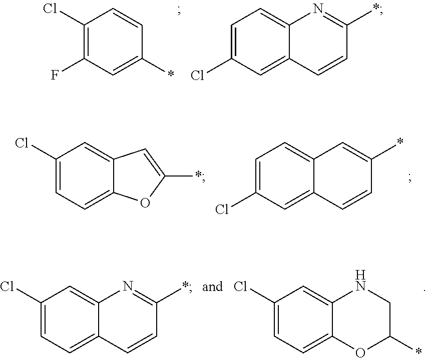

In some embodiments, R² is

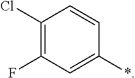

In some embodiments, R² is

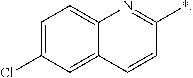

In some embodiments, R² is

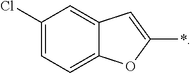

In some embodiments, R² is

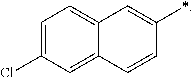

In some embodiments, R² is

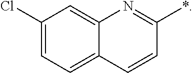

In some embodiments, R² is

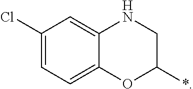

In some embodiments of the compound of formulae (I), (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), the compound has at least 1, 2, 3, 4, 5, or 6 of the following features:

(a) R³, R⁴, R⁵, R⁶, R⁷, R⁸, R⁹, and R¹² are all hydrogen;
(b) R³ and R¹² are taken together to form a CR¹³R¹⁴ group, wherein R¹³ and R¹⁴ are both hydrogen, and R⁴, R⁵, R⁶, R⁷, R, and R⁹ are all hydrogen;
(c) R^{a}, at each occurrence, is hydrogen;
(d) R¹⁰ and R¹¹ are both hydrogen;
(e) L¹ is selected from the group consisting of *1-C(═O)-#1, *1-CH₂-#1, *1-CH₂CH₂-#1, *1-CH₂CH₂CH₂-#1, *1-OCH₂C(═O)-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1, wherein *1 represents the attachment point to R¹ and #1 represents the attachment point to the remainder of the molecule;
(f) L² is selected from the group consisting of #2-C(═O)-*2, #2-CH₂-*2, #2-CH₂CH₂-*2, #2—CH₂CH₂CH₂-*2, #2-C(═O)CH₂O—*2, #2-C(═O)CH₂CH₂O—*2, #2-C(═O)CH₂CH₂CH₂O-*2, #2—CH₂CH(OH)CH₂O—*2, #2-CH₂O-*2, #2-CH₂CH₂O-*2, and #2-CH₂CH₂CH₂O-*2, wherein *2 represents the attachment point to R² and #2 represents the attachment point to the remainder of the molecule;
(g) R¹ is a substituent selected from the group consisting of:

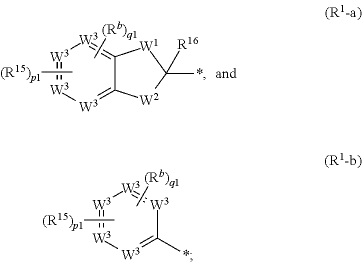

(h) R¹ is a substituent selected from the group consisting of:

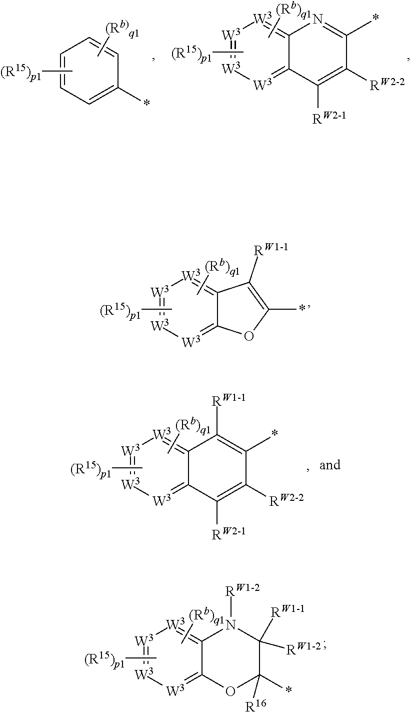

(i) R¹ is a substituent selected from the group consisting of:

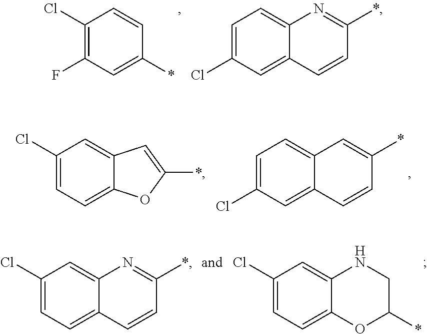

(j) R² is a substituent selected from the group consisting of:

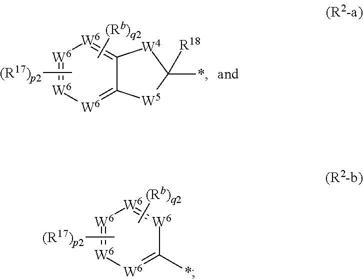

(k) R² is a substituent selected from the group consisting of:

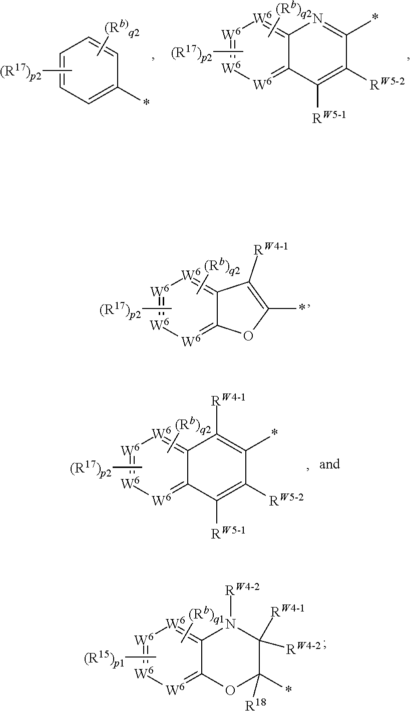

(l) R² is a substituent selected from the group consisting of:

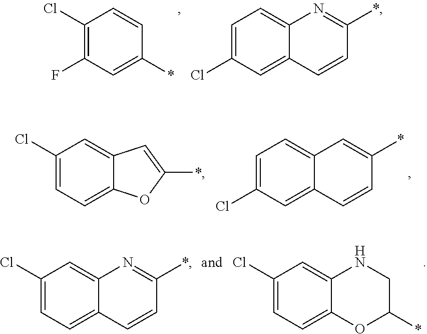

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to X of formula (I) may be combined with every description, variation, embodiment or aspect of R¹, R², R³, R⁴, R⁵, R⁶, R⁷, R⁸, R⁹, R¹⁰, R¹¹, R¹², R¹³, R¹⁴, R^{a}, L¹, L², Y, and Z the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae. For example, all descriptions, variations, embodiments or aspects of formula (I), where applicable, apply equally to any of formulae (II), (III), (IV), (IV-a), (IV-b), (IV-c), (IV-d), (IV-e), (IV-f), (IV-g), (IV-h), (V), (V-a), (V-b), (V-c), (V-d), (V-e), (V-f), (VI), (VI-a), (VI-b), (VI-c), (VI-d), (VII), (VII-a), (VII-b), (VII-c), and (VII-d), as detailed herein, and are equally described, the same as if each and every description, variation, embodiment or aspect were separately and individually listed for all formulae.

Also provided are salts of compounds referred to herein, such as pharmaceutically acceptable salts. The present disclosure also includes any or all of the stereochemical forms, including any enantiomeric or diastereomeric forms, and any tautomers or other forms of the compounds described. Thus, if a particular stereochemical form, such as a specific enantiomeric form or diastereomeric form, is depicted for a given compound, then it is understood that any or all stereochemical forms, including any enantiomeric or diastereomeric forms, and any tautomers or other forms of any of that same compound are herein described and embraced by the invention.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

In some embodiments, provided is compound selected from compounds in Table 1, or a stereoisomer, tautomer, solvate, prodrug or salt thereof. Although certain compounds described in Table 1 are presented as specific stereoisomers and/or in a non-stereochemical form, it is understood that any or all stereochemical forms, including any enantiomeric or diastereomeric forms, and any tautomers or other forms of any of the compounds of Table 1 are herein described.

| Compound | Structure |
|---|---|
| 1 | 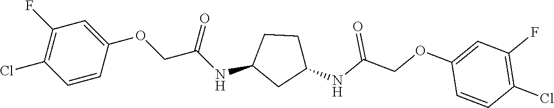 [SMILES: O=C(COc1ccc(Cl)c(F)c1)N[C@H]1CC[C@H](NC(=O)COc2ccc(Cl)c(F)c2)C1] |
| 2 | 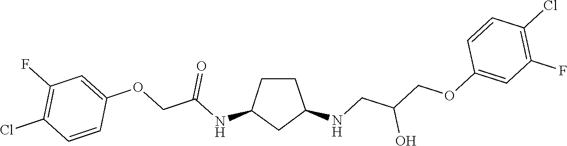 [SMILES: O=C(COc1ccc(Cl)c(F)c1)N[C@H]1CC[C@@H](NCC(O)COc2ccc(Cl)c(F)c2)C1] |
| 3 | 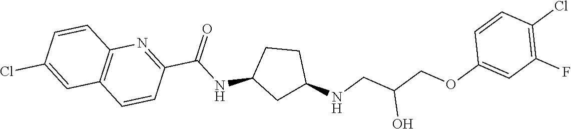 [SMILES: O=C(N[C@H]1CC[C@@H](NCC(O)COc2ccc(Cl)c(F)c2)C1)c1ccc2cc(Cl)ccc2n1] |
| 4 | 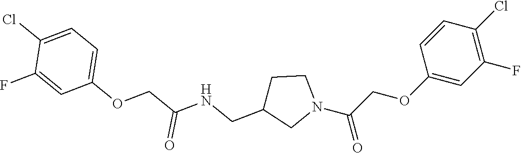 [SMILES: O=C(COc1ccc(Cl)c(F)c1)NCC1CCN(C(=O)COc2ccc(Cl)c(F)c2)C1] |
| 5 | 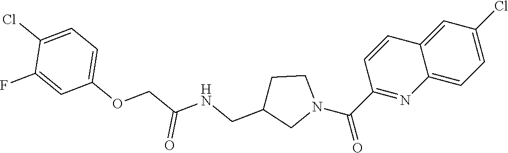 [SMILES: O=C(COc1ccc(Cl)c(F)c1)NCC1CCN(C(=O)c2ccc3cc(Cl)ccc3n2)C1] |
| 6 | 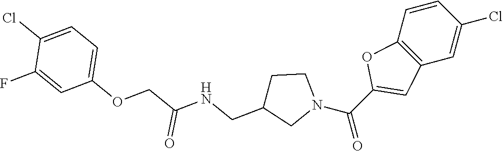 [SMILES: O=C(COc1ccc(Cl)c(F)c1)NCC1CCN(C(=O)c2cc3cc(Cl)ccc3o2)C1] |
| 7 | 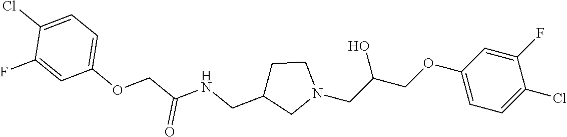 [SMILES: O=C(COc1ccc(Cl)c(F)c1)NCC1CCN(CC(O)COc2ccc(Cl)c(F)c2)C1] |
| 8 | 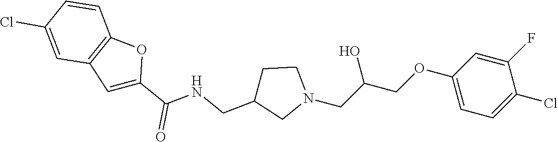 [SMILES: O=C(NCC1CCN(CC(O)COc2ccc(Cl)c(F)c2)C1)c1cc2cc(Cl)ccc2o1] |
| 9 | 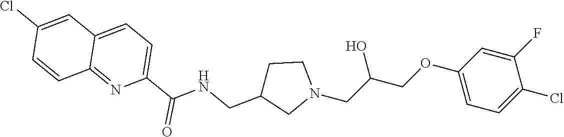 [SMILES: O=C(NCC1CCN(CC(O)COc2ccc(Cl)c(F)c2)C1)c1ccc2cc(Cl)ccc2n1] |
| 10 | 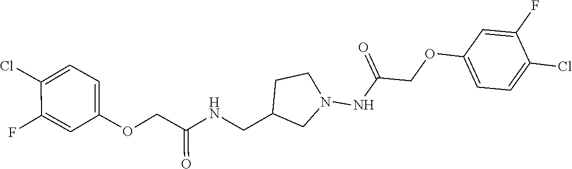 [SMILES: O=C(COc1ccc(Cl)c(F)c1)NCC1CCN(NC(=O)COc2ccc(Cl)c(F)c2)C1] |
| 11 | 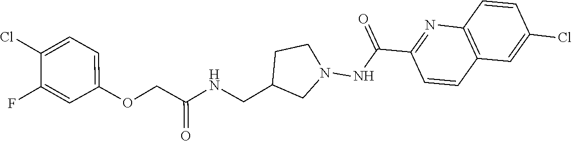 [SMILES: O=C(COc1ccc(Cl)c(F)c1)NCC1CCN(NC(=O)c2ccc3cc(Cl)ccc3n2)C1] |
| 12 | 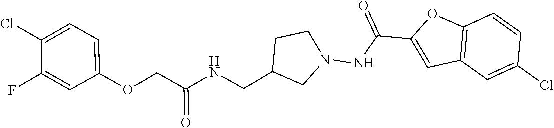 [SMILES: O=C(COc1ccc(Cl)c(F)c1)NCC1CCN(NC(=O)c2cc3cc(Cl)ccc3o2)C1] |
| 13 | 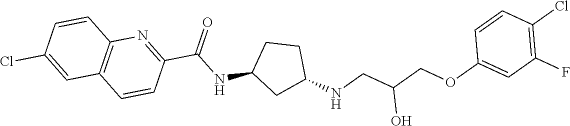 [SMILES: O=C(N[C@H]1CC[C@H](NCC(O)COc2ccc(Cl)c(F)c2)C1)c1ccc2cc(Cl)ccc2n1] |
| 14 | 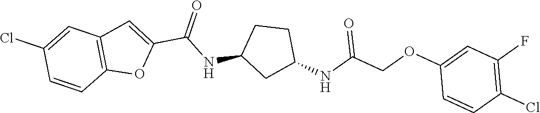 [SMILES: O=C(COc1ccc(Cl)c(F)c1)N[C@H]1CC[C@H](NC(=O)c2cc3cc(Cl)ccc3o2)C1] |
| 15 | 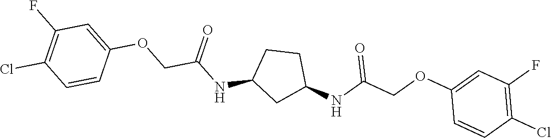 [SMILES: O=C(COc1ccc(Cl)c(F)c1)N[C@@H]1CC[C@H](NC(=O)COc2ccc(Cl)c(F)c2)C1] |
| 16 | 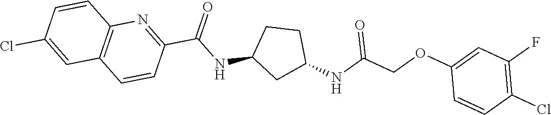 [SMILES: O=C(COc1ccc(Cl)c(F)c1)N[C@H]1CC[C@H](NC(=O)c2ccc3cc(Cl)ccc3n2)C1] |
| 17 | 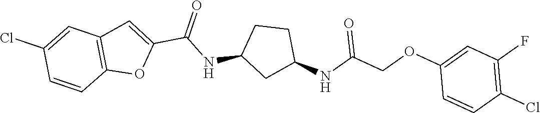 [SMILES: O=C(COc1ccc(Cl)c(F)c1)N[C@@H]1CC[C@H](NC(=O)c2cc3cc(Cl)ccc3o2)C1] |
| 18 | 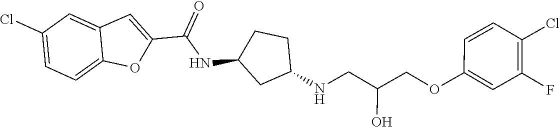 [SMILES: O=C(N[C@H]1CC[C@H](NCC(O)COc2ccc(Cl)c(F)c2)C1)c1cc2cc(Cl)ccc2o1] |
| 19 | 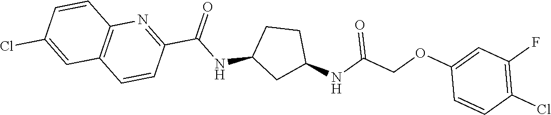 [SMILES: O=C(COc1ccc(Cl)c(F)c1)N[C@@H]1CC[C@H](NC(=O)c2ccc3cc(Cl)ccc3n2)C1] |
| 20 | 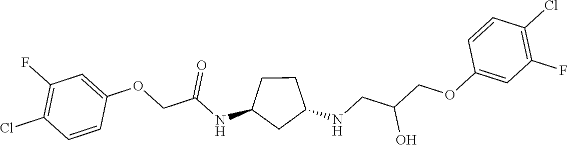 [SMILES: O=C(COc1ccc(Cl)c(F)c1)N[C@H]1CC[C@H](NCC(O)COc2ccc(Cl)c(F)c2)C1] |
| 21 | 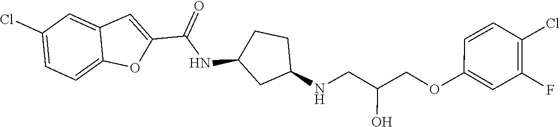 [SMILES: O=C(N[C@H]1CC[C@@H](NCC(O)COc2ccc(Cl)c(F)c2)C1)c1cc2cc(Cl)ccc2o1] |
| 22 | 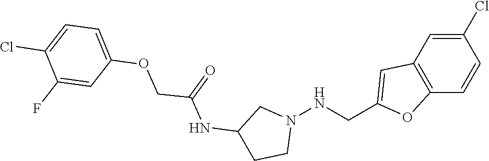 [SMILES: O=C(COc1ccc(Cl)c(F)c1)NC1CCN(NCc2cc3cc(Cl)ccc3o2)C1] |
| 23 | 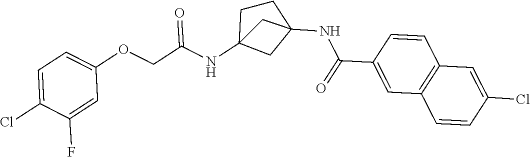 [SMILES: O=C(COc1ccc(Cl)c(F)c1)NC12CCC(NC(=O)c3ccc4cc(Cl)ccc4c3)(C1)C2] |
| 24 | 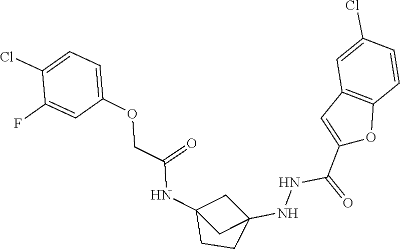 [SMILES: O=C(COc1ccc(Cl)c(F)c1)NC12CCC(NNC(=O)c3cc4cc(Cl)ccc4o3)(C1)C2] |
| 25 | 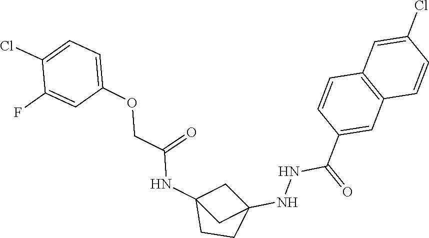 [SMILES: O=C(COc1ccc(Cl)c(F)c1)NC12CCC(NNC(=O)c3ccc4cc(Cl)ccc4c3)(C1)C2] |
| 26 | 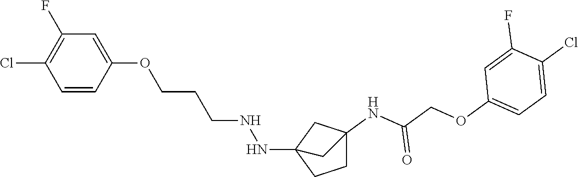 [SMILES: O=C(COc1ccc(Cl)c(F)c1)NC12CCC(NNCCCOc3ccc(Cl)c(F)c3)(C1)C2] |
| 27 | 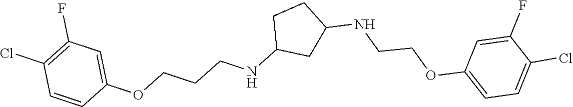 [SMILES: Fc1cc(OCCCNC2CCC(NCCOc3ccc(Cl)c(F)c3)C2)ccc1Cl] |
| 28 | 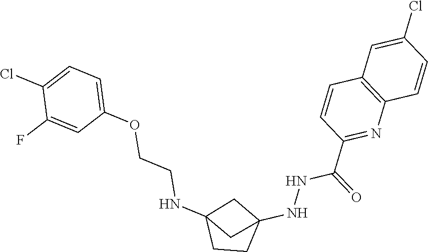 [SMILES: O=C(NNC12CCC(NCCOc3ccc(Cl)c(F)c3)(C1)C2)c1ccc2cc(Cl)ccc2n1] |
| 29 | 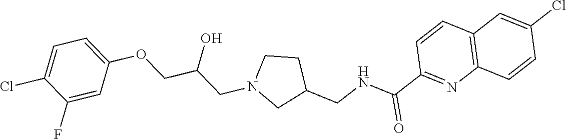 [SMILES: O=C(NCC1CCN(CC(O)COc2ccc(Cl)c(F)c2)C1)c1ccc2cc(Cl)ccc2n1] |
| 30 | 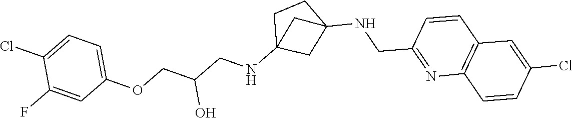 [SMILES: OC(CNC12CCC(NCc3ccc4cc(Cl)ccc4n3)(C1)C2)COc1ccc(Cl)c(F)c1] |
| 31 | 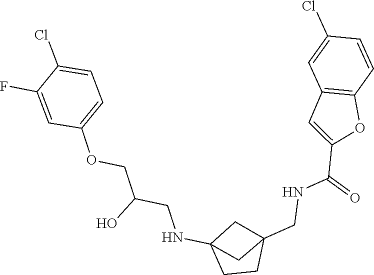 [SMILES: O=C(NCC12CCC(NCC(O)COc3ccc(Cl)c(F)c3)(C1)C2)c1cc2cc(Cl)ccc2o1] |
| 32 | 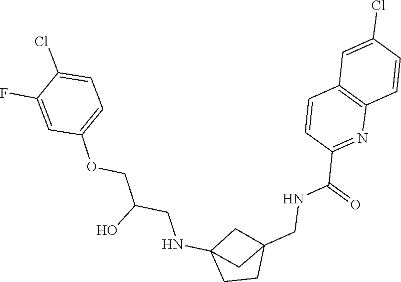 [SMILES: O=C(NCC12CCC(NCC(O)COc3ccc(Cl)c(F)c3)(C1)C2)c1ccc2cc(Cl)ccc2n1] |
| 33 | 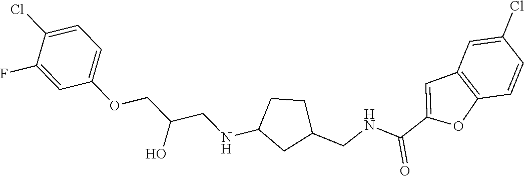 [SMILES: O=C(NCC1CCC(NCC(O)COc2ccc(Cl)c(F)c2)C1)c1cc2cc(Cl)ccc2o1] |
| 34 | 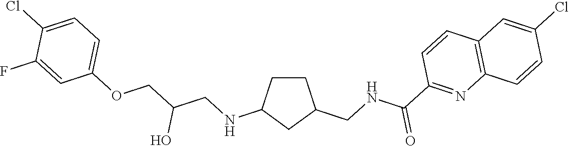 [SMILES: O=C(NCC1CCC(NCC(O)COc2ccc(Cl)c(F)c2)C1)c1ccc2cc(Cl)ccc2n1] |
| 35 | 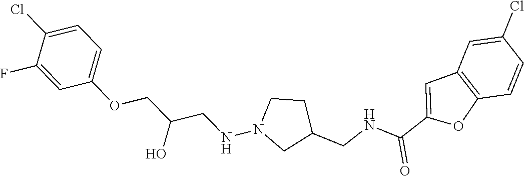 [SMILES: O=C(NCC1CCN(NCC(O)COc2ccc(Cl)c(F)c2)C1)c1cc2cc(Cl)ccc2o1] |
| 36 | 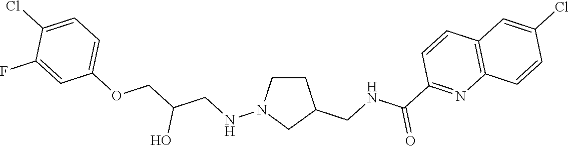 [SMILES: O=C(NCC1CCN(NCC(O)COc2ccc(Cl)c(F)c2)C1)c1ccc2cc(Cl)ccc2n1] |
| 37 | 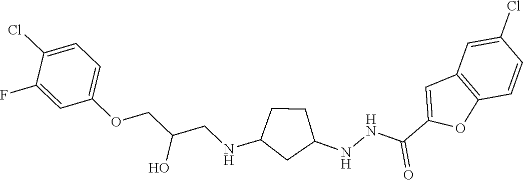 [SMILES: O=C(NNC1CCC(NCC(O)COc2ccc(Cl)c(F)c2)C1)c1cc2cc(Cl)ccc2o1] |
| 38 | 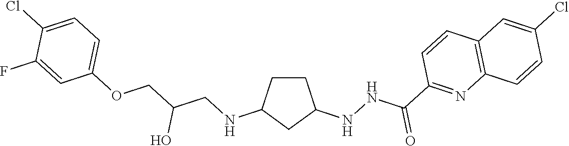 [SMILES: O=C(NNC1CCC(NCC(O)COc2ccc(Cl)c(F)c2)C1)c1ccc2cc(Cl)ccc2n1] |
| 39 | 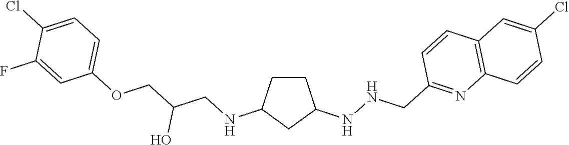 [SMILES: OC(CNC1CCC(NNCc2ccc3cc(Cl)ccc3n2)C1)COc1ccc(Cl)c(F)c1] |
| 40 | 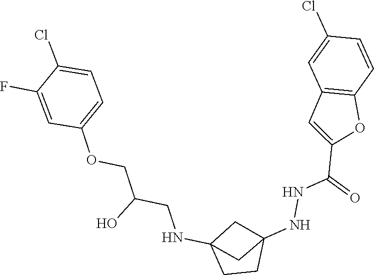 [SMILES: O=C(NNC12CCC(NCC(O)COc3ccc(Cl)c(F)c3)(C1)C2)c1cc2cc(Cl)ccc2o1] |
| 41 | 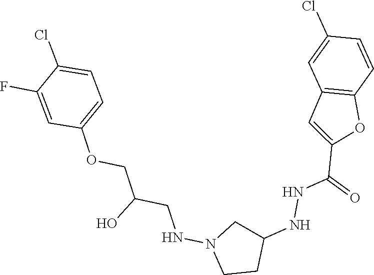 [SMILES: O=C(NNC1CCN(NCC(O)COc2ccc(Cl)c(F)c2)C1)c1cc2cc(Cl)ccc2o1] |
| 42 | 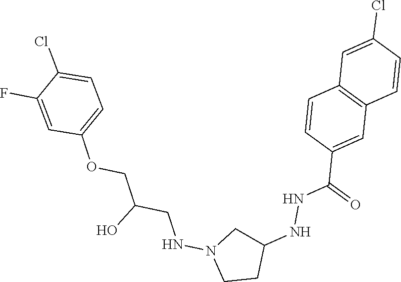 [SMILES: O=C(NNC1CCN(NCC(O)COc2ccc(Cl)c(F)c2)C1)c1ccc2cc(Cl)ccc2c1] |
| 43 | 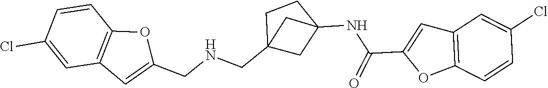 [SMILES: O=C(NC12CCC(CNCc3cc4cc(Cl)ccc4o3)(C1)C2)c1cc2cc(Cl)ccc2o1] |
| 44 | 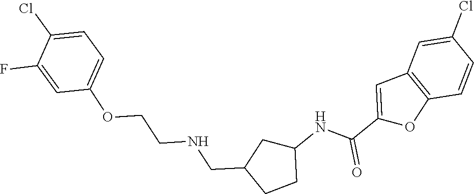 [SMILES: O=C(NC1CCC(CNCCOc2ccc(Cl)c(F)c2)C1)c1cc2cc(Cl)ccc2o1] |
| 45 | 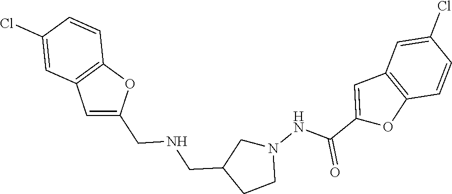 [SMILES: O=C(NN1CCC(CNCc2cc3cc(Cl)ccc3o2)C1)c1cc2cc(Cl)ccc2o1] |
| 46 | 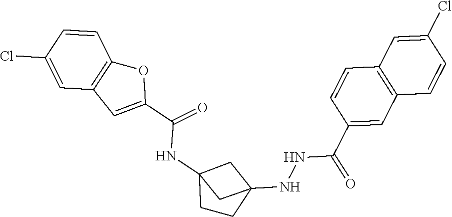 [SMILES: O=C(NNC12CCC(NC(=O)c3cc4cc(Cl)ccc4o3)(C1)C2)c1ccc2cc(Cl)ccc2c1] |
| 47 | 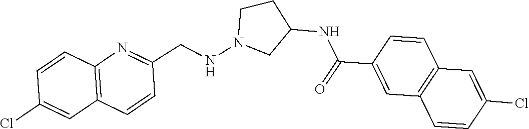 [SMILES: O=C(NC1CCN(NCc2ccc3cc(Cl)ccc3n2)C1)c1ccc2cc(Cl)ccc2c1] |
| 48 | 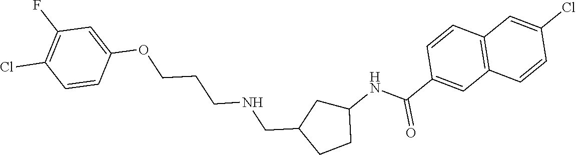 [SMILES: O=C(NC1CCC(CNCCCOc2ccc(Cl)c(F)c2)C1)c1ccc2cc(Cl)ccc2c1] |
| 49 | 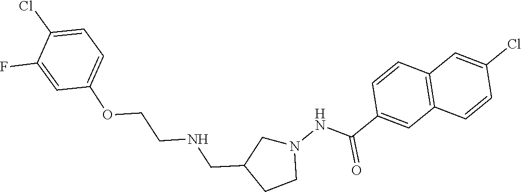 [SMILES: O=C(NN1CCC(CNCCOc2ccc(Cl)c(F)c2)C1)c1ccc2cc(Cl)ccc2c1] |
| 50 | 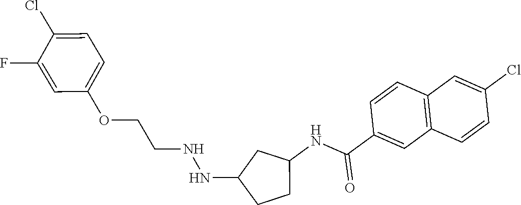 [SMILES: O=C(NC1CCC(NNCCOc2ccc(Cl)c(F)c2)C1)c1ccc2cc(Cl)ccc2c1] |
| 51 | 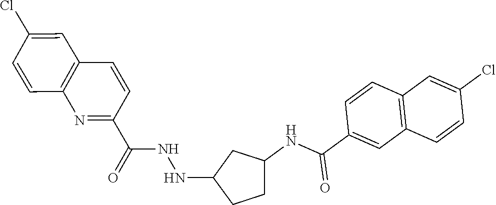 [SMILES: O=C(NC1CCC(NNC(=O)c2ccc3cc(Cl)ccc3n2)C1)c1ccc2cc(Cl)ccc2c1] |
| 52 | 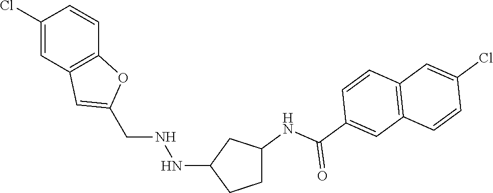 [SMILES: O=C(NC1CCC(NNCc2cc3cc(Cl)ccc3o2)C1)c1ccc2cc(Cl)ccc2c1] |
| 53 | 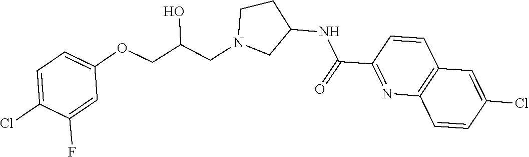 [SMILES: O=C(NC1CCN(CC(O)COc2ccc(Cl)c(F)c2)C1)c1ccc2cc(Cl)ccc2n1] |
| 54 | 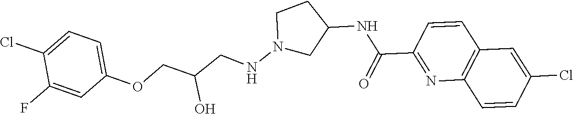 [SMILES: O=C(NC1CCN(NCC(O)COc2ccc(Cl)c(F)c2)C1)c1ccc2cc(Cl)ccc2n1] |
| 55 | 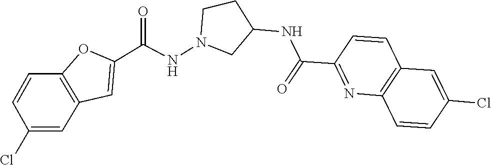 [SMILES: O=C(NC1CCN(NC(=O)c2cc3cc(Cl)ccc3o2)C1)c1ccc2cc(Cl)ccc2n1] |
| 56 | 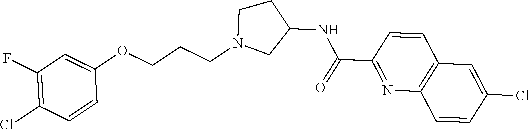 [SMILES: O=C(NC1CCN(CCCOc2ccc(Cl)c(F)c2)C1)c1ccc2cc(Cl)ccc2n1] |
| 57 | 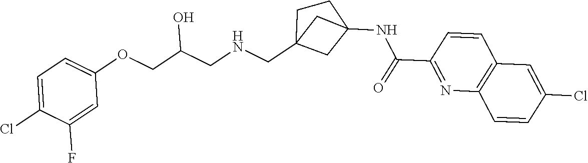 [SMILES: O=C(NC12CCC(CNCC(O)COc3ccc(Cl)c(F)c3)(C1)C2)c1ccc2cc(Cl)ccc2n1] |
| 58 | 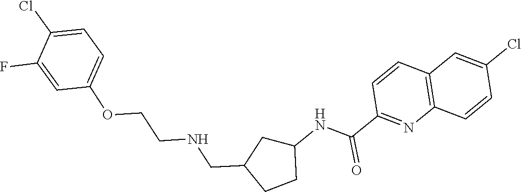 [SMILES: O=C(NC1CCC(CNCCOc2ccc(Cl)c(F)c2)C1)c1ccc2cc(Cl)ccc2n1] |
| 59 | 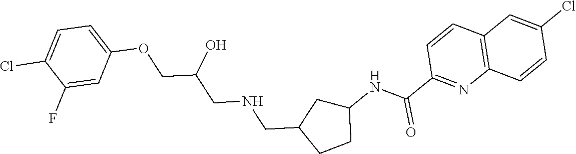 [SMILES: O=C(NC1CCC(CNCC(O)COc2ccc(Cl)c(F)c2)C1)c1ccc2cc(Cl)ccc2n1] |
| 60 | 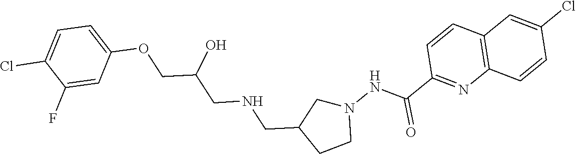 [SMILES: O=C(NN1CCC(CNCC(O)COc2ccc(Cl)c(F)c2)C1)c1ccc2cc(Cl)ccc2n1] |
| 61 | 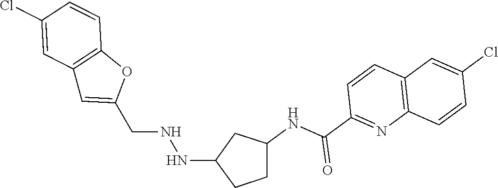 [SMILES: O=C(NC1CCC(NNCc2cc3cc(Cl)ccc3o2)C1)c1ccc2cc(Cl)ccc2n1] |
| 62 | 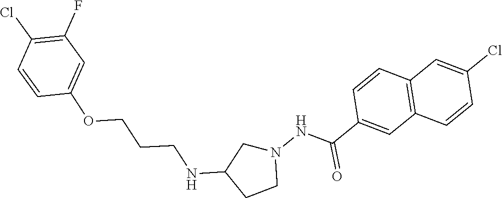 [SMILES: O=C(NN1CCC(NCCCOc2ccc(Cl)c(F)c2)C1)c1ccc2cc(Cl)ccc2c1] |
| 63 | 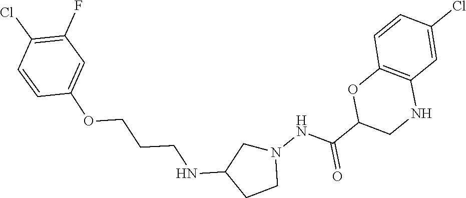 [SMILES: O=C(NN1CCC(NCCCOc2ccc(Cl)c(F)c2)C1)C1CNc2cc(Cl)ccc2O1] |
| 64 | 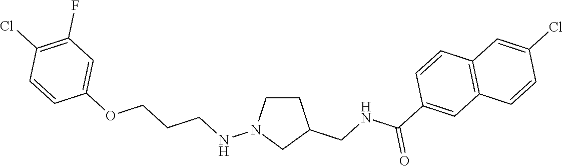 [SMILES: O=C(NCC1CCN(NCCCOc2ccc(Cl)c(F)c2)C1)c1ccc2cc(Cl)ccc2c1] |
| 65 | 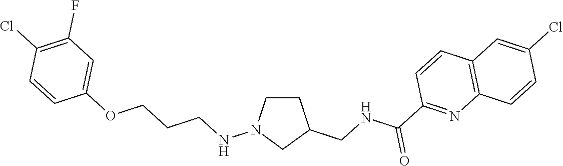 [SMILES: O=C(NCC1CCN(NCCCOc2ccc(Cl)c(F)c2)C1)c1ccc2cc(Cl)ccc2n1] |
| 66 | 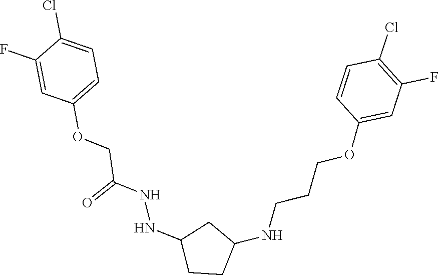 [SMILES: O=C(COc1ccc(Cl)c(F)c1)NNC1CCC(NCCCOc2ccc(Cl)c(F)c2)C1] |
| 67 | 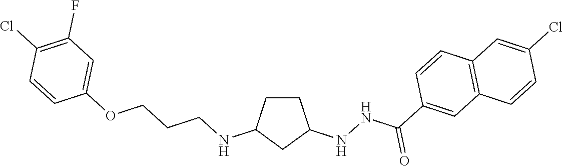 [SMILES: O=C(NNC1CCC(NCCCOc2ccc(Cl)c(F)c2)C1)c1ccc2cc(Cl)ccc2c1] |
| 68 | 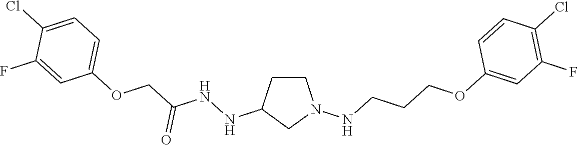 [SMILES: O=C(COc1ccc(Cl)c(F)c1)NNC1CCN(NCCCOc2ccc(Cl)c(F)c2)C1] |
| 69 | 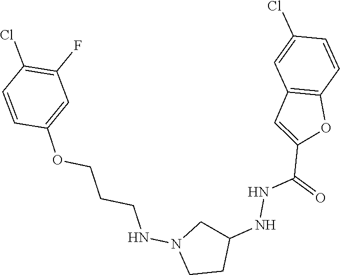 [SMILES: O=C(NNC1CCN(NCCCOc2ccc(Cl)c(F)c2)C1)c1cc2cc(Cl)ccc2o1] |
| 70 | 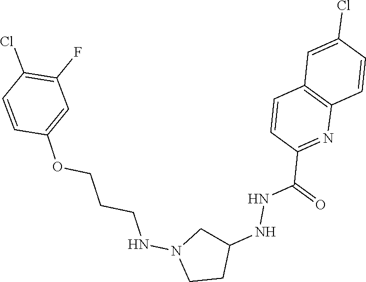 [SMILES: O=C(NNC1CCN(NCCCOc2ccc(Cl)c(F)c2)C1)c1ccc2cc(Cl)ccc2n1] |
| 71 | 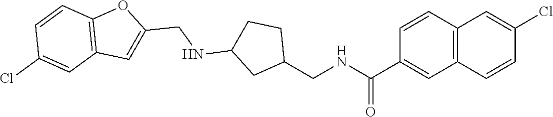 [SMILES: O=C(NCC1CCC(NCc2cc3cc(Cl)ccc3o2)C1)c1ccc2cc(Cl)ccc2c1] |
| 72 | 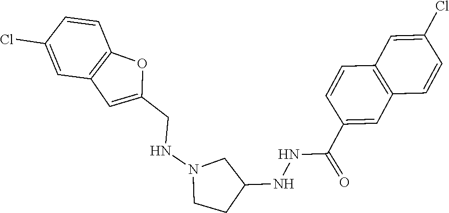 [SMILES: O=C(NNC1CCN(NCc2cc3cc(Cl)ccc3o2)C1)c1ccc2cc(Cl)ccc2c1] |
| 73 | 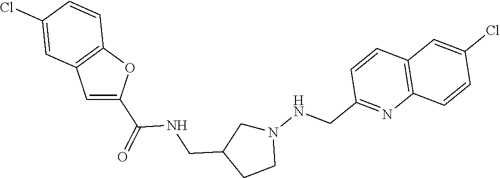 [SMILES: O=C(NCC1CCN(NCc2ccc3cc(Cl)ccc3n2)C1)c1cc2cc(Cl)ccc2o1] |
| 74 | 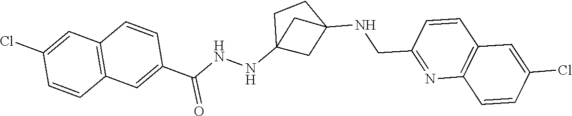 [SMILES: O=C(NNC12CCC(NCc3ccc4cc(Cl)ccc4n3)(C1)C2)c1ccc2cc(Cl)ccc2c1] |
| 75 | 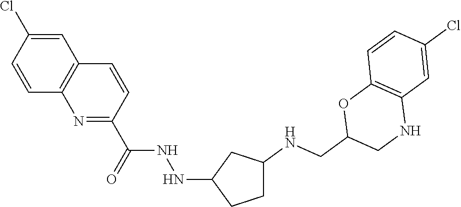 [SMILES: O=C(NNC1CCC(NCC2CNc3cc(Cl)ccc3O2)C1)c1ccc2cc(Cl)ccc2n1] |
| 76 | 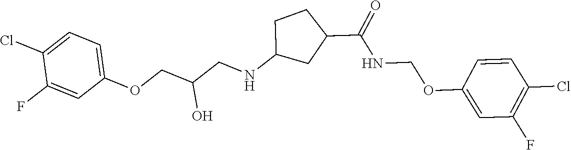 [SMILES: O=C(NCOc1ccc(Cl)c(F)c1)C1CCC(NCC(O)COc2ccc(Cl)c(F)c2)C1] |
| 77 | 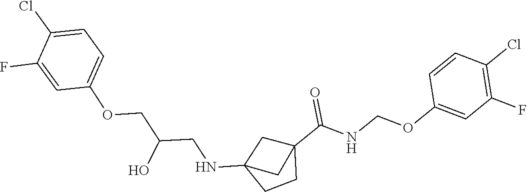 [SMILES: O=C(NCOc1ccc(Cl)c(F)c1)C12CCC(NCC(O)COc3ccc(Cl)c(F)c3)(C1)C2] |
| 78 | 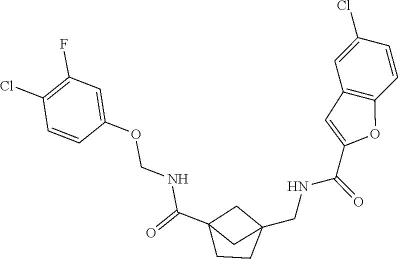 [SMILES: O=C(NCC12CCC(C(=O)NCOc3ccc(Cl)c(F)c3)(C1)C2)c1cc2cc(Cl)ccc2o1] |
| 79 | 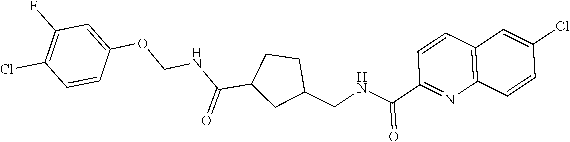 [SMILES: O=C(NCC1CCC(C(=O)NCOc2ccc(Cl)c(F)c2)C1)c1ccc2cc(Cl)ccc2n1] |
| 80 | 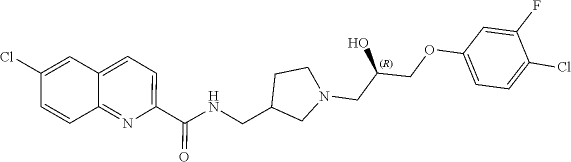 |
| 81 | 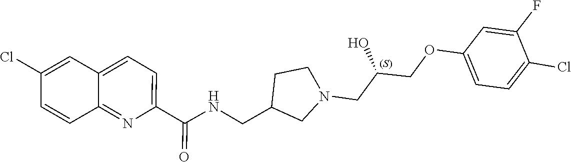 |
| 82 | 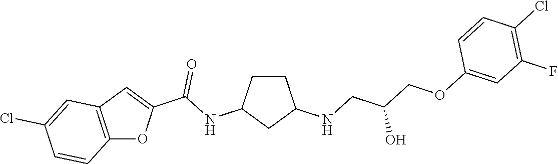 [SMILES: O=C(NC1CCC(NC[C@@H](O)COc2ccc(Cl)c(F)c2)C1)c1cc2cc(Cl)ccc2o1] |
| 83 | 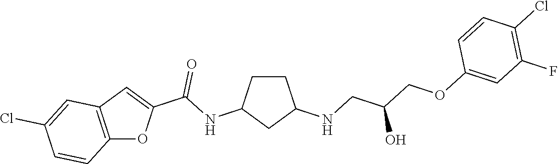 [SMILES: O=C(NC1CCC(NC[C@H](O)COc2ccc(Cl)c(F)c2)C1)c1cc2cc(Cl)ccc2o1] |
| 84 | 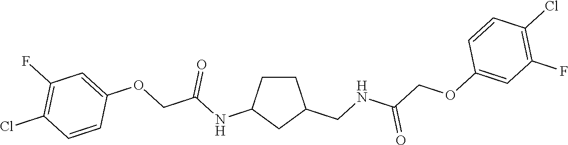 [SMILES: O=C(COc1ccc(Cl)c(F)c1)NCC1CCC(NC(=O)COc2ccc(Cl)c(F)c2)C1] |
| 85 | 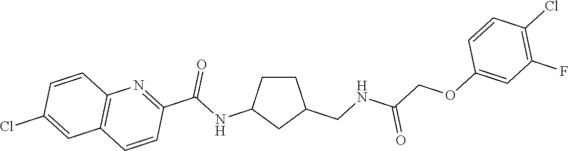 [SMILES: O=C(COc1ccc(Cl)c(F)c1)NCC1CCC(NC(=O)c2ccc3cc(Cl)ccc3n2)C1] |
| 86 | 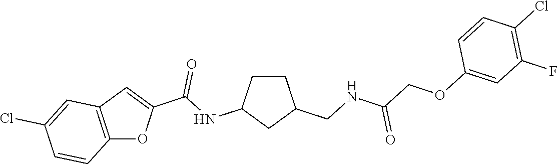 [SMILES: O=C(COc1ccc(Cl)c(F)c1)NCC1CCC(NC(=O)c2cc3cc(Cl)ccc3o2)C1] |
| 87 | 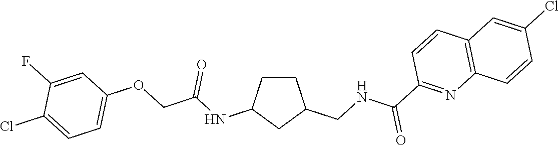 [SMILES: O=C(COc1ccc(Cl)c(F)c1)NC1CCC(CNC(=O)c2ccc3cc(Cl)ccc3n2)C1] |
| 88 | 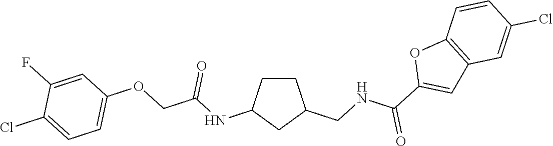 [SMILES: O=C(COc1ccc(Cl)c(F)c1)NC1CCC(CNC(=O)c2cc3cc(Cl)ccc3o2)C1] |
| 89 | 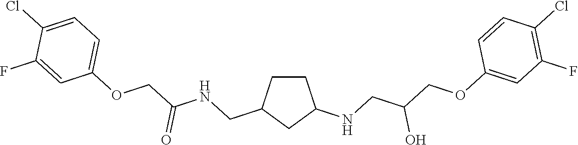 [SMILES: O=C(COc1ccc(Cl)c(F)c1)NCC1CCC(NCC(O)COc2ccc(Cl)c(F)c2)C1] |
| 90 | 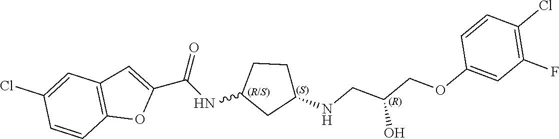 |
| 91 | 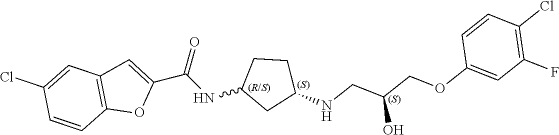 |
| 92 | 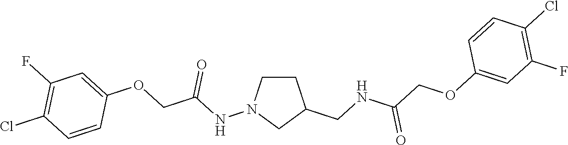 [SMILES: O=C(COc1ccc(Cl)c(F)c1)NCC1CCN(NC(=O)COc2ccc(Cl)c(F)c2)C1] |
| 93 | 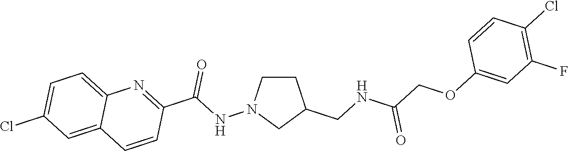 [SMILES: O=C(COc1ccc(Cl)c(F)c1)NCC1CCN(NC(=O)c2ccc3cc(Cl)ccc3n2)C1] |
| 94 | 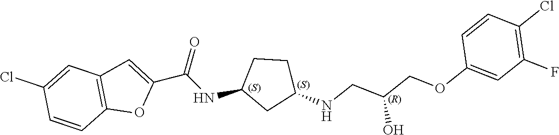 |
| 95 | 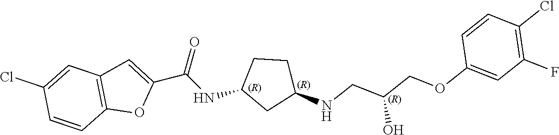 |
| 96 | 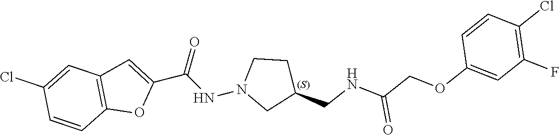 |
| 97 | 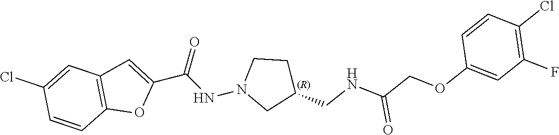 |
| 98 | 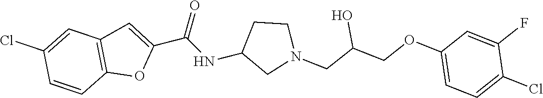 [SMILES: O=C(NC1CCN(CC(O)COc2ccc(Cl)c(F)c2)C1)c1cc2cc(Cl)ccc2o1] |
| 99 | 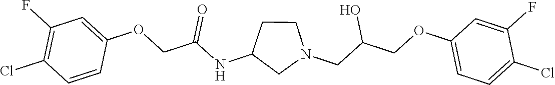 [SMILES: O=C(COc1ccc(Cl)c(F)c1)NC1CCN(CC(O)COc2ccc(Cl)c(F)c2)C1] |
| 100 | 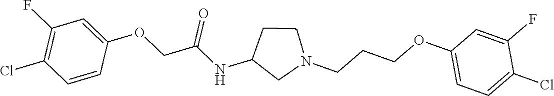 [SMILES: O=C(COc1ccc(Cl)c(F)c1)NC1CCN(CCCOc2ccc(Cl)c(F)c2)C1] |
| 101 | 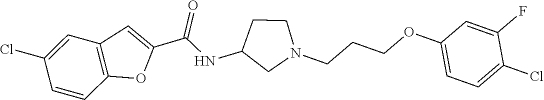 [SMILES: O=C(NC1CCN(CCCOc2ccc(Cl)c(F)c2)C1)c1cc2cc(Cl)ccc2o1] |

### Pharmaceutical Compositions and Formulations

Pharmaceutical compositions of any of the compounds detailed herein are embraced by this disclosure. Thus, the present disclosure includes pharmaceutical compositions comprising a compound as detailed herein or a salt thereof and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be formulated as a 10 mg tablet.

Compositions comprising a compound provided herein are also described. In one variation, the composition comprises a compound or salt thereof and a pharmaceutically acceptable carrier or excipient. In another variation, a composition of substantially pure compound is provided. In some embodiments, the composition is for use as a human or veterinary medicament. In some embodiments, the composition is for use in a method described herein. In some embodiments, the composition is for use in the treatment of a disease or disorder described herein.

### Methods of Use and Uses

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease or disorder in an individual in need thereof comprising administering a compound describes herein or any embodiment, variation, or aspect thereof, or a pharmaceutically acceptable salt thereof. In some embodiments, the compound, pharmaceutically acceptable salt thereof, or composition is administered to the individual according to a dosage and/or method of administration described herein.

The compounds or salts thereof described herein and compositions described herein are believed to be effective for treating a variety of diseases and disorders. In some embodiments, a compound or salt thereof described herein or a composition described herein may be used in a method of treating a disease or disorder mediated by an integrated stress response (ISR) pathway. In some embodiments, the disease or disorder is mediated by eukaryotic translation initiation factor 2α (eIF2α) or eukaryotic translation initiation factor 2B (eIF2B). In some embodiments, the disease or disorder is mediated by phosphorylation of eIF2α and/or the guanine nucleotide exchange factor (GEF) activity of eIF2B.

In some embodiments, a compound or salt thereof described herein or a composition described herein may be used in a method of treating a disease or disorder, wherein the disease or disorder is a neurodegenerative disease, an inflammatory disease, an autoimmune disease, a metabolic syndrome, a cancer, a vascular disease, a musculoskeletal disease (such as a myopathy), an ocular disease, or a genetic disorder.

In some embodiments, the disease or disorder is a neurodegenerative disease. In some embodiments, the neurodegenerative disease is vanishing white matter disease, childhood ataxia with CNS hypomyelination, intellectual disability syndrome, Alzheimer's disease, prion disease, Creutzfeldt-Jakob disease, Parkinson's disease, amyotrophic lateral sclerosis (ALS) disease, Pelizaeus-Merzbacher disease, a cognitive impairment, a traumatic brain injury, a postoperative cognitive dysfunction (PCD), a neuro-otological syndrome, hearing loss, Huntington's disease, stroke, chronic traumatic encephalopathy, spinal cord injury, dementia, frontotemporal dementia (FTD), depression, or a social behavior impairment. In some embodiments, the cognitive impairment is triggered by ageing, radiation, sepsis, seizure, heart attack, heart surgery, liver failure, hepatic encephalopathy, anesthesia, brain injury, brain surgery, ischemia, chemotherapy, cancer treatment, critical illness, concussion, fibromyalgia, or depression. In some embodiments, the neurodegenerative disease is Alzheimer's disease. In some embodiments, the neurodegenerative disease is ageing-related cognitive impairment. In some embodiments, the neurodegenerative disease is a traumatic brain injury.

In some embodiments, a compound or salt thereof described herein or a composition described herein may be used in a method of treating Alzheimer's disease. In some embodiments, neurodegeneration, cognitive impairment, and/or amyloidogenesis is decreased.

In some embodiments, the disease or disorder is an inflammatory disease. In some embodiments, the inflammatory disease is arthritis, psoriatic arthritis, psoriasis, juvenile idiopathic arthritis, asthma, allergic asthma, bronchial asthma, tuberculosis, chronic airway disorder, cystic fibrosis, glomerulonephritis, membranous nephropathy, sarcoidosis, vasculitis, ichthyosis, transplant rejection, interstitial cystitis, atopic dermatitis, or inflammatory bowel disease. In some embodiments, the inflammatory bowel disease is Crohn' disease, ulcerative colitis, or celiac disease.

In some embodiments, the disease or disorder is an autoimmune disease. In some embodiments, the autoimmune disease is systemic lupus erythematosus, type 1 diabetes, multiple sclerosis, or rheumatoid arthritis.

In some embodiments, the disease or disorder is a metabolic syndrome. In some embodiments, the metabolic syndrome is alcoholic liver steatosis, obesity, glucose intolerance, insulin resistance, hyperglycemia, fatty liver, dyslipidemia, hyperlipidemia, hyperhomocysteinemia, or type 2 diabetes.

In some embodiments, the disease or disorder is a cancer. In some embodiments, the cancer is pancreatic cancer, breast cancer, kidney cancer, bladder cancer, prostate cancer, testicular cancer, urothelial cancer, endometrial cancer, ovarian cancer, cervical cancer, renal cancer, esophageal cancer, gastrointestinal stromal tumor (GIST), multiple myeloma, cancer of secretory cells, thyroid cancer, gastrointestinal carcinoma, chronic myeloid leukemia, hepatocellular carcinoma, colon cancer, melanoma, malignant glioma, glioblastoma, glioblastoma multiforme, astrocytoma, dysplastic gangliocytoma of the cerebellum, Ewing's sarcoma, rhabdomyosarcoma, ependymoma, medulloblastoma, ductal adenocarcinoma, adenosquamous carcinoma, nephroblastoma, acinar cell carcinoma, neuroblastoma, or lung cancer. In some embodiments, the cancer of secretory cells is non-Hodgkin's lymphoma, Burkitt's lymphoma, chronic lymphocytic leukemia, monoclonal gammopathy of undetermined significance (MGUS), plasmacytoma, lymphoplasmacytic lymphoma or acute lymphoblastic leukemia.

In some embodiments, the disease or disorder is a musculoskeletal disease (such as a myopathy). In some embodiments, the musculoskeletal disease is a myopathy, a muscular dystrophy, a muscular atrophy, a muscular wasting, or sarcopenia. In some embodiments, the muscular dystrophy is Duchenne muscular dystrophy (DMD), Becker's disease, myotonic dystrophy, X-linked dilated cardiomyopathy, spinal muscular atrophy (SMA), or metaphyseal chondrodysplasia, Schmid type (MCDS). In some embodiments, the myopathy is a skeletal muscle atrophy. In some embodiments, the musculoskeletal disease (such as the skeletal muscle atrophy) is triggered by ageing, chronic diseases, stroke, malnutrition, bedrest, orthopedic injury, bone fracture, cachexia, starvation, heart failure, obstructive lung disease, renal failure, Acquired Immunodeficiency Syndrome (AIDS), sepsis, an immune disorder, a cancer, ALS, a burn injury, denervation, diabetes, muscle disuse, limb immobilization, mechanical unload, myositis, or a dystrophy.

In some embodiments, the disease or disorder is a genetic disorder, such as Down syndrome or MEHMO syndrome (Mental retardation, Epileptic seizures, Hypogenitalism, Microcephaly, and Obesity).

In some embodiments, a compound or salt thereof described herein or a composition described herein may be used in a method of treating musculoskeletal disease. In some embodiments, skeletal muscle mass, quality and/or strength are increased. In some embodiments, synthesis of muscle proteins is increased. In some embodiments, skeletal muscle fiber atrophy is inhibited.

In some embodiments, the disease or disorder is a vascular disease. In some embodiments, the vascular disease is atherosclerosis, abdominal aortic aneurism, carotid artery disease, deep vein thrombosis, Buerger's disease, chronic venous hypertension, vascular calcification, telangiectasia or lymphoedema.

In some embodiments, the disease or disorder is an ocular disease. In some embodiments, the ocular disease is glaucoma, age-related macular degeneration, inflammatory retinal disease, retinal vascular disease, diabetic retinopathy, uveitis, rosacea, Sjogren's syndrome, or neovascularization in proliferative retinopathy.

In some embodiments, provided herein is a method of inhibiting an ISR pathway. The compounds or salts thereof described herein and compositions described herein are believed to be effective for inhibiting an ISR pathway. In some embodiments, the method of inhibiting an ISR pathway comprises inhibiting the ISR pathway in a cell by administering or delivering to the cell a compound described herein, or a pharmaceutically acceptable salt thereof, or a pharmaceutical composition described herein. In some embodiments, the method of inhibiting an ISR pathway comprises inhibiting the ISR pathway in an individual by administering to the individual a compound described herein, or a pharmaceutically acceptable salt thereof, or a pharmaceutical composition described herein. Inhibition of the ISR pathway can be determined by methods known in the art, such as western blot, immunohistochemistry, or reporter cell line assays.

In some embodiments, the inhibition of the ISR pathway comprises binding eIF2B. In some embodiments, the inhibition of the ISR pathway comprises increasing protein translation, increasing guanine nucleotide exchange factor (GEF) activity of eIF2B, delaying or preventing apoptosis in a cell, and/or inhibiting translation of one or more mRNAs comprising a 5′ untranslated region (5′UTR) comprising at least one upstream open reading frame (uORF).

In some embodiments, provided herein are methods of increasing protein production using a compound or salt described herein. The protein production is increased relative to the same condition without the compound or salt. Protein production can be increased either in vivo or in vitro. For example, protein production can be increased in vivo by administering the compound or salt to an individual. In some embodiments, protein production is increased in vitro using the compound or salt with a cell-free protein synthesis system (CFPS) or a cell-based protein expression system. The protein produced can be a heterologous protein (e.g., a recombinant protein) or a native protein. Heterologous protein production can be achieved using a recombinant nucleic acid encoding the protein. In some embodiments, the protein produced is an antibody or a fragment thereof. Other exemplary proteins can include, but are not limited to, enzymes, allergenic peptides or proteins (for example, for use as a vaccine), recombinant protein, cytokines, peptides, hormones, erythropoietin (EPO), interferons, granulocyte-colony stimulating factor (G-CSF), anticoagulants, and clotting factors. The increase in protein production can be determined by methods known in the art, such as western blot or immunohistochemistry.

Cell-free protein synthesis (CFPS) systems are generally known, and include cellular machinery for protein expression in an in vitro environment. In some embodiments, the CFPS system includes a cellular extract (such as a eukaryotic cellular extract), which includes protein expression machinery. In some embodiment, the cellular machinery in the CFPS system comprises eukaryotic cellular machinery, such as eukaryotic initiation factor 2 (eIF2) and/or eukaryotic initiation factor 2B (eIF2B), or one or more subunits thereof.

In some embodiments, there is a cell-free protein synthesis (CFPS) system comprising eukaryotic initiation factor 2 (eIF2) and a nucleic acid encoding a protein with a compound or salt as described herein. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, erythropoietin (EPO), interferons, granulocyte-colony stimulating factor (G-CSF), anticoagulants, and clotting factors. In some embodiments, the CFPS system comprises a cell extract comprising the eIF2. In some embodiments, the CFPS system further comprises eIF2B.

In some embodiments, there is a method of producing a protein, comprising contacting a cell-free protein synthesis (CFPS) system comprising eukaryotic initiation factor 2 (eIF2) and a nucleic acid encoding a protein with a compound or salt thereof as described herein. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, erythropoietin (EPO), interferons, granulocyte-colony stimulating factor (G-CSF), anticoagulants, and clotting factors. In some embodiments, the CFPS system comprises a cell extract comprising the eIF2. In some embodiments, the CFPS system further comprises eIF2B. In some embodiments, the method comprises purifying the protein.

In some embodiments, there is a method of producing a protein, comprising contacting a eukaryotic cell comprising a nucleic acid encoding the protein with a compound or salt as described herein. In some embodiments, the method comprises culturing the cell in an in vitro culture medium comprising the compound or salt. In some embodiments, the nucleic acid encoding the protein is a recombinant nucleic acid. In some embodiments, the eukaryotic cell is a human embryonic kidney (HEK) cell or a Chinese hamster ovary (CHO) cell. In other embodiments, the eukaryotic cell is a yeast cell (such as Saccharomyces cerevisiae or Pichia pastoris), a wheat germ cell, an insect cell, a rabbit reticulocyte, a cervical cancer cell (such as a HeLa cell), a baby hamster kidney cell (such as BHK21 cells), a murine myeloma cell (such as NSO or Sp2/0 cells), an HT-1080 cell, a PER.C6 cell, a plant cell, a hybridoma cell, or a human blood derived leukocyte. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, erythropoietin (EPO), interferons, granulocyte-colony stimulating factor (G-CSF), anticoagulants, and clotting factors. In some embodiments, the method comprises purifying the protein.

In some embodiments, there is a method of culturing a eukaryotic cell comprising a nucleic acid encoding a protein, comprising contacting the eukaryotic cell with an in vitro culture medium comprising a compound or salt as described herein. In some embodiments, the nucleic acid encoding the protein is a recombinant nucleic acid. In some embodiments, the eukaryotic cell is a human embryonic kidney (HEK) cell or a Chinese hamster ovary (CHO) cell. In other embodiments, the eukaryotic cell is a yeast cell (such as Saccharomyces cerevisiae or Pichia pastoris), a wheat germ cell, an insect cell, a rabbit reticulocyte, a cervical cancer cell (such as a HeLa cell), a baby hamster kidney cell (such as BHK21 cells), a murine myeloma cell (such as NSO or Sp2/0 cells), an HT-1080 cell, a PER.C6 cell, a plant cell, a hybridoma cell, or a human blood derived leukocyte. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, erythropoietin (EPO), interferons, granulocyte-colony stimulating factor (G-CSF), anticoagulants, and clotting factors. In some embodiments, the method comprises purifying the protein.

In some embodiments, there is an in vitro cell culture medium, comprising the compound or salt described herein, and nutrients for cellular growth. In some embodiments, the culture medium comprises a eukaryotic cell comprising a nucleic acid encoding a protein. In some embodiments, the culture medium further comprises a compound for inducing protein expression. In some embodiments, the nucleic acid encoding the protein is a recombinant nucleic acid. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, erythropoietin (EPO), interferons, granulocyte-colony stimulating factor (G-CSF), anticoagulants, and clotting factors. In some embodiments, the eukaryotic cell is a human embryonic kidney (HEK) cell or a Chinese hamster ovary (CHO) cell. In other embodiments, the eukaryotic cell is a yeast cell (such as Saccharomyces cerevisiae or Pichia pastoris), a wheat germ cell, an insect cell, a rabbit reticulocyte, a cervical cancer cell (such as a HeLa cell), a baby hamster kidney cell (such as BHK21 cells), a murine myeloma cell (such as NSO or Sp2/0 cells), an HT-1080 cell, a PER.C6 cell, a plant cell, a hybridoma cell, or a human blood derived leukocyte.

In some embodiments, provided herein is a method of increasing protein translation in a cell or cell free expression system. In some embodiments, the cell was stressed prior to administration of the compound, salt thereof, or composition. In some embodiments, protein translation is increased by at least about 10%, 20%, 30%, 40%, 50%, 60%, 70%, 80%, 90%, 95%, 98%, 100%, 125%, 150%, 175%, 200%, 250%, or 300% or more. In some embodiments, protein translation is increased by about 10% to about 300% (such as about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 125%, about 125% to about 150%, about 150% to about 175%, about 175% to about 200%, about 200% to about 250%, or about 250% to about 300%) In some embodiments, protein translation is increased as compared to prior to the administration of the compounds, salt thereof, or composition. In some embodiments, protein translation is increased as compared to an unstressed cell, a basal condition where cells are not subjected to a specific stress that activates the ISR. In some embodiments, protein translation is increased as compared to a stressed cell where ISR is active.

Some of the compounds described herein increase protein synthesis in a cell without full inhibition of ATF4 translation, under ISR-stressed or non-ISR stressed conditions. Despite ATF4 participation in various pathologies, the ATF4 protein is an important factor for restoring cellular homeostasis in stressed cells, for example during oxidative stress response, cholesterol metabolism, protein folding amino acid synthesis, and autophagy. Thus, for certain treatments, it may be preferable to limit ATF4 inhibition. In some embodiments, the compound is used to increase protein synthesis by about 10% or more, about 20% or more, about 30% or more, about 40% or more, about 50% or more, about 60% or more, about 70% or more, about 80% or more, about 90% or more, about 100% or more, about 125% or more, about 150% or more, about 175% or more, about 200% or more, about 250% or more, or about 300% or more wherein ATF4 protein expression is inhibited by about 75% or less, about 50% or less, about 40% or less, about 30% or less, about 20% or less, about 10% or less, or about 5% or less. In some embodiments the compound is used to increase protein synthesis by about 10% to about 300% (such as about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 125%, about 125% to about 150%, about 150% to about 175%, about 175% to about 200%, about 200% to about 250%, or about 250% to about 300%), wherein ATF4 protein expression is inhibited by about 75% or less (such as about 50% or less, about 40% or less, about 30% or less, about 20% or less, about 10% or less, or about 5% or less).

In some embodiments, provided herein is a method of increasing protein translation in a cell. In some embodiments, the cell was stressed prior to administration of the compound, salt thereof, or composition. In some embodiments, protein translation is increased by at least about 10%, 20%, 30%, 40%, 50%, 60%, 70%, 80%, 90%, 95%, 98%, 100%, 125%, 150%, 175%, 200%, 250%, or 300% or more. In some embodiments, protein translation is increased as compared to prior to the administration of the compounds, salt thereof, or composition. In some embodiments, protein translation is increased as compared to an unstressed cell, a basal condition where cells are not subjected to a specific stress that activates the ISR. In some embodiments, protein translation is increased as compared to a stressed cell where ISR is active.

In some embodiments, provided herein is a method of increasing guanine nucleotide exchange factor (GEF) activity of eIF2B in cells. In some embodiments, provided herein is a method of delaying or preventing apoptosis in a cell. In some embodiments, provided herein is a method of inhibiting translation of one or more mRNAs comprising a 5′ untranslated region (5′UTR) that contains at least one upstream open reading frame (uORF), encoding proteins with translational preferences, including but not limited to ATF4, ATF2, ATF5, CHOP, GADD34, BACE-1, C/EBPt, or MAP1LC3B. In some embodiments, the mRNA encodes ATF4, BACE-1, GADD34, or CHOP. In some embodiments, the mRNA encodes ATF4.

In some embodiments, expression of ATF4, BACE-1, GADD34 or CHOP is inhibited. In some embodiments, expression of ATF4 is inhibited. In some embodiments, expression of Aβ is inhibited. ATF4 increases expression of, among others, GADD45A, CDKN1A, and EIF4EBP1, which encode DDIT-1, p21, and 4E-BP1, respectively. These proteins induce musculoskeletal disease (such as skeletal muscle atrophy), and can be modulated by inhibiting expression of ATF4. Accordingly, in some embodiments, expression of one or more of CDKN1A, GADD45A, or EIF4EBP1 is inhibited.

In some embodiments, the compound, salt thereof, or composition inhibits translation of one or more mRNAs comprising a 5′ untranslated region (5′UTR) comprising at least one upstream open reading frame (uORF) with an IC₅₀ of less than about 1 μM, such as less than about 750 nM, 600 nM, 500 nM, 300 nM, 200 nM, 100 nM, 80 nM, 60 nM, 40 nM, 25 nM, or less. In some embodiments, the compound, salt thereof, or composition inhibits translation of one or more mRNAs comprising a 5′ untranslated region (5′UTR) comprising at least one upstream open reading frame (uORF) with an IC₅₀ between about 1 nM and 1 μM, such as between about 10 nM and 600 nM, 15 nM and 200 nM, or 20 nM and 180 nM.

In some embodiments, the compound, salt thereof, or composition inhibits expression of ATF4 with an IC₅₀ of less than about 1 μM, such as less than about 750 nM, 600 nM, 500 nM, 300 nM, 200 nM, 100 nM, 80 nM, 60 nM, 40 nM, 25 nM, or less. In some embodiments, the compound, salt thereof, or composition inhibits expression of ATF4 with an IC₅₀ between about 1 nM and 1 μM, such as between about 2 nM and 800 nM, 10 nM and 600 nM, 15 nM and 200 nM, or 20 nM and 180 nM.

In some aspects, the half maximal inhibitory concentration (IC₅₀) is a measure of the effectiveness of a substance in inhibiting a specific biological or biochemical function. In some aspects, the IC₅₀ is a quantitative measure that indicates how much of an inhibitor is needed to inhibit a given biological process or component of a process such as an enzyme, cell, cell receptor or microorganism by half. Methods of determining IC₅₀ in vitro and in vivo are known in the art.

In some embodiments, the individual is a mammal. In some embodiments, the individual is a primate, bovine, ovine, porcine, equine, canine, feline, rabbit, or rodent. In some embodiments, the individual is a human. In some embodiments, the individual has any of the diseases or disorders disclosed herein. In some embodiments, the individual is a risk for developing any of the diseases or disorders disclosed herein.

In some embodiments, the individual is human. In some embodiments, the human is at least about or is about any of 21, 25, 30, 35, 40, 45, 50, 55, 60, 65, 70, 75, 80, or 85 years old. In some embodiments, the human is a child. In some embodiments, the human is less than about or about any of 21, 18, 15, 12, 10, 8, 6, 5, 4, 3, 2, or 1 years old.

Also provided herein are uses of a compound described herein or a pharmaceutically acceptable salt thereof, or a pharmaceutical composition described herein, in the manufacture of a medicament. In some embodiments, the manufacture of a medicament is for the treatment of a disorder or disease described herein. In some embodiments, the manufacture of a medicament is for the prevention and/or treatment of a disorder or disease mediated by an ISR pathway. In some embodiments, the manufacture of a medicament is for the prevention and/or treatment of a disorder or disease mediated by eIF2α or eIF2B. In some embodiments, the manufacture of a medicament is for the prevention and/or treatment of a disorder or disease mediated by phosphorylation of eIF2α and/or the GEF activity of eIF2B.

### Combinations

In certain aspects, a compound described herein is administered to an individual for treatment of a disease in combination with one or more additional pharmaceutical agents that can treat the disease. For example, in some embodiments, an effective amount of the compound is administered to an individual for the treatment of cancer in combination with one or more additional anticancer agents.

In some embodiments, activity of the additional pharmaceutical agent (such as additional anticancer agent) is inhibited by an activated ISR pathway. An ISR inhibitor, such as one of the compounds described herein, can inhibit the ISR pathway to enhance functionality of the additional pharmaceutical agent. By way of example, certain BRAF inhibitors (e.g., vemurafenib or dabrafenib) activate the ISR pathway in BRAF-mutated melanoma cells (e.g., BRAF with a V600F mutation) through the expression of ATF4. In some embodiments, there is a method of treating cancer comprising administering to an individual with cancer an effective amount of a compound described herein in combination with an effective amount of a BRAF inhibitor. In some embodiments, there is a method of treating a BRAF-mutated melanoma comprising administering to an individual with a BRAF-mutated melanoma an effective amount of a compound described herein in combination with an effective amount of a BRAF inhibitor. In some embodiments, there is a method of treating a BRAF-mutated melanoma comprising administering to an individual with a BRAF-mutated melanoma an effective amount of a compound described herein in combination with an effective amount of vemurafenib or dabrafenib.

As another example, certain anticancer agents (such as ubiquitin-proteasome pathway inhibitors (such as bortezomib), Cox-2 inhibitors (e.g., celecoxib), platinum-based antineoplastic drugs (e.g., cisplatin), anthracyclines (e.g. doxorubicin), or topoisomerase inhibitors (e.g., etoposide)) are used to treat cancer, but may have limited functionality against solid tumors. Resistance in certain solid tumors (e.g., breast cancers) has been associated with ATF4 stabilization and induction of autophagy. In some embodiments, an effective amount of an ISR inhibitor compound as described herein is administered to an individual with cancer to increase sensitivity to one or more anticancer agents.

In some embodiments, there is a method of treating a refractory cancer (such as a solid tumor) in an individual, comprising administering to the individual an effective amount of a compound described herein in combination with an effective amount of an anticancer agent. In some embodiments, there is a method of treating a refractory cancer (such as a solid tumor) in an individual, comprising administering to the individual an effective amount of a compound described herein in combination with an effective amount of an ubiquitin-proteasome pathway inhibitor (e.g., bortezomib), a Cox-2 inhibitor (e.g., celecoxib), a platinum-based antineoplastic drug (e.g., cisplatin), an anthracycline (e.g. doxorubicin), or a topoisomerase inhibitor (e.g., etoposide). In some embodiments, the refractory cancer is breast cancer. In some embodiments, the refractory cancer is melanoma.

In some embodiments, a compound described herein is used to treat cancer in combination with one or more anti-cancer agents, such as an anti-neoplastic agent, an immune checkpoint inhibitor, or any other suitable anti-cancer agent. Exemplary immune checkpoint inhibitors include anti-PD-1, anti-PD-L1, anti GITR, anti-OX-40, anti-LAG3, anti-TIM-3, anti-41BB, anti-CTLA-4 antibodies. Exemplary anti-neoplastic agents can include, for example, anti-microtubule agents, platinum coordination complexes, alkylating agents, topoisomerase II inhibitors, topoisomerase I inhibitors, antimetabolites, antibiotic agents, hormones and hormonal analogs, signal transduction pathway inhibitors, non-receptor tyrosine kinase angiogenesis inhibitors, proteasome inhibitors, and inhibitors of cancer metabolism. Other anti-cancer agents can include one or more of an immuno-stimulant, an antibody or fragment thereof (e.g., an anti-CD20, anti-HER2, anti-CD52, or anti-VEGF antibody or fragment thereof), or an immunotoxin (e.g., an anti-CD33 antibody or fragment thereof, an anti-CD22 antibody or fragment thereof, a calicheamicin conjugate, or a pseudomonas exotoxin conjugate).

ATF4-mediated expression of CHOP has also been shown to regulate the function and accumulation of myeloid-derived suppressor cells (MDSCs) in tumors. MDSCs in tumors reduce the ability to prime T cell function and reduce antitumoral or anticancer responses. Certain immunotherapeutic agents (such as anti-PD-1, anti PD-L1, anti-GITR, anti-OX-40, anti-LAG3, anti-TIM-3, anti-41BB, or anti-CTLA-4 antibodies) have been used to boost the immune response against cancer. ATF4-mediated expression of AXL has been associated with poor response to anti-PD1 therapy in melanoma. In some embodiments, an effective amount of an ISR inhibitor compound as described herein is administered to an individual with cancer to increase sensitivity to one or more immunotherapeutic agents. In some embodiments, there is a method of treating a refractory cancer (such as a melanoma) in an individual, comprising administering to the individual an effective amount of a compound described herein in combination with an effective amount of an immunotherapeutic agent (e.g. anti-PD-1, anti PD-L1, anti-GITR, anti-OX-40, anti-LAG3, anti-TIM-3, anti-41BB, or anti-CTLA-4 antibodies). In some embodiments, the refractory cancer is melanoma.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

### Articles of Manufacture and Kits

The present disclosure further provides articles of manufacture comprising a compound described herein or a salt thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein or a salt thereof. The kits may be used for any one or more of the uses described herein, and, accordingly, may contain instructions for the treatment of any disease or described herein, for example for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High-Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate.

Chromatography, recrystallization and other conventional separation procedures may also be used with intermediates or final products where it is desired to obtain a particular isomer of a compound or to otherwise purify a product of a reaction.

General methods of preparing compounds according to the present disclosure are depicted in the schemes below.

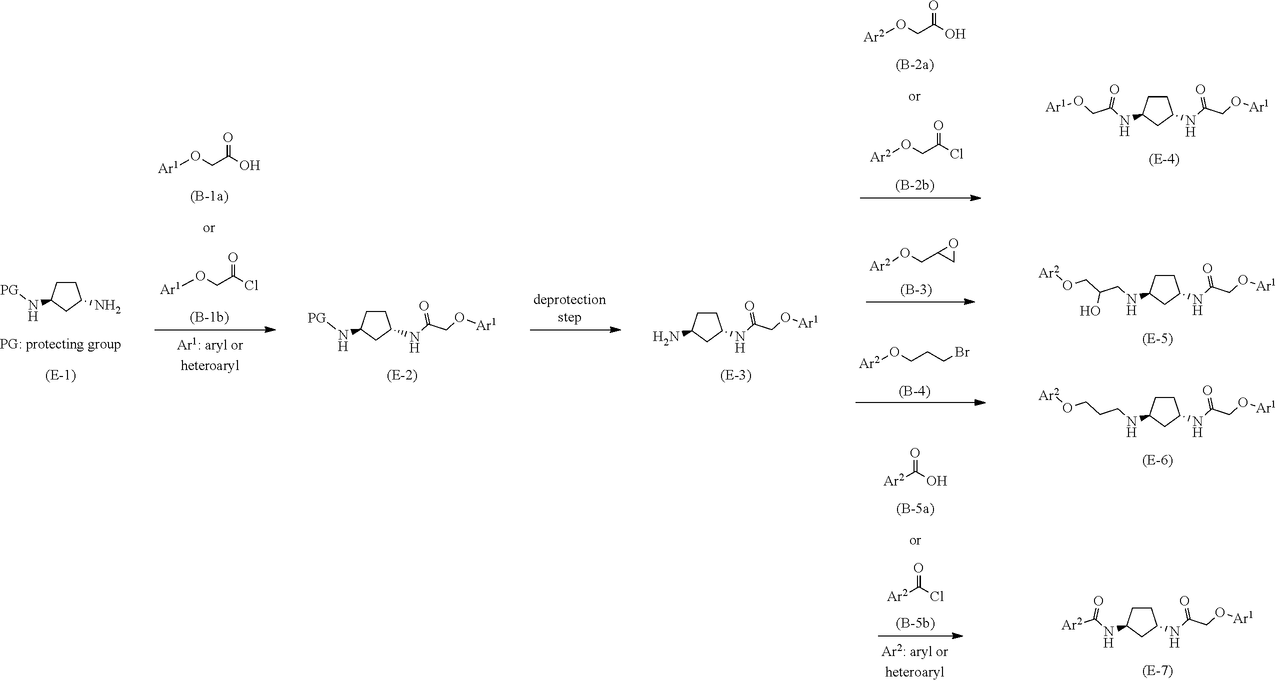

Compounds disclosed herein, such as compounds of formula (E-4), (E-5), (E-6), and (E-7), for example, can be synthesized according to the general method described in the scheme above. A compound of formula (E-1) is reacted with a carboxylic acid (B-1a), or a carboxylic acid derivative (e.g. an acyl chloride of formula (B-1b)), under suitable conditions to give a compound of formula (E-2). The compound of formula (E-2) is deprotected to give a compound of formula (E-3). The compound of formula (E-3) is reacted with a carboxylic acid (B-2a), or a carboxylic acid derivative (e.g. an acyl chloride of formula (B-2b), to give a compound of formula (E-4). The compound of formula (E-3) is reacted with an oxirane derivative of formula (B-3) to give a compound of formula (E-5). The compound of formula (E-3) is reacted with a haloalkyl derivative, such as a bromoalkyl compound of formula (B-4), to give a compound of formula (E-6). The compound of formula (E-3) is reacted with a carboxylic acid (B-5a), or a carboxylic acid derivative (e.g. an acyl chloride of formula (B-5b)), to give a compound of formula (E-7).

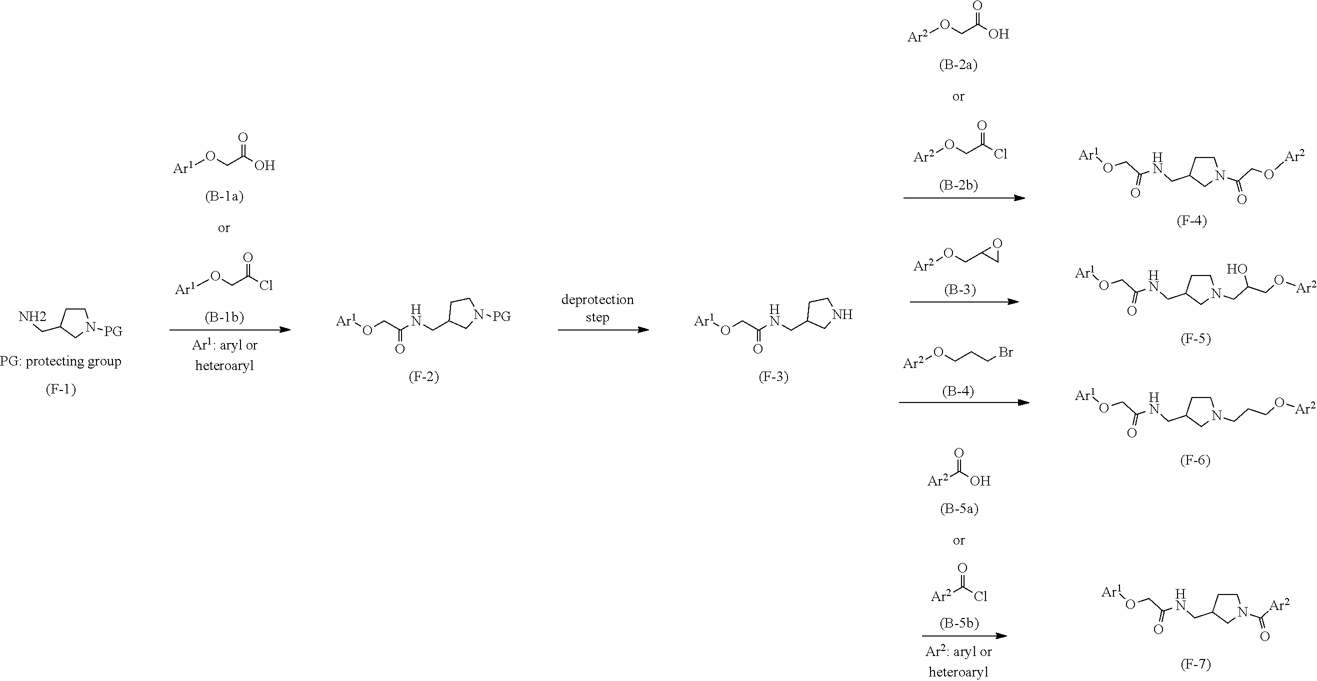

Compounds disclosed herein, such as compounds of formula (F-4), (F-5), (F-6), and (F-7), for example, can be synthesized according to the general method described in the scheme above. A compound of formula (F-1) is reacted with a carboxylic acid (B-1a), or a carboxylic acid derivative (e.g. an acyl chloride of formula (B-1b)), under suitable conditions to give a compound of formula (F-2). The compound of formula (F-2) is deprotected to give a compound of formula (F-3). The compound of formula (F-3) is reacted with a carboxylic acid (B-2a), or a carboxylic acid derivative (e.g. an acyl chloride of formula (B-2b), to give a compound of formula (F-4). The compound of formula (F-3) is reacted with an oxirane derivative of formula (B-3) to give a compound of formula (F-5). The compound of formula (F-3) is reacted with a haloalkyl derivative, such as a bromoalkyl compound of formula (B-4), to give a compound of formula (F-6). The compound of formula (F-3) is reacted with a carboxylic acid (B-5a), or a carboxylic acid derivative (e.g. an acyl chloride of formula (B-5b)), to give a compound of formula (F-7).

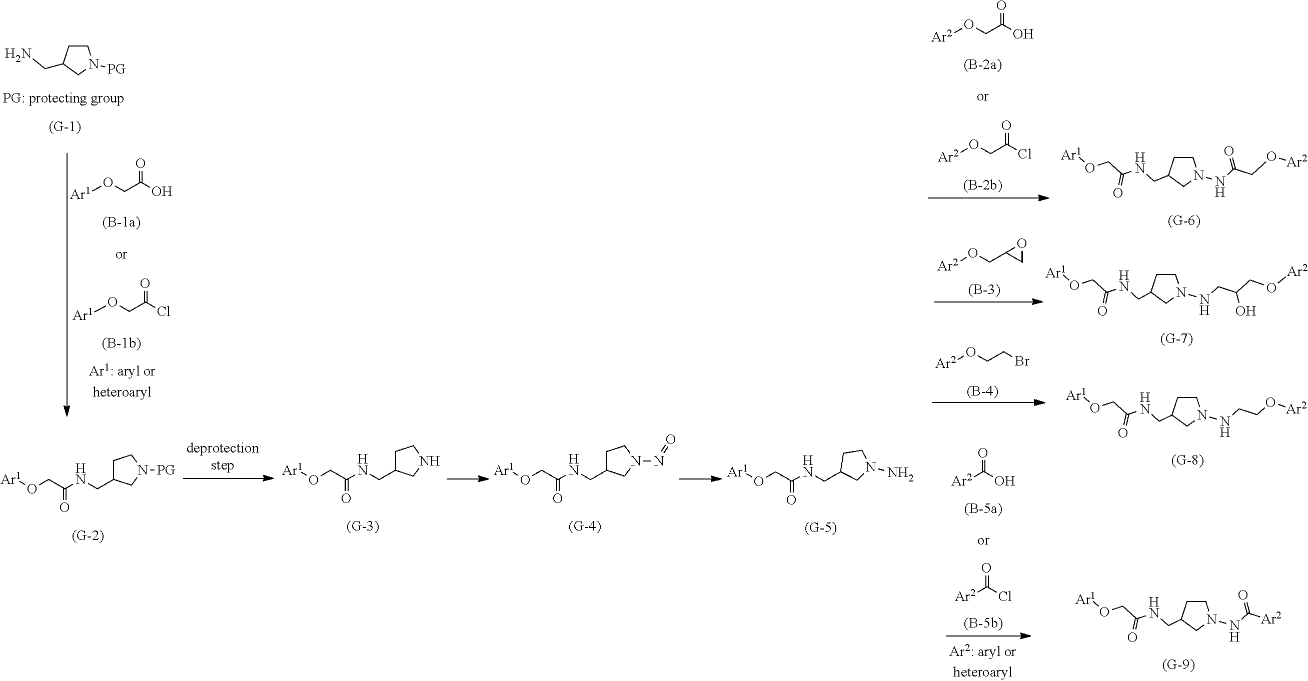

Compounds disclosed herein, such as compounds of formula (G-6), (G-7), (G-8), and (G-9), for example, can be synthesized according to the general method described in the scheme above. A compound of formula (G-1) is reacted with a carboxylic acid (B-1a), or a carboxylic acid derivative (e.g. an acyl chloride of formula (B-1b)), under suitable conditions to give a compound of formula (G-2). The compound of formula (G-2) is deprotected to give a compound of formula (G-3). The compound of formula (G-3) is subjected to nitrosation conditions (e.g. reacted with sodium nitrite) under suitable conditions to give a compound of formula (G-4). The compound of formula (G-4) is reduced (e.g. with Zn dust) under suitable conditions to give a compound of formula (G-5). The compound of formula (G-5) is reacted with a carboxylic acid (B-2a), or a carboxylic acid derivative (e.g. an acyl chloride of formula (B-2b), to give a compound of formula (G-6). The compound of formula (G-5) is reacted with an oxirane derivative of formula (B-3) to give a compound of formula (G-7). The compound of formula (G-5) is reacted with a haloalkyl derivative, such as a bromoalkyl compound of formula (B-4), to give a compound of formula (G-8). The compound of formula (G-5) is reacted with a carboxylic acid (B-5a), or a carboxylic acid derivative (e.g. an acyl chloride of formula (B-5b)), to give a compound of formula (G-9).

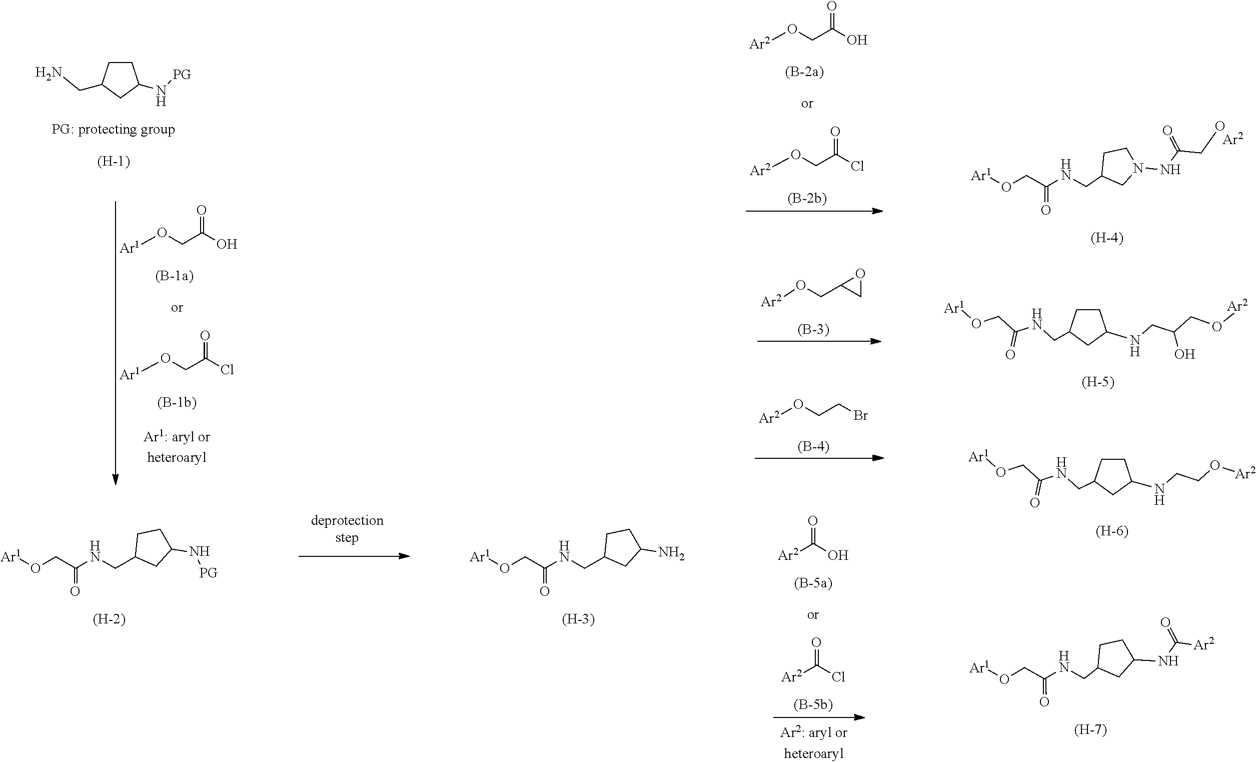

Compounds disclosed herein, such as compounds of formula (H-4), (H-5), (H-6), and (H-7), for example, can be synthesized according to the general method described in the scheme above. A compound of formula (H-1) is reacted with a carboxylic acid (B-1a), or a carboxylic acid derivative (e.g. an acyl chloride of formula (B-1b)), under suitable conditions to give a compound of formula (H-2). The compound of formula (H-2) is deprotected to give a compound of formula (H-3). The compound of formula (H-3) is reacted with a carboxylic acid (B-2a), or a carboxylic acid derivative (e.g. an acyl chloride of formula (B-2b), to give a compound of formula (H-4). The compound of formula (H-3) is reacted with an oxirane derivative of formula (B-3) to give a compound of formula (H-5). The compound of formula (H-3) is reacted with a haloalkyl derivative, such as a bromoalkyl compound of formula (B-4), to give a compound of formula (H-6). The compound of formula (H-3) is reacted with a carboxylic acid (B-5a), or a carboxylic acid derivative (e.g. an acyl chloride of formula (B-5b)), to give a compound of formula (H-7).

### ENUMERATED EMBODIMENTS

### Embodiment 1

A compound of formula (I)

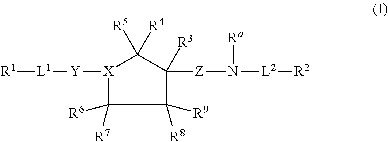

or a pharmaceutically acceptable salt thereof,
wherein:
X is N or CR¹²;
Y is a bond, NR^{a}, or NR^{a}NR^{a}; provided that:
   (a) when X is N, then Y is a bond or NR^{a}; and
   (b) when X is CR¹², then Y is NR^{a} or NR^{a}NR^{a};
Z is a bond, C(═O), CR¹⁰R¹¹, or NR^{a};
L¹ is selected from the group consisting of *1-C(═O)-#1, *1-CH₂-#1, *1-CH₂CH₂-#1, *1-CH₂CH₂CH₂-#1, *1-OCH₂C(═O)-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1;
   wherein *1 represents the attachment point to R¹ and #1 represents the attachment point to the remainder of the molecule;
L² is selected from the group consisting of #2-C(═O)-*2, #2-CH₂-*2, #2-CH₂CH₂-*2, #2-CH₂CH₂CH₂-*2, #2-C(═O)CH₂O—*2, #2-C(═O)CH₂CH₂O—*2, #2-C(═O)CH₂CH₂CH₂O-*2, #2-CH₂CH(OH)CH₂O—*2, #2-CH₂O-*2, #2-CH₂CH₂O-*2, and #2-CH₂CH₂CH₂O-*2;
   wherein *2 represents the attachment point to R² and #2 represents the attachment point to the remainder of the molecule;
R¹ is selected from the group consisting of:
   C₆-C₁₄ aryl substituted with one or more halo groups and optionally substituted with one or more R^{b}; and
   5-14 membered heteroaryl substituted with one or more halo groups and optionally substituted with one or more R^{b};
R² is selected from the group consisting of:
   C₆-C₁₄ aryl substituted with one or more halo groups and optionally substituted one or more R^{b}; and
   5-14 membered heteroaryl substituted with one or more halo groups and optionally substituted one or more R^{b};
R³ is hydrogen, halogen, or C₁-C₆ alkyl; or R³ and R¹² are taken together to form a CR¹³R¹⁴ group;
R⁴, R⁵, R⁶, R⁷, R⁸, and R⁹, independently of each other, are selected from the group consisting of hydrogen, halogen, and C₁-C₆ alkyl;
R¹⁰ and R¹¹, independently of each other, are selected from the group consisting of hydrogen, halogen, and C₁-C₆ alkyl;
R¹² is hydrogen, halogen, or C₁-C₆ alkyl; or R³ and R¹² are taken together to form a CR¹³R¹⁴ group;
R¹³ and R¹⁴, independently of each other, are selected from the group consisting of hydrogen, halogen, and C₁-C₆ alkyl;
R^{a}, independently at each occurrence, is hydrogen or C₁-C₆ alkyl;
R^{b}, independently at each occurrence, is selected from the group consisting of NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{c}R^{d}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{14-a}R^{14-b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{c}R^{d}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl),
   wherein R^{c} and R^{d} are taken together with the nitrogen atom to which they are attached to form a 3-10 membered heterocycle; and
   provided that:
   (i) when X is CR¹², Y is NR^{a}, Z is a bond, L¹ is *1-CH₂-#1, and L² is #2-CH₂-*2; then either:
      (i-1) at least one of R³, R⁴, and R⁵ is hydrogen or halogen; or
      (i-2) R³ and R¹² are taken together to form a CR¹³R¹⁴ group;
   (ii) when X is CR¹², Y is NR^{a}, Z is a bond, and R³ and R¹² are taken together to form CR¹³R¹⁴; then either:
      (ii-1) L¹ is selected from the group consisting of *1-C(═O)-#1, *1-CH₂-#1, *1-CH₂CH₂-#1, *1-CH₂CH₂CH₂-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1; or
      (ii-2) L² is selected from the group consisting of #2-C(═O)-*2, #2-CH₂-*2, #2-CH₂CH₂-*2, #2-CH₂CH₂CH₂-*2, #2-C(═O)CH₂CH₂O—*2, #2-C(═O)CH₂CH₂CH₂O-*2, #2-CH₂CH(OH)CH₂O—*2, #2-CH₂O-*2, #2-CH₂CH₂O-*2, and #2-CH₂CH₂CH₂O-*2; and
   (iii) when X is N and Y is a bond; then:
      L¹ is selected from the group consisting of *1-OCH₂CH(OH)CH₂-#1, *1-OCH₂-#1, *1-OCH₂CH₂-#1, and *1-OCH₂CH₂CH₂-#1; and
         further provided that, when Z is CR¹⁰R¹¹, then at least one of R¹ and R² is substituted by two or more halo groups.

### Embodiment 2

The compound of embodiment 1, or a pharmaceutically acceptable salt thereof, wherein the compound of formula (I) is a compound of formula (II)

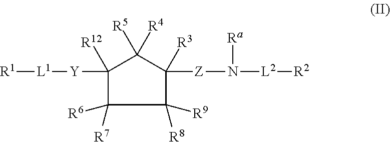

provided that:
(i) when Y is NR^{a}, Z is a bond, L¹ is *1-CH₂-#1, and L² is #2-CH₂-*2; then either:
   (i-1) at least one of R³, R⁴, and R⁵ is hydrogen or halogen; or
   (i-2) R³ and R¹² are taken together to form a CR¹³R¹⁴ group; and
(ii) when Y is NR^{a}, Z is a bond, and R³ and R¹² are taken together to form CR¹³R¹⁴; then either:
   (ii-1) L¹ is selected from the group consisting of *1-C(═O)-#1, *1-CH₂-#1, *1-CH₂CH₂-#1, *1-CH₂CH₂CH₂-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1; or
   (ii-2) L² is selected from the group consisting of #2-C(═O)-*2, #2-CH₂-*2, #2-CH₂CH₂-*2, #2-CH₂CH₂CH₂-*2, #2-C(═O)CH₂CH₂O—*2, #2-C(═O)CH₂CH₂CH₂O-*2, #2-CH₂CH(OH)CH₂O—*2, #2-CH₂O-*2, #2-CH₂CH₂O-*2, and #2-CH₂CH₂CH₂O-*2.

### Embodiment 3

The compound of embodiment 2, or a pharmaceutically acceptable salt thereof, wherein the compound of formula (II) is a compound of formula (IV)

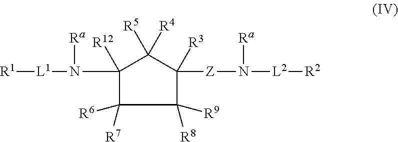

provided that:
(i) when Z is a bond, L¹ is *1-CH₂-#1, and L² is #2-CH₂-*2; then either:
   (i-1) at least one of R³, R⁴, and R⁵ is hydrogen or halogen; or
   (i-2) R³ and R¹² are taken together to form a CR¹³R¹⁴ group; and
(ii) when Z is a bond, and R³ and R¹² are taken together to form CR¹³R¹⁴; then either:
   (ii-1) L¹ is selected from the group consisting of *1-C(═O)-#1, *1-CH₂-#1, *1-CH₂CH₂-#1, *1-CH₂CH₂CH₂-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1; or
   (ii-2) L² is selected from the group consisting of #2-C(═O)-*2, #2-CH₂-*2, #2-CH₂CH₂-*2, #2-CH₂CH₂CH₂-*2, #2-C(═O)CH₂CH₂O—*2, #2-C(═O)CH₂CH₂CH₂O-*2, #2-CH₂CH(OH)CH₂O—*2, #2-CH₂O-*2, #2-CH₂CH₂O-*2, and #2-CH₂CH₂CH₂O-*2.

### Embodiment 4

The compound of embodiment 3, or a pharmaceutically acceptable salt thereof, wherein the compound of formula (IV) is a compound of formula (IV-a)

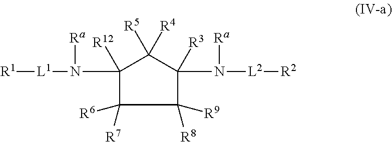

wherein:
R³ is hydrogen, halogen, or C₁-C₆ alkyl;
R¹² is hydrogen, halogen, or C₁-C₆ alkyl; and
provided that when L¹ is *1-CH₂-#1, and L² is #2-CH₂-*2; then at least one of R³, R⁴, and R⁵ is hydrogen or halogen.

### Embodiment 5

The compound of embodiment 3, or a pharmaceutically acceptable salt thereof, wherein the compound of formula (IV) is a compound of formula (IV-b)

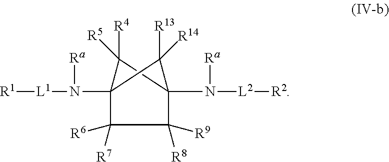

### Embodiment 6

The compound of embodiment 3, or a pharmaceutically acceptable salt thereof, wherein the compound of formula (IV) is a compound of formula (IV-c)

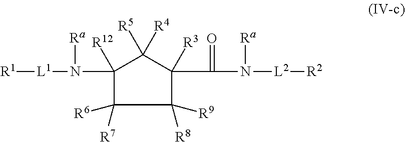

wherein:
R³ is hydrogen, halogen, or C₁-C₆ alkyl; and
R¹² is hydrogen, halogen, or C₁-C₆ alkyl.

### Embodiment 7

The compound of embodiment 3, or a pharmaceutically acceptable salt thereof, wherein the compound of formula (IV) is a compound of formula (IV-d)

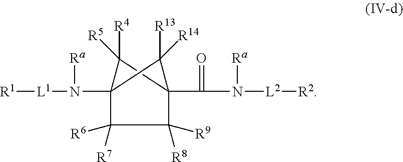

### Embodiment 8

The compound of embodiment 3, or a pharmaceutically acceptable salt thereof, wherein the compound of formula (IV) is a compound of formula (IV-e)

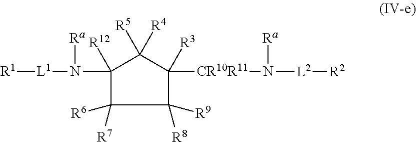

wherein:
R³ is hydrogen, halogen, or C₁-C₆ alkyl; and
R¹² is hydrogen, halogen, or C₁-C₆ alkyl.

### Embodiment 9

The compound of embodiment 3, or a pharmaceutically acceptable salt thereof, wherein the compound of formula (IV) is a compound of formula (IV-f)

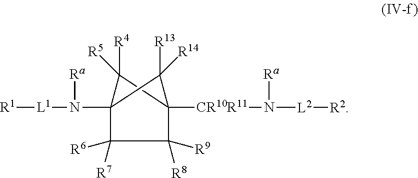

### Embodiment 10

The compound of embodiment 3, or a pharmaceutically acceptable salt thereof, wherein the compound of formula (IV) is a compound of formula (IV-g)

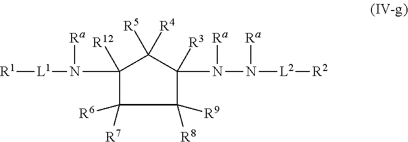

wherein:
R³ is hydrogen, halogen, or C₁-C₆ alkyl; and
R¹² is hydrogen, halogen, or C₁-C₆ alkyl.

### Embodiment 11

The compound of embodiment 3, or a pharmaceutically acceptable salt thereof, wherein the compound of formula (IV) is a compound of formula (IV-h)

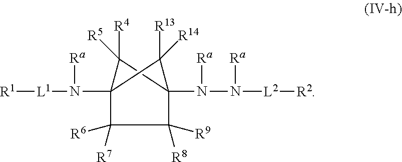

### Embodiment 12

The compound of embodiment 2, or a pharmaceutically acceptable salt thereof, wherein the compound of formula (II) is a compound of formula (V)

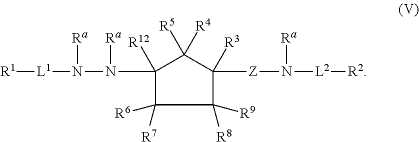

### Embodiment 13

The compound of embodiment 12, or a pharmaceutically acceptable salt thereof, wherein the compound of formula (V) is a compound of formula (V-a)

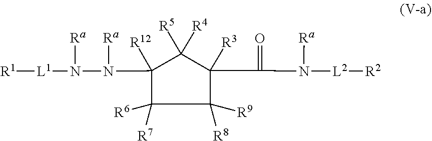

wherein:
R³ is hydrogen, halogen, or C₁-C₆ alkyl; and
R¹² is hydrogen, halogen, or C₁-C₆ alkyl.

### Embodiment 14

The compound of embodiment 12, or a pharmaceutically acceptable salt thereof, wherein the compound of formula (V) is a compound of formula (V-b)

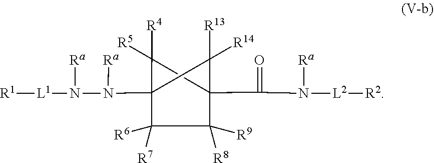

### Embodiment 15

The compound of embodiment 12, or a pharmaceutically acceptable salt thereof, wherein the compound of formula (V) is a compound of formula (V-c)

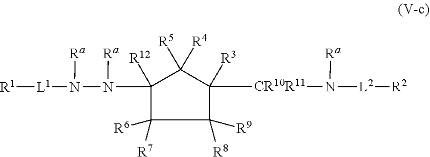

wherein:
R³ is hydrogen, halogen, or C₁-C₆ alkyl; and
R¹² is hydrogen, halogen, or C₁-C₆ alkyl.

### Embodiment 16

The compound of embodiment 12, or a pharmaceutically acceptable salt thereof, wherein the compound of formula (V) is a compound of formula (V-d)

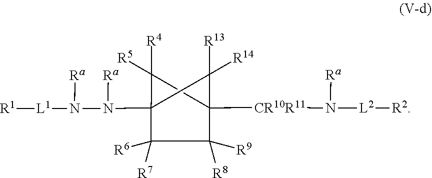

### Embodiment 17

The compound of embodiment 12, or a pharmaceutically acceptable salt thereof, wherein the compound of formula (V) is a compound of formula (V-e)

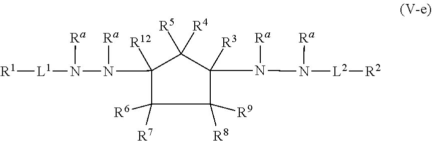

wherein:
R³ is hydrogen, halogen, or C₁-C₆ alkyl; and
R¹² is hydrogen, halogen, or C₁-C₆ alkyl.

### Embodiment 18

The compound of embodiment 12, or a pharmaceutically acceptable salt thereof, wherein the compound of formula (V) is a compound of formula (V-f)

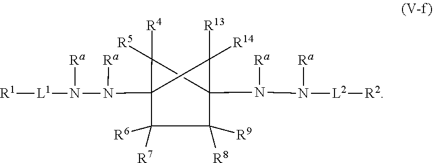

### Embodiment 19

The compound of embodiment 1, or a pharmaceutically acceptable salt thereof, wherein the compound of formula (I) is a compound of formula (III)

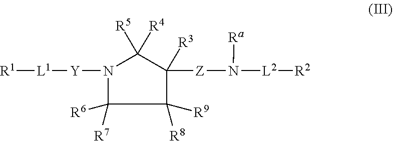

provided that when Y is a bond; then:
   L¹ is selected from the group consisting of *1-OCH₂CH(OH)CH₂-#1, *1-OCH₂-#1, *1-OCH₂CH₂-#1, and *1-OCH₂CH₂CH₂-#1; and
      further provided that, when Z is CR¹⁰R¹¹, then at least one of R¹ and R² is substituted by two or more halo groups.

### Embodiment 20

The compound of embodiment 19, or a pharmaceutically acceptable salt thereof, wherein the compound of formula (III) is a compound of formula (VI)

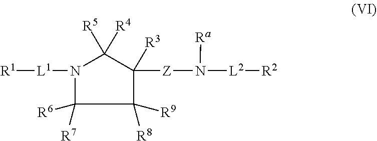

provided that L¹ is selected from the group consisting of *1-OCH₂CH(OH)CH₂-#1, *1-OCH₂-#1, *1-OCH₂CH₂-#1, and *1-OCH₂CH₂CH₂-#1; and
   further provided that, when Z is CR¹⁰R¹¹, then at least one of R¹ and R² is substituted by two or more halo groups.

### Embodiment 21

The compound of embodiment 20, or a pharmaceutically acceptable salt thereof, wherein the compound of formula (VI) is a compound of formula (VI-a)

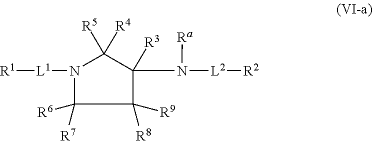

provided that L¹ is selected from the group consisting of *1-OCH₂CH(OH)CH₂-#1, *1-OCH₂-#1, *1-OCH₂CH₂-#1, and *1-OCH₂CH₂CH₂-#1.

### Embodiment 22

The compound of embodiment 20, or a pharmaceutically acceptable salt thereof, wherein the compound of formula (VI) is a compound of formula (VI-b)

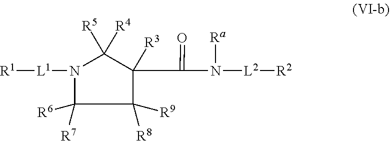

provided that L¹ is selected from the group consisting of *1-OCH₂CH(OH)CH₂-#1, *1-OCH₂-#1, *1-OCH₂CH₂-#1, and *1-OCH₂CH₂CH₂-#1.

### Embodiment 23

The compound of embodiment 20, or a pharmaceutically acceptable salt thereof, wherein the compound of formula (VI) is a compound of formula (VI-c)

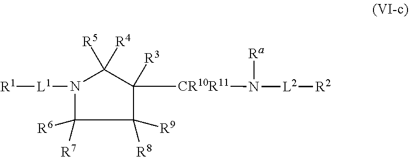

provided that L¹ is selected from the group consisting of *1-OCH₂CH(OH)CH₂-#1, *1-OCH₂-#1, *1-OCH₂CH₂-#1, and *1-OCH₂CH₂CH₂-#1; and at least one of R¹ and R² is substituted by two or more halo groups.

### Embodiment 24

The compound of embodiment 20, or a pharmaceutically acceptable salt thereof, wherein the compound of formula (VI) is a compound of formula (VI-d)

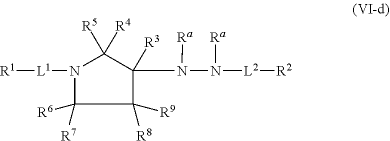

provided that L¹ is selected from the group consisting of *1-OCH₂CH(OH)CH₂-#1, *1-OCH₂-#1, *1-OCH₂CH₂-#1, and *1-OCH₂CH₂CH₂-#1.

### Embodiment 25

The compound of embodiment 19, or a pharmaceutically acceptable salt thereof, wherein the compound of formula (III) is a compound of formula (VII)

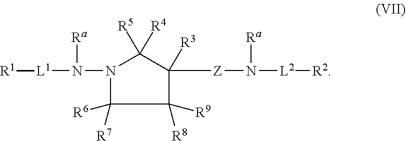

### Embodiment 26

The compound of embodiment 25, or a pharmaceutically acceptable salt thereof, wherein the compound of formula (VII) is a compound of formula (VII-a)

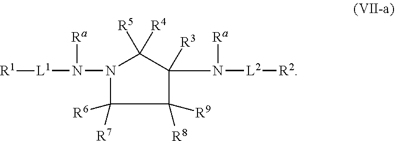

### Embodiment 27

The compound of embodiment 25, or a pharmaceutically acceptable salt thereof, wherein the compound of formula (VII) is a compound of formula (VII-b)

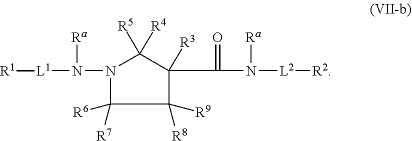

### Embodiment 28

The compound of embodiment 25, or a pharmaceutically acceptable salt thereof, wherein the compound of formula (VII) is a compound of formula (VII-c)

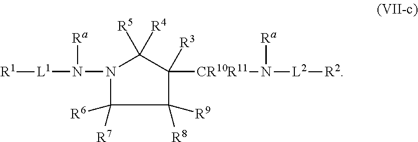

### Embodiment 29

The compound of embodiment 25, or a pharmaceutically acceptable salt thereof, wherein the compound of formula (VII) is a compound of formula (VII-d)

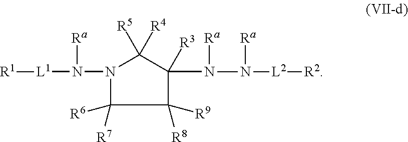

### Embodiment 30

A compound selected from the group consisting of a compound of Table 1, or a pharmaceutically acceptable salt thereof.

### Embodiment 31

A pharmaceutical composition comprising a compound of any of the preceding embodiments, or a pharmaceutically acceptable salt thereof, and a pharmaceutically acceptable carrier.

### Embodiment 32

A method of treating a disease or disorder mediated by an integrated stress response (ISR) pathway in an individual in need thereof comprising administering to the individual a therapeutically effective amount of a compound of any one of embodiments 1 to 30, or a pharmaceutically acceptable salt thereof, or a therapeutically effective amount of a pharmaceutical composition of embodiment 31.

### Embodiment 33

The method of embodiment 32, wherein the compound, the pharmaceutically acceptable salt, or the pharmaceutical composition is administered in combination with a therapeutically effective amount of one or more additional anti-cancer agents.

### Embodiment 34

The method of embodiment 32, wherein the disease or disorder is mediated by phosphorylation of eIF2α and/or the guanine nucleotide exchange factor (GEF) activity of eIF2B.

### Embodiment 35

The method of any one of embodiments 32-34, wherein the disease or disorder is mediated by a decrease in protein synthesis.

### Embodiment 36

The method of any one of embodiments 32-35, wherein the disease or disorder is mediated by the expression of ATF4, CHOP or BACE-1.

### Embodiment 37

The method of any of embodiments 32-36, wherein the disease or disorder is a neurodegenerative disease, an inflammatory disease, an autoimmune disease, a metabolic syndrome, a cancer, a vascular disease, an ocular disease, or a musculoskeletal disease.

### Embodiment 38

The method of embodiment 37, wherein the disease is vanishing white matter disease, childhood ataxia with CNS hypomyelination, intellectual disability syndrome, Alzheimer's disease, prion disease, Creutzfeldt-Jakob disease, Parkinson's disease, amyotrophic lateral sclerosis (ALS) disease, cognitive impairment, frontotemporal dementia (FTD), traumatic brain injury, postoperative cognitive dysfunction (PCD), neuro-otological syndromes, hearing loss, Huntington's disease, stroke, chronic traumatic encephalopathy, spinal cord injury, dementias or cognitive impairment, arthritis, psoriatic arthritis, psoriasis, juvenile idiopathic arthritis, asthma, allergic asthma, bronchial asthma, tuberculosis, chronic airway disorder, cystic fibrosis, glomerulonephritis, membranous nephropathy, sarcoidosis, vasculitis, ichthyosis, transplant rejection, interstitial cystitis, atopic dermatitis or inflammatory bowel disease, Crohn's disease, ulcerative colitis, celiac disease, systemic lupus erythematosus, type 1 diabetes, multiple sclerosis, rheumatoid arthritis, alcoholic liver steatosis, obesity, glucose intolerance, insulin resistance, hyperglycemia, fatty liver, dyslipidemia, hyperlipidemia, type 2 diabetes, pancreatic cancer, breast cancer, kidney cancer, bladder cancer, prostate cancer, testicular cancer, urothelial cancer, endometrial cancer, ovarian cancer, cervical cancer, renal cancer, esophageal cancer, gastrointestinal stromal tumor (GIST), multiple myeloma, cancer of secretory cells, thyroid cancer, gastrointestinal carcinoma, chronic myeloid leukemia, hepatocellular carcinoma, colon cancer, melanoma, malignant glioma, glioblastoma, glioblastoma multiforme, astrocytoma, dysplastic gangliocytoma of the cerebellum, Ewing's sarcoma, rhabdomyosarcoma, ependymoma, medulloblastoma, ductal adenocarcinoma, adenosquamous carcinoma, nephroblastoma, acinar cell carcinoma, lung cancer, non-Hodgkin's lymphoma, Burkitt's lymphoma, chronic lymphocytic leukemia, monoclonal gammopathy of undetermined significance (MGUS), plasmocytoma, lymphoplasmacytic lymphoma, acute lymphoblastic leukemia, Pelizaeus-Merzbacher disease, atherosclerosis, abdominal aortic aneurism, carotid artery disease, deep vein thrombosis, Buerger's disease, chronic venous hypertension, vascular calcification, telangiectasia or lymphoedema, glaucoma, age-related macular degeneration, inflammatory retinal disease, retinal vascular disease, diabetic retinopathy, uveitis, rosacea, Sjogren's syndrome or neovascularization in proliferative retinopathy, hyperhomocysteinemia, skeletal muscle atrophy, myopathy, muscular dystrophy, muscular wasting, sarcopenia, Duchenne muscular dystrophy (DMD), Becker's disease, myotonic dystrophy, X-linked dilated cardiomyopathy, or spinal muscular atrophy (SMA).

### Embodiment 39

A method of producing a protein, comprising contacting a eukaryotic cell comprising a nucleic acid encoding the protein with the compound or salt of any one of embodiments 1-30.

### Embodiment 40

The method of embodiment 39, comprising culturing the cell in an in vitro culture medium comprising the compound or salt.

### Embodiment 41

A method of culturing a eukaryotic cell comprising a nucleic acid encoding a protein, comprising contacting the eukaryotic cell with an in vitro culture medium comprising a compound or salt of any one of embodiments 1-30.

### Embodiment 42

The method of any one of embodiments 39-41, wherein the nucleic acid encoding the protein is a recombinant nucleic acid.

### Embodiment 43

The method of any one of embodiments 39-42, wherein the cell is a human embryonic kidney (HEK) cell or a Chinese hamster ovary (CHO) cell.

### Embodiment 44

A method of producing a protein, comprising contacting a cell-free protein synthesis (CFPS) system comprising eukaryotic initiation factor 2 (eIF2) and a nucleic acid encoding a protein with the compound or salt of any one of embodiments 1-30.

### Embodiment 45

The method of any one of embodiments 39-44, wherein the protein is an antibody or a fragment thereof.

### Embodiment 46

The method of any one of embodiments 39-45, comprising purifying the protein.

### Embodiment 47

An in vitro cell culture medium, comprising the compound or salt of any one of embodiments 1-30 and nutrients for cellular growth.

### Embodiment 48

The cell culture medium of embodiment 47, comprising a eukaryotic cell comprising a nucleic acid encoding a protein.

### Embodiment 49

The cell culture medium of embodiment 47 or 48, further comprising a compound for inducing protein expression.

### Embodiment 50

The cell culture medium of any one of embodiments 47-49, wherein the nucleic acid encoding the protein is a recombinant nucleic acid.

### Embodiment 51

The cell culture medium of any one of embodiments 47-50, wherein the protein is an antibody or a fragment thereof.

### Embodiment 52

The cell culture medium of any one of embodiments 47-51, wherein the eukaryotic cell is a human embryonic kidney (HEK) cell or a Chinese hamster ovary (CHO) cell.

### Embodiment 53

A cell-free protein synthesis (CFPS) system comprising eukaryotic initiation factor 2 (eIF2) and a nucleic acid encoding a protein with the compound or salt of any one of embodiments 1-30.

### Embodiment 54

The CFPS system of embodiment 53, comprising a eukaryotic cell extract comprising eIF2.

### Embodiment 55

The CFPS system of embodiment 53 or 54, further comprising eIF2B.

### Embodiment 56

The CFPS system of any one of embodiments 53-55, wherein the protein is an antibody or a fragment thereof.

### EXAMPLES

Although the invention has been described and illustrated with a certain degree of particularity, it is understood that the present disclosure has been made only by way of example, and that numerous changes in the combination and arrangement of parts can be resorted to by those skilled in the art without departing from the spirit and scope of the invention, as defined by the claims.

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

In some cases, stereoisomers are separated to give single enantiomers or diastereomers as single, unknown stereoisomers, and are arbitrarily drawn as single isomers. Where appropriate, information is given on separation method and elution time and order. In the biological examples, compounds tested were prepared in accordance to the synthetic procedures described therein. For any given compound of unknown absolute stereochemistry for which a stereochemistry has been arbitrarily assigned and for which a specific rotation and/or chiral HPLC elution time has been measured, biological data reported for that compound was obtained using the enantiomer or diastereoisomer associated with said specific rotation and/or chiral HPLC elution time.

In some cases, optical rotation was determined on Jasco DIP-360 digital polarimeter at a wavelength of 589 nm (sodium D line) and are reported as [at]D for a given temperature T (expressed in ° C.). Where appropriate, information is given on solvent and concentration (expressed as g/100 mL).

Abbreviations:
br. s. Broad singlet
chloroform-d Deuterated chloroform
methanol-d₄ Deuterated methanol
DIAD Diisopropyl azodicarboxylate
DCM Dichloromethane
DEA Diethylamine
DIPEA Diisopropylethylamine
DMF N,N-Dimethylformamide
DMSO-d₆ Deuterated dimethylsulfoxide
d Doublet
EDC.HCl 1-Ethyl-3-(3-dimethylaminopropyl)carbodiimide hydrochloric acid
EtOAc Ethyl acetate
EtOH Ethanol
g Gram
HATU (O-(7-azabenzotriazol-1-yl)-N,N,N′,N′-tetramethyluronium hexafluorophosphate)
HOBT Hydroxybenzotriazole
HPLC High Performance Liquid Chromatography
L Litre
LCMS Liquid Chromatography Mass Spectrometry
MeCN Acetonitrile
MeOH Methanol
mg Milligram
mL Millilitre
mmol Millimoles
m multiplet
NMR Nuclear Magnetic Resonance
q quartet
RT Room temperature
s singlet
SFC Supercritical Fluid Chromatography
TFA trifluoroacetic acid
THF Tetrahydrofuran
TLC Thin layer chromatography
t triplet

### Example 1

### Synthesis of N,N′-((1S,3S)-cyclopentane-1,3-diyl)bis(2-(4-chloro-3-fluorophenoxy)acetamide)

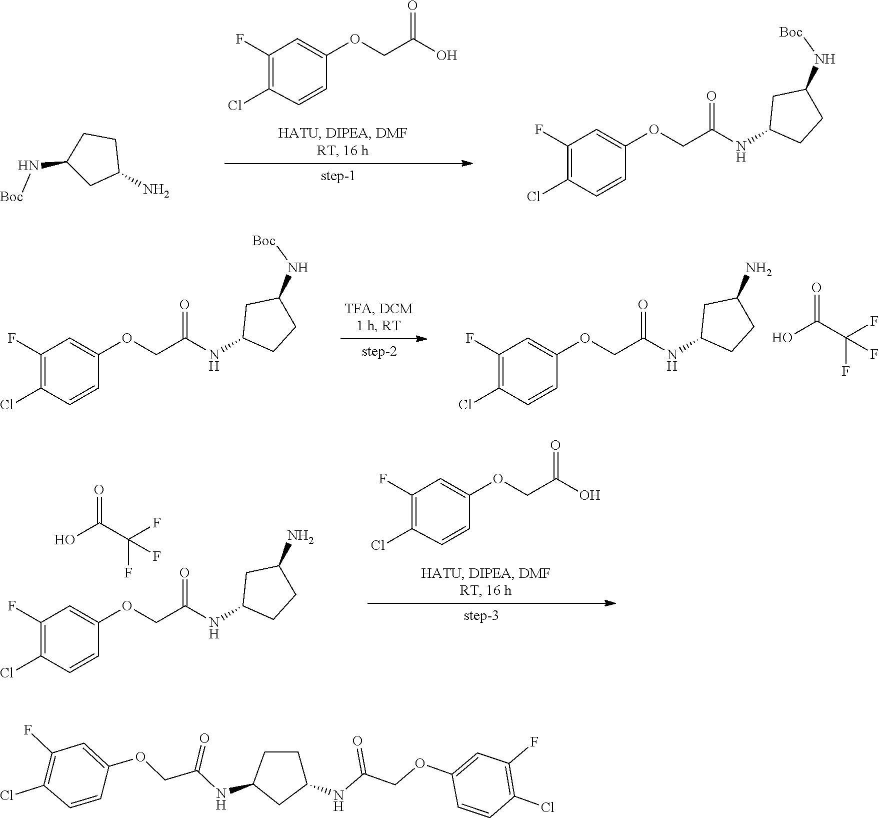

### Step-1: Synthesis of tert-butyl ((1S,3S)-3-(2-(4-chloro-3-fluorophenoxy)acetamido)cyclopentyl)carbamate

To a stirred solution of 2-(4-chloro-3-fluorophenoxy)acetic acid (1.000 g, 4.9 mmol, 1.0 equiv) in DMF (10 ml) was added tert-butyl ((1S,3S)-3-aminocyclopentyl)carbamate (0.900 g, 4.9 mmol, 1.2 equiv), HATU (2.793 g, 7.35 mmol, 1.5 equiv) and DIPEA (1.265 g, 9.8 mmol, 2.0 equiv). The reaction mixture was allowed to stir at RT for 16 h. Product formation was confirmed by LCMS. The reaction mixture was diluted with water and a precipitate was formed. The resulting solid precipitate was filtered off and washed with ice water, dried under vacuum to obtain tert-butyl ((1S,3S)-3-(2-(4-chloro-3-fluorophenoxy)acetamido)cyclopentyl)carbamate (0.700 g, 36% Yield ) as an off-white solid. LCMS 387.1 [M+H]⁺, ¹H NMR (400 MHz, DMSO-d₆) δ 8.07 (d, J=7.9 Hz, 1H), 7.49 (t, J=8.8 Hz, 1H), 7.06 (dd, J=2.6, 11.4 Hz, 1H), 6.97-6.76 (m, 2H), 4.48 (s, 2H), 4.24-4.08 (m, 1H), 3.90 (d, J=6.6 Hz, 1H), 1.92 (dd, J=5.5, 9.4 Hz, 2H), 1.68 (t, J=7.0 Hz, 2H), 1.37 (s, 8H).

### Step-2: Synthesis of N-((1S,3S)-3-aminocyclopentyl)-2-(4-chloro-3-fluorophenoxy)acetamide 2,2,2-trifluoroacetate

To a stirred solution of tert-butyl ((1S,3S)-3-(2-(4-chloro-3-fluorophenoxy)acetamido)cyclopentyl)carbamate (0.700 g, 1.81 mmol, 1.0 equiv) in DCM (15 ml) was added TFA (2 ml). The reaction mixture was allowed to stir at RT for 16 h. Reaction progress was monitored by NMR. After completion of reaction the reaction mixture was concentrated under reduced pressure. Crude compound was crystallized by trituration in pentane and then in petroleum ether to obtain N-((1S,3S)-3-aminocyclopentyl)-2-(4-chloro-3-fluorophenoxy)acetamide 2,2,2-trifluoroacetate (900 mg, quantitative yield). LCMS 287.1 [M+H]⁺, ¹H NMR (400 MHz, DMSO-d₆) δ 8.17 (d, J=7.5 Hz, 1H), 7.79 (br. s., 2H), 7.50 (t, J=8.8 Hz, 1H), 7.06 (dd, J=2.6, 11.4 Hz, 1H), 6.84 (d, J=7.0 Hz, 1H), 4.51 (s, 2H), 4.30 (d, J=7.5 Hz, 1H), 3.62 (br. s., 1H), 2.07 (br. s., 1H), 2.00 (d, J=6.1 Hz, 1H), 1.92-1.73 (m, 2H), 1.52 (d, J=12.3 Hz, 2H).

### Step-3: Synthesis of N,N′-((1S,3S)-cyclopentane-1,3-diyl)bis(2-(4-chloro-3-fluorophenoxy)acetamide)

To a stirred solution of 2-(4-chloro-3-fluorophenoxy)acetic acid (0.128 g, 0.63 mmol, 1.0 equiv) in DMF (4 ml) were added N-((1S,3S)-3-aminocyclopentyl)-2-(4-chloro-3-fluorophenoxy)acetamide 2,2,2-trifluoroacetate (0.304 g, 0.76 mmol, 1.2 equiv), HATU (0.361 g, 0.95 mmol, 1.5 equiv) and DIPEA (0.163 g, 1.26 mmol, 2.0 equiv). The reaction mixture was allowed to stir at RT for 16 h. Product formation was confirmed by LCMS. The reaction mixture was diluted with water and a precipitate was formed. The resulting solid precipitate was filtered and washed with ice water, dried under vacuum to obtain N,N′-((1S,3S)-cyclopentane-1,3-diyl)bis(2-(4-chloro-3-fluorophenoxy)acetamide) (Compound 1—0.170 g, 58% Yield) as an off-white solid. LCMS 473.1 [M+H]⁺, ¹H NMR (400 MHz, DMSO-d₆) δ 8.13 (d, J=7.7 Hz, 1H), 7.49 (t, J=8.8 Hz, 1H), 7.06 (dd, J=11.5, 2.9 Hz, 1H), 6.84 (dd, J=9.0, 2.9 Hz, 1H), 4.49 (s, 2H), 4.25 (p, J=6.9 Hz, 1H), 1.98 (p, J=9.6 Hz, 1H), 1.75 (t, J=7.1 Hz, 1H), 1.44 (q, J=9.2, 7.9 Hz, 1H).

### Example 2

### Synthesis of 5-chloro-N-((1S,3S)-3-(2-(4-chloro-3-fluorophenoxy)acetamido)cyclopentyl)benzofuran-2-carboxamide

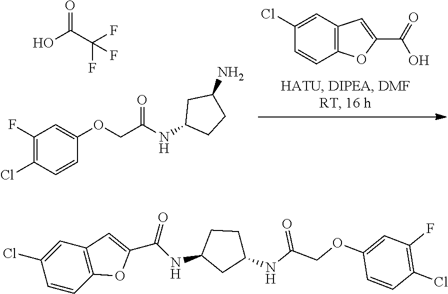 [SMILES: O=C(O)C(F)(F)F ; O=C(O)c1cc2cc(Cl)ccc2o1 ; N[C@H]1CC[C@H](NC(=O)COc2ccc(Cl)c(F)c2)C1 ; O=C(COc1ccc(Cl)c(F)c1)N[C@H]1CC[C@H](NC(=O)c2cc3cc(Cl)ccc3o2)C1]

To a stirred solution of 5-chlorobenzofuran-2-carboxylic acid (0.124 g, 0.63 mmol, 1.0 equiv) in DMF (4 ml) was added N-((1S,3S)-3-aminocyclopentyl)-2-(4-chloro-3-fluorophenoxy)acetamide 2,2,2-trifluoroacetate (0.304 g, 0.76 mmol, 1.2 equiv), HATU (0.361 g, 0.95 mmol, 1.5 equiv) and DIPEA (0.163 g, 1.26 mmol, 2.0 equiv). The reaction mixture was allowed to stir at RT for 16 h. Product formation was confirmed by LCMS. The reaction mixture was diluted with water and a precipitate was formed. The resulting solid precipitate was filtered and washed with ice water, dried under vacuum to obtain 5-chloro-N-((1S,3S)-3-(2-(4-chloro-3-fluorophenoxy)acetamido)cyclopentyl)benzofuran-2-carboxamide (Compound 14—0.070 g, 24% Yield) as an off-white solid. LCMS 464.1 [M+H]⁺, ¹H NMR (400 MHz, DMSO-d₆) δ 8.77 (d, J=7.6 Hz, 1H), 8.17 (d, J=7.5 Hz, 1H), 7.88 (d, J=2.3 Hz, 1H), 7.70 (d, J=8.8 Hz, 1H), 7.53 (s, 1H), 7.52-7.45 (m, 2H), 7.08 (dd, J=11.4, 2.9 Hz, 1H), 6.86 (dd, J=9.0, 2.9 Hz, 1H), 4.52 (s, 2H), 4.44 (p, J=7.3 Hz, 1H), 4.33 (h, J=6.9 Hz, 1H), 2.06 (tp, J=12.6, 7.3, 6.1 Hz, 2H), 1.87 (qt, J=13.3, 6.8 Hz, 2H), 1.66-1.42 (m, 2H).

### Example 3

### Synthesis of 6-chloro-N-((1S,3S)-3-(2-(4-chloro-3-fluorophenoxy)acetamido)cyclopentyl)quinoline-2-carboxamide

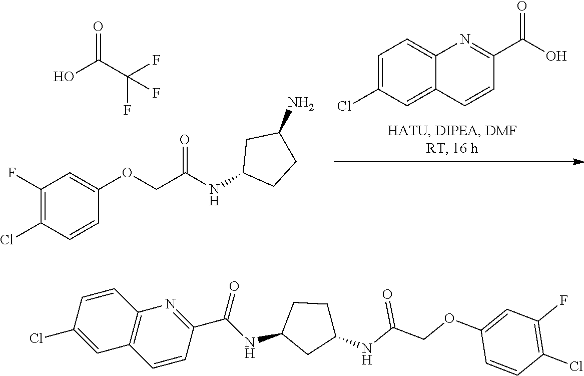 [SMILES: N[C@H]1CC[C@H](NC(=O)COc2ccc(Cl)c(F)c2)C1 ; O=C(O)C(F)(F)F ; O=C(O)c1ccc2cc(Cl)ccc2n1 ; O=C(COc1ccc(Cl)c(F)c1)N[C@H]1CC[C@H](NC(=O)c2ccc3cc(Cl)ccc3n2)C1]

To a stirred solution of 6-chloroquinoline-2-carboxylic acid (0.200 g, 0.97 mmol, 1.0 equiv) in DMF (6 ml) was added N-((1S,3S)-3-aminocyclopentyl)-2-(4-chloro-3-fluorophenoxy)acetamide 2,2,2-trifluoroacetate (0.466 g, 1.16 mmol, 1.2 equiv), HATU (0.570 g, 1.5 mmol, 1.5 equiv) and DIPEA (0.250 g, 1.94 mmol, 2.0 equiv). The reaction mixture was allowed to stir at RT for 16 h. Product formation was confirmed by LCMS. The reaction mixture was diluted with water (20 mL) and extracted with ethyl acetate (30 mL×3). Combined organic layer was washed with water (30 mL×6). Organic layer was dried over anhydrous sodium sulfate, filtered and concentrated under reduced pressure. Crude material was purified by reversed phase HPLC to obtain pure 6-chloro-N-((1S,3S)-3-(2-(4-chloro-3-fluorophenoxy)acetamido)cyclopentyl)quinoline-2-carboxamide (Compound 16—0.260 g, 59% Yield) as a white solid. LCMS 476.2 [M+H]⁺, ¹H NMR (400 MHz, DMSO-d₆) δ 8.86 (d, J=8.2 Hz, 1H), 8.53 (d, J=8.6 Hz, 1H), 8.24 (d, J=2.4 Hz, 1H), 8.18 (t, J=8.6 Hz, 3H), 7.88 (dd, J=9.1, 2.4 Hz, 1H), 7.50 (t, J=8.9 Hz, 1H), 7.08 (dd, J=11.4, 2.8 Hz, 1H), 6.90-6.82 (m, 1H), 4.52 (s, 3H), 4.36 (h, J=7.3 Hz, 1H), 2.16-1.82 (m, 4H), 1.68 (dq, J=13.0, 8.1 Hz, 1H), 1.59-1.45 (m, 1H).

### Example 4

### Synthesis of 5-chloro-N-((1S,3S)-3-((3-(4-chloro-3-fluorophenoxy)-2-hydroxypropyl)amino)cyclopentyl)benzofuran-2-carboxamide

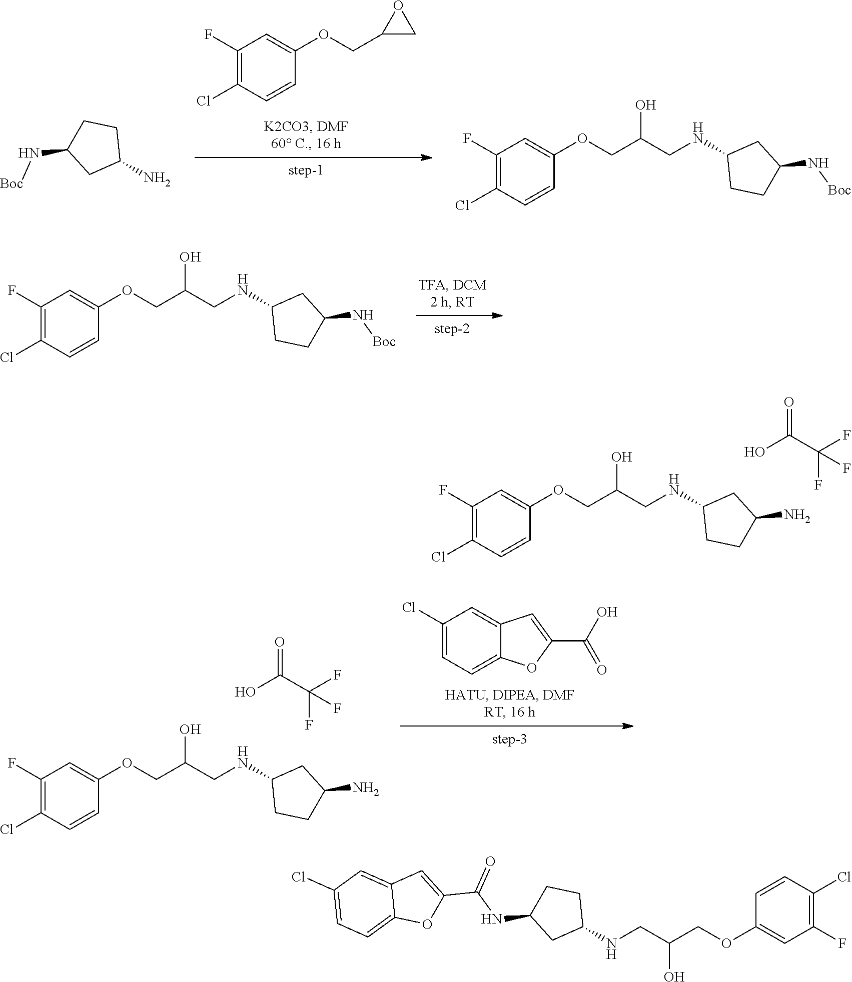

### Step-1: Synthesis of tert-butyl ((1S,3S)-3-((3-(4-chloro-3-fluorophenoxy)-2-hydroxypropyl)amino)cyclopentyl)carbamate

To a stirred solution of tert-butyl ((1S,3S)-3-aminocyclopentyl)carbamate (0.990 g, 4.95 mmol, 1.0 equiv) in DMF (10 ml) was added K₂CO₃ (1.366 g, 9.9 mmol, 2.0 equiv) followed by the addition of 2-((4-chloro-3-fluorophenoxy)methyl)oxirane (1.000 g, 4.95 mmol, 1.0 equiv). The reaction mixture was allowed to stir at 60° C. for 16 h. Product formation was confirmed by LCMS. The reaction mixture was diluted with water and a precipitate was formed. The resulting solid precipitate was filtered and washed with ice water, dried under vacuum to obtain tert-butyl ((1S,3S)-3-((3-(4-chloro-3-fluorophenoxy)-2-hydroxypropyl)amino)cyclopentyl)carbamate (1.100 g). LCMS 403.3 [M+H]⁺.

### Step-2: Synthesis of 1-(((1S,3S)-3-aminocyclopentyl)amino)-3-(4-chloro-3-fluorophenoxy)propan-2-ol 2,2,2-trifluoroacetate

To a stirred solution of tert-butyl ((1S,3S)-3-((3-(4-chloro-3-fluorophenoxy)-2-hydroxypropyl)amino)cyclopentyl)carbamate (1.100 g, 2.74 mmol, 1.0 equiv) in DCM (10 ml) was added TFA (2 ml). The reaction mixture was allowed to stir at RT for 16 h. Reaction progress was monitored by LCMS and NMR. After completion of reaction, reaction mixture was concentrated under reduced pressure. Crude compound was crystallized by trituration in pentane and then in petroleum ether to obtain pure 1-(((1S,3S)-3-aminocyclopentyl)amino)-3-(4-chloro-3-fluorophenoxy)propan-2-ol 2,2,2-trifluoroacetate (1.100 g, quantitative yield). LCMS 303.2 [M+H]⁺.

### Step-3: Synthesis of 5-chloro-N-((1S,3S)-3-((3-(4-chloro-3-fluorophenoxy)-2-hydroxypropyl)amino)cyclopentyl)benzofuran-2-carboxamide

To a stirred solution of 5-chlorobenzofuran-2-carboxylic acid (0.140 g, 0.7 mmol, 1.0 equiv) in DMF (4 ml) was added 1-(((1S,3S)-3-aminocyclopentyl)amino)-3-(4-chloro-3-fluorophenoxy)propan-2-ol 2,2,2-trifluoroacetate (0.350 g, 0.84 mmol, 1.2 equiv), HATU (0.399 g, 1.05 mmol, 1.5 equiv) and DIPEA (0.181 g, 1.4 mmol, 2.0 equiv). The reaction mixture was allowed to stir at RT for 16 h. Product formation was confirmed by LCMS. The reaction mixture was diluted with water (20 mL) and extracted with ethyl acetate (30 mL×3). Combined organic layer was washed with water (30 mL×6). Organic layer was dried over anhydrous sodium sulfate, filtered and concentrated under reduced pressure. Crude material was purified by reversed phase HPLC to obtain 5-chloro-N-((1S,3S)-3-((3-(4-chloro-3-fluorophenoxy)-2-hydroxypropyl)amino)cyclopentyl)benzofuran-2-carboxamide (Compound 18—0.090 g, 40.36%) as an off-white solid. LCMS 481.2 [M+H]⁺, ¹H NMR (400 MHz, DMSO-d₆) δ 8.66 (d, J=7.7 Hz, 1H), 7.86 (d, J=2.3 Hz, 1H), 7.69 (d, J=8.8 Hz, 1H), 7.55-7.34 (m, 3H), 7.07 (dd, J=11.7, 2.9 Hz, 1H), 6.84 (dd, J=9.0, 2.8 Hz, 1H), 5.01 (s, 1H), 4.45-4.26 (m, 1H), 4.00 (dt, J=9.3, 3.0 Hz, 1H), 3.86 (dt, J=21.4, 5.9 Hz, 2H), 3.17 (q, J=6.1 Hz, 1H), 2.63 (d, J=13.3 Hz, 1H), 1.97 (dtt, J=33.0, 12.8, 7.0 Hz, 2H), 1.74 (t, J=6.5 Hz, 2H), 1.66 (d, J=9.6 Hz, 1H), 1.54 (dq, J=11.8, 8.0 Hz, 1H), 1.40-1.27 (m, 1H).

### Example 5

### Synthesis of 2-(4-chloro-3-fluorophenoxy)-N-((1S,3S)-3-((3-(4-chloro-3-fluorophenoxy)-2-hydroxypropyl)amino)cyclopentyl)acetamide

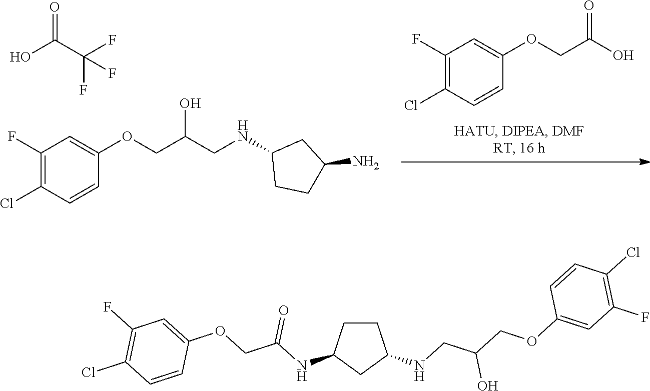 [SMILES: N[C@H]1CC[C@H](NCC(O)COc2ccc(Cl)c(F)c2)C1 ; O=C(O)C(F)(F)F ; O=C(O)COc1ccc(Cl)c(F)c1 ; O=C(COc1ccc(Cl)c(F)c1)N[C@H]1CC[C@H](NCC(O)COc2ccc(Cl)c(F)c2)C1]

To a stirred solution of 2-(4-chloro-3-fluorophenoxy)acetic acid (0.120 g, 0.6 mmol, 1.0 equiv) in DMF (3 ml) was added 1-(((1S,3S)-3-aminocyclopentyl)amino)-3-(4-chloro-3-fluorophenoxy)propan-2-ol 2,2,2-trifluoroacetate (0.300 g, 0.72 mmol, 1.2 equiv), HATU (0.342 g, 0.90 mmol, 1.5 equiv) and DIPEA (0.155 g, 1.2 mmol, 2.0 equiv). The reaction mixture was allowed to stir at RT for 16 h. Product formation was confirmed by LCMS. The reaction mixture was diluted with water (20 mL) and extracted with ethyl acetate (30 mL×3). Combined organic layer was washed with water (25 mL×6). Organic layer was dried over anhydrous sodium sulfate, filtered and concentrated under reduced pressure. Crude material was purified by reversed phase HPLC to obtain 2-(4-chloro-3-fluorophenoxy)-N-((1S,3S)-3-((3-(4-chloro-3-fluorophenoxy)-2-hydroxypropyl)amino)cyclopentyl)acetamide (Compound 20—0.020 g, 6.96%) as an off-white solid. LCMS 489.2 [M+H]⁺, ¹H NMR (400 MHz, DMSO-d₆) δ 8.03 (d, J=7.7 Hz, 1H), 7.47 (dt, J=16.9, 8.8 Hz, 2H), 7.06 (dd, J=11.3, 2.8 Hz, 2H), 6.88-6.80 (m, 2H), 4.48 (s, 2H), 4.20 (h, J=7.2 Hz, 1H), 3.99 (dd, J=10.0, 4.0 Hz, 1H), 3.86 (dt, J=19.1, 5.9 Hz, 2H), 3.14 (p, J=6.1 Hz, 1H), 2.67-2.51 (m, 1H), 2.00-1.81 (m, 3H), 1.64 (th, J=13.0, 6.0 Hz, 2H), 1.34 (dddd, J=30.6, 22.7, 15.3, 11.6 Hz, 2H).

### Example 6

### Synthesis of 6-chloro-N-((1S,3S)-3-((3-(4-chloro-3-fluorophenoxy)-2-hydroxypropyl)amino)cyclopentyl)quinoline-2-carboxamide

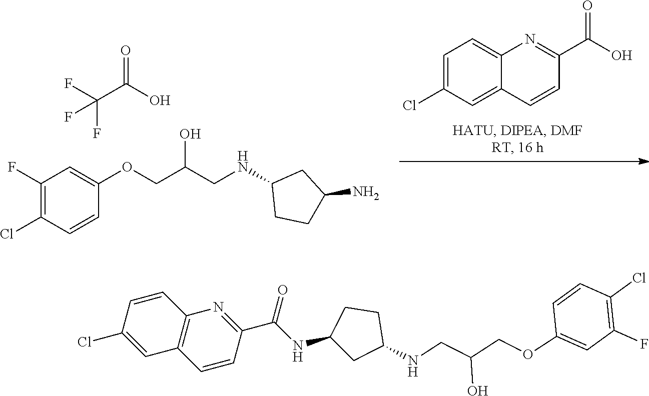

To a stirred 6-chloroquinoline-2-carboxylic acid (0.180 g, 0.87 mmol, 1.0 equiv) in DMF (5 ml) were added 1-(((1S,3S)-3-aminocyclopentyl)amino)-3-(4-chloro-3-fluorophenoxy)propan-2-ol 2,2,2-trifluoroacetate (0.434 g, 1.044 mmol, 1.2 equiv), HATU (0.496 g, 1.305 mmol, 1.5 equiv) and DIPEA (0.225 g, 1.74 mmol, 2.0 equiv). The reaction mixture was allowed to stir at RT for 16 h. Product formation was confirmed by LCMS. The reaction mixture was diluted with water (25 mL) and extracted with ethyl acetate (40 mL×3). Combined organic layer was washed with water (30 mL×6). Organic layer was dried over anhydrous sodium sulfate, filtered and concentrated under reduced pressure. Crude material was purified by reversed phase HPLC to obtain 6-chloro-N-((1S,3S)-3-((3-(4-chloro-3-fluorophenoxy)-2-hydroxypropyl)amino)cyclopentyl)quinoline-2-carboxamide (Compound 13—0.060 g, 14%) as an off-white solid. LCMS 492.2 [M+H]⁺, ¹H NMR (400 MHz, DMSO-d₆) δ 8.72 (d, J=8.2 Hz, 1H), 8.53 (d, J=8.6 Hz, 1H), 8.24 (d, J=2.5 Hz, 1H), 8.17 (dd, J=8.9, 2.7 Hz, 2H), 7.88 (dd, J=9.0, 2.5 Hz, 1H), 7.45 (t, J=8.9 Hz, 1H), 7.07 (dd, J=11.7, 2.9 Hz, 1H), 6.84 (dd, J=9.2, 2.9 Hz, 1H), 5.04 (s, 1H), 4.46 (h, J=7.8 Hz, 1H), 4.01 (dt, J=9.8, 3.2 Hz, 1H), 3.88 (dt, J=17.7, 5.9 Hz, 2H), 3.23 (p, J=5.9, 5.5 Hz, 1H), 2.74-2.51 (m, 2H), 2.04 (tdd, J=20.0, 9.5, 5.7 Hz, 2H), 1.82 (q, J=6.7 Hz, 2H), 1.62 (dq, J=11.8, 8.1 Hz, 1H), 1.37 (dt, J=15.3, 7.2 Hz, 1H), 1.23 (s, 1H).

### Example 7

### Synthesis of N,N′-((1R,3S)-cyclopentane-1,3-diyl)bis(2-(4-chloro-3-fluorophenoxy)acetamide)

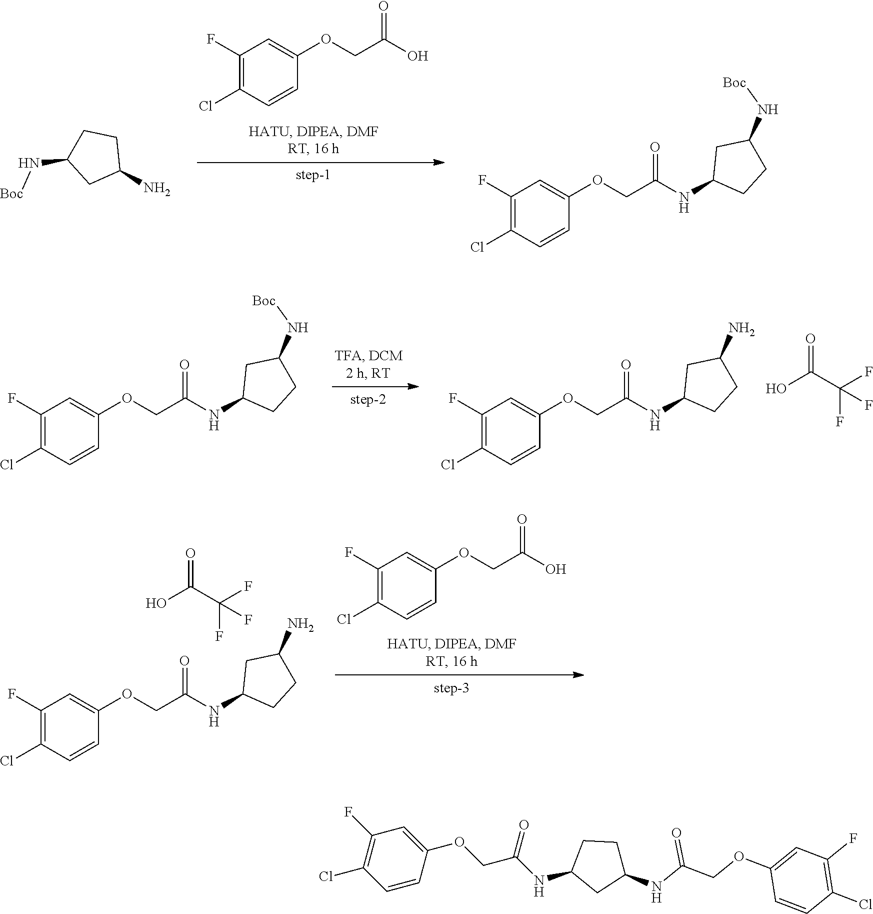

### Step-1: Synthesis of tert-butyl ((1S,3R)-3-(2-(4-chloro-3-fluorophenoxy)acetamido)cyclopentyl)carbamate

To a stirred solution of 2-(4-chloro-3-fluorophenoxy)acetic acid (0.202 g, 1.0 mmol, 1.0 equiv) in DMF (10 ml) was added tert-butyl ((1S,3R)-3-aminocyclopentyl)carbamate (0.202 g, 1.0 mmol, 1.0 equiv), HATU (0.57 g, 1.5 mmol, 1.0 equiv) and DIPEA (2.0 equiv). The reaction mixture was allowed to stir at RT for overnight. Product formation was confirmed by LCMS. The reaction mixture was diluted with water (50 mL) which results in to precipitates. The resulting solid was filtered off and washed with ice water, dried under vacuum to obtain tert-butyl ((1S,3R)-3-(2-(4-chloro-3-fluorophenoxy)acetamido)cyclopentyl)carbamate (0.32 g, 83.68% Yield) as a white solid. LCMS 387.1 [M+H]⁺.

### Step-2: Synthesis of N-((1R,3S)-3-aminocyclopentyl)-2-(4-chloro-3-fluorophenoxy)acetamide 2,2,2-trifluoroacetate

To a stirred solution of tert-butyl ((1S,3R)-3-(2-(4-chloro-3-fluorophenoxy)acetamido)cyclopentyl)carbamate (0.32 g, 0.83 mmol, 1.0 equiv) in DCM (15 ml) was added TFA (0.3 ml). The reaction mixture was allowed to stir at RT for 16 h. Reaction progress was monitored by NMR. After completion of reaction the reaction mixture was concentrated under reduced pressure. Crude compound was crystallized in pentane and then in petroleum ether to obtain N-((1R,3S)-3-aminocyclopentyl)-2-(4-chloro-3-fluorophenoxy)acetamide 2,2,2-trifluoroacetate (0.25 g, 75% Yield) as an off-white solid. LCMS 287.1 [M+H]⁺.

### Step-3: Synthesis of N,N′-((1R,3S)-cyclopentane-1,3-diyl)bis(2-(4-chloro-3-fluorophenoxy)acetamide)

To a stirred solution of 2-(4-chloro-3-fluorophenoxy)acetic acid (0.126 g, 0.625 mmol, 1.0 equiv) in DMF (7 ml) were added N-((1R,3S)-3-aminocyclopentyl)-2-(4-chloro-3-fluorophenoxy)acetamide 2,2,2-trifluoroacetate (0.25 g, 0.625 mmol, 1.0 equiv), HATU (0.36 g, 0.937 mmol, 1.5 equiv) and DIPEA (0.5 mL, 2.5 mmol, 4.0 equiv). The reaction mixture was allowed to stir at RT for 16 h. Product formation was confirmed by LCMS. The reaction mixture was diluted with water which results in to precipitate. The resulting solid precipitate was filtered and washed with ice water, dried under vacuum to obtain N,N′-((1R,3S)-cyclopentane-1,3-diyl)bis(2-(4-chloro-3-fluorophenoxy)acetamide) (Compound 15—0.17 g, 58% Yield) as a white solid. LCMS 473.1 [M+H]⁺. ¹H NMR (400 MHz, DMSO-d₆) δ 8.19 (d, J=7.0 Hz, 2H), 7.49 (t, J=8.8 Hz, 3H), 7.05 (d, J=2.6 Hz, 1H), 7.08 (d, J=2.6 Hz, 1H), 6.85 (dd, J=9.0, 1.5 Hz, 3H), 4.51 (s, 4H), 4.05 (d, J=6.6 Hz, 2H), 2.09-2.27 (m, 2H), 1.70-1.87 (m, 3H), 1.60 (d, J=5.3 Hz, 2H), 1.33-1.49 ppm (m, 2H).

### Example 8

### Synthesis of 5-chloro-N-((1S,3R)-3-(2-(4-chloro-3-fluorophenoxy)acetamido)cyclopentyl)benzofuran-2-carboxamide

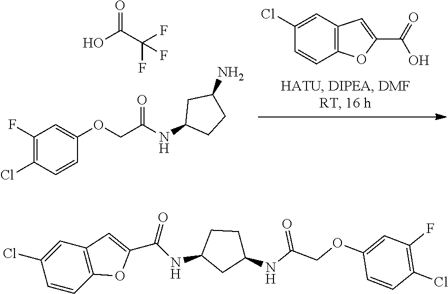

To a stirred solution of 5-chlorobenzofuran-2-carboxylic acid (0.070 g, 0.356 mmol, 1.0 equiv) in DMF (5 ml) were added N-((1R,3S)-3-aminocyclopentyl)-2-(4-chloro-3-fluorophenoxy)acetamide 2,2,2-trifluoroacetate (0.157 g, 0.391 mmol, 1.1 equiv), HATU (0.203 g, 0.534 mmol, 1.5 equiv) and DIPEA (0.3 mL, 1.42 mmol, 4.0 equiv). The reaction mixture was allowed to stir at RT for 16 h. Product formation was confirmed by LCMS. The reaction mixture was diluted with water which results in to precipitate. The resulting solid precipitate was filtered and washed with ice water, dried under vacuum and washed with diethyl ether to obtain 5-chloro-N-((1S,3R)-3-(2-(4-chloro-3-fluorophenoxy)acetamido)cyclopentyl)benzofuran-2-carboxamide (Compound 17—0.095 g, 57% Yield) as a white solid. LCMS 465.2 [M+H]⁺. ¹H NMR (400 MHz, DMSO-d₆) δ 8.81 (d, J=7.5 Hz, 1H), 8.22 (d, J=7.0 Hz, 1H), 7.87 (d, J=1.8 Hz, 1H), 7.69 (d, J=8.8 Hz, 1H), 7.55-7.42 (m, 2H), 7.09 (dd, J=2.9, 11.6 Hz, 1H), 6.86 (d, J=9.2 Hz, 1H), 4.53 (s, 2H), 4.33-4.16 (m, 1H), 4.16-4.01 (m, 1H), 2.39-2.22 (m, 1H), 2.00-1.79 (m, 2H), 1.74 (d, J=8.3 Hz, 1H), 1.70-1.51 (m, 2H).

### Example 9

### Synthesis of 6-chloro-N-((1S,3R)-3-(2-(4-chloro-3-fluorophenoxy)acetamido)cyclopentyl)quinoline-2-carboxamide

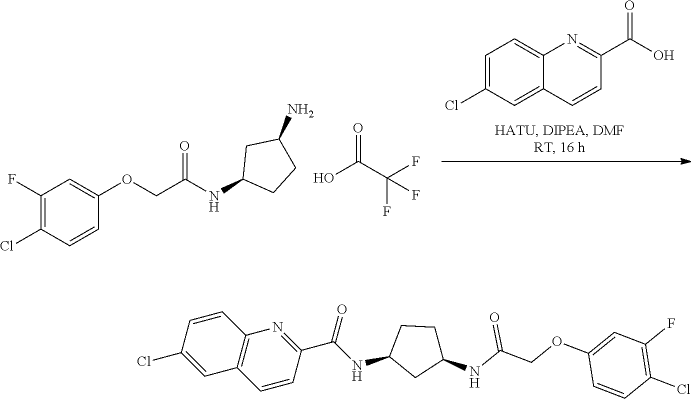

To a stirred solution of 5-chlorobenzofuran-2-carboxylic acid (0.070 g, 0.338 mmol, 1.0 equiv) in DMF (5 ml) was added N-((1R,3S)-3-aminocyclopentyl)-2-(4-chloro-3-fluorophenoxy)acetamide 2,2,2-trifluoroacetate (0.148 g, 0.372 mmol, 1.1 equiv), HATU (0.193 g, 0.534 mmol, 1.5 equiv) and DIPEA (0.25 mL, 1.352 mmol, 4.0 equiv). The reaction mixture was allowed to stir at RT for 16 h. Product formation was confirmed by LCMS. The reaction mixture was diluted with water which results in to precipitate. The resulting solid precipitate was filtered and washed with ice water, dried under vacuum and washed with diethyl ether to obtain 6-chloro-N-((1S,3R)-3-(2-(4-chloro-3-fluorophenoxy)acetamido)cyclopentyl)quinoline-2-carboxamide (Compound 19—0.080 g, 50% Yield) as a white solid. LCMS 476.2 [M+H]⁺. ¹H NMR (400 MHz, DMSO-d₆) δ 8.91 (d, J=7.9 Hz, 1H), 8.55 (d, J=8.8 Hz, 1H), 8.36-8.23 (m, 1H), 8.23-8.07 (m, 2H), 7.88 (dd, J=2.4, 9.0 Hz, 1H), 7.48 (t, J=8.8 Hz, 1H), 7.08 (dd, J=2.6, 11.4 Hz, 1H), 6.86 (d, J=9.2 Hz, 1H), 4.55 (s, 2H), 4.32 (dd, J=6.8, 14.7 Hz, 1H), 4.20-4.02 (m, 1H), 2.38-2.23 (m, 2H), 2.01-1.73 (m, 3H), 1.73-1.57 (m, 2H).

### Example 10

### Synthesis of 5-chloro-N-((1S,3R)-3-((3-(4-chloro-3-fluorophenoxy)-2-hydroxypropyl)amino)cyclopentyl)benzofuran-2-carboxamide

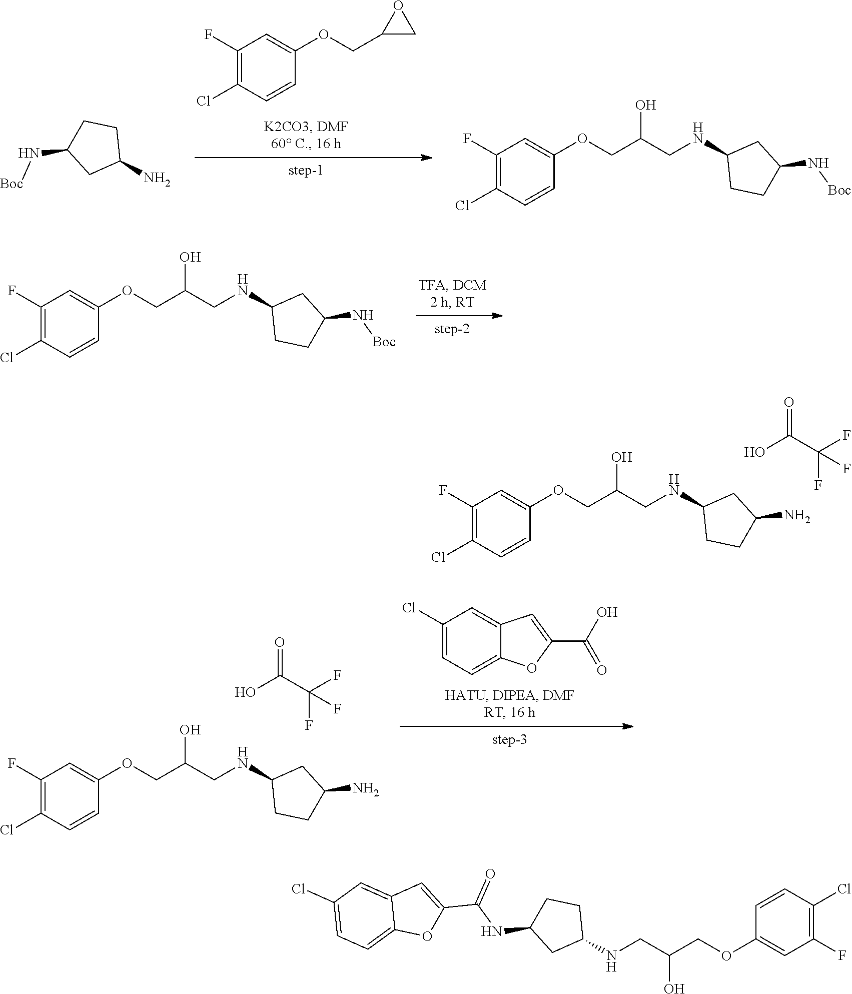

### Step-1: Synthesis of tert-butyl ((1S,3R)-3-((3-(4-chloro-3-fluorophenoxy)-2-hydroxypropyl)amino)cyclopentyl)carbamate

To a stirred solution of tert-butyl ((1S,3R)-3-aminocyclopentyl)carbamate (0.2 g, 1 mmol, 1.0 equiv) in DMF (10 ml) was added K₂CO₃ (0.27 g, 1 mmol, 2.0 equiv) followed by the addition of 2-((4-chloro-3-fluorophenoxy)methyl)oxirane (0.2 g, 2 mmol, 1.0 equiv). The reaction mixture was allowed to stir at 60° C. for overnight. Product formation was confirmed by LCMS. The reaction mixture was diluted with water which results in to precipitate. The resulting solid precipitate was filtered and washed with ice water, dried under vacuum to obtain tert-butyl ((1S,3R)-3-((3-(4-chloro-3-fluorophenoxy)-2-hydroxypropyl)amino)cyclopentyl)carbamate (0.29 g, 72% yield) as a white solid. LCMS 403.2 [M+H]⁺.

### Step-2: Synthesis of 1-(((1R,3S)-3-aminocyclopentyl)amino)-3-(4-chloro-3-fluorophenoxy)propan-2-ol 2,2,2-trifluoroacetate

To a stirred solution of tert-butyl ((1S,3R)-3-((3-(4-chloro-3-fluorophenoxy)-2-hydroxypropyl)amino)cyclopentyl)carbamate (0.29 g, 0.721 mmol, 1.0 equiv) in DCM (10 ml) was added TFA (0.5 ml). The reaction mixture was allowed to stir at RT for overnight. Reaction progress was monitored by LCMS and NMR. After completion of reaction, reaction mixture was concentrated under reduced pressure to obtain 1-(((1R,3S)-3-aminocyclopentyl)amino)-3-(4-chloro-3-fluorophenoxy)propan-2-ol 2,2,2-trifluoroacetate (0.3 g, 100% yield) as yellow semi solid. LCMS 303.1 [M+H]⁺.

### Step-3: Synthesis of 5-chloro-N-((1S,3R)-3-((3-(4-chloro-3-fluorophenoxy)-2-hydroxypropyl)amino)cyclopentyl)benzofuran-2-carboxamide

To a stirred solution of 5-chlorobenzofuran-2-carboxylic acid (0.095 g, 0.480 mmol, 1.0 equiv) in DMF (5 mL) was added 1-(((1R,3S)-3-aminocyclopentyl)amino)-3-(4-chloro-3-fluorophenoxy)propan-2-ol 2,2,2-trifluoroacetate (0.2 g, 0.480 mmol, 1.0 equiv), HATU (0.274 g, 0.72 mmol, 1.5 equiv) and DIPEA (0.4 mL, mmol, 1.0 equiv). The reaction mixture was allowed to stir at RT for 16 h. Product formation was confirmed by LCMS. The reaction mixture was diluted with water (50 mL) and extracted with ethyl acetate (30 mL×3). Combined organic layer was washed with water (30 mL×6). Organic layer was dried over anhydrous sodium sulfate, filtered and concentrated under reduced pressure. Crude material was purified by combiflash chromatography (10% MeOH in DCM as an eluent) to obtain 5-chloro-N-((1S,3R)-3-((3-(4-chloro-3-fluorophenoxy)-2-hydroxypropyl)amino)cyclopentyl)benzofuran-2-carboxamide (Compound 21—0.070 g, 30% Yield) as a white solid. LCMS 481.2 [M+H]⁺. ¹H NMR (DMSO-d₆, 400 MHz) δ 8.82 (d, J=7.5 Hz, 1H), 7.86 (s, 1H), 7.67 (d, J=8.8 Hz, 1H), 7.33-7.56 (m, 3H), 7.02-7.13 (m, 1H), 6.84 (d, J=8.8 Hz, 1H), 4.26 (br. s., 1H), 3.86-4.12 (m, 3H), 3.45 (br. s., 1H), 2.99 (br. s., 1H), 2.87 (br. s., 1H), 1.84-2.06 (m, 2H), 1.77 (br. s., 2H), 1.65 ppm (br. s., 1H).

### Example 11

### Synthesis of 2-(4-chloro-3-fluorophenoxy)-N-((1S,3R)-3-((3-(4-chloro-3-fluorophenoxy)-2-hydroxypropyl)amino)cyclopentyl)acetamide

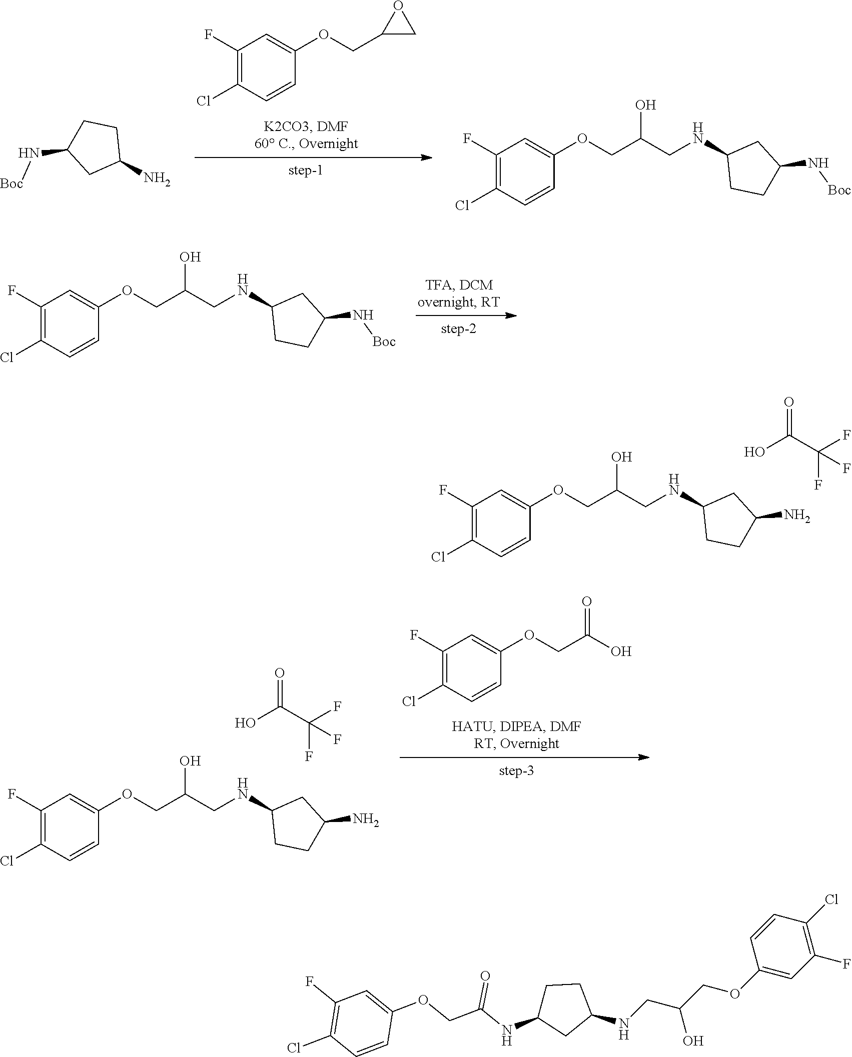

### Step-1: Synthesis of tert-butyl ((1S,3R)-3-((3-(4-chloro-3-fluorophenoxy)-2-hydroxypropyl)amino)cyclopentyl)carbamate

To a stirred solution of tert-butyl ((1S,3R)-3-aminocyclopentyl)carbamate (0.2 g, 1 mmol, 1.0 equiv) in DMF (10 ml) was added K₂CO₃ (0.27 g, 1 mmol, 2.0 equiv) followed by the addition of 2-((4-chloro-3-fluorophenoxy)methyl)oxirane (0.2 g, 2 mmol, 1.0 equiv). The reaction mixture was allowed to stir at 60° C. for overnight. Product formation was confirmed by LCMS. The reaction mixture was diluted with water which results in to precipitate. The resulting solid precipitate was filtered and washed with ice water, dried under vacuum to obtain tert-butyl ((1S,3R)-3-((3-(4-chloro-3-fluorophenoxy)-2-hydroxypropyl)amino)cyclopentyl)carbamate (0.29 g, 72% Yield) as a white solid. LCMS 403.2 [M+H]⁺

### Step-2: Synthesis of 1-(((1R,3S)-3-aminocyclopentyl)amino)-3-(4-chloro-3-fluorophenoxy)propan-2-ol 2,2,2-trifluoroacetate

To a stirred solution of tert-butyl ((1S,3R)-3-((3-(4-chloro-3-fluorophenoxy)-2-hydroxypropyl)amino)cyclopentyl)carbamate (0.29 g, 0.721 mmol, 1.0 equiv) in DCM (10 ml) was added TFA (0.5 ml). The reaction mixture was allowed to stir at RT for overnight. Reaction progress was monitored by LCMS and NMR. After completion of reaction, reaction mixture was concentrated under reduced pressure to obtain 1-(((1R,3S)-3-aminocyclopentyl)amino)-3-(4-chloro-3-fluorophenoxy)propan-2-ol 2,2,2-trifluoroacetate (0.300 g, 100% yield) as a yellow semi solid. LCMS 303.1 [M+H]⁺

### Step-3: Synthesis of 2-(4-chloro-3-fluorophenoxy)-N-((1S,3R)-3-((3-(4-chloro-3-fluorophenoxy)-2-hydroxypropyl)amino)cyclopentyl)acetamide

To a stirred solution of 2-(4-chloro-3-fluorophenoxy)acetic acid (0.2 g, 0.47 mmol, 1.0 equiv) in DMF (5 ml) was added 1-(((1R,3S)-3-aminocyclopentyl)amino)-3-(4-chloro-3-fluorophenoxy)propan-2-ol 2,2,2-trifluoroacetate (0.09 g, 0.479 mmol, 1.0 equiv), HATU (0.364 g, 0.95 mmol, 2.0 equiv) and DIPEA (0.1 ml, 0.958 mmol, 2.0 equiv). The reaction mixture was allowed to stir at RT for overnight. Product formation was confirmed by LCMS. The reaction mixture was diluted with water (20 mL) and extracted with ethyl acetate (30 mL×3). Combined organic layer was washed with water (25 mL×6). Organic layer was dried over anhydrous sodium sulfate, filtered and concentrated under reduced pressure. Crude material obtained was purified by reversed phase HPLC to obtain 2-(4-chloro-3-fluorophenoxy)-N-((1S,3R)-3-((3-(4-chloro-3-fluorophenoxy)-2-hydroxypropyl)amino)cyclopentyl)acetamide (Compound 2—0.047 g, 20% Yield) as an off-white solid. LCMS 489.2 [M+H]⁺; ¹H NMR (400 MHz, DMSO-d₆) δ 8.14 (d, J=7.45 Hz, 1H) 7.33-7.51 (m, 2H) 6.95-7.10 (m, 2H) 6.75-6.88 (m, 2H) 4.47 (s, 2H) 4.13 (br. s., 1H) 3.98 (br. s., 1H) 3.89 (d, J=7.89 Hz, 2H) 3.11 (br. s., 2H) 2.67 (br. s., 1H) 2.02 (br. s., 1H) 1.79 (d, J=6.14 Hz, 2H) 1.54 (d, J=5.70 Hz, 2H) 1.35 (br. s., 2H).

### Example 12

### Synthesis of 6-chloro-N-((1S,3R)-3-((3-(4-chloro-3-fluorophenoxy)-2-hydroxypropyl)amino)cyclopentyl)quinoline-2-carboxamide

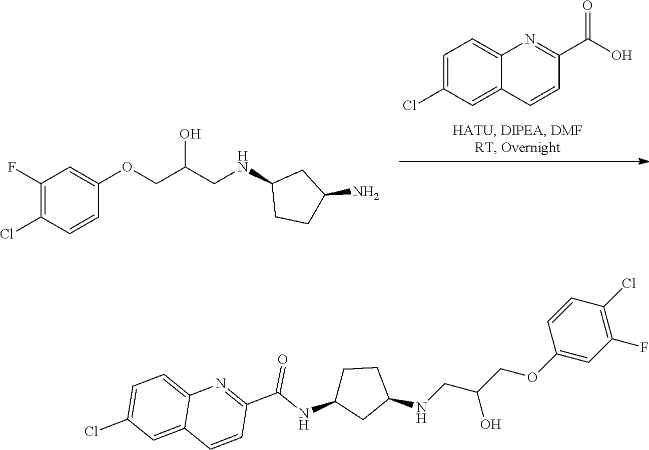 [SMILES: O=C(O)c1ccc2cc(Cl)ccc2n1 ; N[C@H]1CC[C@@H](NCC(O)COc2ccc(Cl)c(F)c2)C1 ; O=C(N[C@H]1CC[C@@H](NCC(O)COc2ccc(Cl)c(F)c2)C1)c1ccc2cc(Cl)ccc2n1]

To a stirred solution of 6-chloroquinoline-2-carboxylic acid (0.050 g, 0.242 mmol, 1.0 equiv) in DMF (3 ml) was added 1-(((1R,3S)-3-aminocyclopentyl)amino)-3-(4-chloro-3-fluorophenoxy)propan-2-ol 2,2,2-trifluoroacetate (0.100 g, 0.242 mmol, 1.0 equiv), HATU (0.138 g, 0.363 mmol, 1.5 equiv) and DIPEA (0.125 g, 0.968 mmol, 4.0 equiv). The reaction mixture was allowed to stir at RT for overnight. Product formation was confirmed by LCMS. The reaction mixture was diluted with water (20 mL) and extracted with ethyl acetate (30 mL×3). Combined organic layer was washed with water (25 mL×6). Organic layer was dried over anhydrous sodium sulfate, filtered and concentrated under reduced pressure. Crude material obtained was purified by reversed phase HPLC to obtain 2-(4-chloro-3-fluorophenoxy)-N-((1S,3R)-3-((3-(4-chloro-3-fluorophenoxy)-2-hydroxypropyl)amino)cyclopentyl)acetamide (Compound 3—0.020 g, 16% Yield) as an off-white solid. LCMS 492.3 [M+H]⁺. ¹H NMR (400 MHz, DMSO-d₆) δ 9.11 (br. s., 1H), 8.52 (dd, J=4.2, 8.6 Hz, 1H), 8.24-8.11 (m, 1H), 8.11-7.99 (m, 1H), 7.86-7.71 (m, 1H), 7.35 (br. s., 1H), 6.76-6.57 (m, 1H), 4.42 (br. s., 1H), 4.17-3.90 (m, 3H), 2.91-2.67 (m, 2H), 1.98-1.87 (m, 1H), 1.83 (d, J=7.0 Hz, 1H), 1.75 (br. s., 2H).

### Example 13

### Synthesis of 2-(4-chloro-3-fluorophenoxy)-N-((1-(2-(4-chloro-3-fluorophenoxy)acetyl)pyrrolidin-3-yl)methyl)acetamide

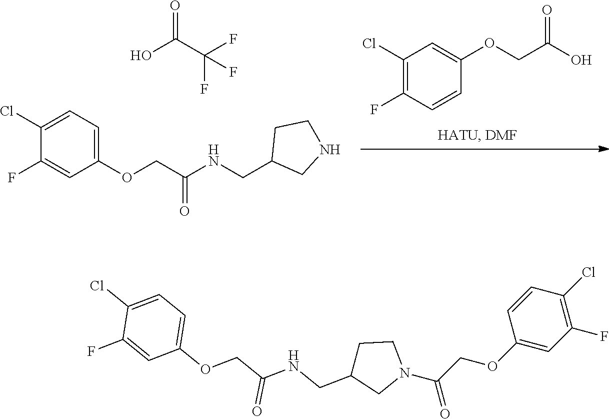 [SMILES: O=C(O)C(F)(F)F ; O=C(O)COc1ccc(F)c(Cl)c1 ; O=C(COc1ccc(Cl)c(F)c1)NCC1CCNC1 ; O=C(COc1ccc(Cl)c(F)c1)NCC1CCN(C(=O)COc2ccc(Cl)c(F)c2)C1]

To a stirred solution of 2-(4-chloro-3-fluorophenoxy)acetic acid (0.35 g, 0.875 mmol, 1.0 equiv) in DMF (5 ml) were added 2-(4-chloro-3-fluorophenoxy)-N-(pyrrolidin-3-ylmethyl)acetamide 2,2,2-trifluoroacetate (0.35 g, 0.875 mmol, 1.0 equiv), HATU (0.5 g, 1.31 mmol, 1.5 equiv) and DIPEA (0.6 mL, 3.52 mmol, 4.0 equiv). The reaction mixture was allowed to stir at RT for overnight. Product formation was confirmed by LCMS. The reaction mixture was diluted with water (50 mL) and extracted with ethyl acetate (30 mL×3). Combined organic layer was washed with water (30 mL×4). Organic layer was dried over anhydrous sodium sulfate, filtered and concentrated. Crude material obtained was purified by reversed phase HPLC to obtain 2-(4-chloro-3-fluorophenoxy)-N-((1-(2-(4-chloro-3-fluorophenoxy)acetyl)pyrrolidin-3-yl)methyl)acetamide (Compound 4—0.170 g, 41% Yield) as a white solid. LCMS 473.1 [M+H]⁺; ¹H NMR (DMSO-d₆, 400 MHz) δ 8.29 (d, J=7.9 Hz, 1H), 7.39-7.54 (m, 2H), 6.95-7.14 (m, 2H), 6.75-6.92 (m, 2H), 4.69-4.84 (m, 2H), 4.55 (d, J=4.4 Hz, 2H), 3.39-3.60 (m, 3H), 3.12-3.29 (m, 3H), 3.02 (dd, J=12.3, 7.0 Hz, 1H), 2.20-2.36 (m, 2H), 1.96 (d, J=6.6 Hz, 1H), 1.86 (d, J=6.1 Hz, 1H), 1.65 (br. s., 1H), 1.55 ppm (br. s., 1H).

### Example 14

### Synthesis of 2-(4-chloro-3-fluorophenoxy)-N-((1-(6-chloroquinoline-2-carbonyl)pyrrolidin-3-yl)methyl)acetamide

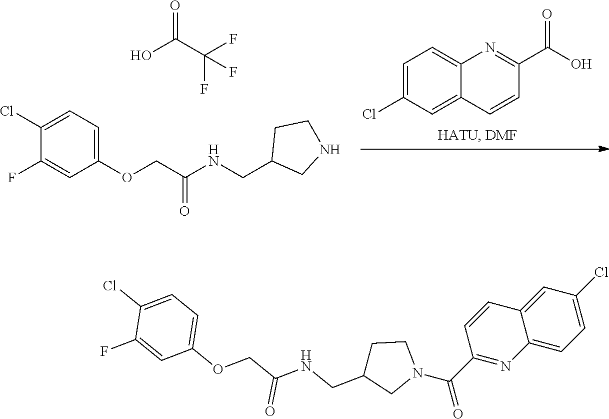 [SMILES: O=C(COc1ccc(Cl)c(F)c1)NCC1CCNC1 ; O=C(O)C(F)(F)F ; O=C(O)c1ccc2cc(Cl)ccc2n1 ; O=C(COc1ccc(Cl)c(F)c1)NCC1CCN(C(=O)c2ccc3cc(Cl)ccc3n2)C1]

To a stirred solution of 6-chloroquinoline-2-carboxylic acid (1.0 equiv) in DMF (4 ml) is added 2-(4-chloro-3-fluorophenoxy)-N-(pyrrolidin-3-ylmethyl)acetamide 2,2,2-trifluoroacetate (1.2 equiv), HATU (1.5 equiv) and DIPEA (2.0 equiv). The reaction mixture is allowed to stir at RT for 16 h. Product formation is confirmed by LCMS. The reaction mixture is diluted with water and a precipitate is formed. The resulting solid precipitate is filtered and washed with ice water, dried under vacuum to obtain 2-(4-chloro-3-fluorophenoxy)-N-((1-(6-chloroquinoline-2-carbonyl)pyrrolidin-3-yl)methyl)acetamide (Compound 5).

### Example 15

### Synthesis of 2-(4-chloro-3-fluorophenoxy)-N-((1-(5-chlorobenzofuran-2-carbonyl)pyrrolidin-3-yl)methyl)acetamide

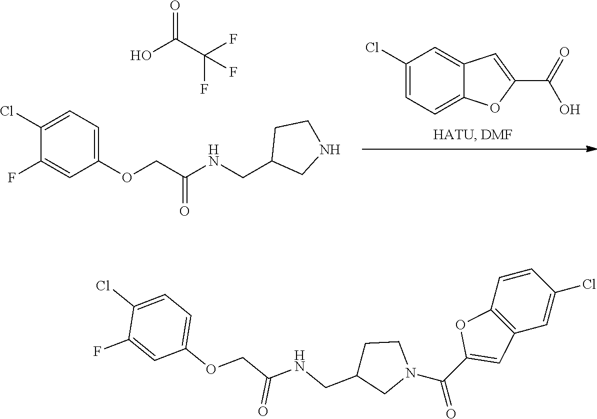 [SMILES: O=C(COc1ccc(Cl)c(F)c1)NCC1CCNC1 ; O=C(O)C(F)(F)F ; O=C(O)c1cc2cc(Cl)ccc2o1 ; O=C(COc1ccc(Cl)c(F)c1)NCC1CCN(C(=O)c2cc3cc(Cl)ccc3o2)C1]

To a stirred solution of 6-chlorobenzofuran-2-carboxylic acid (1.0 equiv) in DMF (4 ml) are added 2-(4-chloro-3-fluorophenoxy)-N-(pyrrolidin-3-ylmethyl)acetamide 2,2,2-trifluoroacetate (1.2 equiv), HATU (1.5 equiv) and DIPEA (2.0 equiv). The reaction mixture is allowed to stir at RT for 16 h. Product formation is confirmed by LCMS. The reaction mixture is diluted with water and a precipitate is formed. The resulting solid precipitate is filtered and washed with ice water, dried under vacuum to obtain 2-(4-chloro-3-fluorophenoxy)-N-((1-(5-chlorobenzofuran-2-carbonyl)pyrrolidin-3-yl)methyl)acetamide (Compound 6).

### Example 16

### Synthesis of 2-(4-chloro-3-fluorophenoxy)-N-((1-(3-(4-chloro-3-fluorophenoxy)-2-hydroxypropyl)pyrrolidin-3-yl)methyl)acetamide

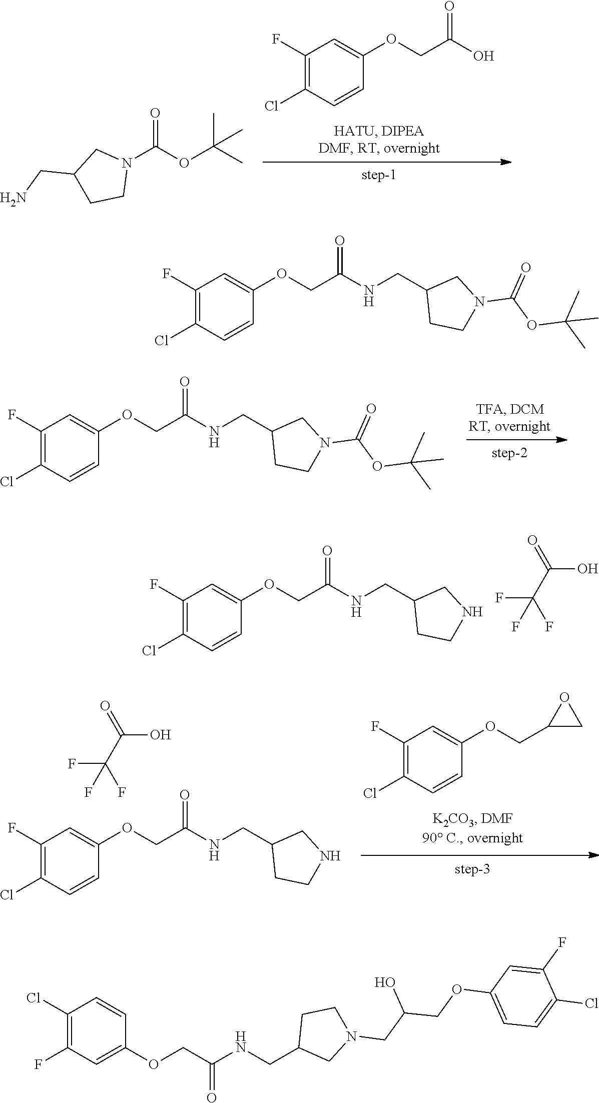

### Step-1: Synthesis of tert-butyl 3-((2-(4-chloro-3-fluorophenoxy)acetamido)methyl)pyrrolidine-1-carboxylate

To a solution of tert-butyl 3-(aminomethyl)pyrrolidine-1-carboxylate (1000 mg, 5.0 mmol, 1.0 equiv) in DMF (5 mL) was added 2-(4-chloro-3-fluorophenoxy)acetic acid (1025 mg, 5.0 mmol, 1.0 equiv) and HATU (3800 mg, 10.0 mmol, 2.0 equiv) at RT. The resulting reaction mixture was stir for 10 min, DIPEA (2.59 mL, 15.0 mmol, 3.0 equiv) was added. The reaction mixture was allowed to stir at RT overnight. Product formation was confirmed by LCMS. The reaction mixture was diluted with water (50 mL) and extracted with ethyl acetate (100 mL×2). Combined organic extracts were washed with water (50 mL×4), dried over anhydrous Na₂SO₄ and concentrated to obtained tert-butyl 3-((2-(4-chloro-3-fluorophenoxy)acetamido)methyl)pyrrolidine-1-carboxylate (1000 mg, 51% Yield) as a yellow semi solid. LCMS 387.1 [M+H]⁺

### Step-2 Synthesis of 2-(4-chloro-3-fluorophenoxy)-N-(pyrrolidin-3-ylmethyl)acetamide 2,2,2-trifluoroacetate

To a stirred solution of tert-butyl 3-((2-(4-chloro-3-fluorophenoxy)acetamido)methyl)pyrrolidine-1-carboxylate (1000 mg, 2.58 mmol, 1.0 equiv) in DCM (15 mL), was added TFA (5 mL). The resultant reaction mixture was stirred at RT for overnight under nitrogen atmosphere. Reaction was monitored by TLC and LCMS. After completion of reaction, the reaction mixture was concentrated under reduced pressure to obtain 2-(4-chloro-3-fluorophenoxy)-N-(pyrrolidin-3-ylmethyl)acetamide 2,2,2-trifluoroacetate (800 mg, 77% Yield ) as a brown semi solid. LCMS 287.1 [M+H]⁺

### Step-3 Synthesis of 2-(4-chloro-3-fluorophenoxy)-N-((1-(3-(4-chloro-3-fluorophenoxy)-2-hydroxypropyl)pyrrolidin-3-yl)methyl)acetamide

To a stirred solution of 2-(4-chloro-3-fluorophenoxy)-N-(pyrrolidin-3-ylmethyl)acetamide 2,2,2-trifluoroacetate (800 mg, 2.08 mmol, 1.0 equiv), was added 2-((4-chloro-3-fluorophenoxy)methyl)oxirane (424 mg, 2.08 mmol, 1.0 equiv) in DMF (5 mL), was added K₂CO₃ (575 mg, 4.16 mmol, 2.0 equiv). The resultant reaction mixture was heated at 90° C. for overnight. Reaction was monitored by TLC and LCMS. After completion of reaction, the reaction mixture was diluted with water (200 mL) and extracted with EtOAc (200 mL) and washed with water and brine solution (2×150 mL) and dried over anhydrous sodium sulfate and concentrated under reduced pressure to obtain crude which was purified by reversed-phase HPLC to obtain 2-(4-chloro-3-fluorophenoxy)-N-((1-(3-(4-chloro-3-fluorophenoxy)-2-hydroxypropyl)pyrrolidin-3-yl)methyl)acetamide (Compound 7—200 mg, 20% Yield) as a light yellow solid. LCMS 489.3 [M+H]⁺; ¹H NMR (400 MHz, DMSO-d₆) δ 8.21 (br. s., 1H) 7.35-7.57 (m, 2H) 7.03 (s, 1H) 7.06 (s, 1H) 6.83 (br. s., 2H) 4.53 (s, 2H) 4.00 (d, J=7.89 Hz, 1H) 3.91 (d, J=7.45 Hz, 2H) 3.35 (br. s., 2H) 3.06-3.20 (m, 2H) 2.74 (br. s., 4H) 2.34 (br. s., 1H) 1.84 (br. s., 1H) 1.45 (br. s., 1H).

### Example 17

### Synthesis of 5-chloro-N-((1-(3-(4-chloro-3-fluorophenoxy)-2-hydroxypropyl)pyrrolidin-3-yl)methyl)benzofuran-2-carboxamide

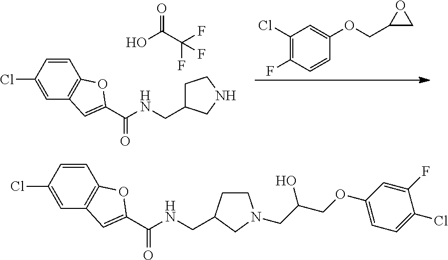

To a stirred solution of 5-chloro-N-(pyrrolidin-3-ylmethyl)benzofuran-2-carboxamide 2,2,2-trifluoroacetate (1.0 equiv) in DMF (10 ml) is added K₂CO₃ (2.0 equiv) followed by the addition of 2-((4-chloro-3-fluorophenoxy)methyl)oxirane (1.0 equiv). The reaction mixture is allowed to stir at 60° C. for 16 h. Product formation is confirmed by LCMS. The reaction mixture is diluted with water which results in a precipitate. The resulting solid precipitate is filtered and washed with ice water, dried under vacuum to obtain 5-chloro-N-((1-(3-(4-chloro-3-fluorophenoxy)-2-hydroxypropyl)pyrrolidin-3-yl)methyl)benzofuran-2-carboxamide (Compound 8).

### Example 18

### Synthesis of 6-chloro-N-((1-(3-(4-chloro-3-fluorophenoxy)-2-hydroxypropyl)pyrrolidin-3-yl)methyl)quinoline-2-carboxamide

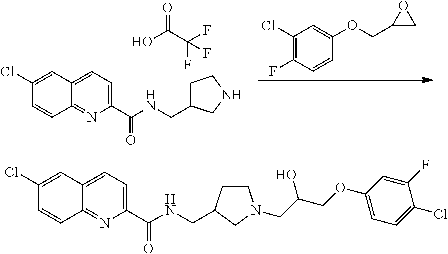

To a stirred solution of 6-chloro-N-(pyrrolidin-3-ylmethyl)quinoline-2-carboxamide 2,2,2-trifluoroacetate (1.0 equiv) in DMF (10 ml) is added K₂CO₃ (2.0 equiv) followed by the addition of 2-((4-chloro-3-fluorophenoxy)methyl)oxirane (1.0 equiv). The reaction mixture is allowed to stir at 60° C. for 16 h. Product formation is confirmed by LCMS. The reaction mixture is diluted with water and a precipitate is formed. The resulting solid precipitate is filtered and washed with ice water, dried under vacuum to obtain 6-chloro-N-((1-(3-(4-chloro-3-fluorophenoxy)-2-hydroxypropyl)pyrrolidin-3-yl)methyl)quinoline-2-carboxamide (Compound 9).

### Example 19

### Synthesis of 2-(4-chloro-3-fluorophenoxy)-N-(3-((2-(4-chloro-3-fluorophenoxy)acetamido)methyl)pyrrolidin-1-yl)acetamide

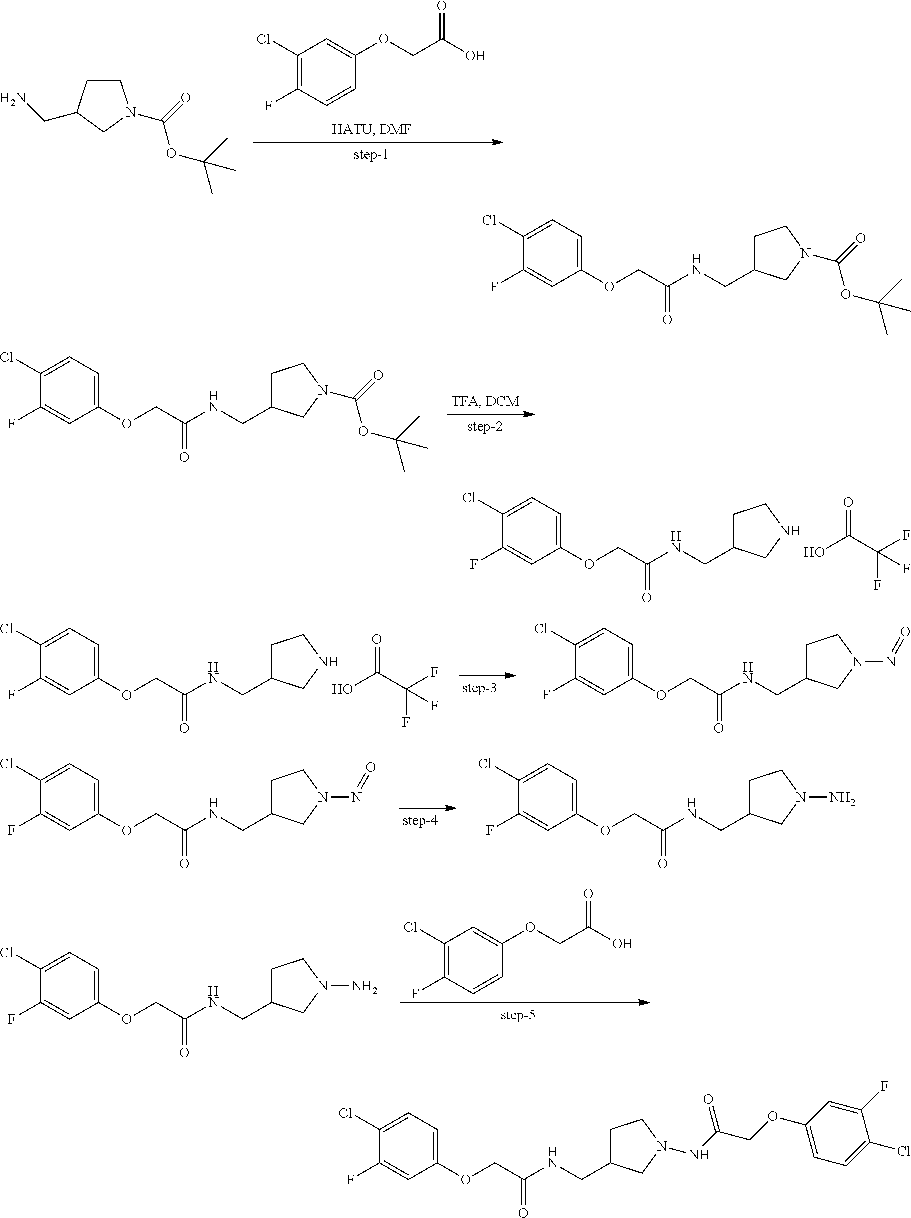

### Step-1: tert-butyl 3-((2-(4-chloro-3-fluorophenoxy)acetamido)methyl)pyrrolidine-1-carboxylate

To a stirred solution of tert-butyl 3-(aminomethyl)pyrrolidine-1-carboxylate (1.0 equiv) in DMF (10 mL) is added 2-(4-chloro-3-fluorophenoxy)acetic acid (1.0 equiv) and HATU (2.0 equiv) at RT. The resulting reaction mixture is stir for 10 minutes and DIPEA (3.00 equiv) was added. The reaction mixture is allowed to stir at RT for overnight. Product formation is confirmed by LCMS. The reaction mixture is diluted with water (50 mL) and extracted with ethyl acetate (100 mL×2). Combined organic extracts are washed with water (20 mL×4), dried over anhydrous sodium sulfate and concentrated to obtain tert-butyl 3-((2-(4-chloro-3-fluorophenoxy)acetamido)methyl)pyrrolidine-1-carboxylate.

### Step-2: Synthesis of 2 2-(4-chloro-3-fluorophenoxy)-N-(pyrrolidin-3-ylmethyl)acetamide 2,2,2-trifluoroacetate

To a stirred solution of tert-butyl 3-((2-(4-chloro-3-fluorophenoxy)acetamido)methyl)pyrrolidine-1-carboxylate (2.5 mmol) in DCM (15 mL) is added TFA (1 mL). The resultant reaction mixture is stirred at RT for overnight. Progress of the reaction is monitored by NMR spectroscopy. After completion of the reaction, the reaction mixture is concentrated under reduced pressure to obtain 2-(4-chloro-3-fluorophenoxy)-N-(pyrrolidin-3-ylmethyl)acetamide 2,2,2-trifluoroacetate.

### Step-3: 2-(4-chloro-3-fluorophenoxy)-N-((1-nitrosopyrrolidin-3-yl)methyl)acetamide

To a stirred solution of 2-(4-chloro-3-fluorophenoxy)-N-(pyrrolidin-3-ylmethyl)acetamide 2,2,2-trifluoroacetate (1.0 equiv) in water (30 mL) is added acetic acid (10 mL) and sodium nitrite (4.0 equiv) at RT. The reaction mixture is allowed to stir at RT overnight. Product formation is confirmed by LCMS. The reaction mixture is diluted with water (50 mL) and a precipitate is formed. The resulting solid is filtered off, washed with water (20 mL×4) and dried under vacuum to obtain to 2-(4-chloro-3-fluorophenoxy)-N-((1-nitrosopyrrolidin-3-yl)methyl)acetamide.

### Step-4: N-((1-aminopyrrolidin-3-yl)methyl)-2-(4-chloro-3-fluorophenoxy)acetamide

To a solution of 2-(4-chloro-3-fluorophenoxy)-N-((1-nitrosopyrrolidin-3-yl)methyl)acetamide (1.0 equiv) in water (5 mL) is added acetic acid (1 mL) and Zn dust (10.0 equiv) at RT. The reaction mixture is allowed to stir at RT overnight. Product formation is confirmed by LCMS. The reaction mixture is filtered through Celite®. The resulting filtrate is basified by liquid ammonia and extracted with ethyl acetate (50 mL×2). Combined organic layer is washed with water (20 mL×4), dried over anhydrous sodium sulfate and concentrated to obtain N-((1-aminopyrrolidin-3-yl)methyl)-2-(4-chloro-3-fluorophenoxy)acetamide.

### Step-5: Synthesis of 2-(4-chloro-3-fluorophenoxy)-N-(3-((2-(4-chloro-3-fluorophenoxy)acetamido)methyl)pyrrolidin-1-yl)acetamide

To a solution of N-((1-aminopyrrolidin-3-yl)methyl)-2-(4-chloro-3-fluorophenoxy)acetamide (1.0 equiv) in DMF (5 mL) is added 2-(3-chloro-4-fluorophenoxy)acetic acid (1.0 equiv) and HATU (2.0 equiv) at RT. The resulting reaction mixture is stirred for 10 minutes and DIPEA (3.0 equiv) is added. The reaction mixture is allowed to stir at RT overnight. Product formation is confirmed by LCMS. The reaction mixture is diluted with water (20 mL) and extracted with ethyl acetate (50 mL×2). Combined organic layer is washed with water (20 mL×4), dried over anhydrous sodium sulfate and concentrated. The crude product is purified by reverse phase HPLC to obtain 2-(4-chloro-3-fluorophenoxy)-N-(3-((2-(4-chloro-3-fluorophenoxy)acetamido)methyl)pyrrolidin-1-yl)acetamide (Compound 10).

### Example 20

### Synthesis of 6-chloro-N-(3-((2-(4-chloro-3-fluorophenoxy)acetamido)methyl)pyrrolidin-1-yl)quinoline-2-carboxamide

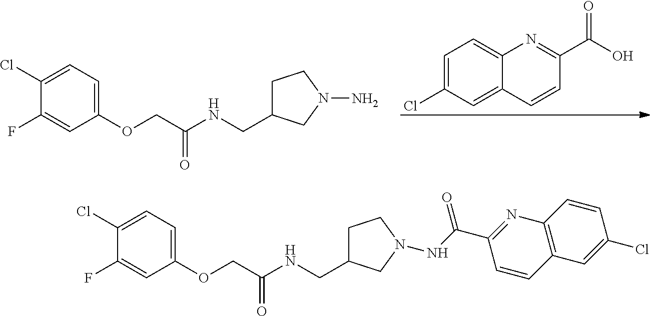 [SMILES: NN1CCC(CNC(=O)COc2ccc(Cl)c(F)c2)C1 ; O=C(O)c1ccc2cc(Cl)ccc2n1 ; O=C(COc1ccc(Cl)c(F)c1)NCC1CCN(NC(=O)c2ccc3cc(Cl)ccc3n2)C1]

To a solution of N-((1-aminopyrrolidin-3-yl)methyl)-2-(4-chloro-3-fluorophenoxy)acetamide (1.0 equiv) in DMF (5 mL) is added 6-chloroquinoline-2-carboxylic acid (1.0 equiv) and HATU (2.0 equiv) at RT. The resulting reaction mixture is stirred for 10 minutes and DIPEA (3.0 equiv) is added. The reaction mixture is allowed to stir at RT overnight. Product formation is confirmed by LCMS. The reaction mixture is diluted with water (20 mL) and extracted with ethyl acetate (50 mL×2). Combined organic layer is washed with water (20 mL×4), dried over anhydrous sodium sulfate and concentrated. The crude product is purified by reverse phase HPLC to obtain 6-chloro-N-(3-((2-(4-chloro-3-fluorophenoxy)acetamido)methyl)pyrrolidin-1-yl)quinoline-2-carboxamide (Compound 11)

### Example 21

### Synthesis of 5-chloro-N-(3-((2-(4-chloro-3-fluorophenoxy)acetamido)methyl)pyrrolidin-1-yl)benzofuran-2-carboxamide

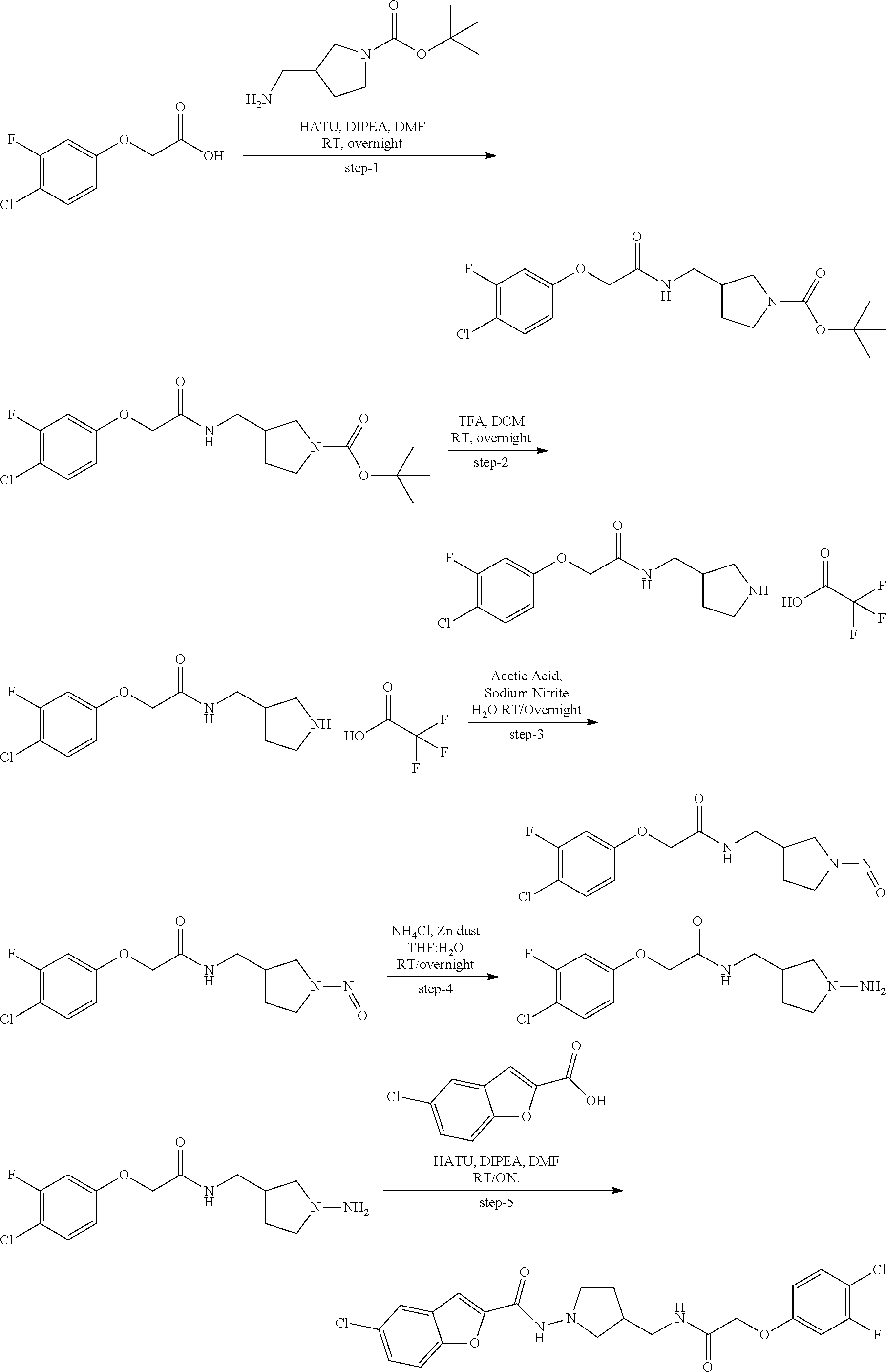

### Step-1: Synthesis of tert-butyl 3-((2-(4-chloro-3-fluorophenoxy)acetamido)methyl)pyrrolidine-1-carboxylate

To a solution of 2-(4-chloro-3-fluorophenoxy)acetic acid (1000 mg, 4.9 mmol, 1.0 equiv) in DMF (10 mL) was added tert-butyl 3-(aminomethyl)pyrrolidine-1-carboxylate (990 mg, 4.9 mmol, 1.0 equiv) and HATU (3700 mg, 9.9 mmol, 2.0 equiv) at RT. The reaction mixture was stirred for 10 minutes and then DIPEA (1.6 mL, 9.9 mmol, 2.0 equiv) was added. The reaction mixture was allowed to stir at RT overnight. Product formation was confirmed by LCMS. The reaction mixture was diluted with water (100 mL) and extracted with ethyl acetate (100 mL×2). Combined organic extracts were washed with water (100 mL×3), dried over anhydrous Na2SO4 and concentrated. Which gives to obtain tert-butyl 3-((2-(4-chloro-3-fluorophenoxy)acetamido)methyl)pyrrolidine-1-carboxylate (2300 mg, as a viscous yellow solid). LCMS 386.14 [M+H]⁺; ¹H NMR (400 MHz, DMSO-d₆) δ 8.25 (br. s., 1H), 7.45-7.51 (m, 1H), 7.06 (d, J=11.40 Hz, 1H), 6.85 (d, J=8.33 Hz, 1H), 4.54 (s, 2H), 3.11-3.20 (m, 2H), 2.89 (s, 2H), 2.65-2.78 (m, 3H), 1.83 (br. s., 1H), 1.51 (d, J=9.21 Hz, 1H), 1.38 (s, 9H).

### Step-2: Synthesis of 2-(4-chloro-3-fluorophenoxy)-N-(pyrrolidin-3-ylmethyl)acetamide trifluoroacetate salt

To a stirred solution of tert-butyl 3-((2-(4-chloro-3-fluorophenoxy)acetamido)methyl)pyrrolidine-1-carboxylate (2300 mg, 5.95 mmol, 1.0 equiv) in DCM (10 mL) was added TFA (2 mL) at RT. the reaction mixture was allowed to stir at RT overnight. DCM and excess of TFA was removed under reduced pressure to obtain 2-(4-chloro-3-fluorophenoxy)-N-(pyrrolidin-3-ylmethyl)acetamideas trifluoroacetate salt (Quantitative Yield) as a yellow semi solid. LCMS 287.2 [M+H]⁺; ¹H NMR (400 MHz, DMSO-d₆) δ 8.33 (t, J=5.70 Hz, 1H), 7.50 (t, J=8.77 Hz, 1H), 7.07 (dd, J=2.85, 11.18 Hz, 1H), 6.85 (dd, J=2.63, 8.77 Hz, 1H), 4.55 (s, 2H), 3.38 (q, J=7.02 Hz, 1H), 3.04-3.26 (m, 4H), 2.75-2.90 (m, 2H), 2.73 (s, 1H), 1.89-2.02 (m, 1H), 1.58 (dd, J=8.11, 12.94 Hz, 1H)

### Step-3: Synthesis of 2-(4-chloro-3-fluorophenoxy)-N-((1-nitrosopyrrolidin-3-yl)methyl)acetamide

To a solution of 2-(4-chloro-3-fluorophenoxy)-N-(pyrrolidin-3-ylmethyl)acetamideas trifluoroacetate salt (1800 mg, 4.5 mmol, 1.0 eq.) in water (20 mL) was added acetic acid (15 mL) and sodium nitrite (1200 mg, 18.0 mmol, 4.0 eq.) at RT. The reaction mixture was allowed to stir at RT overnight. Product formation was confirmed by LCMS. The reaction mixture was diluted with water (100 mL) and extracted with ethyl acetate (100 mL×2). Combined organic extracts were washed with water, brine and NaHCO₃ (100 mL×3), dried over anhydrous Na2SO4 and concentrated obtain to 2-(4-chloro-3-fluorophenoxy)-N-((1-nitrosopyrrolidin-3-yl)methyl)acetamide (1000 mg, 75% Yield) as a yellow semi solid. LCMS 316.0 [M+H]⁺; ¹H NMR (400 MHz, DMSO-d6) δ 8.32 (d, J=17.98 Hz, 1H), 7.48-7.54 (m, 1H), 6.98-7.13 (m, 1H), 6.78-6.88 (m, 1H), 4.56 (d, J=6.14 Hz, 2H), 4.28-4.41 (m, 1H), 4.15 (br. s., 1H), 3.93 (br. s., 1H), 3.54-3.64 (m, 1H), 3.41 (d, J=7.45 Hz, 1H), 3.10-3.22 (m, 2H), 1.98-2.06 (m, 1H), 1.64-1.76 (m, 1H).

### Step-4: Synthesis of N-((1-aminopyrrolidin-3-yl)methyl)-2-(4-chloro-3-fluorophenoxy)acetamide

To a stirred solution of 2-(4-chloro-3-fluorophenoxy)-N-((1-nitrosopyrrolidin-3-yl)methyl)acetamide (500 mg, 1.58 mmol, 1.0 equiv) in THF: H2O (07:10 mL) was added NH₄Cl (1370 mg, 25.39 mmol, 16.0 equiv) and then Zn dust (872 mg, 12.64 mmol, 8.0 equiv) was added portion wise. After addition, the reaction mixture was stirred at RT for overnight. Progress of the reaction was monitored by LCMS. Reaction mixture was diluted with water (100 mL) and filter out on Celite bed and filtrate was extracted with DCM (100 mL×2). Organic layer was separated and dried over anhydrous Na2SO₄ and concentrated under reduced pressure, to obtain N-((1-aminopyrrolidin-3-yl)methyl)-2-(4-chloro-3-fluorophenoxy)acetamide (250 mg,) as an yellow semi solid. LCMS 302.1 [M+H]⁺; ¹H NMR (400 MHz, DMSO-d₆) δ 8.19 (br. s., 1H), 7.49 (t, J=8.99 Hz, 1H), 7.06 (td, J=3.07, 11.40 Hz, 1H), 6.80-6.90 (m, 1H), 4.45-4.57 (m, 2H), 3.07-3.20 (m, 2H), 2.70-2.86 (m, 2H), 2.24-2.32 (m, 1H), 1.71-1.86 (m, 4H), 1.30-1.48 (m, 2H).

### Step-5: Synthesis of 5-chloro-N-(3-((2-(4-chloro-3-fluorophenoxy)acetamido)methyl)pyrrolidin-1-yl)benzofuran-2-carboxamide)

To a solution of N-((1-aminopyrrolidin-3-yl)methyl)-2-(4-chloro-3-fluorophenoxy)acetamide (125 mg, 0.41 mmol, 1.0 eq.) in DMF (05 mL) was added 5-chlorobenzofuran-2-carboxylic acid (80 mg, 0.41 mmol, 1.0 eq.) and HATU (313 mg, 0.82 mmol, 2.0 eq.) at RT. The resulting reaction mixture was stir for 10 minutes; DIPEA (0.15 mL, 0.82 mmol, 2.0 eq.) was added. The reaction mixture was allowed to stir at RT overnight. Product formation was confirmed by LCMS. The reaction mixture was diluted with water (50 mL) and extracted with ethyl acetate (100 mL×2). Combined organic extracts were washed with water (50 mL×4), dried over anhydrous Na2SO4 and concentrated. The crude product which was purified by reverse phase HPLC to 5-chloro-N-(3-((2-(4-chloro-3-fluorophenoxy)acetamido) methyl)pyrrolidin-1-yl)benzofuran-2-carboxamide (Compound 12—25 mg, 12% Yield) as a white solid. LCMS 480.2 [M+H]⁺; ¹H NMR (400 MHz, DMSO-d₆) δ 9.77 (s, 1H), 8.25 (br. s., 1H), 7.87 (d, J=1.75 Hz, 1H), 7.69 (d, J=8.77 Hz, 1H), 7.42-7.55 (m, 2H), 7.07 (dd, J=2.63, 11.40 Hz, 1H), 6.86 (d, J=7.45 Hz, 1H), 4.54 (s, 2H), 3.16 (br. s., 2H), 2.85-3.07 (m, 3H), 2.09 (s, 1H), 1.91 (br. s., 2H), 1.48 (br. s., 2H).

### Example 22

### Synthesis of 6-chloro-N-((1-((R)-3-(4-chloro-3-fluorophenoxy)-2-hydroxypropyl)pyrrolidin-3-yl)methyl)quinoline-2-carboxamide

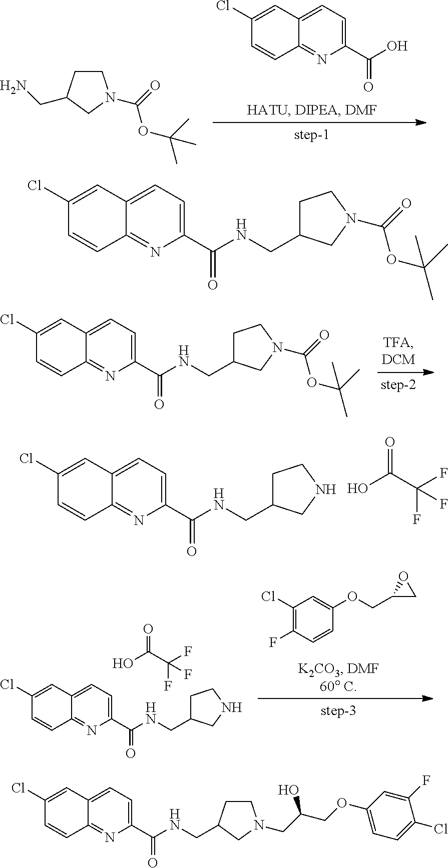

### Step-1: Synthesis of tert-butyl 3-((6-chloroquinoline-2-carboxamido)methyl)pyrrolidine-1-carboxylate

To a stirred solution of 6-chloroquinoline-2-carboxylic acid (0.510 g, 2.5 mmol, 1.0 equiv) in DMF (5 ml) was added tert-butyl 3-(aminomethyl)pyrrolidine-1-carboxylate (0.50 g, 2.5 mmol, 1.0 equiv), HATU (1.4 g, 3.75 mmol, 1.5 equiv) and DIPEA (0.2 mL, 1.0 mmol, 4.0 equiv). The reaction mixture was allowed to stir at RT for overnight. Product formation was confirmed by LCMS. The reaction mixture was diluted with water (50 mL) and extracted with ethyl acetate (30 mL×3). Combined organic layer was washed with water (30 mL×6). Organic layer was dried over anhydrous sodium sulfate, filtered and concentrated to obtain crude. Crude material was purified by flash chromatography (5% MeOH in DCM as eluent) to obtain tert-butyl 3-((6-chloroquinoline-2-carboxamido)methyl)pyrrolidine-1-carboxylate (0.9 g, 93% Yield ) as an yellow semi solid. LCMS 390.2 [M+H]⁺; ¹H NMR (400 MHz, DMSO-d₆) δ 9.11 (br. s., 1H), 8.55 (d, J=8.8 Hz, 1H), 8.32-8.09 (m, 3H), 7.89 (dd, J=2.2, 9.0 Hz, 1H), 3.37 (d, J=6.8 Hz, 2H), 3.22 (d, J=8.3 Hz, 1H), 3.04 (dd, J=7.1, 10.5 Hz, 1H), 1.91 (br. s., 1H), 1.64 (d, J=6.8 Hz, 1H), 1.38 (s, 9H).

### Step-2: Synthesis of 6-chloro-N-(pyrrolidin-3-ylmethyl)quinoline-2-carboxamide 2,2,2-trifluoroacetate

To a stirred solution of tert-butyl 3-((6-chloroquinoline-2-carboxamido)methyl)pyrrolidine-1-carboxylate (0.9 g, 1.036 mmol, 1.0 equiv) in DCM (10 ml) was added TFA (0.5 ml). The reaction mixture was allowed to stir at RT for overnight. Reaction progress was monitored by NMR. After completion of reaction the reaction mixture was concentrated under reduced pressure to obtain 6-chloro-N-(pyrrolidin-3-ylmethyl)quinoline-2-carboxamide 2,2,2-trifluoroacetate (0.9 g, 96% Yield) as a brown oil. LCMS 290.2 [M+H]⁺

### Step-3: Synthesis of 6-chloro-N-((1-((R)-3-(4-chloro-3-fluorophenoxy)-2-hydroxypropyl)pyrrolidin-3-yl)methyl)quinoline-2-carboxamide

To a stirred solution of 6-chloro-N-(pyrrolidin-3-ylmethyl)quinoline-2-carboxamide 2,2,2-trifluoroacetate (0.2 g, 0.49 mmol, 1.0 equiv) in DMF (10 ml) was added K₂CO₃ (0.135 g, 0.98 mmol, 1.0 equiv) followed by the addition of (R)-2-((3-chloro-4-fluorophenoxy)methyl)oxirane (0.1 g, 0.49 mmol, 1.0 equiv). The reaction mixture was allowed to stir at 60° C. for overnight. Product formation was confirmed by LCMS. The reaction mixture was diluted with water (50 mL) and extracted with ethyl acetate (30 mL×3). Combined organic layer was washed with water (30 mL×6). Organic layer was dried over anhydrous sodium sulfate, filtered and concentrated under reduced pressure. Crude material was purified by combiflash chromatography (5% MeOH in DCM as an eluent) to obtain 6-chloro-N-((1-((R)-3-(4-chloro-3-fluorophenoxy)-2-hydroxypropyl)pyrrolidin-3-yl)methyl)quinoline-2-carboxamide (Compound 80—0.05 g, 21% Yield) as an yellow semi solid. LCMS 492.3 [M+H]⁺; ¹H NMR (DMSO-d₆, 400 MHz) δ 9.05 (d, J=8.8 Hz, 1H), 8.53 (dd, J=8.8, 1.8 Hz, 1H), 8.23 (d, J=1.8 Hz, 1H), 8.16-8.21 (m, 1H), 8.06-8.16 (m, 1H), 7.85 (dd, J=9.0, 2.4 Hz, 1H), 7.36-7.49 (m, 1H), 6.95-7.10 (m, 1H), 6.70-6.84 (m, 1H), 4.91-5.06 (m, 1H), 4.68 (t, J=5.7 Hz, 1H), 3.98-4.06 (m, 1H), 3.82-3.96 (m, 2H), 3.36 (d, J=6.1 Hz, 1H), 2.67 (br. s., 2H), 1.88 (br. s., 2H), 1.52 (br. s., 2H).

### Example 23

### Synthesis of 6-chloro-N-((1-((S)-3-(4-chloro-3-fluorophenoxy)-2-hydroxypropyl)pyrrolidin-3-yl)methyl)quinoline-2-carboxamide

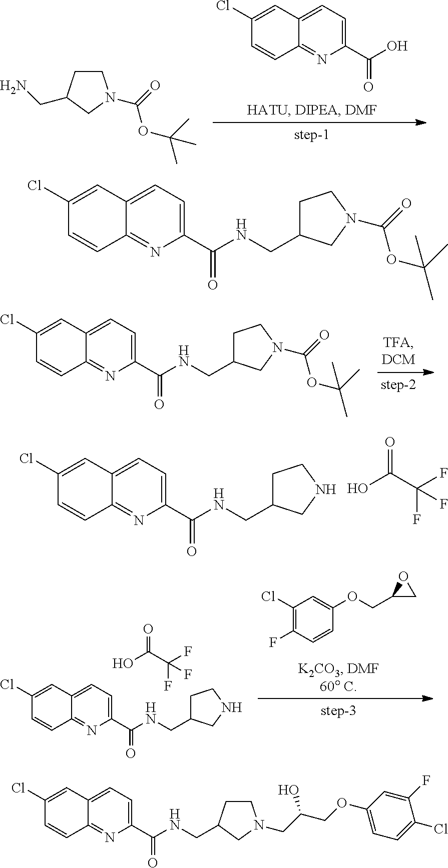

### Step-1: Synthesis of tert-butyl 3-((6-chloroquinoline-2-carboxamido)methyl)pyrrolidine-1-carboxylate

To a stirred solution of 6-chloroquinoline-2-carboxylic acid (0.510 g, 2.5 mmol, 1.0 equiv) in DMF (5 ml) was added tert-butyl 3-(aminomethyl)pyrrolidine-1-carboxylate (0.50 g, 2.5 mmol, 1.0 equiv), HATU (1.4 g, 3.75 mmol, 1.5 equiv) and DIPEA (0.2 mL, 1.0 mmol, 4.0 equiv). The reaction mixture was allowed to stir at RT for overnight. Product formation was confirmed by LCMS. The reaction mixture was diluted with water (50 mL) and extracted with ethyl acetate (30 mL×3). Combined organic layer was washed with water (30 mL×6). Organic layer was dried over anhydrous sodium sulfate, filtered and concentrated to obtain crude. Crude material was purified by flash chromatography (5% MeOH in DCM as eluent) to obtain tert-butyl 3-((6-chloroquinoline-2-carboxamido)methyl)pyrrolidine-1-carboxylate (0.9 g, 93% Yield ) as an yellow semi solid. LCMS 390.2 [M+H]⁺; ¹H NMR (400 MHz, DMSO-d₆) δ 9.11 (br. s., 1H), 8.55 (d, J=8.8 Hz, 1H), 8.32-8.09 (m, 3H), 7.89 (dd, J=2.2, 9.0 Hz, 1H), 3.37 (d, J=6.8 Hz, 2H), 3.22 (d, J=8.3 Hz, 1H), 3.04 (dd, J=7.1, 10.5 Hz, 1H), 1.91 (br. s., 1H), 1.64 (d, J=6.8 Hz, 1H), 1.38 (s, 9H).

### Step-2: Synthesis of 6-chloro-N-(pyrrolidin-3-ylmethyl)quinoline-2-carboxamide 2,2,2-trifluoroacetate

To a stirred solution of tert-butyl 3-((6-chloroquinoline-2-carboxamido)methyl)pyrrolidine-1-carboxylate (0.9 g, 1.036 mmol, 1.0 equiv) in DCM (10 ml) was added TFA (0.5 ml). The reaction mixture was allowed to stir at RT for overnight. Reaction progress was monitored by NMR. After completion of reaction the reaction mixture was concentrated under reduced pressure to obtain 6-chloro-N-(pyrrolidin-3-ylmethyl)quinoline-2-carboxamide 2,2,2-trifluoroacetate (0.9 g, 96% Yield) as a brown oil. LCMS 290.2 [M+H]⁺

### Step-3: Synthesis of 6-chloro-N-((1-((S)-3-(4-chloro-3-fluorophenoxy)-2-hydroxypropyl)pyrrolidin-3-yl)methyl)quinoline-2-carboxamide

To a stirred solution of 6-chloro-N-(pyrrolidin-3-ylmethyl)quinoline-2-carboxamide 2,2,2-trifluoroacetate (0.2 g, 0.49 mmol, 1.0 equiv) in DMF (10 ml) was added K₂CO₃ (0.135 g, 0.98 mmol, 1.0 equiv) followed by the addition of (S)-2-((3-chloro-4-fluorophenoxy)methyl)oxirane (0.1 g, 0.49 mmol, 1.0 equiv). The reaction mixture was allowed to stir at 60° C. for overnight. Product formation was confirmed by LCMS. The reaction mixture was diluted with water (50 mL) and extracted with ethyl acetate (30 mL×3). Combined organic layer was washed with water (30 mL×6). Organic layer was dried over anhydrous sodium sulfate, filtered and concentrated under reduced pressure. Crude material was purified by flash chromatography (5% MeOH in DCM as an eluent) to obtain 6-chloro-N-((1-((S)-3-(4-chloro-3-fluorophenoxy)-2-hydroxypropyl)pyrrolidin-3-yl)methyl)quinoline-2-carboxamide (Compound 81-0.05 g, 21% Yield) as an yellow semi solid. LCMS 492.3 [M+H]⁺; ¹H NMR (DMSO-d₆, 400 MHz) δ 9.05 (br. s., 1H), 8.53 (d, J=7.0 Hz, 1H), 8.23 (s, 1H), 8.17-8.21 (m, 1H), 8.07-8.17 (m, 1H), 7.80-7.92 (m, 1H), 7.41 (br. s., 1H), 7.01 (d, J=7.9 Hz, 1H), 6.79 (br. s., 1H), 4.91 (br. s., 2H), 4.02 (d, J=7.9 Hz, 1H), 3.91 (br. s., 2H), 1.91 (br. s., 2H), 1.51 (br. s., 2H), 1.35 (s, 1H), 1.23 ppm (br. s., 2H).

### Example 24

### Synthesis of 5-chloro-N-(3-(((R)-3-(4-chloro-3-fluorophenoxy)-2-hydroxypropyl)amino)cyclopentyl)benzofuran-2-carboxamide

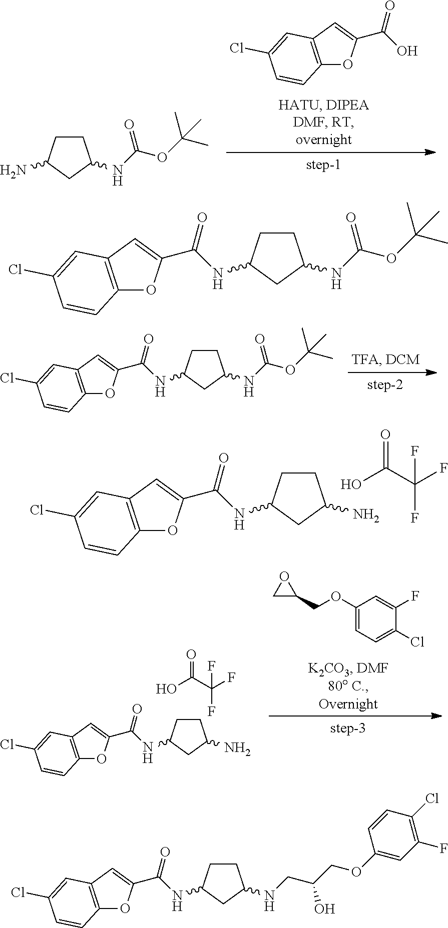

### Step-1. Synthesis of tert-butyl (3-(5-chlorobenzofuran-2-carboxamido)cyclopentyl)carbamate

To a solution of tert-butyl (3-aminocyclopentyl)carbamate hydrochloride (1000 mg, 4.2 mmol, 1.0 equiv) in DMF (05 mL) was added 5-chlorobenzofuran-2-carboxylic acid (834 mg, 4.2 mmol, 1.0 equiv) and HATU (834 mg, 8.4 mmol, 2.0 equiv) at RT. The reaction mixture was stirred for 10 minutes and then DIPEA (2.2 mL, 12.6 mmol, 3.0 equiv) was added. The resultant reaction mixture was allowed to stir at RT for overnight. Progress of the reaction was monitored by LCMS. The reaction mixture was diluted with water (200 mL) and precipitated solid was filtered off and dried under vacuum to obtain tert-butyl (3-(5-chlorobenzofuran-2-carboxamido)cyclopentyl)carbamate (1200 mg, 75% Yield) as an off-white solid. LCMS 379.1 [M+H]⁺; ¹H NMR (400 MHz, DMSO-d₆) δ 8.74 (d, J=7.45 Hz, 1H), 7.87 (d, J=2.19 Hz, 1H), 7.69 (d, J=8.77 Hz, 1H), 7.35-7.60 (m, 2H), 6.99 (d, J=7.02 Hz, 1H), 4.24 (dd, J=7.67, 15.13 Hz, 1H), 3.79 (d, J=6.58 Hz, 1H), 2.69-2.82 (m, 1H), 2.23-2.33 (m, 1H), 1.87 (dd, J=5.26, 12.28 Hz, 1H), 1.77 (dd, J=6.36, 12.06 Hz, 1H), 1.51-1.72 (m, 2H), 1.28-1.51 (m, 9H).

### Step-2: Synthesis of N-(3-aminocyclopentyl)-5-chlorobenzofuran-2-carboxamide 2,2,2-trifluoroacetate

To a stirred solution of tert-butyl (3-(5-chlorobenzofuran-2-carboxamido)cyclopentyl)carbamate (500 mg, 1.31 mmol, 1.0 equiv) in DCM (15 mL) was added TFA (5 mL) at RT. the reaction mixture was allowed to stir at RT overnight. DCM and excess of TFA was removed under reduced pressure to obtain N-(3-aminocyclopentyl)-5-chlorobenzofuran-2-carboxamide 2,2,2-trifluoroacetate (300 mg, 100% Yield) as a viscous solid. ¹H NMR (400 MHz, DMSO-d₆) δ 8.82 (d, J=7.45 Hz, 1H), 7.89 (d, J=2.19 Hz, 2H), 7.70 (d, J=8.77 Hz, 1H), 7.44-7.61 (m, 2H), 4.20-4.30 (m, 1H), 3.34 (m, 1H), 2.29-2.39 (m, 2H), 1.91-2.02 (m, 2H), 1.70-1.82 (m, 2H).

### Step-3: Synthesis of 5-chloro-N-(3-(((R)-3-(4-chloro-3-fluorophenoxy)-2-hydroxypropyl)amino)cyclopentyl)benzofuran-2-carboxamide

To a stirred solution of N-(3-aminocyclopentyl)-5-chlorobenzofuran-2-carboxamide 2,2,2-trifluoroacetate (100 mg, 0.26 mmol, 1.0 equiv) in DMF (05 ml) was added K₂CO₃ (72 mg, 0.52 mmol, 2.0 equiv) followed by the addition of (R)-2-((4-chloro-3-fluorophenoxy)methyl)oxirane (54 mg, 0.26 mmol, 1.0 equiv). The reaction mixture was allowed to stir at 80° C. for overnight. Product formation was confirmed by LCMS. The reaction mixture was diluted with water (100 mL) and extracted with ethyl acetate (100 mL). Combined organic layer was washed with brine and NaHCO₃ (100 mL×2). Organic layer was dried over anhydrous sodium sulfate, filtered and concentrated under reduced pressure. Crude material was purified by normal phase flash chromatography (0-5% MeOH in DCM as an eluent) to obtain 5-chloro-N-(3-(((R)-3-(4-chloro-3-fluorophenoxy)-2-hydroxypropyl)amino)cyclopentyl)benzofuran-2-carboxamide (Compound 82—40 mg, 32% ) as a white solid. LCMS 481.2 [M+H]⁺; ¹H NMR (400 MHz, DMSO-d₆) δ 8.79-8.86 (m, 1H) 7.84 (br. s., 1H) 7.62-7.70 (m, 1H) 7.36-7.57 (m, 3H) 7.04 (t, J=10.96 Hz, 1H) 6.82 (d, J=8.33 Hz, 1H) 4.28 (br. s., 1H) 4.03 (d, J=5.70 Hz, 1H) 3.95 (br. s., 2H) 3.20 (br. s., 1H) 2.73 (br. s., 1H) 2.12 (br. s., 2H) 1.86 (br. s., 2H) 1.70 (br. s., 2H) 1.60 (br. s., 2H).

### Example 25

### Synthesis of 5-chloro-N-(3-(((S)-3-(4-chloro-3-fluorophenoxy)-2-hydroxypropyl)amino)cyclopentyl)benzofuran-2-carboxamide

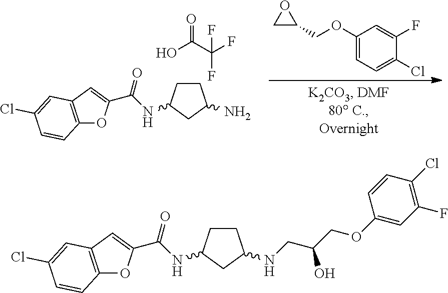

To a stirred solution of N-(3-aminocyclopentyl)-5-chlorobenzofuran-2-carboxamide 2,2,2-trifluoroacetate (100 mg, 0.26 mmol, 1.0 equiv) in DMF (05 ml) was added K₂CO₃ (72 mg, 0.52 mmol, 2.0 equiv) followed by the addition of (S)-2-((4-chloro-3-fluorophenoxy)methyl)oxirane (54 mg, 0.26 mmol, 1.0 equiv). The reaction mixture was allowed to stir at 80° C. for overnight. Product formation was confirmed by LCMS. The reaction mixture was diluted with water (100 mL) and extracted with ethyl acetate (100 mL). Combined organic layer was washed with brine and NaHCO₃ (100 mL×2). Organic layer was dried over anhydrous sodium sulfate, filtered and concentrated under reduced pressure. Crude material was purified by normal phase flash chromatography (0-5% MeOH in DCM as an eluent) to obtain 5-chloro-N-(3-(((S)-3-(4-chloro-3-fluorophenoxy)-2-hydroxypropyl)amino)cyclopentyl)benzofuran-2-carboxamide (Compound 83—30 mg, 24% ) a an white solid. LCMS 481.2 [M+H]⁺; ¹H NMR (400 MHz, DMSO-d₆) δ 8.79-8.86 (m, 1H) 7.84 (br. s., 1H) 7.62-7.70 (m, 1H) 7.36-7.57 (m, 3H) 7.04 (t, J=10.96 Hz, 1H) 6.82 (d, J=8.33 Hz, 1H) 4.28 (br. s., 1H) 4.03 (d, J=5.70 Hz, 1H) 3.95 (br. s., 2H) 3.20 (br. s., 1H) 2.73 (br. s., 1H) 2.12 (br. s., 2H) 1.86 (br. s., 2H) 1.70 (br. s., 2H) 1.60 (br. s., 2H).

### Example 26

### Synthesis of 2-(4-chloro-3-fluorophenoxy)-N-((3-(2-(4-chloro-3-fluorophenoxy)acetamido)cyclopentyl)methyl)acetamide

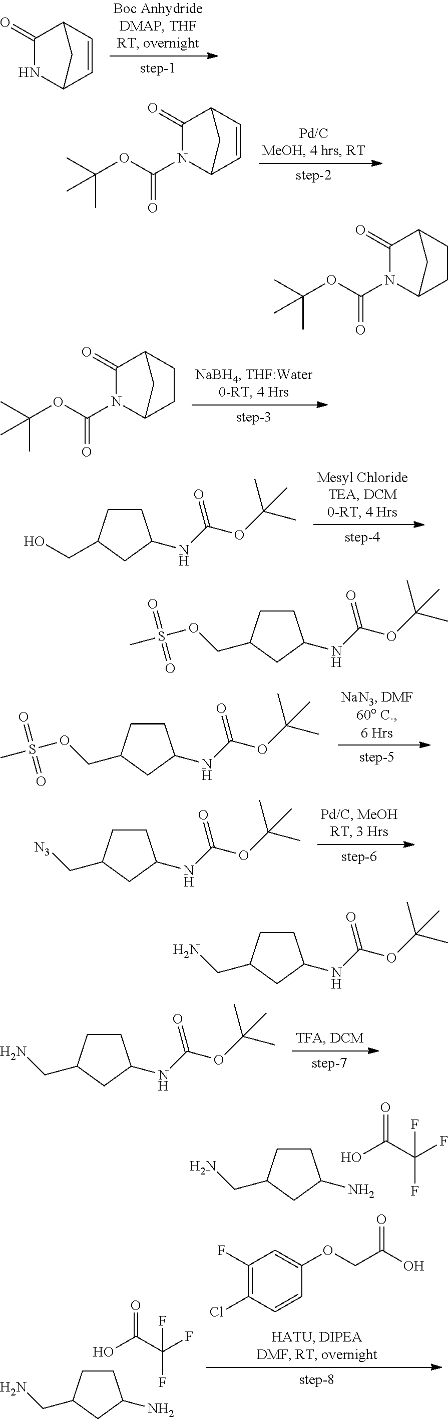

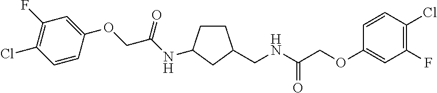 [SMILES: O=C(COc1ccc(Cl)c(F)c1)NCC1CCC(NC(=O)COc2ccc(Cl)c(F)c2)C1]

### Step-1: Synthesis of tert-butyl 3-oxo-2-azabicyclo[2.2.1]hept-5-ene-2-carboxylate

To a stirred solution of 2-azabicyclo[2.2.1]hept-5-en-3-one (10.0 g, 91.74 mmol, 1.0 equiv) in THF (200 mL) was added DMAP (1.11 g, 9.17 mmol, 0.1 equiv), triethyl amine (39 mL, 275.22 mmol, 3.0 equiv) followed by the addition of di-tert-butyl dicarbonate (24.0 g, 110 mmol, 1.2 equiv). The resulting reaction mixture was allowed to stir at RT for overnight. Product formation was confirmed by TLC & NMR spectroscopy. After completion of reaction, solvent was evaporated under reduced pressure and the residue was diluted with EtOAc (800 ml) and washed with water (3×500 ml) and brine (400 ml). The EtOAc extract was dried over Na2SO4 and evaporated under reduced pressure. Crude product was purified by flash chromatography (0-30% Ethyl acetate in Hexane as an eluent) to obtain tert-butyl 3-oxo-2-azabicyclo[2.2.1]hept-5-ene-2-carboxylate (19.0 g, 100% Yield) as a white solid. ¹H NMR (400 MHz, Chloroform-d) δ 6.89 (dd, J=2.2, 5.3 Hz, 1H), 6.65 (ddd, J=1.3, 3.3, 5.0 Hz, 1H), 4.95 (d, J=1.8 Hz, 1H), 3.43-3.29 (m, 1H), 2.41-2.27 (m, 1H), 2.24-2.08 (m, 1H), 1.50 (s, 9H).

### Step-2: Synthesis of tert-butyl 3-oxo-2-azabicyclo[2.2.1]heptane-2-carboxylate

Tert-butyl 3-oxo-2-azabicyclo[2.2.1]hept-5-ene-2-carboxylate (10.0 g, 4.78 mmol) was dissolved in MeOH (200 mL) was added Pd/C (1.3 g). Hydrogen gas was bubbled in to the mixture for 4 hrs. Product formation was confirmed by NMR spectroscopy. After completion of reaction the reaction mixture was filtered through Celite bed and filtrate was concentrated to obtain tert-butyl 3-oxo-2-azabicyclo[2.2.1]heptane-2-carboxylate (9.0 g, 90% Yield) as a white solid. ¹H NMR (400 MHz, Chloroform-d) δ 4.53 (s, 1H), 2.91-2.81 (m, 1H), 1.93-1.84 (m, 1H), 1.84-1.71 (m, 1H), 1.58 (br. s., 2H), 1.52 (s, 9H), 1.43-1.32 (m, 2H).

### Step-3: Synthesis of Cis-tert-butyl (3-(hydroxymethyl)cyclopentyl)carbamate

To a stirred solution of tert-butyl 3-oxo-2-azabicyclo[2.2.1]heptane-2-carboxylate (4.5 g, 21.31 mmol, 1.0 equiv) in THF:Water (25:20 mL) was added NaBH4 (1.60 g, 42.26 mmol, 2.0 equiv) portion wise at 0° C. The resulting reaction mixture was allowed to stir at RT for 4 h. Product formation was confirmed by TLC & NMR spectroscopy. After completion of reaction mixture was quenched with 1 M HCl and extracted with EtOAc (200 mL×2). Combined ethyl acetate layer was dried over Na2SO4 and evaporated under reduced pressure. Crude product was crystallized in n-pentane to obtain Cis-tert-butyl (3-(hydroxymethyl)cyclopentyl)carbamate (3.0 g, 65% Yield) as a white solid. ¹H NMR (400 MHz, Chloroform-d) δ 4.71 (br. s., 1H), 3.94 (br. s., 1H), 3.57 (d, J=4.4 Hz, 2H), 2.31-2.06 (m, 2H), 1.99-1.84 (m, 1H), 1.80-1.68 (m, 1H), 1.52-1.36 (m, 10H), 1.16-1.00 (m, 1H).

### Step-4: Synthesis of (3-((tert-butoxycarbonyl)amino)cyclopentyl)methyl methanesulfonate

To a solution of Cis-tert-butyl (3-(hydroxymethyl)cyclopentyl)carbamate (3.0 g, 13.9 mmol, 1.0 equiv ) in DCM (60 mL) was added TEA (4.02 mL, 27.9 mmol, 2.0 equiv) followed by the addition of methane sulfonyl chloride (1.4 mL, 18.13 mmol, 1.3 equiv) at 0° C. The reaction mixture was allowed to stir at RT for 4 h. Product formation was confirmed by NMR spectroscopy. After the completion of reaction the reaction mixture was quenched aq. Sodium bicarbonate and extracted with DCM (50 mL×3). Combined DCM layer was dried over Na2SO4 and evaporated under reduced pressure. Crude product was crystallized in n-pentane to obtain (3-((tert-butoxycarbonyl)amino)cyclopentyl)methyl methanesulfonate (2.8 g, 70% Yield) as a white solid. ¹H NMR (400 MHz, Chloroform-d) δ 4.52 (br. s., 1H), 4.14 (d, J=6.6 Hz, 2H), 3.96 (br. s., 1H), 3.01 (s, 3H), 2.43-2.19 (m, 2H), 2.06-1.94 (m, 1H), 1.90-1.71 (m, 1H), 1.58-1.32 (m, 12H), 1.16 (td, J=8.6, 12.7 Hz, 1H).

### Step-5: Synthesis of tert-butyl (3-(azidomethyl)cyclopentyl)carbamate

To a solution of (3-((tert-butoxycarbonyl)amino)cyclopentyl)methyl methanesulfonate (2.8 g, 9.55 mmol, 1.0 equiv ) in DMF (45 mL) was added NaN3 (1.24 g, 19.2 mmol, 2.0 equiv). The reaction mixture was heated 60° C. for 6 h. Product formation was confirmed by NMR spectroscopy. After the completion of reaction the reaction mixture was diluted with water and extracted with ethyl acetate (100 mL×3). Combined organic layer was washed with water (100 mL×3), dried over Na2SO4 and evaporated under reduced pressure to obtain tert-butyl (3-(azidomethyl)cyclopentyl)carbamate (2.5 g, 100% Yield) as an oil. ¹H NMR (400 MHz, Chloroform-d) d=4.55 (br. s., 1H), 3.94 (br. s., 1H), 3.28 (d, J=6.1 Hz, 2H), 2.34-2.12 (m, 2H), 2.00-1.86 (m, 1H), 1.86-1.68 (m, 1H), 1.52-1.35 (m, 9H), 1.26 (t, J=7.0 Hz, 1H), 1.16-1.00 (m, 1H).

### Step-6: Synthesis of tert-butyl (3-(aminomethyl)cyclopentyl)carbamate

Tert-butyl (3-(azidomethyl)cyclopentyl)carbamate (0.500 g, 2.78 mmol) was dissolved in MeOH (10 mL) was added Pd/C (0.030 g). Hydrogen gas was bubbled in to the mixture for 3 hrs. Product formation was confirmed by NMR spectroscopy. After completion of reaction the reaction mixture was filtered through Celite bed and filtrate was concentrated to obtain tert-butyl (3-(aminomethyl)cyclopentyl)carbamate (0.250 g, 56% Yield) as a colorless oil. ¹H NMR (400 MHz, Chloroform-d) δ 4.75 (br. s., 1H), 4.00-3.83 (m, 1H), 3.47 (s, 1H), 2.72-2.59 (m, 2H), 2.31-2.12 (m, 2H), 2.09-1.87 (m, 3H), 1.87-1.68 (m, 2H), 1.55-1.32 (m, 12H), 1.00 (td, J=8.3, 12.7 Hz, 1H).

### Step-7: Synthesis of 3-(aminomethyl)cyclopentan-1-amine 2,2,2-trifluoroacetate

To a solution of tert-butyl (3-(aminomethyl)cyclopentyl)carbamate (0.200 g, 0.934 mmol) in DCM (5 mL) was added TFA (0.5 mL). The resulting reaction mixture was allowed to stir at RT overnight. Reaction mixture was concentrated to obtain 3-(aminomethyl)cyclopentan-1-amine 2,2,2-trifluoroacetate (0.130 g) as an oil which was directly used for next step.

### Step-8: Synthesis of 2-(4-chloro-3-fluorophenoxy)-N-((3-(2-(4-chloro-3-fluorophenoxy)acetamido)cyclopentyl)methyl)acetamide

To a stirred solution of 2-(4-chloro-3-fluorophenoxy)acetic acid (0.100 g, 0.490 mmol, 1.0 equiv) in DMF (5 mL) was added 3-(aminomethyl)cyclopentan-1-amine 2,2,2-trifluoroacetate (0.067 g, 0.294 mmol, 0.6 equiv), HATU (0.279 g, 0.735 mmol, 1.5 equiv) and DIPEA (0.25 mL, 1.47 mmol, 3.0 equiv). The reaction mixture was allowed to stir at RT for 16 h. Product formation was confirmed by LCMS. The reaction mixture was diluted with water and extracted with ethyl acetate (50 mL×2). Combined organic layer was washed with water (25 mL×4), dried over anhydrous Na2SO4 and concentrated. Crude product was purified by flash chromatography (5% MeOH in DCM as an eluent) to obtain 2-(4-chloro-3-fluorophenoxy)-N-((3-(2-(4-chloro-3-fluorophenoxy)acetamido)cyclopentyl)methyl)acetamide (Compound 84—0.030 g, 13% Yield) as a white solid. LCMS 487 [M+H]⁺; ¹H NMR (400 MHz, DMSO-d₆) δ 8.17 (br. s., 1H), 8.08 (d, J=7.5 Hz, 1H), 7.61-0.41 (m, 2H), 7.13-6.97 (m, 2H), 6.84 (d, J=8.3 Hz, 2H), 4.51 (d, J=18.9 Hz, 4H), 4.05 (dd, J=7.9, 14.9 Hz, 1H), 3.11 (t, J=6.1 Hz, 2H), 2.15-1.87 (m, 3H), 1.81 (dd, J=6.1, 11.8 Hz, 1H), 1.70-1.51 (m, 2H), 1.51-1.40 (m, 1H), 1.36 (d, J=5.3 Hz, 1H), 1.15-0.97 (m, 2H).

### Example 27

### Synthesis of 6-chloro-N-(3-((2-(4-chloro-3-fluorophenoxy)acetamido)methyl)cyclopentyl)quinoline-2-carboxamide

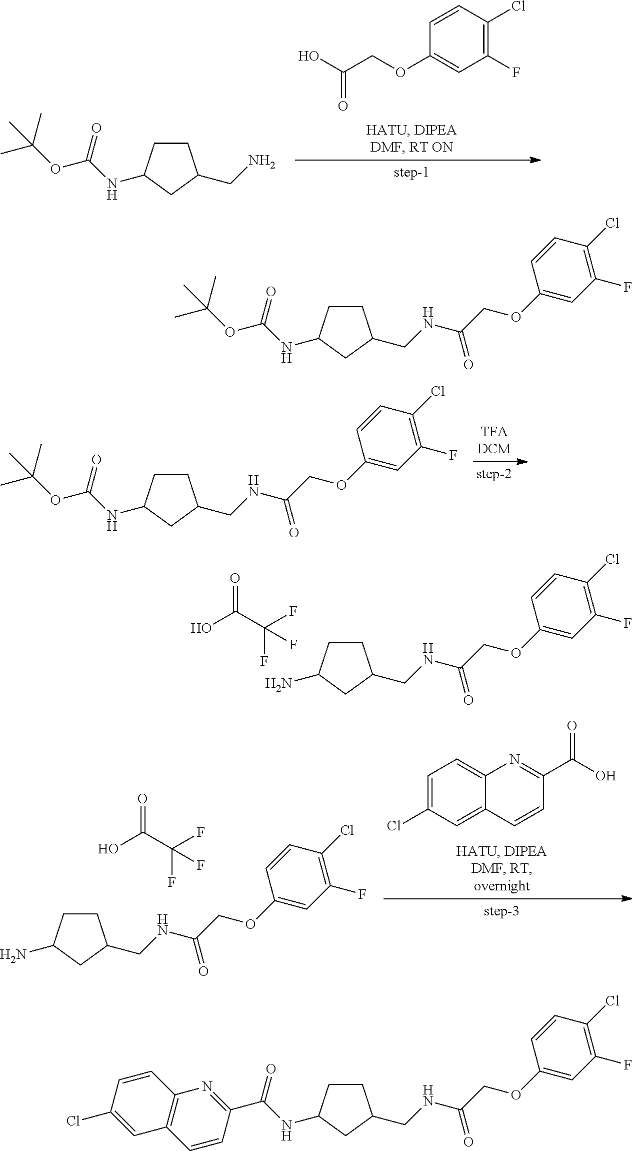

### Step-1: Synthesis of tert-butyl (3-((2-(4-chloro-3-fluorophenoxy)acetamido)methyl)cyclopentyl)carbamate

To a stirred solution of 2-(4-chloro-3-fluorophenoxy)acetic acid (0.100 g, 0.490 mmol, 1.0 equiv) in DMF (1.5 ml) was added tert-butyl (3-(aminomethyl)cyclopentyl)carbamate (0.104 g, 490 mmol, 1.0 equiv), HATU (0.372 g, 0.980 mmol, 2.0 equiv) and DIPEA (0.27 mL, 1.47 mmol, 3.0 equiv). The reaction mixture was allowed to stir at RT for 16 h. Product formation was confirmed by LCMS. The reaction mixture was diluted with water and extracted with ethyl acetate (50 mL×2). Combined organic layer was washed with water (20 mL×4), dried over anhydrous Na2SO4 and concentrated. The crude product was purified by flash chromatography to obtain tert-butyl (3-((2-(4-chloro-3-fluorophenoxy)acetamido)methyl)cyclopentyl)carbamate (0.100 g, 51% Yield ) as an off-white solid. LCMS 401.1 [M+H]⁺; ¹H NMR (400 MHz, DMSO-d₆) δ 8.11 (br. s., 1H), 7.49 (t, J=8.8 Hz, 1H), 7.06 (dd, J=2.6, 11.4 Hz, 1H), 6.94-6.75 (m, 2H), 4.52 (s, 2H), 3.70 (d, J=7.5 Hz, 1H), 3.14-3.00 (m, 2H), 2.06-1.89 (m, 2H), 1.83-1.66 (m, 1H), 1.44-1.35 (m, 9H), 1.03-0.91 (m, 1H).

### Step-2: Synthesis of N-((3-aminocyclopentyl)methyl)-2-(4-chloro-3-fluorophenoxy)acetamide 2,2,2-trifluoroacetate

To a stirred solution of tert-butyl (3-((2-(4-chloro-3-fluorophenoxy)acetamido)methyl)cyclopentyl)carbamate (0.600 g, 1.50 mmol, 1.0 equiv) in DCM (10 ml) was added TFA (0.9 ml). The reaction mixture was allowed to stir at RT for 16 h. Reaction progress was monitored by NMR. After completion of reaction the reaction mixture was concentrated under reduced pressure. Crude compound was crystallized by trituration in pentane and then in petroleum ether to obtain N-((3-aminocyclopentyl)methyl)-2-(4-chloro-3-fluorophenoxy)acetamide 2,2,2-trifluoroacetate (0.600 g, Quant. Yield); LCMS 300.1 [M+H]⁺; ¹H NMR (400 MHz, DMSO-d₆) δ 8.23 (t, J=5.7 Hz, 1H), 7.83 (br. s., 2H), 7.50 (t, J=9.0 Hz, 1H), 7.06 (dd, J=2.6, 11.4 Hz, 1H), 6.94-6.75 (m, 1H), 4.53 (s, 2H), 3.44 (d, J=4.8 Hz, 1H), 3.13 (t, J=5.9 Hz, 2H), 2.15-1.95 (m, 2H), 1.88 (dd, J=7.7, 12.5 Hz, 1H), 1.73-1.51 (m, 2H), 1.40 (dd, J=7.9, 11.4 Hz, 1H), 1.22-0.96 (m, 1H).

### Step-3: Synthesis of 6-chloro-N-(3-((2-(4-chloro-3-fluorophenoxy)acetamido)methyl)cyclopentyl)quinoline-2-carboxamide

To a stirred solution of N-((3-aminocyclopentyl)methyl)-2-(4-chloro-3-fluorophenoxy)acetamide 2,2,2-trifluoroacetate (0.100 g, 0.333 mmol, 1.0 equiv) in DMF (4 ml) were added 6-chloroquinoline-2-carboxylic acid (0.082 g, 0.400 mmol, 1.2 equiv), DMAP (0.101 g, 0.833 mmol, 2.5 equiv) and EDC.HCl (0.127 g, 0.666 mmol, 2.0 equiv). The reaction mixture was allowed to stir at RT for 16 h. Product formation was confirmed by LCMS. The reaction mixture was diluted with water which results in to precipitate. The resulting solid precipitate was filtered and washed with ice water, dried under vacuum to obtain 6-chloro-N-(3-((2-(4-chloro-3-fluorophenoxy)acetamido)methyl)cyclopentyl)quinoline-2-carboxamide (Compound 85—0.040 g, 33% Yield) as an off-white solid. LCMS 490.3 [M+H]⁺; ¹H NMR (400 MHz, DMSO-d₆) δ 8.75 (d, J=7.9 Hz, 1H), 8.53 (d, J=8.8 Hz, 1H), 8.24 (d, J=2.2 Hz, 1H), 8.16 (dd, J=5.9, 9.0 Hz, 2H), 7.88 (dd, J=2.2, 9.2 Hz, 1H), 7.48 (t, J=9.0 Hz, 1H), 7.06 (dd, J=2.6, 11.4 Hz, 1H), 6.92-6.73 (m, 1H), 4.54 (s, 2H), 4.41-4.15 (m, 1H), 3.18 (t, J=5.9 Hz, 2H), 2.21-2.01 (m, 2H), 1.91 (d, J=6.1 Hz, 1H), 1.77-1.55 (m, 2H), 1.46 (d, J=3.9 Hz, 1H), 1.42-1.20 (m, 1H).

### Example 28

### Synthesis of 5-chloro-N-(3-((2-(4-chloro-3-fluorophenoxy)acetamido)methyl)cyclopentyl)benzofuran-2-carboxamide

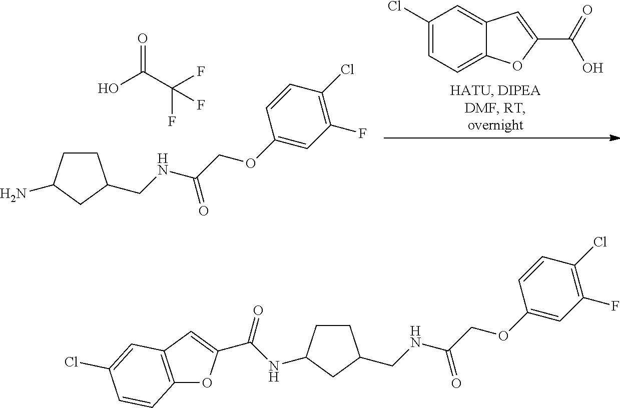 [SMILES: NC1CCC(CNC(=O)COc2ccc(Cl)c(F)c2)C1 ; O=C(O)C(F)(F)F ; O=C(O)c1cc2cc(Cl)ccc2o1 ; O=C(COc1ccc(Cl)c(F)c1)NCC1CCC(NC(=O)c2cc3cc(Cl)ccc3o2)C1]

To a stirred solution of N-((3-aminocyclopentyl)methyl)-2-(4-chloro-3-fluorophenoxy)acetamide 2,2,2-trifluoroacetate (0.100 g, 0.241 mmol, 1.0 equiv) in DMF (4 ml) were added 5-chlorobenzofuran-2-carboxylic acid (0.065 g, 0.241 mmol, 1.2 equiv), DMAP (0.101 g, 0.833 mmol, 2.5 equiv) and EDC.HCl (0.127 g, 0.666 mmol, 2.0 equiv). The reaction mixture was allowed to stir at RT for 16 h. Product formation was confirmed by LCMS. The reaction mixture was diluted with water which results in to precipitate. The resulting solid precipitate was filtered and washed with ice water, dried under vacuum to obtain 6-chloro-N-(3-((2-(4-chloro-3-fluorophenoxy)acetamido)methyl)cyclopentyl)quinoline-2-carboxamide (Compound 86—0.040 g, 35% Yield) as an off-white solid. LCMS 479.3 [M+H]⁺; ¹H NMR (400 MHz, DMSO-d₆) δ 8.70 (d, J=7.9 Hz, 1H), 8.18 (br. s., 1H), 7.86 (d, J=1.8 Hz, 1H), 7.68 (d, J=8.8 Hz, 1H), 7.55-7.42 (m, 2H), 7.07 (dd, J=3.1, 11.4 Hz, 1H), 6.85 (d, J=8.8 Hz, 1H), 4.54 (s, 2H), 4.22 (d, J=6.6 Hz, 1H), 3.15 (t, J=5.9 Hz, 2H), 2.23-2.00 (m, 2H), 1.88 (br. s., 1H), 1.64 (d, J=7.5 Hz, 2H), 1.42 (br. s., 1H), 1.25 (d, J=12.3 Hz, 1H).

### Example 29

### Synthesis of 6-chloro-N-((3-(2-(4-chloro-3-fluorophenoxy)acetamido)cyclopentyl)methyl)quinoline-2-carboxamide

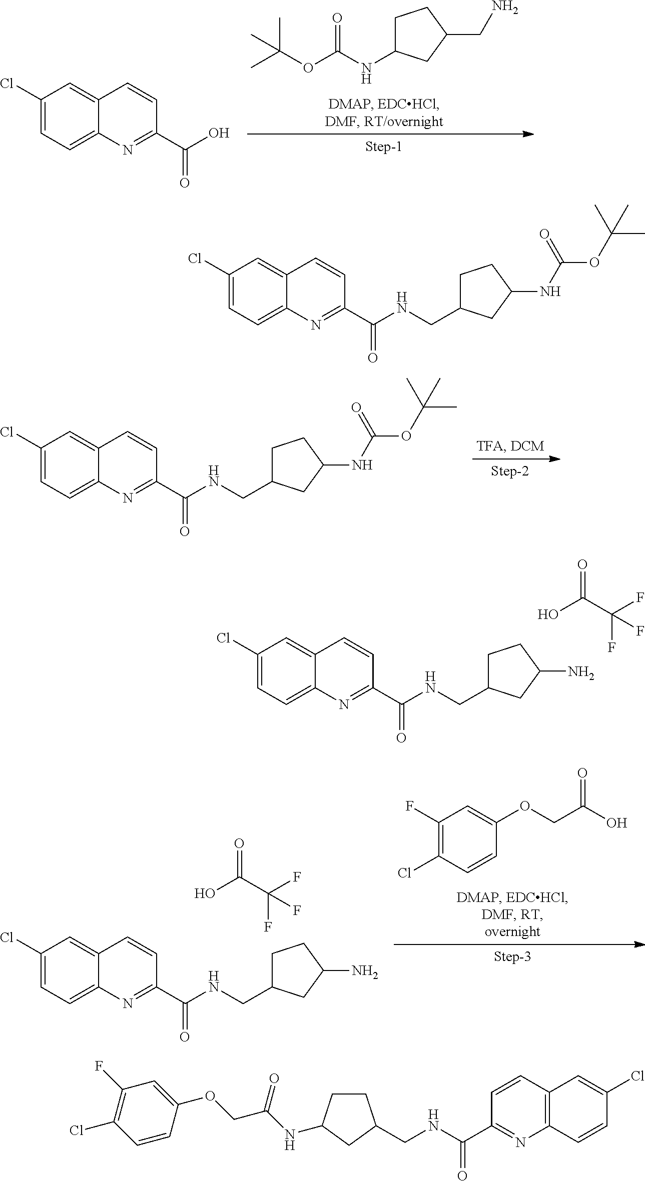

### Step-1: Synthesis of tert-butyl (3-((6-chloroquinoline-2-carboxamido)methyl)cyclopentyl)carbamate

To a solution of 6-chloroquinoline-2-carboxylic acid (0.500 g, 2.41 mmol, 1.0 equiv) in DMF (20 mL) was added tert-butyl (3-(aminomethyl)cyclopentyl)carbamate (0.518 g, 2.41 mmol, 1.0 equiv) and DMAP (0.737 g, 6.03 mmol, 2.5 equiv) at RT. The reaction mixture was stirred for 10 minutes and then EDC.HCl (0.926 g, 4.83 mmol, 2.0 equiv) was added. The reaction mixture was allowed to stir at RT overnight. Product formation was confirmed by LCMS. The reaction mixture was diluted with water (50 mL) and extracted with ethyl acetate (100 mL×2). Combined organic extracts were washed with water (50 mL×4), dried over anhydrous Na2SO4 and concentrated to obtain tert-butyl (3-((6-chloroquinoline-2-carboxamido)methyl)cyclopentyl)carbamate (0.500 g, 50% Yield) as an off-white solid. LCMS 403.1 [M+H]⁺; ¹H NMR (400 MHz, DMSO-d₆) δ 8.94 (t, J=5.92 Hz, 1H), 8.54 (d, J=8.33 Hz, 1H), 8.16-8.25 (m, 2H), 7.88 (dd, J=2.41, 8.99 Hz, 1H), 6.87 (d, J=7.45 Hz, 1H), 3.68-3.80 (m, 1H), 3.32-3.39 (m, 2H), 2.94 (s, 1H), 2.20 (td, J=7.56, 15.57 Hz, 1H), 1.93-2.04 (m, 1H), 1.78 (br. s., 1H), 1.58-1.70 (m, 2H), 1.33-1.51 (m, 9H), 1.14 (td, J=8.93, 12.39 Hz, 2H).

### Step-2: Synthesis of N-((3-aminocyclopentyl)methyl)-6-chloroquinoline-2-carboxamide trifluoroacetate salt

To a stirred solution of tert-butyl (3-((6-chloroquinoline-2-carboxamido)methyl)cyclopentyl)carbamate (0.500 g, 1.31 mmol, 1.0 equiv) in DCM (15 mL) was added TFA (05 mL) at RT. the reaction mixture was allowed to stir at RT overnight. DCM and excess of TFA was removed under reduced pressure to obtain N-((3-aminocyclopentyl)methyl)-6-chloroquinoline-2-carboxamide trifluoroacetate salt (0.200 g, Quantitative Yield) as an of white solid. LCMS 303.1 [M+H]⁺; ¹H NMR (400 MHz, DMSO-d₆) δ 8.94 (t, J=5.92 Hz, 1H), 8.54 (d, J=8.33 Hz, 1H), 8.16-8.25 (m, 2H), 7.88 (dd, J=2.41, 8.99 Hz, 1H), 6.87 (d, J=7.45 Hz, 1H), 3.68-3.80 (m, 1H), 3.32-3.39 (m, 2H), 2.94 (s, 1H), 2.20 (td, J=7.56, 15.57 Hz, 1H), 1.93-2.04 (m, 1H), 1.78 (br. s., 1H), 1.58-1.70 (m, 2H), 1.14 (td, J=8.93, 12.39 Hz, 2H).

### Step-3: Synthesis of 6-chloro-N-((3-(2-(4-chloro-3-fluorophenoxy)acetamido)cyclopentyl)methyl)quinoline-2-carboxamide

To a solution of N-((3-aminocyclopentyl)methyl)-6-chloroquinoline-2-carboxamide trifluoroacetate salt (0.200 g, 0.46 mmol, 1.0 equiv) in DMF (5 mL) was added 2-(4-chloro-3-fluorophenoxy)acetic acid (0.114 g, 0.55 mmol, 1.2 equiv) and DMAP (0.201 g, 1.65 mmol, 2.5 equiv) at RT. The reaction mixture was stirred for 10 minutes and then EDC.HCl (0.253 g, 1.32 mmol, 2.0 equiv) was added. The reaction mixture was allowed to stir at RT overnight. Product formation was confirmed by LCMS. The reaction mixture was diluted with water (50 mL) and extracted with ethyl acetate (100 mL×2). Combined organic extracts were washed with water (50 mL×4), dried over anhydrous Na2SO4 and concentrated. The crude product which was purified by normal phase of column chromatography to obtain 6-chloro-N-((3-(2-(4-chloro-3-fluorophenoxy)acetamido)cyclopentyl)methyl)quinoline-2-carboxamide (Compound 87—0.150 g, 67% Yield) as a white solid. LCMS 490.3 [M+H]⁺; ¹H NMR (400 MHz, DMSO-d₆) δ 8.99 (t, J=6.14 Hz, 1H), 8.54 (d, J=8.77 Hz, 1H), 8.06-8.34 (m, 4H), 7.88 (dd, J=2.41, 8.99 Hz, 1H), 7.46-7.57 (m, 1H), 7.07 (dd, J=2.63, 11.40 Hz, 1H), 6.84 (dd, J=1.75, 8.77 Hz, 1H), 4.49 (s, 2H), 4.05-4.14 (m, 1H), 3.35-3.44 (m, 2H), 2.23-2.31 (m, 1H), 1.99-2.08 (m, 1H), 1.79-1.87 (m, 1H), 1.66-1.71 (m, 1H), 1.44-1.58 (m, 2H), 1.15-1.28 (m, 1H).

### Example 30

### Synthesis of 5-chloro-N-((3-(2-(4-chloro-3-fluorophenoxy)acetamido)cyclopentyl)methyl)benzofuran-2-carboxamide

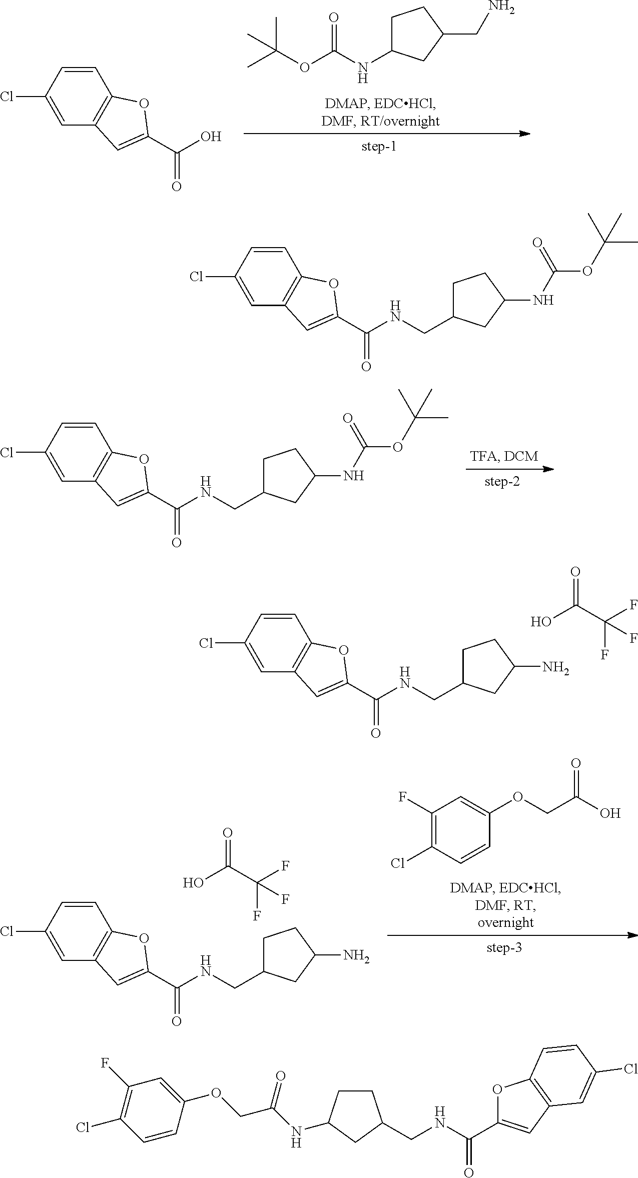

### Step-1: Synthesis of tert-butyl (3-((5-chlorobenzofuran-2-carboxamido)methyl)cyclopentyl)carbamate

To a solution of 5-chlorobenzofuran-2-carboxylic acid (0.500 g, 2.54 mmol, 1.0 equiv) in DMF (20 mL) was added tert-butyl (3-(aminomethyl)cyclopentyl)carbamate (0.545 g, 2.54 mmol, 1.0 equiv) and DMAP (0.776 g, 6.35 mmol, 2.5 equiv) at RT. The reaction mixture was stirred for 10 minutes and then EDC.HCl (0.975 g, 5.08 mmol, 2.0 equiv) was added. The reaction mixture was allowed to stir at RT overnight. Product formation was confirmed by LCMS. The reaction mixture was diluted with water (50 mL) and extracted with ethyl acetate (100 mL×2). Combined organic extracts were washed with water (50 mL×4), dried over anhydrous Na2SO4 and concentrated. to obtain tert-butyl (3-((5-chlorobenzofuran-2-carboxamido)methyl)cyclopentyl)carbamate (0.500 g, 50% Yield) as an off-white solid. LCMS 392.1 [M+H]⁺; ¹H NMR (400 MHz, DMSO-d₆) δ 8.79 (br. s., 1H), 7.87 (d, J=2.19 Hz, 1H), 7.69 (d, J=8.77 Hz, 1H), 7.42-7.55 (m, 2H), 6.86 (d, J=7.02 Hz, 1H), 3.72 (d, J=7.45 Hz, 1H), 3.23 (dq, J=6.58, 13.30 Hz, 2H), 2.09-2.17 (m, 1H), 1.93-2.04 (m, 1H), 1.76 (d, J=7.02 Hz, 1H), 1.54-1.63 (m, 1H), 1.39-1.50 (m, 2H), 1.37 (s, 9H), 1.03-1.13 (m, 1H).

### Step-2: Synthesis of N-((3-aminocyclopentyl)methyl)-5-chlorobenzofuran-2-carboxamidetrifluoroacetate salt

To a stirred solution of tert-butyl (3-((5-chlorobenzofuran-2-carboxamido)methyl)cyclopentyl)carbamate (0.500 g, 1.31 mmol, 1.0 equiv) in DCM (15 mL) was added TFA (02 mL) at RT. the reaction mixture was allowed to stir at RT overnight. DCM and excess of TFA was removed under reduced pressure to obtain N-((3-aminocyclopentyl)methyl)-5-chlorobenzofuran-2-carboxamide trifluoroacetate salt (0.200 g, Quantitative Yield) as an of white solid. LCMS 292.1 [M+H]⁺; ¹H NMR (400 MHz, DMSO-d₆) δ 8.79 (br. s., 1H), 7.87 (d, J=2.19 Hz, 1H), 7.69 (d, J=8.77 Hz, 1H), 7.42-7.55 (m, 2H), 6.86 (d, J=7.02 Hz, 1H), 3.72 (d, J=7.45 Hz, 1H), 3.23 (dq, J=6.58, 13.30 Hz, 2H), 2.09-2.17 (m, 1H), 1.93-2.04 (m, 1H), 1.76 (d, J=7.02 Hz, 1H), 1.54-1.63 (m, 1H), 1.39-1.50 (m, 2H), 1.03-1.13 (m, 1H).

### Step-3: Synthesis of 5-chloro-N-((3-(2-(4-chloro-3-fluorophenoxy)acetamido)cyclopentyl)methyl)benzofuran-2-carboxamide

To a solution of N-((3-aminocyclopentyl)methyl)-5-chlorobenzofuran-2-carboxamide trifluoroacetate salt (0.200 g, 0.49 mmol, 1.0 equiv) in DMF (5 mL) was added 2-(4-chloro-3-fluorophenoxy)acetic acid (0.120 g, 0.59 mmol, 1.2 equiv) and DMAP (0.203 g, 1.71 mmol, 2.5 equiv) at RT. The reaction mixture was stirred for 10 minutes and then EDC.HCl (0.262 g, 1.36 mmol, 2.0 equiv) was added. The reaction mixture was allowed to stir at RT overnight. Product formation was confirmed by LCMS. The reaction mixture was diluted with water (50 mL) and extracted with ethyl acetate (100 mL×2). Combined organic extracts were washed with water (50 mL×4), dried over anhydrous Na2SO4 and concentrated. The crude product which was purified by normal phase of column chromatography to obtain 5-chloro-N-((3-(2-(4-chloro-3-fluorophenoxy)acetamido)cyclopentyl)methyl)benzofuran-2-carboxamide (Compound 88—0.130 g, 65% Yield) as a white solid. LCMS 478.0 [M+H]⁺; ¹H NMR (400 MHz, DMSO-d₆) δ 8.84 (t, J=5.92 Hz, 1H), 8.11 (d, J=7.89 Hz, 1H), 7.87 (d, J=2.19 Hz, 1H), 7.69 (d, J=9.21 Hz, 1H), 7.42-7.57 (m, 3H), 7.07 (dd, J=2.63, 11.40 Hz, 1H), 6.85 (d, J=1.75 Hz, 1H), 4.49 (s, 2H), 4.05-4.12 (m, 1H), 3.27 (t, J=6.58 Hz, 2H), 2.12-2.20 (m, 1H), 2.04 (dd, J=7.24, 12.50 Hz, 1H), 1.83 (dd, J=5.48, 12.06 Hz, 1H), 1.67 (dd, J=5.92, 12.06 Hz, 1H), 1.39-1.57 (m, 2H), 1.19 (td, J=8.88, 12.50 Hz, 1H).

### Example 31

### Synthesis of 2-(4-chloro-3-fluorophenoxy)-N-((3-((3-(4-chloro-3-fluorophenoxy)-2-hydroxypropyl)amino)cyclopentyl)methyl)acetamide

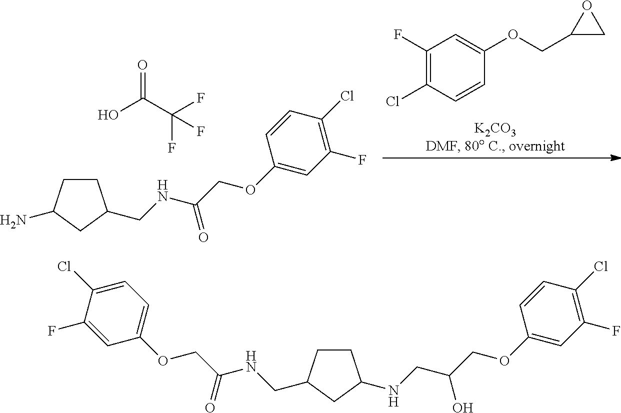

To a stirred solution of N-((3-aminocyclopentyl)methyl)-2-(4-chloro-3-fluorophenoxy)acetamide 2,2,2-trifluoroacetate (0.200 g, 0.483 mmol, 1.0 equiv) in DMF (5 ml) was added K₂CO₃ (0.229 g, 1.66 mmol, 2.5 equiv) followed by the addition of 2-((4-chloro-3-fluorophenoxy)methyl)oxirane (0.148 g, 0.733 mmol, 1.0 equiv). The reaction mixture was allowed to stir at 80° C. for overnight. Product formation was confirmed by LCMS. The reaction mixture was diluted with water (50 mL) and extracted with ethyl acetate (30 mL×3). Combined organic layer was washed with water (30 mL×6). Organic layer was dried over anhydrous sodium sulfate, filtered and concentrated under reduced pressure. Crude material was purified by flash chromatography (5% MeOH in DCM as an eluent) to obtain 2-(4-chloro-3-fluorophenoxy)-N-((3-((3-(4-chloro-3-fluorophenoxy)-2-hydroxypropyl)amino)cyclopentyl)methyl)acetamide (Compound 89—0.050 g, 21% Yield) as an off-white solid. LCMS 503.3 [M+H]⁺; ¹H NMR (400 MHz, DMSO-d₆) δ 8.19 (br. s., 1H), 7.60-7.34 (m, 2H), 7.04 (d, J=2.6 Hz, 1H), 7.07 (d, J=2.6 Hz, 1H), 6.89-6.73 (m, 2H), 5.11 (br. s., 1H), 4.52 (s, 2H), 3.98 (d, J=7.9 Hz, 1H), 3.94-3.77 (m, 2H), 3.48-3.19 (m, 3H), 3.19-2.90 (m, 3H), 2.65 (d, J=8.8 Hz, 1H), 2.61-2.52 (m, 2H), 2.11-1.82 (m, 3H), 1.80-1.66 (m, 1H), 1.66-1.51 (m, 1H), 1.44-1.26 (m, 2H), 1.03-0.93 (m, 1H).

### Example 32

### Synthesis of 6-chloro-N-((3-((3-(4-chloro-3-fluorophenoxy)-2-hydroxypropyl)amino)cyclopentyl)methyl)quinoline-2-carboxamide

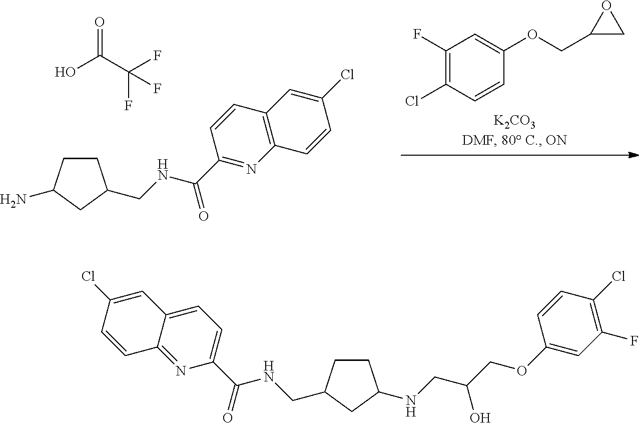 [SMILES: NC1CCC(CNC(=O)c2ccc3cc(Cl)ccc3n2)C1 ; O=C(O)C(F)(F)F ; Fc1cc(OCC2CO2)ccc1Cl ; O=C(NCC1CCC(NCC(O)COc2ccc(Cl)c(F)c2)C1)c1ccc2cc(Cl)ccc2n1]

To a stirred solution of N-((3-aminocyclopentyl)methyl)-6-chloroquinoline-2-carboxamide 2,2,2-trifluoroacetate (0.200 g, 0.480 mmol, 1.0 equiv) in DMF (5 ml) was added K₂CO₃ (0.227 g, 01.65 mmol, 2.5 equiv) followed by the addition of 2-((4-chloro-3-fluorophenoxy)methyl)oxirane (0.146 g, 0.726 mmol, 1.0 equiv). The reaction mixture was allowed to stir at 80° C. for overnight. Product formation was confirmed by LCMS. The reaction mixture was diluted with water (50 mL) and extracted with ethyl acetate (30 mL×3). Combined organic layer was washed with water (30 mL×6). Organic layer was dried over anhydrous sodium sulfate, filtered and concentrated under reduced pressure. Crude material was purified by flash chromatography (5% MeOH in DCM as an eluent) to obtain 6-chloro-N-((3-((3-(4-chloro-3-fluorophenoxy)-2-hydroxypropyl)amino)cyclopentyl)methyl)quinoline-2-carboxamide (Compound 34—0.090 g, 37% Yield) as an off-white solid. LCMS 506.2 [M+H]⁺; ¹H NMR (400 MHz, DMSO-d₆) δ 9.03 (d, J=6.6 Hz, 1H), 8.54 (d, J=8.3 Hz, 1H), 8.31-8.06 (m, 2H), 7.87 (dd, J=2.2, 9.2 Hz, 1H), 7.44 (t, J=8.6 Hz, 1H), 7.04 (dd, J=2.6, 11.4 Hz, 1H), 6.81 (d, J=9.6 Hz, 1H), 5.19 (br. s., 1H), 4.08-3.93 (m, 1H), 3.90 (d, J=6.1 Hz, 1H), 3.13 (br. s., 1H), 2.72 (br. s., 1H), 2.67 (br. s., 1H), 2.33 (br. s., 1H), 2.30-2.21 (m, 1H), 2.02 (d, J=4.8 Hz, 1H), 1.78 (br. s., 1H), 1.66 (dd, J=8.6, 15.6 Hz, 1H), 1.48 (br. s., 1H), 1.16 (br. s., 1H).

### Example 33

### Synthesis of 5-chloro-N-((3-((3-(4-chloro-3-fluorophenoxy)-2-hydroxypropyl)amino)cyclopentyl)methyl)benzofuran-2-carboxamide

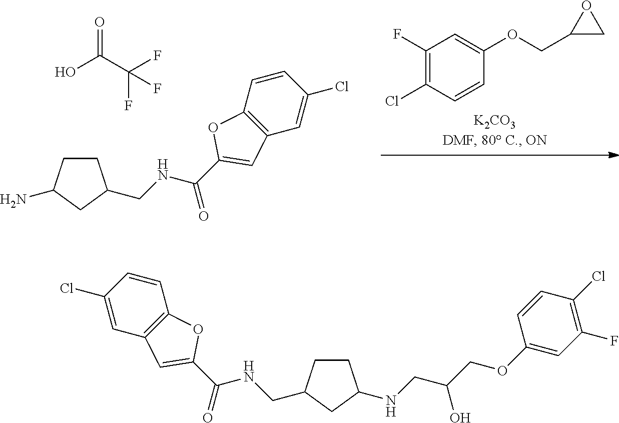 [SMILES: NC1CCC(CNC(=O)c2cc3cc(Cl)ccc3o2)C1 ; O=C(O)C(F)(F)F ; Fc1cc(OCC2CO2)ccc1Cl ; O=C(NCC1CCC(NCC(O)COc2ccc(Cl)c(F)c2)C1)c1cc2cc(Cl)ccc2o1]

To a stirred solution of N-((3-aminocyclopentyl)methyl)-5-chlorobenzofuran-2-carboxamide 2,2,2-trifluoroacetate (0.200 g, 0.492 mmol, 1.0 equiv) in DMF (5 ml) was added K₂CO₃ (0.236 g, 1.70 mmol, 2.5 equiv) followed by the addition of 2-((4-chloro-3-fluorophenoxy)methyl)oxirane (0.152 g, 0.753 mmol, 1.1 equiv). The reaction mixture was allowed to stir at 80° C. for overnight. Product formation was confirmed by LCMS. The reaction mixture was diluted with water (50 mL) and extracted with ethyl acetate (30 mL×3). Combined organic layer was washed with water (30 mL×6). Organic layer was dried over anhydrous sodium sulfate, filtered and concentrated under reduced pressure. Crude material was purified by flash chromatography (5% MeOH in DCM as an eluent) to obtain 5-chloro-N-((3-((3-(4-chloro-3-fluorophenoxy)-2-hydroxypropyl)amino)cyclopentyl)methyl)benzofuran-2-carboxamide (Compound 33—0.100 g, 41% Yield) as an off-white solid. LCMS 495.2 [M+H]⁺; ¹H NMR (400 MHz, DMSO-d₆) δ 8.92 (br. s., 1H), 7.86 (s, 1H), 7.69 (d, J=8.8 Hz, 1H), 7.54-7.36 (m, 2H), 7.05 (d, J=11.4 Hz, 1H), 6.81 (d, J=8.8 Hz, 1H), 5.13 (br. s., 1H), 3.99 (d, J=5.7 Hz, 1H), 3.89 (d, J=6.1 Hz, 2H), 3.25 (t, J=5.7 Hz, 2H), 3.07 (br. s., 1H), 2.67 (br. s., 1H), 2.61 (br. s., 1H), 2.30-2.09 (m, 2H), 1.99 (dd, J=6.6, 13.6 Hz, 2H), 1.73 (br. s., 1H), 1.71-1.54 (m, 1H), 1.43 (br. s., 2H), 1.09 (dd, J=7.5, 12.3 Hz, 1H).

### Example 34

### Synthesis of 5-chloro-N-((3S)-3-(((R)-3-(4-chloro-3-fluorophenoxy)-2-hydroxypropyl)amino)cyclopentyl)benzofuran-2-carboxamide

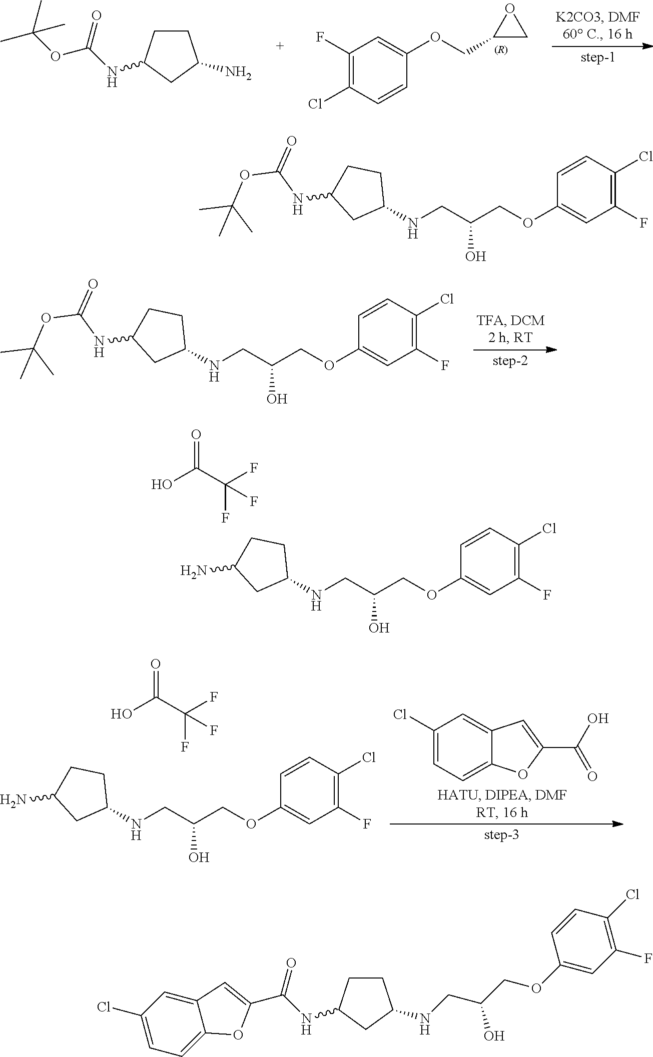

### Step-1: Synthesis of tert-butyl ((3S)-3-(((R)-3-(4-chloro-3-fluorophenoxy)-2-hydroxypropyl)amino)cyclopentyl)carbamate

To a stirred solution tert-butyl ((3S)-3-aminocyclopentyl)carbamate (1.18 g, 5.94 mmol, 1.2 equiv) in DMF (10 mL) was added K₂CO₃ (1.366 g, 9.90 mmol, 2.0 equiv) followed by the addition of (R)-2-((4-chloro-3-fluorophenoxy)methyl)oxirane (1.000 g, 4.95 mmol, 1.0 equiv). The reaction mixture was heated at 60° C. for 16 h. Product formation was confirmed by LCMS. The reaction mixture was diluted with water which results in to precipitate. The resulting solid precipitate was filtered off and washed with ice-cold water, dried under vaccume to obtain tert-butyl ((3S)-3-(((R)-3-(4-chloro-3-fluorophenoxy)-2-hydroxypropyl)amino)cyclopentyl)carbamate (0.900 g 45% Yield) as an off white solid. LCMS 403.3 [M+H]⁺

### Step-2: Synthesis of (2R)-1-(((1S)-3-aminocyclopentyl)amino)-3-(4-chloro-3-fluorophenoxy)propan-2-ol 2,2,2-trifluoroacetate

To a stirred solution of tert-butyl ((3S)-3-(((R)-3-(4-chloro-3-fluorophenoxy)-2-hydroxypropyl)amino)cyclopentyl)carbamate (0.900 g, 2.24 mmol, 1.0 equiv) in DCM (10 mL) was added TFA (1.5 mL). The reaction mixture was allowed to stir at RT for 16 h. Reaction progress was monitored by LCMS and NMR. After completion of reaction, reaction mixture was concentrated under reduced pressure. Crude compound was crystallized in diethyl ether to obtain pure (2R)-1-(((1S)-3-aminocyclopentyl)amino)-3-(4-chloro-3-fluorophenoxy)propan-2-ol 2,2,2-trifluoroacetate (0.760 g, 80% Yield) as an off white solid. LCMS 303.2 [M+H]⁺

### Step-3: Synthesis of 5-chloro-N-((3S)-3-(((R)-3-(4-chloro-3-fluorophenoxy)-2-hydroxypropyl)amino)cyclopentyl)benzofuran-2-carboxamide

To a stirred solution of 5-chlorobenzofuran-2-carboxylic acid (0.173 g, 0.880 mmol, 1.0 equiv) in DMF (5 mL) was added (2R)-1-(((1S)-3-aminocyclopentyl)amino)-3-(4-chloro-3-fluorophenoxy)propan-2-ol 2,2,2-trifluoroacetate (0.367 g, 0.880 mmol, 1.0 equiv), HATU (0.502 g, 1.32 mmol, 1.5 equiv) and DIPEA (0.227 g, 1.76 mmol, 2.0 equiv). The reaction mixture was allowed to stir at RT for 16 h. Product formation was confirmed by LCMS. The reaction mixture was diluted with water (20 mL) and extracted with ethyl acetate (30 mL×3). Combined organic layer was washed with water (20 mL×5). Organic layer was dried over anhydrous sodium sulfate, filtered and concentrated under reduced pressure. Crude product was purified by reversed phase HPLC to obtain 5-chloro-N-((3S)-3-(((R)-3-(4-chloro-3-fluorophenoxy)-2-hydroxypropyl)amino)cyclopentyl)benzofuran-2-carboxamide (Compound 90—0.065 g, 15% Yield) as an off-white solid. LCMS 481.2 [M+H]⁺; ¹H NMR (400 MHz, DMSO-d₆) δ 8.66 (d, J=7.45 Hz, 1H) 7.86 (d, J=2.19 Hz, 1H) 7.69 (d, J=9.21 Hz, 1H) 7.38-7.58 (m, 3H) 7.07 (dd, J=11.62, 2.41 Hz, 1H) 6.84 (dd, J=8.77, 2.19 Hz, 1H) 5.03 (br. s., 1H) 4.31-4.49 (m, 1H) 3.98-4.06 (m, 1H) 3.80-3.98 (m, 2H) 3.07-3.28 (m, 2H) 2.64 (dd, J=11.84, 4.82 Hz, 1H) 2.56 (d, J=4.82 Hz, 1H) 1.87-2.12 (m, 2H) 1.67-1.82 (m, 2H) 1.47-1.61 (m, 1H) 1.26-1.39 (m, 1H).

### Example 35

### Synthesis of 5-chloro-N-((3S)-3-(((S)-3-(4-chloro-3-fluorophenoxy)-2-hydroxypropyl)amino)cyclopentyl)benzofuran-2-carboxamide

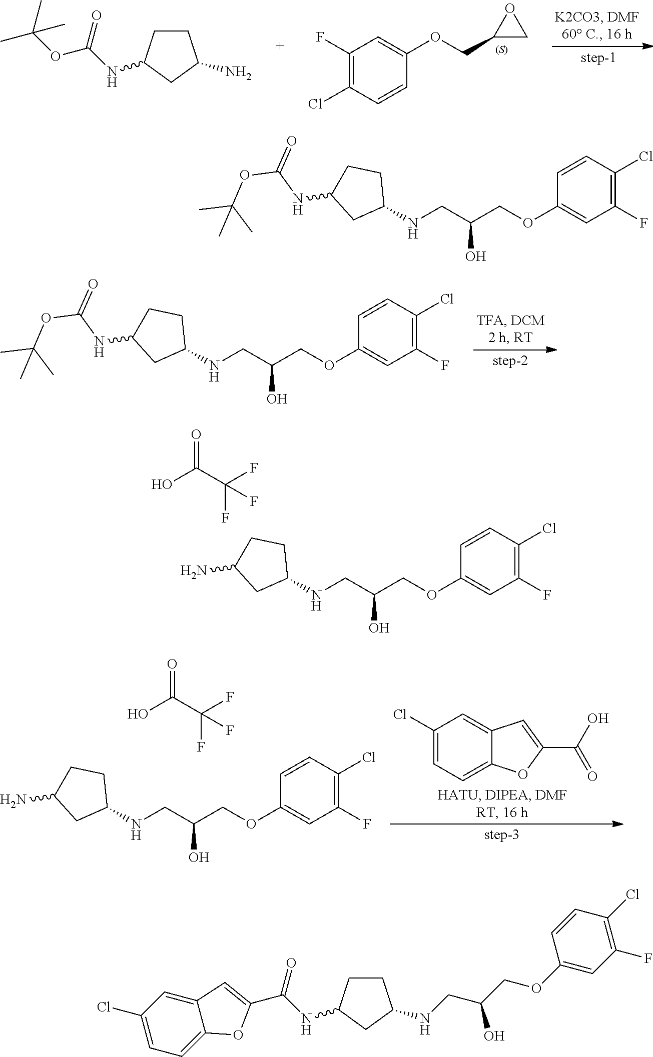

### Step-1: Synthesis of tert-butyl ((3S)-3-(((S)-3-(4-chloro-3-fluorophenoxy)-2-hydroxypropyl)amino)cyclopentyl)carbamate

To a stirred solution tert-butyl ((3S)-3-aminocyclopentyl)carbamate (1.18 g, 5.94 mmol, 1.2 equiv) in DMF (10 mL) was added K₂CO₃ (1.366 g, 9.90 mmol, 2.0 equiv) followed by the addition of (R)-2-((4-chloro-3-fluorophenoxy)methyl)oxirane (1.000 g, 4.95 mmol, 1.0 equiv). The reaction mixture was heated at 60° C. for 16 h. Product formation was confirmed by LCMS. The reaction mixture was diluted with water which results in to precipitate. The resulting solid precipitate was filtered off and washed with ice-cold water, dried under vaccume to obtain tert-butyl ((3S)-3-(((S)-3-(4-chloro-3-fluorophenoxy)-2-hydroxypropyl)amino)cyclopentyl)carbamate (0.800 g 40% Yield) as an off white solid. LCMS 403.3 [M+H]⁺

### Step-2: Synthesis of (2S)-1-(((1S)-3-aminocyclopentyl)amino)-3-(4-chloro-3-fluorophenoxy)propan-2-ol 2,2,2-trifluoroacetate

To a stirred solution of tert-butyl ((1S,3S)-3-((3-(4-chloro-3-fluorophenoxy)-2-hydroxypropyl)amino)cyclopentyl)carbamate (0.800 g, 2.24 mmol, 1.0 equiv) in DCM (10 mL) was added TFA (1.2 mL). The reaction mixture was allowed to stir at RT for 16 h. Reaction progress was monitored by LCMS and NMR. After completion of reaction, reaction mixture was concentrated under reduced pressure. Crude compound was crystallized in diethyl ether to obtain pure (2S)-1-(((1S)-3-aminocyclopentyl)amino)-3-(4-chloro-3-fluorophenoxy)propan-2-ol 2,2,2-trifluoroacetate (0.730 g, 88% Yield) as an off white solid. LCMS 303.2 [M+H]⁺

### Step-3: Synthesis of 5-chloro-N-((3S)-3-(((S)-3-(4-chloro-3-fluorophenoxy)-2-hydroxypropyl)amino)cyclopentyl)benzofuran-2-carboxamide

To a stirred solution of 5-chlorobenzofuran-2-carboxylic acid (0.140 g, 0.720 mmol, 1.0 equiv) in DMF (5 mL) was added (2S)-1-(((1S)-3-aminocyclopentyl)amino)-3-(4-chloro-3-fluorophenoxy)propan-2-ol 2,2,2-trifluoroacetate (0.300 g, 0.720 mmol, 1.0 equiv), HATU (0.410 g, 1.08 mmol, 1.5 equiv) and DIPEA (0.186 g, 1.44 mmol, 2.0 equiv). The reaction mixture was allowed to stir at RT for 16 h. Product formation was confirmed by LCMS. The reaction mixture was diluted with water (20 mL) and extracted with ethyl acetate (30 mL×3). Combined organic layer was washed with water (20 mL×5). Organic layer was dried over anhydrous sodium sulfate, filtered and concentrated under reduced pressure. Crude product was purified by reversed phase HPLC to obtain 5-chloro-N-((3S)-3-(((S)-3-(4-chloro-3-fluorophenoxy)-2-hydroxypropyl)amino)cyclopentyl)benzofuran-2-carboxamide (Compound 91—0.104 g, 30% Yield) as an off-white solid. LCMS 481.2 [M+H]⁺; ¹H NMR (400 MHz, DMSO-d₆) δ 8.66 (d, J=7.45 Hz, 1H) 7.86 (d, J=2.19 Hz, 1H) 7.69 (d, J=9.21 Hz, 1H) 7.38-7.58 (m, 3H) 7.07 (dd, J=11.62, 2.41 Hz, 1H) 6.84 (dd, J=8.77, 2.19 Hz, 1H) 5.03 (br. s., 1H) 4.31-4.49 (m, 1H) 3.98-4.06 (m, 1H) 3.80-3.98 (m, 2H) 3.07-3.28 (m, 2H) 2.64 (dd, J=11.84, 4.82 Hz, 1H) 2.56 (d, J=4.82 Hz, 1H) 1.87-2.12 (m, 2H) 1.67-1.82 (m, 2H) 1.47-1.61 (m, 1H) 1.26-1.39 (m, 1H).

### Example 36

### Synthesis of 2-(4-chloro-3-fluorophenoxy)-N-(3-((2-(4-chloro-3-fluorophenoxy) acetamido) methyl)pyrrolidin-1-yl)acetamide

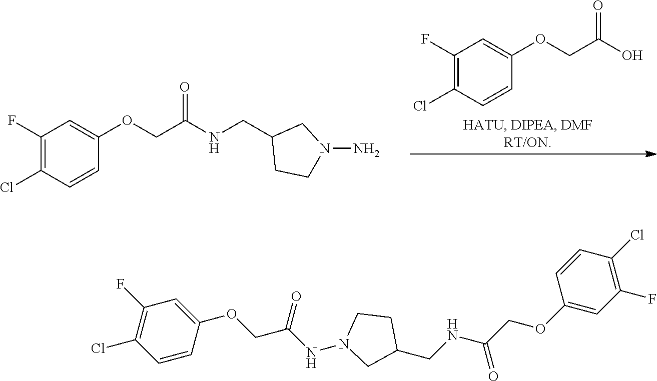 [SMILES: NN1CCC(CNC(=O)COc2ccc(Cl)c(F)c2)C1 ; O=C(O)COc1ccc(Cl)c(F)c1 ; O=C(COc1ccc(Cl)c(F)c1)NCC1CCN(NC(=O)COc2ccc(Cl)c(F)c2)C1]

To a solution of N-((1-aminopyrrolidin-3-yl)methyl)-2-(4-chloro-3-fluorophenoxy)acetamide (200 mg, 0.66 mmol, 1.0 eq) in DMF (05 mL) was added 2-(4-chloro-3-fluorophenoxy)acetic acid (134 mg, 0.66 mmol, 1.0 eq) and HATU (501 mg, 1.32 mmol, 2.0 eq) at RT. The resulting reaction mixture was stir for 10 min. followed by the addition of DIPEA (0.4 mL, 1.98 mmol, 3.0 eq). The reaction mixture was allowed to stir at RT overnight. Product formation was confirmed by LCMS. The reaction mixture was diluted with water (50 mL) and extracted with ethyl acetate (100 mL×2). Combined organic extracts were washed with water (50 mL×4), dried over anhydrous Na2SO4 and concentrated. The crude product which was purified by reversed phase of HPLC to obtain 2-(4-chloro-3-fluorophenoxy)-N-(3-((2-(4-chloro-3-fluorophenoxy) acetamido) methyl)pyrrolidin-1-yl)acetamide (Compound 92—60 mg, 19% Yield) as a white solid. LCMS 487.09 [M+H]⁺; ¹H NMR (400 MHz, DMSO-d₆) δ 9.09 (br. s., 1H) 8.65 (br. s., 1H) 8.20 (br. s., 1H) 7.47 (t, J=8.33 Hz, 2H) 7.03 (s, 1H) 7.05 (s, 1H) 6.83 (br. s., 1H) 4.90 (d, J=12.28 Hz, 2H) 4.51 (br. s., 2H) 4.45 (br. s., 1H) 3.11 (br. s., 2H) 2.83 (d, J=6.58 Hz, 1H) 2.62 (d, J=17.98 Hz, 2H) 1.81 (br. s., 1H) 1.41 (br. s., 1H).

### Example 37

### Synthesis of 6-chloro-N-(3-((2-(4-chloro-3-fluorophenoxy)acetamido)methyl)pyrrolidin-1-yl)quinoline-2-carboxamide

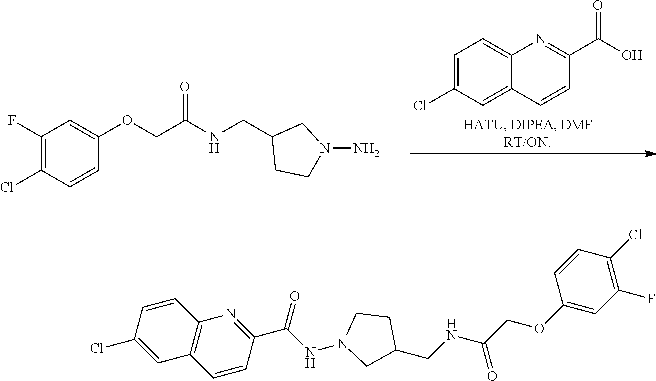 [SMILES: NN1CCC(CNC(=O)COc2ccc(Cl)c(F)c2)C1 ; O=C(O)c1ccc2cc(Cl)ccc2n1 ; O=C(COc1ccc(Cl)c(F)c1)NCC1CCN(NC(=O)c2ccc3cc(Cl)ccc3n2)C1]

To a solution of N-((1-aminopyrrolidin-3-yl)methyl)-2-(4-chloro-3-fluorophenoxy)acetamide (200 mg, 0.66 mmol, 1.0 eq) in DMF (05 mL) was added 6-chloroquinoline-2-carboxylic acid (137 mg, 0.66 mmol, 1.0 eq) and HATU (501 mg, 1.32 mmol, 2.0 eq) at RT. The resulting reaction mixture was stir for 10 min. followed by the addition of DIPEA (0.4 mL, 1.98 mmol, 3.0 eq). The reaction mixture was allowed to stir at RT overnight. Product formation was confirmed by LCMS. The reaction mixture was diluted with water (50 mL) and extracted with ethyl acetate (100 mL×2). Combined organic extracts were washed with water (50 mL×4), dried over anhydrous Na2SO4 and concentrated. The crude product was purified by reversed phase of HPLC to obtain 6-chloro-N-(3-((2-(4-chloro-3-fluorophenoxy)acetamido)methyl)pyrrolidin-1-yl)quinoline-2-carboxamide (Compound 93—130 mg, 40% Yield) as a white solid. LCMS 490.10 [M+H]⁺; ¹H NMR (400 MHz, DMSO-d₆) δ 9.74 (s, 1H) 8.51 (d, J=8.33 Hz, 1H) 8.20-8.31 (m, 2H) 8.12 (dd, J=8.77, 2.63 Hz, 2H) 7.86 (d, J=6.58 Hz, 1H) 7.47 (t, J=8.77 Hz, 1H) 7.04-7.11 (m, 1H) 6.85 (d, J=8.33 Hz, 1H) 4.53 (s, 2H) 3.17 (br. s., 2H) 2.97-3.07 (m, 2H) 2.73-2.79 (m, 1H) 2.31 (br. s., 2H) 1.87 (br. s., 1H) 1.49 (d, J=13.59 Hz, 1H).

### Example 38

### Chiral Resolution of trans-5-chloro-N-(3-(((R)-3-(4-chloro-3-fluorophenoxy)-2-hydroxypropyl)amino)cyclopentyl)benzofuran-2-carboxamide

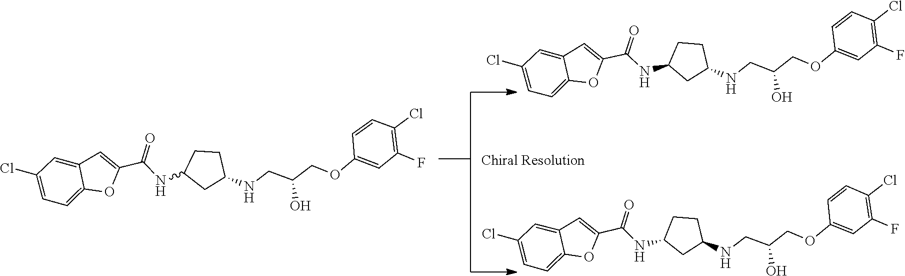 [SMILES: O=C(N[C@H]1CC[C@H](NC[C@@H](O)COc2ccc(Cl)c(F)c2)C1)c1cc2cc(Cl)ccc2o1 ; O=C(NC1CC[C@H](NC[C@@H](O)COc2ccc(Cl)c(F)c2)C1)c1cc2cc(Cl)ccc2o1 ; O=C(N[C@@H]1CC[C@@H](NC[C@@H](O)COc2ccc(Cl)c(F)c2)C1)c1cc2cc(Cl)ccc2o1]

The enantiomers, 5-chloro-N-((1S,3S)-3-(((R)-3-(4-chloro-3-fluorophenoxy)-2-hydroxypropyl)amino)cyclopentyl)benzofuran-2-carboxamide (Compound 94—[α]_{D}²⁰=not available, elution time: 26.78 min) and 5-chloro-N-((1R,3R)-3-(((R)-3-(4-chloro-3-fluorophenoxy)-2-hydroxypropyl)amino)cyclopentyl)benzofuran-2-carboxamide (Compound 95—[α]_{D}²⁰=not available, elution time: 37.3 min), were separated by chiral SFC (Chiralpak-ADH, 20×250 mm, 5 μm). Isocratic program with analytical grade liquid carbon dioxide (food grade) and 0.2% DEA in Methanol HPLC grade. LCMS: 481.4 [M+H]⁺; ¹H NMR (400 MHz, DMSO-d₆) δ 8.66 (d, J=7.89 Hz, 1H) 7.87 (d, J=2.19 Hz, 1H) 7.69 (d, J=8.77 Hz, 1H) 7.36-7.55 (m, 2H) 7.07 (dd, J=11.62, 2.85 Hz, 1H) 6.75-6.89 (m, 1H) 5.00 (br. s., 1H) 4.31-4.47 (m, 1H) 4.01 (dd, J=9.65, 3.95 Hz, 1H) 3.79-3.95 (m, 2H) 3.01-3.23 (m, 1H) 2.53-2.75 (m, 2H) 1.84-2.14 (m, 2H) 1.63-1.82 (m, 2H) 1.54 (dd, J=12.28, 7.89 Hz, 1H) 1.25-1.38 (m, 1H).

### Example 39

### Chiral Resolution of 5-chloro-N-(3-((2-(4-chloro-3-fluorophenoxy)acetamido)methyl)pyrrolidin-1-yl)benzofuran-2-carboxamide

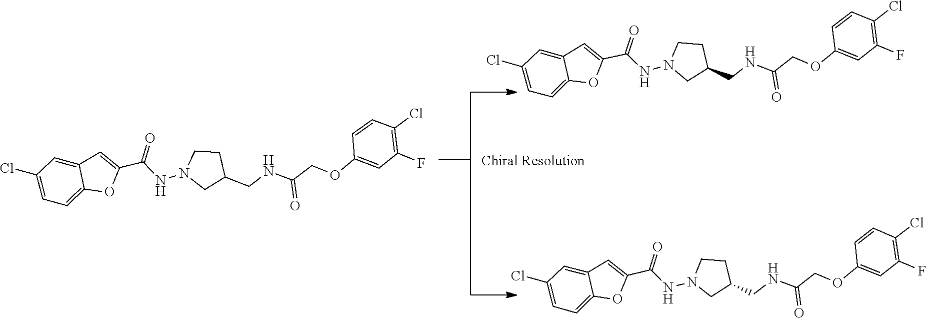 [SMILES: O=C(COc1ccc(Cl)c(F)c1)NC[C@@H]1CCN(NC(=O)c2cc3cc(Cl)ccc3o2)C1 ; O=C(COc1ccc(Cl)c(F)c1)NCC1CCN(NC(=O)c2cc3cc(Cl)ccc3o2)C1 ; O=C(COc1ccc(Cl)c(F)c1)NC[C@H]1CCN(NC(=O)c2cc3cc(Cl)ccc3o2)C1]

The enantiomers, (S)-5-chloro-N-(3-((2-(4-chloro-3-fluorophenoxy)acetamido)methyl)pyrrolidin-1-yl)benzofuran-2-carboxamide (Compound 96—[α]_{D}²⁰=not available, elution time: 16.16 min) and (R)-5-chloro-N-(3-((2-(4-chloro-3-fluorophenoxy)acetamido)methyl)pyrrolidin-1-yl)benzofuran-2-carboxamide (Compound 97—[α]_{D}²⁰=not available, elution time: 29.29 min), were separated by chiral SFC (Chiralpak ADH, 250×20 mm, 5μ). Isocratic program with analytical grade liquid carbon dioxide and 0.2% DEA in Methanol. LCMS: 480.4 [M+H]⁺; ¹H NMR (400 MHz, DMSO-d₆) δ 9.77 (s, 1H) 8.24 (br. s., 1H) 7.86 (s, 1H) 7.69 (d, J=8.77 Hz, 1H) 7.35-7.56 (m, 3H) 7.07 (d, J=11.40 Hz, 1H) 6.86 (d, J=6.14 Hz, 1H) 4.54 (s, 2H) 3.17 (br. s., 2H) 2.87-3.11 (m, 2H) 2.65-2.78 (m, 1H) 2.35 (d, J=19.29 Hz, 2H) 1.88 (br. s., 1H) 1.39-1.57 (m, 1H).

### Example 40

### Synthesis of 5-chloro-N-(1-(3-(4-chloro-3-fluorophenoxy)-2-hydroxypropyl)pyrrolidin-3-yl)benzofuran-2-carboxamide

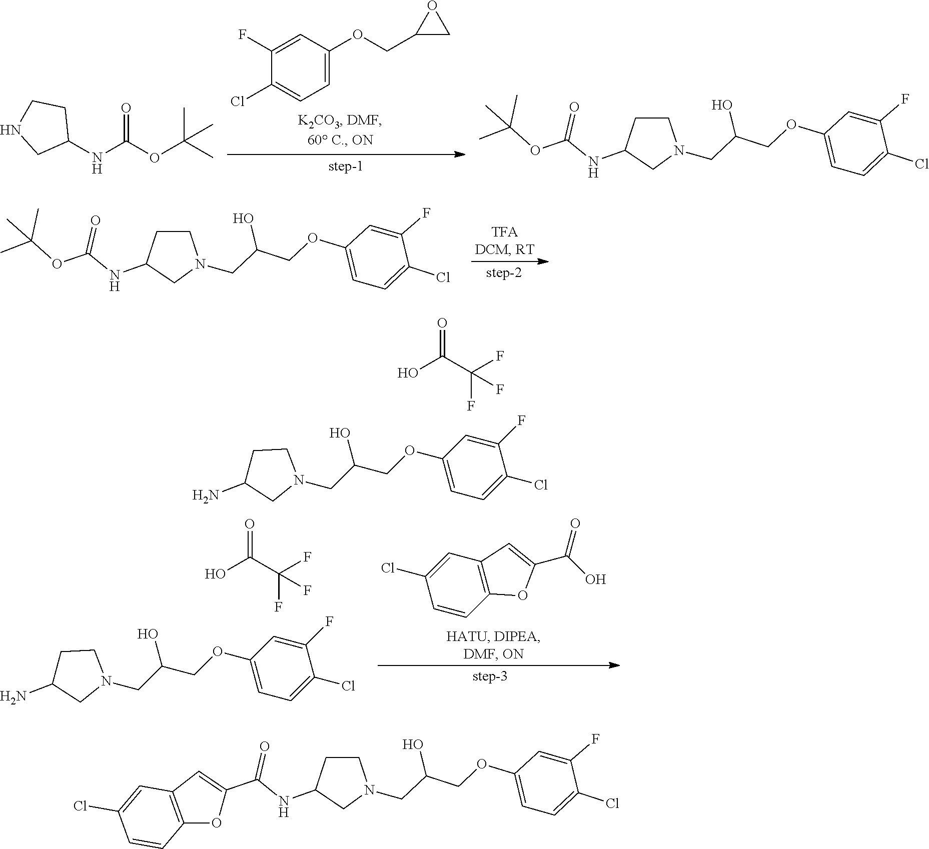

### Step-1: Synthesis of tert-butyl (1-(3-(4-chloro-3-fluorophenoxy)-2-hydroxypropyl)pyrrolidin-3-yl)carbamate

To a stirred solution of tert-butyl pyrrolidin-3-ylcarbamate (200 mg, 1.074 mmol, 1 eq) in DMF (2 mL) was added K2CO3 (296 mg, 2.147 mmol, 2.0 eq) followed by the addition of 2-((4-chloro-3-fluorophenoxy)methyl)oxirane (195 mg, 0.97 mmol, 0.9 eq). The resulting reaction mixture was heated at 60° C. for overnight. Product formation was confirmed by NMR. After completion of reaction, the reaction mixture was poured into ice cold water (50 ml), and extracted with EtOAc (2×30 mL). Organic layer washed with water (5×20 mL), brine solution (1×20 mL), dried over anhydrous sodium sulfate and concentrated under reduced pressure to obtain tert-butyl (1-(3-(4-chloro-3-fluorophenoxy)-2-hydroxypropyl)pyrrolidin-3-yl)carbamate (350 mg, 84% Yield) as a yellow oil. LCMS 389.3 [M+H]⁺, ¹H NMR (400 MHz, DMSO-d₆) δ 7.45 (t, J=9.0 Hz, 1H), 7.05 (d, J=11.8 Hz, 1H), 6.96 (d, J=7.0 Hz, 1H), 6.83 (d, J=7.0 Hz, 1H), 4.90 (d, J=3.9 Hz, 1H), 4.08-3.96 (m, 1H), 3.95-3.73 (m, 2H), 2.89 (s, 1H), 2.82-2.63 (m, 1H), 2.57 (d, J=8.3 Hz, 1H), 2.43-2.30 (m, 2H), 2.08-1.86 (m, 2H), 1.52 (br. s., 2H), 1.37 (s, 9H).

### Step-2: Synthesis of 1-(3-aminopyrrolidin-1-yl)-3-(4-chloro-3-fluorophenoxy)propan-2-ol 2,2,2-trifluoroacetate

To a stirred solution of compound tert-butyl (1-(3-(4-chloro-3-fluorophenoxy)-2-hydroxypropyl)pyrrolidin-3-yl)carbamate (350 mg, 0.90 mmol, 1 eq) in DCM (30 mL), was added TFA (0.4 mL) and the resultant reaction mixture was stirred at RT for overnight under nitrogen atmosphere. Product formation was confirmed by LCMS. After completion of reaction, the reaction mixture was concentrated under reduced pressure to obtain sticky crude compound which was crystallized in diethyl ether, dried under vacuum to obtain 1-(3-aminopyrrolidin-1-yl)-3-(4-chloro-3-fluorophenoxy)propan-2-ol 2,2,2-trifluoroacetate (280 mg, 77% Yield) as a brown solid. LCMS 289.0 [M+H]⁺, ¹H NMR (400 MHz, DMSO-d₆) δ 8.32 (br. s., 2H), 7.50 (t, J=9.0 Hz, 1H), 7.09 (dd, J=2.6, 11.4 Hz, 1H), 6.85 (dd, J=2.0, 9.0 Hz, 1H), 4.18 (br. s., 1H), 4.08-3.82 (m, 4H), 3.64-3.47 (m, 2H), 3.25 (d, J=18.9 Hz, 1H), 3.17 (s, 4H), 2.09-1.86 (m, 1H).

### Step-3: Synthesis of 5-chloro-N-(1-(3-(4-chloro-3-fluorophenoxy)-2-hydroxypropyl)pyrrolidin-3-yl)benzofuran-2-carboxamide

To a stirred solution of 1-(3-aminopyrrolidin-1-yl)-3-(4-chloro-3-fluorophenoxy)propan-2-ol 2,2,2-trifluoroacetate (200 mg, 0.497 mmol, 1 eq) in DMF (5 mL) was added 5-chlorobenzofuran-2-carboxylic acid (98 mg, 0.497 mmol, 1 eq), HATU (283 mg, 0.745 mmol, 1.5 eq) followed by the addition of DIPEA (0.55 mL, 1.988 mmol, 4.0 eq) and the resultant reaction mixture was stirred at RT for overnight. Product formation was confirmed by LCMS. After completion of reaction, the reaction mixture was poured into ice cold water (50 ml) and extracted with EtOAc (2×50 mL). Combined organic layer was washed with water (4×20 mL), brine solution (1×20 mL), dried over anhydrous sodium sulfate and concentrated under reduced pressure. The crude product was purified by flash chromatography (5% MeOH in DCM as an eluent) to obtain 5-chloro-N-(1-(3-(4-chloro-3-fluorophenoxy)-2-hydroxypropyl)pyrrolidin-3-yl)benzofuran-2-carboxamide (Compound 98—85 mg, 36% Yield) as a white solid. LCMS 467.3 [M+H]⁺, ¹H NMR (400 MHz, DMSO-d₆) δ 8.84 (br. s., 1H), 7.88 (d, J=1.8 Hz, 1H), 7.69 (d, J=8.8 Hz, 1H), 7.55 (s, 1H), 7.51-7.30 (m, 2H), 7.14-6.98 (m, 1H), 6.84 (d, J=9.2 Hz, 1H), 5.00 (br. s., 1H), 4.43 (br. s., 1H), 4.11-3.97 (m, 2H), 3.93 (br. s., 1H), 2.87 (br. s., 1H), 2.78-2.64 (m, 2H), 2.18 (br. s., 1H), 1.84 (br. s., 1H), 1.23 (s, 1H), 1.16 (d, J=14.0 Hz, 1H), 0.76 (br. s., 1H).

### Example 41

### Synthesis of 6-chloro-N-(1-(3-(4-chloro-3-fluorophenoxy)-2-hydroxypropyl)pyrrolidin-3-yl)quinoline-2-carboxamide

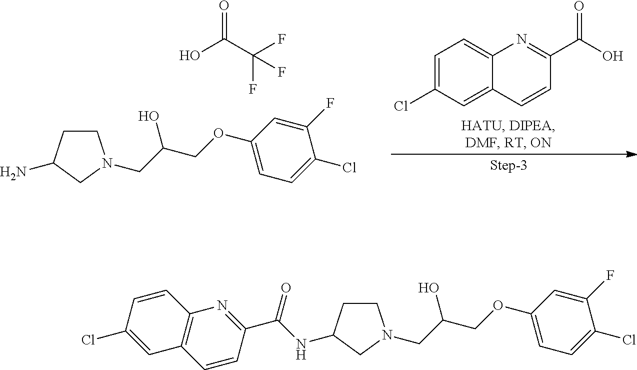

To a stirred solution of 1-(3-aminopyrrolidin-1-yl)-3-(4-chloro-3-fluorophenoxy)propan-2-ol 2,2,2-trifluoroacetate (200 mg, 0.497 mmol, 1 eq) in DMF (5 mL) was added 6-chloroquinoline-2-carboxylic acid (103 mg, 0.497 mmol, 1 eq), HATU (283 mg, 0.745 mmol, 1.5 eq) followed by the addition of DIPEA (0.55 mL, 1.988 mmol, 4.0 eq) and the resultant reaction mixture was stirred at RT for overnight. Product formation was confirmed by LCMS. After completion of reaction, the reaction mixture was poured into ice cold water (50 ml) and extracted with EtOAc (2×50 mL). Combined organic layer washed with water (4×20 mL), brine solution (1×20 mL), dried over anhydrous sodium sulfate and concentrated under reduced pressure. The crude product was purified by flash chromatography (5% MeOH in DCM as an eluent) to obtain 6-chloro-N-(1-(3-(4-chloro-3-fluorophenoxy)-2-hydroxypropyl)pyrrolidin-3-yl)quinoline-2-carboxamide (Compound 53—100 mg, 42.2%) as a white solid. LCMS 478 [M+H]⁺, ¹H NMR (400 MHz, DMSO-d₆) δ 8.89 (br. s., 1H), 8.55 (d, J=8.3 Hz, 1H), 8.26 (d, J=2.2 Hz, 1H), 8.18 (d, J=8.8 Hz, 1H), 8.10 (s, 1H), 7.87 (d, J=7.9 Hz, 1H), 7.48-7.29 (m, 1H), 7.09 (td, J=3.1, 11.4 Hz, 1H), 6.85 (d, J=7.9 Hz, 1H), 5.04 (br. s., 1H), 4.53 (br. s., 1H), 4.05 (d, J=9.6 Hz, 2H), 3.96 (br. s., 1H), 2.69 (s, 3H), 2.25 (br. s., 2H), 1.90 (br. s., 2H), 1.23 (s, 1H).

### Example 42

### Synthesis of 2-(4-chloro-3-fluorophenoxy)-N-(1-(3-(4-chloro-3-fluorophenoxy)-2-hydroxypropyl)pyrrolidin-3-yl)acetamide

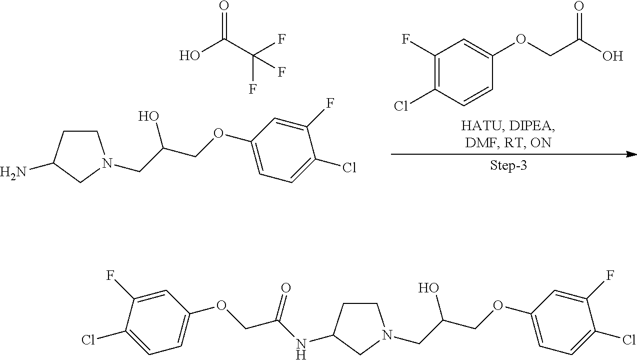

To a stirred solution of 1-(3-aminopyrrolidin-1-yl)-3-(4-chloro-3-fluorophenoxy)propan-2-ol 2,2,2-trifluoroacetate (200 mg, 0.497 mmol, 1 eq) in DMF (5 mL) in DMF wad added 2-(4-chloro-3-fluorophenoxy)acetic acid (101 mg, 0.497 mmol, 1 eq), HATU (283 mg, 0.745 mmol, 1.5 eq) followed by the addition of DIPEA (0.55 mL, 1.988 mmol, 4.0 eq) and the resultant reaction mixture was stirred at RT for overnight. Product formation was confirmed by LCMS. After completion of reaction, the reaction mixture was poured into ice cold water (50 ml) and extracted with EtOAc (2×50 mL). Combined organic layer washed with water (4×20 mL), brine solution (1×20 mL), dried over anhydrous sodium sulfate and concentrated under reduced pressure. The crude product was purified by flash chromatography (5% MeOH in DCM as an eluent) to obtain 2-(4-chloro-3-fluorophenoxy)-N-(1-(3-(4-chloro-3-fluorophenoxy)-2-hydroxypropyl)pyrrolidin-3-yl)acetamide (Compound 99—140 mg, 59% Yield) as a yellow semi solid. LCMS 475.4 [M+H]⁺, ¹H NMR (400 MHz, DMSO-d₆) δ 8.17 (d, J=6.1 Hz, 1H), 7.55-7.35 (m, 2H), 7.20-7.00 (m, 2H), 6.83 (d, J=8.8 Hz, 2H), 4.96 (br. s., 1H), 4.50 (s, 2H), 4.22 (br. s., 1H), 4.01 (dd, J=2.4, 5.9 Hz, 1H), 3.95-3.74 (m, 2H), 2.71 (br. s., 2H), 2.60 (br. s., 1H), 2.33 (br. s., 3H), 2.08 (d, J=5.7 Hz, 1H), 1.62 (br. s., 1H)

### Example 43

### Synthesis of 2-(4-chloro-3-fluorophenoxy)-N-(1-(3-(4-chloro-3-fluorophenoxy)propyl)pyrrolidin-3-yl)acetamide

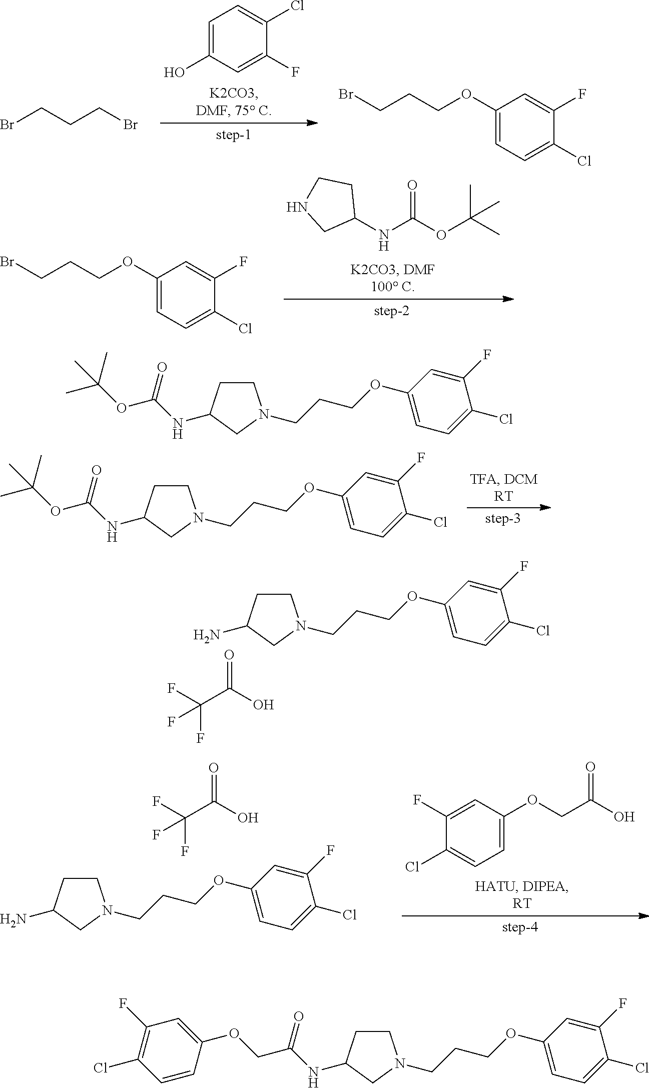

### Step-1: Synthesis of 4-(3-bromopropoxy)-1-chloro-2-fluorobenzene

To a solution of 3-chloro-4-fluorophenol (0.500 g, 3.41 mmols, 1.0 eq) in DMF (5 mL) was added K₂CO₃ (0.942 g, 6.82 mmols, 2.0 eq) followed by the addition of 1,3-dibromopropane (1.74 mL, 17.06 mmols, 5.0 eq). The resulting reaction mixture was heated at 75° C. for 4 h. Product formation was confirmed by NMR spectroscopy. After completion of reaction the reaction mixture was diluted with water (25 mL) and extracted with ethyl acetate (50 mL×2). Combined organic layer was washed with water (20 mL), dried over anhydrous Na2SO₄ and concentrated under reduced pressure to obtain the crude compound which was purified by flash chromatography (0-10% ethyl acetate in hexane as an eluent) to obtain 4-(3-bromopropoxy)-1-chloro-2-fluorobenzene (0.500 g, 54.8% Yield) as a colorless liquid. ¹H NMR (400 MHz, Chloroform-d) δ 7.22-7.30 (m, 1H) 6.72 (dd, J=10.74, 2.85 Hz, 1H) 6.65 (ddd, J=8.88, 2.74, 1.10 Hz, 1H) 4.08 (t, J=5.70 Hz, 2H) 3.46-3.69 (m, 2H) 2.18-2.43 (m, 2H).

### Step-2: Synthesis of tert-butyl (1-(3-(4-chloro-3-fluorophenoxy)propyl)pyrrolidin-3-yl)carbamate

To a solution of tert-butyl pyrrolidin-3-ylcarbamate (0.769 g, 4.13 mmols, 1.0 eq) in DMF (15 mL) was added K₂CO₃ (0.519 g, 3.76 mmols, 1.0 eq) followed by the addition of 4-(3-bromopropoxy)-1-chloro-2-fluorobenzene (1.0 g, 3.76 mmols, 1.0 eq). The resulting reaction mixture was heated at 80° C. for overnight. Product formation was confirmed by NMR spectroscopy. After completion of reaction the reaction mixture was diluted with water (50 mL) and extracted with ethyl acetate (100 mL×2). Combined organic layer was washed with water (20 mL×4), dried over anhydrous Na2SO₄ and concentrated under reduced pressure. Crude product was crystallized in diethyl ether to obtain tert-butyl (1-(3-(4-chloro-3-fluorophenoxy)propyl)pyrrolidin-3-yl)carbamate (0.750 g, 53% Yield) as an off-white solid. LCMS 374.2 [M+H]⁺, ¹H NMR (400 MHz, Chloroform-d) δ 7.19-7.31 (m, 1H) 6.70 (dd, J=10.74, 2.85 Hz, 1H) 6.58-6.66 (m, 1H) 4.89 (br. s., 1H) 4.19 (br. s., 1H) 3.98 (t, J=6.36 Hz, 1H) 2.88 (br. s., 1H) 2.61 (br. s., 2H) 2.17-2.44 (m, 2H) 1.88-2.06 (m, 2H) 1.62 (br. s., 2H) 1.42 (s., 9H) 1.26 (d, J=8.77 Hz, 1H).

### Step-3: Synthesis of 1-(3-(4-chloro-3-fluorophenoxy)propyl)pyrrolidin-3-amine 2,2,2-trifluoroacetate

To a stirred solution tert-butyl (1-(3-(4-chloro-3-fluorophenoxy)propyl)pyrrolidin-3-yl)carbamate (0.250 g, 0.67 mmol, 1.0 eq) in DCM (20 mL) was added trifluoroacetic acid (1 mL) at RT. the reaction mixture was allowed to stir at RT overnight. DCM and excess of trifluoroacetic acid was removed under reduced pressure to obtain 1-(3-(4-chloro-3-fluorophenoxy)propyl)pyrrolidin-3-amine 2,2,2-trifluoroacetate (0.260 g, quantitative yield) as an solid. LCMS 273.2 [M+H]⁺, ¹H NMR (400 MHz, DMSO-d₆) δ 10.72 (br. s., 1H) 8.41 (br. s., 2H) 7.48 (t, J=8.77 Hz, 1H) 7.06 (dd, J=11.40, 3.07 Hz, 1H) 6.83 (dd, J=8.77, 1.75 Hz, 1H) 4.08 (t, J=5.92 Hz, 2H) 3.98 (br. s., 1H) 3.62-3.86 (m, 2H) 3.48-3.62 (m, 1H) 3.27-3.47 (m, 3H) 3.05-3.27 (m, 1H) 2.33 (br. s., 1H) 1.90-2.20 (m, 1H) 1.09 (t, J=7.02 Hz, 2H).

### Step-4: Synthesis of 2-(4-chloro-3-fluorophenoxy)-N-(1-(3-(4-chloro-3-fluorophenoxy)propyl)pyrrolidin-3-yl)acetamide

To a solution of 1-(3-(4-chloro-3-fluorophenoxy)propyl)pyrrolidin-3-amine 2,2,2-trifluoroacetate (0.200 g, 0.73 mmol, 1.0 eq) in DMF (5 mL) was added 2-(4-chloro-3-fluorophenoxy)acetic acid (0.150 g, 0.73 mmol, 1.0 eq) and HATU (0.418 g, 1.102 mmol, 1.5 eq) at RT. The reaction mixture was stirred for 10 minutes and then DIPEA (0.36 mL, 2.20 mmol, 3.0 eq) was added. The resultant reaction mixture was allowed to stir at RT for overnight. Progress of the reaction was monitored by LCMS. The reaction mixture was diluted with water (10 mL) and extracted with ethyl acetate (100 mL×2). Combined organic layer was washed with water (50 mL×4), dried over anhydrous Na2SO₄ and concentrated under reduced pressure to obtain the crude compound which was purified by reversed phase HPLC to 2-(4-chloro-3-fluorophenoxy)-N-(1-(3-(4-chloro-3-fluorophenoxy)propyl)pyrrolidin-3-yl)acetamide (Compound 100—0.060 g, 17% Yield) as an off-white solid. LCMS 459.1 [M+H]⁺, ¹H NMR (400 MHz, DMSO-d₆) δ 8.20 (d, J=7.45 Hz, 1H) 7.45 (t, J=8.99 Hz, 1H) 7.48 (t, J=8.99 Hz, 1H) 7.04 (t, J=2.85 Hz, 1H) 7.07 (t, J=2.85 Hz, 1H) 6.69-6.94 (m, 2H) 4.51 (s, 2H) 4.14-4.34 (m, 1H) 4.03 (t, J=6.14 Hz, 2H) 3.34 (br. s., 2H) 2.67 (br. s., 2H) 2.42 (br. s., 2H) 1.98-2.21 (m, 1H) 1.86 (quin, J=6.69 Hz, 2H) 1.63 (dd, J=11.84, 5.70 Hz, 1H).

### Example 44

### Synthesis of 6-chloro-N-(1-(3-(4-chloro-3-fluorophenoxy)propyl)pyrrolidin-3-yl)quinoline-2-carboxamide

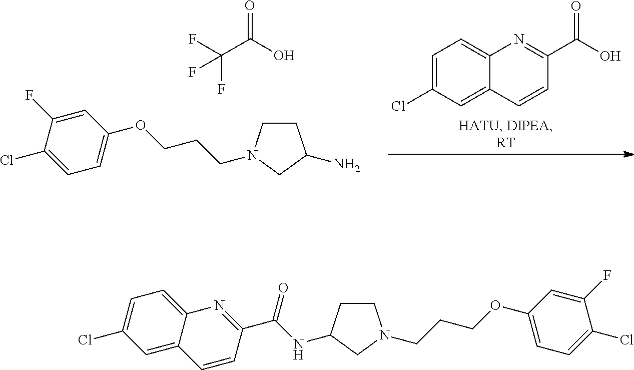 [SMILES: NC1CCN(CCCOc2ccc(Cl)c(F)c2)C1 ; O=C(O)C(F)(F)F ; O=C(O)c1ccc2cc(Cl)ccc2n1 ; O=C(NC1CCN(CCCOc2ccc(Cl)c(F)c2)C1)c1ccc2cc(Cl)ccc2n1]

To a solution of 1-(3-(4-chloro-3-fluorophenoxy)propyl)pyrrolidin-3-amine 2,2,2-trifluoroacetate (0.150 g, 0.55 mmol, 1.0 eq) in DMF (5 mL) was added 6-chloroquinoline-2-carboxylic acid (0.114 g, 0.73 mmol, 1.0 eq) and HATU (0.380 g, 0.82 mmol, 1.5 eq) at RT. The reaction mixture was stirred for 10 minutes and then DIPEA (0.27 mL, 2.20 mmol, 3.0 eq) was added. The resultant reaction mixture was allowed to stir at RT for overnight. Progress of the reaction was monitored by LCMS. The reaction mixture was diluted with water (10 mL) and extracted with ethyl acetate (100 mL×2). Combined organic layer was washed with water (50 mL×4), dried over anhydrous Na2SO₄ and concentrated under reduced pressure to obtain the crude compound which was purified by reversed phase HPLC to 6-chloro-N-(1-(3-(4-chloro-3-fluorophenoxy)propyl)pyrrolidin-3-yl)quinoline-2-carboxamide (Compound 56—0.060 g, 23% Yield) as an off-white solid. LCMS 462.1 [M+H]⁺, ¹H NMR (400 MHz, DMSO-d₆) δ 8.91 (br. s., 1H) 8.56 (d, J=8.77 Hz, 1H) 8.26 (d, J=2.63 Hz, 1H) 8.18 (d, J=8.33 Hz, 1H) 8.13 (d, J=7.45 Hz, 1H) 7.88 (dd, J=8.99, 2.41 Hz, 1H) 7.45 (t, J=8.77 Hz, 1H) 7.08 (dd, J=11.40, 2.63 Hz, 1H) 6.74-6.95 (m, 1H) 4.55 (br. s., 1H) 4.08 (t, J=6.14 Hz, 2H) 2.99 (br. s., 2H) 2.79 (br. s., 2H) 2.67 (br. s., 1H) 2.33 (br. s., 1H) 2.27 (br. s., 1H) 1.97 (br. s., 3H).

### Example 45

### Synthesis of 5-chloro-N-(1-(3-(4-chloro-3-fluorophenoxy)propyl)pyrrolidin-3-yl)benzofuran-2-carboxamide

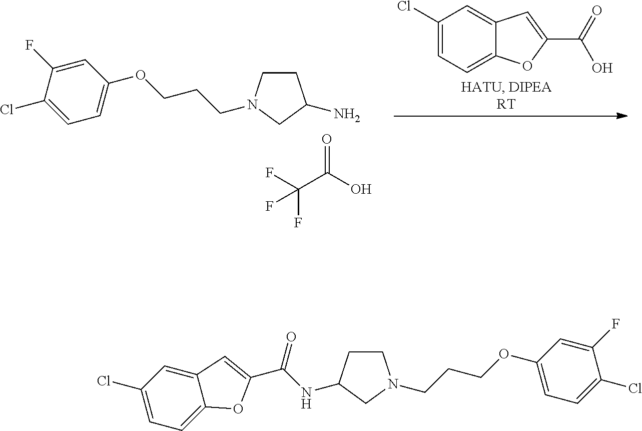

To a solution of 1-(3-(4-chloro-3-fluorophenoxy)propyl)pyrrolidin-3-amine 2,2,2-trifluoroacetate (0.150 g, 0.55 mmol, 1.0 eq) in DMF (5 mL) was added 5-chlorobenzofuran-2-carboxylic acid (0.114 g, 0.73 mmol, 1.0 eq) and HATU (0.380 g, 0.82 mmol, 1.5 eq) at RT. The reaction mixture was stirred for 10 minutes and then DIPEA (0.27 mL, 2.20 mmol, 3.0 eq) was added. The resultant reaction mixture was allowed to stir at RT for overnight. Progress of the reaction was monitored by LCMS. The reaction mixture was diluted with water (10 mL) and extracted with ethyl acetate (100 mL×2). Combined organic layer was washed with water (50 mL×4), dried over anhydrous Na2SO₄ and concentrated under reduced pressure to obtain the crude compound which was purified by reversed phase HPLC to 5-chloro-N-(1-(3-(4-chloro-3-fluorophenoxy)propyl)pyrrolidin-3-yl)benzofuran-2-carboxamide (Compound 101—0.080 g, 24% Yield) as an off-white solid. LCMS 451.10 [M+H]⁺, ¹H NMR (400 MHz, DMSO-d₆) δ 8.97 (br. s., 1H) 7.89 (d, J=2.19 Hz, 1H) 7.70 (d, J=8.77 Hz, 1H) 7.59 (s, 1H) 7.39-7.54 (m, 2H) 7.08 (dd, J=11.40, 2.63 Hz, 1H) 6.76-6.92 (m, 1H) 4.51 (br. s., 1H) 4.07 (t, J=6.14 Hz, 2H) 2.94 (br. s., 5H) 2.33 (br. s., 1H) 2.25 (br. s., 1H) 2.00 (br. s., 3H).

### Example 46

### Synthesis of 5-chloro-N-((1-(3-(4-chloro-3-fluorophenoxy)-2-hydroxypropyl)pyrrolidin-3-yl)methyl)benzofuran-2-carboxamide

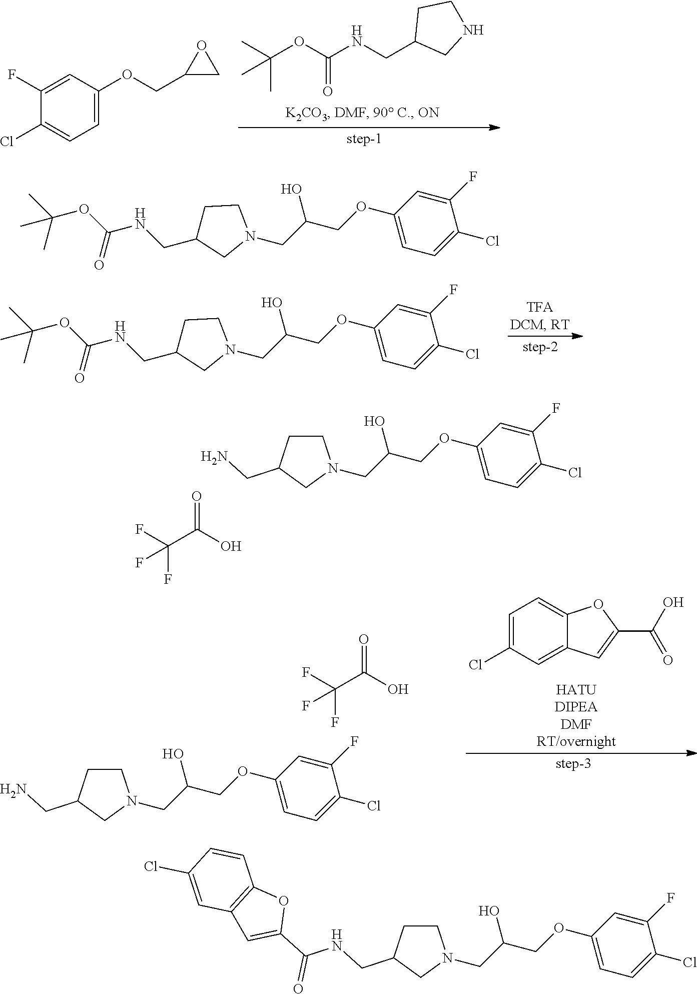

### Step-1: Synthesis of tert-butyl ((1-(3-(4-chloro-3-fluorophenoxy)-2-hydroxypropyl)pyrrolidin-3-yl)methyl)carbamate

To a stirred solution of tert-butyl (pyrrolidin-3-ylmethyl)carbamate (0.500 g, 2.50 mmol, 1.0 eq), and 2-((4-chloro-3-fluorophenoxy)methyl)oxirane (0.606 g, 3.00 mmol, 1.2 eq) in DMF (5 mL), was added K₂CO₃ (0.690 g, 5.00 mmol, 2.0 eq). The resultant reaction mixture was heated at 90° C. for overnight. Reaction was monitored by TLC and LCMS. After completion of reaction, the reaction mixture was diluted with water (40 mL) and extracted with EtOAc (3×50 mL) and washed with water (2×40 mL), brine solution (2×40 mL), dried over anhydrous sodium sulfate and concentrated under reduced pressure to obtain crude. The crude product was purified by flash chromatography (0-5% MeOH in DCM as eluent) to obtain tert-butyl ((1-(3-(4-chloro-3-fluorophenoxy)-2-hydroxypropyl)pyrrolidin-3-yl)methyl)carbamate (0.300 g, 30% Yield) as an off-white solid. LCMS 403.1 [M+H]⁺, ¹H NMR (400 MHz, Chloroform-d) δ 7.22-7.29 (m, 1H) 6.74 (dd, J=10.52, 2.63 Hz, 1H) 6.67 (dd, J=8.77, 2.63 Hz, 1H) 4.76 (br. s., 1H) 4.04-4.15 (m, 1H) 3.95 (d, J=4.82 Hz, 2H) 3.49 (s, 1H) 3.14 (br. s., 2H) 2.79-2.92 (m, 2H) 2.75 (t, J=6.80 Hz, 1H) 2.52-2.65 (m, 2H) 2.34-2.52 (m, 2H) 1.94-2.08 (m, 1H) 1.54 (dd, J=13.15, 6.14 Hz, 1H) 1.44 (s, 9H).

### Step-2: Synthesis of 1-(3-(aminomethyl)pyrrolidin-1-yl)-3-(4-chloro-3-fluorophenoxy)propan-2-ol 2,2,2-trifluoroacetate

To a stirred solution of tert-butyl ((1-(3-(4-chloro-3-fluorophenoxy)-2-hydroxypropyl)pyrrolidin-3-yl)methyl)carbamate (0.300 g, 0.744 mmol, 1.0 eq) in DCM (5 mL), was added TFA (0.5 mL) and the resultant reaction mixture was stirred at RT for overnight under nitrogen atmosphere. Reaction was monitored by TLC and LCMS. After completion of reaction, the reaction mixture was concentrated under reduced pressure. The crude product was crystallized in diethyl ether and dried under vacuum to obtain 1-(3-(aminomethyl)pyrrolidin-1-yl)-3-(4-chloro-3-fluorophenoxy)propan-2-ol 2,2,2-trifluoroacetate (0.250 g, 80% Yield) as an off white solid. LCMS 303.2 [M+H]⁺

### Step-3: Synthesis of 5-chloro-N-((1-(3-(4-chloro-3-fluorophenoxy)-2-hydroxypropyl)pyrrolidin-3-yl)methyl)benzofuran-2-carboxamide

To a solution of 1-(3-(aminomethyl)pyrrolidin-1-yl)-3-(4-chloro-3-fluorophenoxy)propan-2-ol 2,2,2-trifluoroacetate salt (0.250 g, 0.600 mmol, 1.0 eq) in DMF (05 mL) was added 5-chlorobenzofuran-2-carboxylic acid (0.0.118 g, 0.600 mmol, 1.0 eq) and HATU (0.342 g, 0.900 mmol, 1.5 eq) at RT. The reaction mixture was stirred for 10 minutes and then DIPEA (0.232 mL, 1.80 mmol, 3.0 eq) was added. The resultant reaction mixture was allowed to stir at RT for overnight. Progress of the reaction was monitored by LCMS. The reaction mixture was diluted with water (15 mL) and extracted with ethyl acetate (20 mL×2). Combined organic layer was washed with water 20 mL×4), dried over anhydrous Na2SO₄ and concentrated under reduced pressure. The crude product was purified by flash chromatography (0-5% MeOH in DCM as eluent) to obtain 5-chloro-N-((1-(3-(4-chloro-3-fluorophenoxy)-2-hydroxypropyl)pyrrolidin-3-yl)methyl)benzofuran-2-carboxamide (Compound 8—0.080 g, 27% Yield) as an off-white solid. LCMS 481.4 [M+H]⁺, ¹H NMR (400 MHz, DMSO-d₆) δ 8.92 (br. s., 1H) 7.87 (d, J=1.75 Hz, 1H) 7.69 (d, J=8.77 Hz, 1H) 7.35-7.58 (m, 3H) 7.06 (d, J=11.84 Hz, 1H) 6.83 (d, J=9.21 Hz, 1H) 4.01 (d, J=9.65 Hz, 2H) 3.93 (br. s., 1H) 2.68 (d, J=9.21 Hz, 2H) 1.92 (d, J=14.03 Hz, 2H) 1.57 (br. s., 2H) 1.23 (br. s., 1H) 0.93 (d, J=6.58 Hz, 1H).

### Example 47

### Synthesis of 2-(4-chloro-3-fluorophenoxy)-N-((1-(5-chlorobenzofuran-2-carbonyl)pyrrolidin-3-yl)methyl)acetamide

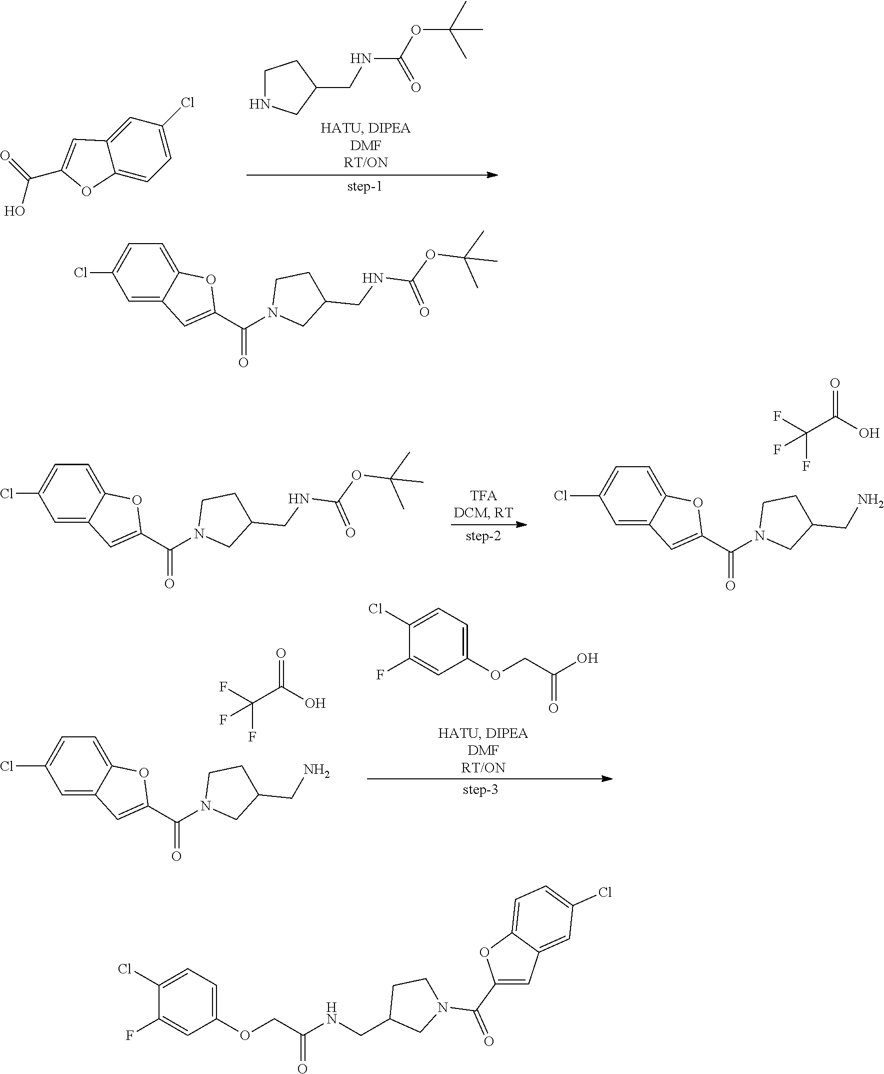

### Step-1: Synthesis of tert-butyl ((1-(5-chlorobenzofuran-2-carbonyl)pyrrolidin-3-yl)methyl)carbamate

To a solution of 5-chlorobenzofuran-2-carboxylic acid (0.250 g, 1.27 mmol, 1.0 eq) in DMF (5 mL) was added tert-butyl (pyrrolidin-3-ylmethyl)carbamate (0.255 g, 1.27 mmol, 1.0 eq) and HATU (0.965 g, 2.54 mmol, 2.0 eq) at RT. The reaction mixture was stirred for 5 minutes and DIPEA (0.4 mL, 1.00 mmol, 2.0 eq) was added. The resultant reaction mixture was allowed to stir at RT for overnight. Progress of the reaction was monitored by LCMS. The reaction mixture was diluted with cold water (20 mL) and extracted with ethyl acetate (60 mL×2). Combined organic layer was washed with cold water (20 mL×4), brine (20 mL×2), dried over anhydrous Na2SO₄ and concentrated under reduced pressure. The crude product was purified by flash chromatography (0-5% MeOH in DCM as an eluent) to obtain tert-butyl ((1-(5-chlorobenzofuran-2-carbonyl)pyrrolidin-3-yl)methyl)carbamate (120 mg, 24.8%) as a brown solid. LCMS 379.0 [M+H]⁺, ¹H NMR (400 MHz, DMSO-d₆) δ 7.83 (d, J=1.75 Hz, 1H) 7.67-7.77 (m, 1H) 7.38-7.55 (m, 2H) 7.03 (d, J=15.79 Hz, 1H) 3.82-3.99 (m, 1H) 3.51-3.67 (m, 2H) 2.91-3.10 (m, 2H) 2.17-2.46 (m, 2H) 1.83-2.05 (m, 1H) 1.54-1.73 (m, 1H), 1.39 (s, 9H).

### Step-2: Synthesis of (3-(aminomethyl)pyrrolidin-1-yl)(5-chlorobenzofuran-2-yl)methanone 2,2,2-trifluoroacetate

To a stirred solution of tert-butyl ((1-(5-chlorobenzofuran-2-carbonyl)pyrrolidin-3-yl)methyl)carbamate (0.06 g, 0.158 mmol, 1 eq) in DCM (30 mL), was added TFA (0.5 mL) and the resultant reaction mixture was stirred at RT for overnight. Reaction was monitored by LCMS. After completion of reaction, the reaction mixture was concentrated under reduced pressure and diluted with water. Aqueous layer washed with ethyl acetate (10 mL). Aqueous layer was separated and freeze dried over lyophilizer to obtain (3-(aminomethyl)pyrrolidin-1-yl)(5-chlorobenzofuran-2-yl)methanone 2,2,2-trifluoroacetate (60 mg, quantitative yield) as a yellow solid. LCMS 278.9 [M+H]⁺, ¹H NMR (400 MHz, DMSO-d₆) δ 7.84 (br. s., 2H) 7.70 (dd, J=8.33, 3.51 Hz, 1H) 7.39-7.52 (m, 1H) 4.01 (br. s., 1H) 3.85 (d, J=10.52 Hz, 1H) 3.68-3.81 (m, 1H) 3.32 (d, J=6.58 Hz, 2H) 2.83-2.99 (m, 2H) 2.59-2.73 (m, 1H) 2.52 (br. s., 1H) 2.06 (br. s., 1H) 1.77 (br. s., 1H).

### Step-3: Synthesis of 2-(4-chloro-3-fluorophenoxy)-N-((1-(5-chlorobenzofuran-2-carbonyl)pyrrolidin-3-yl)methyl)acetamide

To a solution of (3-(aminomethyl)pyrrolidin-1-yl)(5-chlorobenzofuran-2-yl)methanone 2,2,2-trifluoroacetate (0.06 g, 0.153 mmol, 1.0 eq) in DMF (1.0 mL) was added 2-(4-chloro-3-fluorophenoxy)acetic acid (0.031 g, 0.153 mmol, 1.0 eq) and HATU (0.116 g, 0.306 mmol, 2.0 eq) at RT. The reaction mixture was stirred for 5 minutes and then DIPEA (0.039 mL, 1.00 mmol, 2.0 eq) was added. The resultant reaction mixture was allowed to stir at RT for overnight. Progress of the reaction was monitored by LCMS. The reaction mixture was diluted with cold water (20 mL) and extracted with ethyl acetate (60 mL×2). Combined organic layer was washed with cold water (20 mL×4), brine (20 mL×2), dried over anhydrous Na2SO₄ and concentrated under reduced pressure The crude product was purified by flash chromatography (0-5% MeOH in DCM as an eluent) to obtain 2-(4-chloro-3-fluorophenoxy)-N-((1-(5-chlorobenzofuran-2-carbonyl)pyrrolidin-3-yl)methyl)acetamide (Compound 6—45 mg, 75% Yield) as a white solid.

LCMS 465.4 [M+H]⁺, ¹H NMR (400 MHz, DMSO-d₆) δ 8.32 (d, J=6.14 Hz, 1H) 7.83 (d, J=2.19 Hz, 1H) 7.71 (t, J=8.77 Hz, 2H) 7.46-7.54 (m, 1H) 7.36-7.46 (m, 1H) 7.01-7.17 (m, 1H) 6.75-6.92 (m, 1H) 4.56 (d, J=10.09 Hz, 2H) 3.80-3.99 (m, 1H) 3.36-3.66 (m, 2H) 3.04-3.29 (m, 2H) 2.42 (br. s., 1H) 2.01 (br. s., 1H) 1.93 (d, J=5.70 Hz, 1H) 1.60-1.70 (m, 1H).

### Example 48

### Synthesis of 2-(4-chloro-3-fluorophenoxy)-N-((1-(6-chloroquinoline-2-carbonyl)pyrrolidin-3-yl)methyl)acetamide

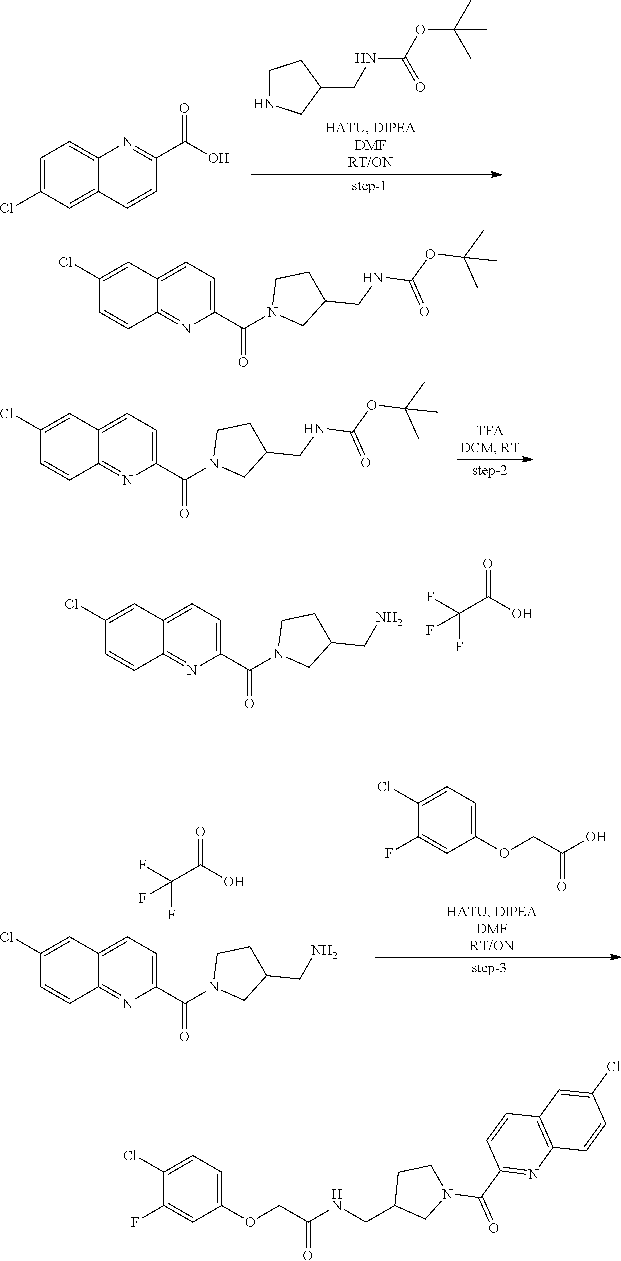

### Step-1: Synthesis of tert-butyl ((1-(6-chloroquinoline-2-carbonyl)pyrrolidin-3-yl)methyl)carbamate

To a solution of 6-chloroquinoline-2-carboxylic acid (0.2 g, 0.966 mmol, 1.0 eq) in DMF (1.5 mL) was added tert-butyl (pyrrolidin-3-ylmethyl)carbamate (0.270 g, 0.966 mmol, 1.0 eq) and HATU (0.734 g, 1.93 mmol, 2.0 eq) at RT. The reaction mixture was stirred for 5 minutes and then DIPEA (0.4 mL, 1.93 mmol, 2.0 eq) was added. The resultant reaction mixture was allowed to stir at RT for overnight. Progress of the reaction was monitored by LCMS. The reaction mixture was diluted with cold water (20 mL) and extracted with ethyl acetate (60 mL×2). Combined organic layer was washed with cold water (20 mL×4), brine (20 mL×2), dried over anhydrous Na2SO₄ and concentrated under reduced pressure. The crude product was purified by flash chromatography (0-5% MeOH in DCM as an eluent) to obtain tert-butyl ((1-(5-chlorobenzofuran-2-carbonyl)pyrrolidin-3-yl)methyl)carbamate (0.100 g, 26% Yield) as a yellow semi solid. LCMS 390.2 [M+H]⁺, ¹H NMR (400 MHz, DMSO-d₆) δ 8.47 (d, J=8.77 Hz, 1H) 8.21 (d, J=2.19 Hz, 1H) 7.99-8.09 (m, 1H) 7.78-7.93 (m, 2H) 7.06 (br. s., 1H), 3.58-3.83 (m, 2H) 3.45-3.57 (m, 2H) 3.39 (dd, J=11.18, 6.36 Hz, 1H) 2.82-3.08 (m, 1H) 2.22-2.40 (m, 1H) 1.97 (dd, J=11.18, 4.60 Hz, 1H) 1.51-1.74 (m, 1H) 1.17-1.31 (s, 9H).

### Step-2: Synthesis of (3-(aminomethyl)pyrrolidin-1-yl)(6-chloroquinolin-2-yl)methanone 2,2,2-trifluoroacetate

To a stirred solution of tert-butyl ((1-(5-chlorobenzofuran-2-carbonyl)pyrrolidin-3-yl)methyl)carbamate (0.1 g, 0.257 mmol, 1 eq) in DCM (10 mL), was added TFA (0.5 mL) and the resultant reaction mixture was stirred at RT for overnight. Reaction was monitored by LCMS. After completion of reaction, the reaction mixture was concentrated under reduced pressure and diluted with water. Aqueous layer washed with ethyl acetate (10 mL). Aqueous layer was separated and freeze dried over lyophilizer to obtain tert-butyl ((1-(6-chloroquinoline-2-carbonyl)pyrrolidin-3-yl)methyl)carbamate 2,2,2-trifluoroacetate (100 mg, 97%) as a brown semi solid. LCMS 290.0 [M+H]⁺

### Step-3: Synthesis of 2-(4-chloro-3-fluorophenoxy)-N-((1-(6-chloroquinoline-2-carbonyl)pyrrolidin-3-yl)methyl)acetamide

To a solution of (3-(aminomethyl)pyrrolidin-1-yl)(6-chloroquinolin-2-yl)methanone 2,2,2-trifluoroacetate (0.05 g, 0.124 mmol, 1.0 eq) in DMF (2 mL) was added 2-(4-chloro-3-fluorophenoxy)acetic acid (0.025 g, 0.124 mmol, 1.0 eq) and HATU (0.094 g, 0.248 mmol, 2.0 eq) at RT. The reaction mixture was stirred for 5 minutes and then DIPEA (0.015 mL, 0.248 mmol, 2.0 eq) was added. The resultant reaction mixture was allowed to stir at RT for overnight. Progress of the reaction was monitored by LCMS. The reaction mixture was diluted with cold water (20 mL) and extracted with ethyl acetate (60 mL×2). Combined organic layer was washed with cold water (20 mL×4), brine (20 mL×2), dried over anhydrous Na2SO4 and concentrated under reduced pressure. The crude product was purified by flash chromatography (0-5% MeOH in DCM as an eluent) to obtain 2-(4-chloro-3-fluorophenoxy)-N-((1-(6-chloroquinoline-2-carbonyl)pyrrolidin-3-yl)methyl)acetamide (Compound 5—60 mg, 100%) as white solid. LCMS 476.3 [M+H]⁺, ¹H NMR (400 MHz, DMSO-d₆) δ 8.47 (d, J=8.77 Hz, 1H) 8.34 (br. s., 1H) 8.18-8.27 (m, 1H) 8.06 (t, J=9.43 Hz, 1H) 7.76-7.92 (m, 1H) 7.50 (t, J=8.99 Hz, 1H) 7.37 (t, J=8.77 Hz, 1H) 6.94-7.01 (m, 1H) 6.77 (d, J=7.89 Hz, 1H) 4.58 (s, 1H) 4.49 (s, 1H) 3.58-3.87 (m, 2H) 3.47-3.58 (m, 1H) 3.41 (dd, J=11.62, 7.24 Hz, 1H) 3.03-3.32 (m, 2H) 2.41 (dd, J=12.72, 6.58 Hz, 1H) 1.83-2.05 (m, 1H) 1.55-1.74 (m, 1H).

### BIOLOGICAL EXAMPLES

### Example B1—ATF4 Expression Inhibition Assay

The ATF4 reporter was prepared by fusing the human full length 5′UTR of ATF4 (NCBI Accession No. BC022088.2) upstream of the firefly luciferase coding sequence lacking the initiator methionine. The fused sequence was cloned into pLenti-EF1a-C-Myc-DDK-IRES-Puro cloning vector (Origen # PS 100085) using standard methods. Virus production was carried out by using Lenti-X™ Packaging Single Shots Protocol (Clonetech #631276). Viral particles were used to transduce HEK293T cells (ATCC # CRL-3216, ATCC Manassas, Va.), which were subsequently selected with puromycin to generate stable cell line. Cells were maintained at 37° C. and 5% CO₂ in DMEM-F12 (Hyclone # SH30023.02) supplemented with 10% heat-inactivated fetal bovine serum (Gibco #16000-044), 2 mM L-glutamine (Gibco #25030-081), 100 U/ml penicillin, and 100 μg/ml streptomycin (Gibco #15140-122).

HEK293T cells carrying the ATF4 luciferase reporter were plated on 96-well plates (Nunc) at 10,000 cells per well. Cells were treated two days after seeding with 100 nM thapsigargin (Tg) (Sigma-Aldrich # T9033) in the presence of different concentrations of selected compounds ranging from 1 nM to 10 μM. Cells without treatment or cells treated with Tg alone were used as controls. Assay plates containing cells were incubated for 3 hours at 37° C.

Luciferase reactions were performed using Luciferase Assay System (Promega # E1501) as specified by the manufacturer. Luminescence was read with an integration time of 1 s and a gain of 110 using a Cytation-5 multi-mode microplate reader (BioTek). Relative luminescence units were normalized to Tg treatment (0% inhibition) and untreated cells (100% inhibition) and the percentage of ATF4 inhibition was calculated.

The half-maximal inhibitory concentration (IC₅₀) for the increasing of ATF4 protein levels is shown in Table 2. Under ISR stressed conditions (resulting from treatment with Tg), ATF4 expression is generally upregulated. Accordingly, inhibition of ATF4 expression as a result of the test compound indicates suppression of the ISR pathway.

TABLE 2

| Compound No. | ATF4 inhbition IC₅₀ (nM) |
|---|---|
| 1 | 856 |
| 2 | >1000 |
| 3 | 248.6 |
| 4 | >100 |
| 5 | >100 |
| 6 | >100 |
| 7 | >100 |
| 8 | >1000 |
| 12 | 3.58 |
| 13 | >100 |
| 14 | 216.7 |
| 15 | >100 |
| 16 | 134 |
| 17 | 102 |
| 18 | 177.6 |
| 19 | >100 |
| 20 | >100 |
| 21 | >1000 |
| 33 | >1000 |
| 34 | >1000 |
| 53 | >1000 |
| 56 | >1000 |
| 80 | >100 |
| 81 | 1000 |
| 82 | >1000 |
| 83 | >1000 |
| 84 | >100 |
| 85 | <1000 |
| 86 | <100 |
| 87 | <1000 |
| 88 | <1000 |
| 89 | >1000 |
| 90 | 57.8 |
| 91 | 1236 |
| 92 | 14.1 |
| 93 | 2.8 |
| 94 | 24.3 |
| 95 | <1000 |
| 96 | 4.97 |
| 97 | 25.0 |
| 98 | >100 |
| 99 | >100 |
| 100 | >100 |
| 101 | >100 |

### Example B2—ATF4 Expression Inhibition Assay

HEK293T cells are maintained at 37° C. and 5% CO₂ in Dulbecco's Modified Eagle's Media (DMEM) supplemented with 10% fetal bovine serum (FBS), 2 mM L-glutamine, 100 U/ml penicillin, and 100 μg/ml streptomycin. After reaching 80% of confluence, cells are detached and seeded on 6 well plates in complete media, allowed to recover overnight and treated for 3 hours with 100 nM thapsigargin (Tg) in the presence of 100 nM or 1 μM concentration of a test compound (percent inhibition assays) or various concentrations ranging from 1 nM to 1 μM (IC₅₀ assay). Cells without treatment or cells treated with Tg alone are used as controls.

After 3 hours of treatment with Tg and the test compound, cells are lysed with SDS-PAGE lysis buffer. Lysates are transferred to 1.5 ml tubes and sonicated for 3 min, and total protein amounts are quantified using BCA Protein Assay Kit (Pierce). Equal amount of proteins is loaded on SDS-PAGE gels. Proteins are transferred onto 0.2 m PVDF membranes (BioRad) and probed with primary antibodies diluted in Tris-buffered saline supplemented with 0.1% Tween 20 and 3% bovine serum albumin.

ATF4 (11815) antibody is used as primary antibody (Cell Signaling Technologies). A horseradish peroxidase (HRP)-conjugated secondary antibody (Rockland) is employed to detect immune-reactive bands using enhanced chemiluminescence (ECL Western Blotting Substrate, Pierce). Quantification of protein bands is done by densitometry using ImageJ.

Percentages of ATF4 inhibition after induction with Tg in the presence of 100 nM or 1 μM of certain test compounds can be reported. Percentage of ATF4 inhibition can be calculated as the percent reduction normalized to Tg treatment (0% inhibition) and untreated cells (100% inhibition). The calculated IC₅₀ for the test compounds can also be reported. Under ISR stressed conditions (resulting from treatment with Tg), ATF4 expression is generally upregulated. Accordingly, inhibition of ATF4 expression as a result of the test compound indicates suppression of the ISR pathway.

### Example B3—Protein Translation Assay

Chinese hamster ovary (CHO) cells were maintained at 37° C. and 5% C₀₂ in Dulbecco's Modified Eagle's Media (DMEM) supplemented with 10% fetal bovine serum, 2 mM L-glutamine, 100 U/ml penicillin, and 100 μg/ml streptomycin. After reaching 80% of confluence, cells were detached and seeded on 6 well plates in complete media, allowed to recover overnight and treated for 2 hours with 1 μM of the test compound (to assess protein synthesis levels in unstressed condition), or for 2 hours with 300 nM Tg in the presence of 1 μM of the test compound (to assess the recovery of protein synthesis in a stressed condition). Cells with Tg alone were used as controls.

After the 2 hours treatments, media were replaced by adding 10 μg/ml puromycin (Sigma Aldrich # P8833) in complete media for 30 min. Media were removed and cells were lysed with SDS-PAGE lysis buffer. Lysates were transferred to 1.5 ml tubes and sonicated for 3 min and total protein amount were quantified using BCA Protein Assay Kit (Pierce). Equal amount of protein (30 μg) was loaded on SDS-PAGE gels. Proteins were transferred onto 0.2 μm PVDF membranes (BioRad) and probed with primary antibodies diluted in Tris-buffered saline supplemented with 0.1% Tween 20 (Merck # S6996184 505) and 3% bovine serum albumin (Rockland # BSA-50).

Puromycin (12D10) (Merck # MABE343) and β-actin (Sigma Aldrich # A5441) antibodies were used as primary antibody. A HRP-conjugated secondary antibody (Rockland) was employed to detect immune-reactive bands using enhanced chemiluminescence (ECL Western Blotting Substrate, Pierce). Quantification of protein bands was done by densitometry using ImageJ software.

Percent increase of protein synthesis in unstressed cells (without Tg treatment) in the presence of media alone or certain test compounds is shown in Table 3. The percentage levels were normalized to the media alone condition, which correspond to 100% protein synthesis. Certain compounds stimulated protein synthesis above baseline, indicating that these test compounds result in increased protein synthesis in unstressed cells.

Percent recovery of protein synthesis in stressed cells (with Tg treatment) due to the test compounds at 1 μM is also shown in Table 3. The levels were normalized to the media alone and Tg alone conditions, which correspond to 100% and 0% respectively.

TABLE 3

| | % Recovery of | % Protein synthesis |
|---|---|---|
| Compound | protein synthesis | relative to untreated |
| No. | (1 μM test compound) | (1 μM test compound) |
| 1 | n.d. | 46.9 |
| 2 | 28.2 | 84.7 |
| 3 | 286 | 176.1 |
| 4 | −53.2 | 76.1 |
| 5 | 0.00 | 94.2 |
| 6 | 0.00 | 81.2 |
| 7 | −15.9 | 121.9 |
| 8 | 31.57 | 107.1 |
| 12 | 13 | 97.7 |
| 13 | 161.6 | 288.2 |
| 14 | n.d. | 65.9 |
| 15 | −4.2 | 110.5 |
| 16 | n.d. | 95.4 |
| 17 | 22 | 78.4 |
| 18 | n.d. | 158.3 |
| 19 | −1.3 | 76.1 |
| 20 | 52.5 | 122.6 |
| 21 | 54.6 | 140.6 |
| 33 | 101.1 | 147 |
| 34 | 122.6 | 166.1 |
| 53 | 37.93 | 101.9 |
| 56 | 32.73 | 100.5 |
| 80 | 23.9 | 152.7 |
| 81 | 38.7 | 133.6 |
| 82 | 176.4 | 159.7 |
| 83 | 175.2 | 128.8 |
| 84 | −87.4 | 80.3 |
| 85 | −94.8 | 88 |
| 86 | −97 | 94.2 |
| 87 | −94.5 | 83.9 |
| 88 | −28.1 | 76.4 |
| 89 | 42.5 | 118.7 |
| 90 | 269.7 | 210.4 |
| 91 | 209.4 | 190.4 |
| 92 | 45.8 | 93.6 |
| 93 | 51.4 | 101.8 |
| 94 | 298.3 | 169.2 |
| 95 | 170.5 | 186.1 |
| 96 | 24.5 | 109.9 |
| 97 | −34.4 | 109.1 |
| 98 | 142.63 | 167.3 |
| 99 | 0.00 | 67.7 |
| 100 | 0.00 | 69.1 |
| 101 | 86.10 | 93.2 |

Data summarized in Tables 2 and 3 show that some compounds have differential activity in ATF4 inhibition and protein synthesis under ISR-inducing conditions. That is, some compounds are able to effectively inhibit ATF4 expression but do not restore protein synthesis. Other compounds effectively restore protein synthesis but do not inhibit ATF4 expression under ISR-inducing conditions. Still other compounds inhibit ATF4 expression and restore protein synthesis.

### Example B4—ATF4 Inhibition Assay Under Aβ Stimulation

Chinese hamster ovary (CHO) cells that stably express human APP751 incorporating the familial Alzheimer's disease mutation V717F are used as a source of Aβ monomer and low-n oligomers. These cells, referred to as 7PA2 CHO cells, are cultured in 100 mm dishes with Dulbecco's modified Eagle's medium (DMEM) containing 10% fetal bovine serum, 2 mM L-glutamine, 100 U/ml penicillin, and 100 μg/ml penicillin, streptomycin and 200 μg/ml G418. Upon reaching 90-100% confluency, cells are washed with 5 mL of glutamine- and serum-free DMEM and incubated for approximately 16 h in 5 mL of the same DMEM. Conditioned media (CM) is collected.

SH-SY5Y cells are maintained at 37° C. and 5% CO₂ in RPMI 1640 media supplemented with 10% fetal bovine serum (FBS), penicillin and streptomycin. After reaching 80% of confluence, cells are detached and seeded on 6 well plates in complete media, allowed to recover 48 h and treated for 16 hours with CM from WT CHO cells or 7PA2 CHO cells in the presences of 1 μM of selected compounds.

After 16 hours treatment, culture media are removed and cells are lysed with SDS-PAGE lysis buffer. Lysates are transferred to 1.5 ml tubes and sonicated for 3 min. Total protein amount is quantified using BCA Protein Assay Kit (Pierce). Equal amount of proteins (30 μg) is loaded on SDS-PAGE gels. Proteins are transferred onto 0.2 μm PVDF membranes (BioRad) and probed with primary antibodies diluted in Tris-buffered saline supplemented with 0.1% Tween 20 and 3% bovine serum albumin.

ATF4 (11815) antibody is used as primary antibody (Cell Signaling Technologies). A β-actin antibody is used as a control primary antibody. An HRP-conjugated secondary antibody (Rockland) is employed to detect immune-reactive bands using enhanced chemiluminescence (ECL Western Blotting Substrate, Pierce). Quantification of protein bands is done by densitometry using ImageJ.

Percent inhibition of ATF4 expression in SH-SY5Y cells after incubation with CM from the 7PA2 CHO cells as a result of the test compounds can be reported. Percentage of ATF4 inhibition is calculated as the percent reduction normalized to CM from 7PA2 CHO cells treatment (0% inhibition) and CM from WT CHO cells treatment (100% inhibition).

### Example B5—Electrophysiology and Long-Term Potentiation

Hippocampal slices are prepared as described in Ardiles et al., Pannexin 1 regulates bidirectional hippocampal synaptic plasticity in adult mice. Front Cell Neurosci, vol. 8, art. 326 (2014). Six to eleven-month-old WT C57BL/6 or transgenic APP/PS 1 mice (Jackson Lab 34829-JAX) are deeply anesthetized with isoflurane and their brains are quickly removed. 5-10 slices (350 μm) from each animal are dissected in ice-cold dissection buffer using a vibratome (Leica VT1200S, Leica Microsystems, Nussloch, Germany). Slices are incubated with 5 μM ISRIB (trans-N,N′-1,4-cyclohexanediylbis[2-(4-chlorophenoxy)-acetamide), a selected compound, or a vehicle (complete medium containing 0.1% DMSO) 20 min before conditioning stimulation. Synaptic responses are evoked by stimulating the Schaffer collaterals with 0.2 ms pulses delivered through concentric bipolar stimulating electrodes, and recorded extracellularly in the stratum radiatum of the CA1 subfield. Long-term potentiation (LTP) is induced by four-theta burst stimulation (TBS) (10 trains of four pulses at 100 Hz; 5 Hz inter-burst interval) delivered at 0.1 Hz. LTP magnitude based on field excitatory postsynaptic potential (fEPSP) is calculated as the average (normalized to baseline) of the responses recorded 60 min after conditioning stimulation. Similar experiments can be performed using a test compound in place of ISRIB.

### Example B6—Learning Memory in Aged Mice

Wild type 19-month old male C57Bl/6J mice are used in an 8-arm radial water maze (RAWM) to measure the hippocampal-mediated learning memory. The maze involves a pool 118.5 cm in diameter and 25 cm high with 8 arms, each 41 cm in length, and an escape platform that can be moved. The pool is filled with water that is rendered opaque by adding white paint (Crayola, 54-2128-053). The escape platform remains hidden during the experiment. Visual cues are placed around the room such that they are visible to animals exploring the maze.

Nine mice are intraperitoneally injected with 5 mg/kg of a test compound formulated in 50% Polyethylene glycol (PEG-400) in distilled water and other 9 animals are intraperitoneally injected with the vehicle 50% PEG-400 in distilled water as a control group. Animals run 6 trials a day for two days. Animals are allowed 1 min to locate the escape platform. On successfully finding the platform, animals will remain for 10 seconds before being returned to their holding cage. On a failed trial, animals are guided to the escape platform and then will be returned to their holding cage 10 seconds later.

Behavioral tests are recorded and scored using a video tracking and analysis setup (Ethovision XT 8.5, Noldus Information Technology). The program automatically analyzes the number of incorrect arm entries (termed number of errors) made per trial. The last three trials are averaged to determine learning memory after training.

At the end of the behavioral test, animals are sacrificed and the hippocampi are extracted and immediately frozen in liquid nitrogen and are stored at −80° C. The frozen samples are then homogenized with a T 10 basic ULTRA-TURRAX (IKa) in ice-cold buffer lysis (Cell Signaling 9803) and protease and phosphatase inhibitors (Roche). Lysates are sonicated for 3 min and centrifuged at 13,000 rpm for 20 minutes at 4° C. Protein concentration in supernatants is determined using BCA Protein Assay Kit (Pierce). Equal amount of protein is loaded on SDS-PAGE gels. Proteins are transferred onto 0.2 m PVDF membranes (BioRad) and probed with primary antibodies diluted in Tris-buffered saline supplemented with 0.1% Tween 20 and 3% bovine serum albumin.

ATF4 (11815) antibody (Cell Signaling Technologies) and β-actin (Sigma-Aldrich) antibodies are used as primary antibodies. A HRP-conjugated secondary antibody (Rockland) is employed to detect immune-reactive bands using enhanced chemiluminescence (ECL Western Blotting Substrate, Pierce). Quantification of protein bands is done by densitometry using ImageJ software.

Results of RAWM task and levels of ATF4 expression normalized to β-actin expression in hippocampi can be reported.

### Example B7—Learning Memory, Long-Term Memory and Social Behavior after Traumatic Brain Injury (TBI)

Wild type three-month-old male C57Bl/6J mice are randomly assigned to TBI or sham surgeries. Animals are anesthetized and maintained at 2% isoflurane and secured to a stereotaxic frame with nontraumatic ear bars. The hair on their scalp is removed, and eye ointment and betadine are applied to their eyes and scalp, respectively. A midline incision is made to expose the skull. A unilateral TBI is induced in the right parietal lobe using the controlled cortical impact model (Nat Neurosci. 2014 August; 17(8): 1073-82). Mice receive a 3.5-mm diameter craniectomy, a removal of part of the skull, using an electric microdrill. The coordinates of the craniectomy are: anteroposterior, −2.00 mm and mediolateral, +2.00 mm with respect to bregma. After the craniectomy, the contusion is induced using a 3-mm convex tip attached to an electromagnetic impactor (Leica). The contusion depth is set to 0.95 mm from dura with a velocity of 4.0 m/s sustained for 300 ms. These injury parameters are chosen to target, but not penetrate, the hippocampus. Sham animals received craniectomy surgeries but without the focal injury. After focal TBI surgery, the scalp is sutured and the animal is allowed to recover in an incubation chamber set to 37° C. Animals are returned to their home cage after showing normal walking and grooming behavior. Recovery from the surgical procedures as exhibited by normal behavior and weight maintenance is monitored throughout the duration of the experiments.

After 28 days post injury (dpi), animals are tested on the RAWM assay (see Example B6, above). Animals run 12 trials during learning test and 4 trials during memory test. Last three trials from learning test and all four trials from memory test are averaged to determine learning memory (learning test) and long-term memory (memory test).

Animals are intraperitoneally injected with 5 mg/kg of a test compound formulated in 50% PEG-400 in distilled water (n=10) or vehicle (50% PEG-400 in distilled water; n=10 for TBI group and n=8 for sham group) starting the day prior to behavior tests (27 dpi), after each of the final trials of the learning-test days (28 and 29 dpi) and before the social behavior test (42 dpi, see below) for a total of four injections. No injections is given when long-term memory is tested on day 35 dpi.

To quantitate social tendencies of the treated mice, the time spent with a novel conspecific mouse is measured in a Crawley's three-chamber box (J Vis Exp. 2011; (48): 2473). Treated animals are left to explore all three empty chambers freely for 10 min for habituation. A social pair mouse is placed in the housing cage at one side of the apparatus and treated animals in opposite chamber so that the mouse can freely explore the entire apparatus for 10 min. The time spent with the never-before-met animal is recorded. Direct contact between the treated mouse and the housing cage or stretching of the body of the subject mouse in an area 3-5 cm around the housing cage is counted as an active contact.

Learning memory, long-term memory, and social behavior after TBI in mice are reported.

### Example B8—Fasting-Induced Muscle Atrophy

Wild type eight-weeks-old male Balb/c mice obtained from the vivarium Fundación Ciencia & Vida Chile (Santiago, Chile) are used. Mice are housed in independent plastic cages in a room maintained at 25° C. with a 12-h: 12-h light:dark cycle.

Twenty-four hours before and during the 2 days of fasted procedures, animals receive oral administration via feeding tubes (15 gauge) of vehicle (50% Polyethylene glycol 400 (Sigma-Aldrich P3265) in distilled water or 10 mg/kg of test compound formulated in vehicle solution.

After 2 days of fasting the animals are sacrificed and muscles are removed from both hindlimbs. Mice with feed and water ad libitum are used as control.

For in vivo measurements of protein synthesis, puromycin (Sigma-Aldrich, P8833) is prepared at 0.04 μmol/g body weight in a volume of 200 μL of PBS, and subsequently administered into the animals via IP injection, 30 min prior to muscle collection.

Upon collection, muscles are immediately frozen in liquid nitrogen and then stored at −80° C. The frozen muscles are then homogenized with a T 10 basic ULTRA-TURRAX (IKa) in ice-cold buffer lysis (Cell Signaling 9803) and protease and phosphatase inhibitors (Roche). Lysates are sonicated for 3 min and centrifuged at 13,000 rpm for 20 minutes at 4° C. Protein concentration in supernatants is determined using BCA Protein Assay Kit (Pierce). Equal amount of proteins is loaded on SDS-PAGE gels. Proteins are transferred onto 0.2 um PVDF membranes (BioRad) and probed with primary antibodies diluted in Tris-buffered saline supplemented with 0.1% Tween 20 and 3% bovine serum albumin.

Puromycin (12D10) (Merck Millipore) and β-actin (Sigma-Aldrich) antibodies are used as primary antibodies. A HRP-conjugated secondary antibody (Rockland) is employed to detect immune-reactive bands using enhanced chemiluminescence (ECL Western Blotting Substrate, Pierce). Quantification of protein bands is done by densitometry using ImageJ software.

For immunohistochemical analysis of cross-sectional area (CSA), muscles from control (Fed) and fasted animals are submerged individually in optimal cutting temperature (OCT) compound (Tissue-Tek; Sakura) at resting length, and frozen in isopentane cooled with liquid nitrogen. Cross-sections (10-μm thick) from the mid-belly of the muscles are obtained with a cryostat (Leica) and immunostained with puromycin antibody (12D10) (Merck Millipore). A HRP-polymer conjugated secondary antibody (Biocare Medical, MM620L) followed by diaminobenzidine substrate incubation (ImmPACT DAB—Vector, SK-4105) are employed to detect puromycinylated structures in CSA.

Percent of protein synthesis in fasted muscles is reported. The levels are normalized to β-actin expression and percentage is calculated as the percent relative to protein synthesis levels from control mice (Fed) which correspond to 100%.

Muscle fiber CSA are visualized with a Zeiss Axio Lab.A1 microscope and an Axiocam (Zeiss) digital camera. Puromycin staining in CSA can be reported.

### Example B9—Immobilization-Induced Muscle Atrophy

Wild type eight-weeks-old male Balb/c mice obtained from the vivarium Fundación Ciencia & Vida Chile (Santiago, Chile) are used. Mice are housed in independent plastic cages, fed ad libitum in a room maintained at 25° C. with a 12-h: 12-h light:dark cycle.

Twenty-four hours before and during the 3 days of immobilization procedures, animals receive oral administration via feeding tubes (15 gauge) of vehicle (50% Polyethylene glycol 400 (Sigma-Aldrich P3265) in distilled water or 10 mg/kg of test compound formulated in vehicle.

One hindlimb is immobilized with a plastic stick placed over and under the limb and fixed with a medical adhesive bandage. Animals are daily monitored. The immobilization procedure prevents movement of the immobilized leg alone. After 3 days, the animals are sacrificed and gastrocnemius, quadriceps and tibialis anterior muscles are removed from both hindlimbs, the contralateral, non-immobilized leg being used as an internal control.

For in vivo measurements of protein synthesis, puromycin (Sigma-Aldrich, P8833) is prepared at 0.04 μmol/g body weight in a volume of 200 μL of PBS, and subsequently administered into the animals via intraperitoneal injection, 30 min prior to muscle collection.

Upon collection, muscles are immediately frozen in liquid nitrogen and stored at −80° C. The frozen muscles are then homogenized with a T 10 basic ULTRA-TURRAX (IKa) in ice-cold buffer lysis (Cell Signaling 9803) and protease and phosphatase inhibitors (Roche). Lysates are sonicated for 3 min and centrifuged at 13,000 rpm for 20 minutes at 4° C. Protein concentration in supernatants is determined using BCA Protein Assay Kit (Pierce). Equal amount of protein is loaded on SDS-PAGE gels. Proteins are transferred onto 0.2 um PVDF membranes (BioRad) and probed with primary antibodies diluted in Tris-buffered saline supplemented with 0.1% Tween 20 and 3% bovine serum albumin.

Puromycin (12D10) (Merck Millipore) and β-actin (Sigma-Aldrich) antibodies are used as primary antibodies. A HRP-conjugated secondary antibody (Rockland) is employed to detect immune-reactive bands using enhanced chemiluminescence (ECL Western Blotting Substrate, Pierce). Quantification of protein bands is done by densitometry using ImageJ software.

Percent of protein synthesis in mobile and immobile hind limbs sections from gastrocnemius, tibialis anterior and quadriceps can be reported. The levels are normalized to β-actin expression and percentage is calculated as the percent relative to protein synthesis levels from mobile limb of control mice (vehicle-treated) which correspond to 100%.

### Example B10—Cachexia-Induced Muscle Atrophy

Wild type six-weeks-old male Balb/c mice obtained from the vivarium Fundación Ciencia & Vida Chile (Santiago, Chile) are used. Mice are housed in independent plastic cages in a room maintained at 25° C. with a 12-h: 12-h light:dark cycle.

1×10⁶ CT26 colon carcinoma cell line (ATCC # CRL-2638, ATCC Manassas, Va.) are injected subcutaneously in the right lower flank of each animal for induction of cachexia-induced muscle atrophy as described (Nat Commun. 2012 Jun. 12; 3:896). Non-injected animals are used as controls. At day 6 post tumor-cell injection, animals are randomized into two groups and treated with 10 mg/kg of test compound formulated in 50% Polyethylene glycol (PEG-400) in distilled water, or with vehicle (50% PEG-400 in distilled water) by daily oral gavage for 13 days.

For in vivo measurements of protein synthesis, 30 min before the study ends, animals are injected intraperitoneally with puromycin (Sigma-Aldrich, P8833) at 0.04 μmol/g body weight in a volume of 200 μL of PBS. After 13 days of daily dosage, the animals are sacrificed and gastrocnemius, quadriceps and tibialis anterior muscles are dissected and weighed from both hindlimbs to assess muscle atrophy.

Upon collection, muscles are immediately frozen in liquid nitrogen and then stored at −80° C. The frozen muscles are then homogenized with a T 10 basic ULTRA-TURRAX (IKa) in ice-cold buffer lysis (Cell Signaling 9803) and protease and phosphatase inhibitors (Roche). Lysates are sonicated for 3 min and centrifuged at 13,000 rpm for 20 minutes at 4° C. Protein concentration in supernatants is determined using BCA Protein Assay Kit (Pierce). Equal amount of protein is loaded on SDS-PAGE gels. Proteins are transferred onto 0.2 um PVDF membranes (BioRad) and probed with primary antibodies diluted in Tris-buffered saline supplemented with 0.1% Tween 20 and 3% bovine serum albumin.

Puromycin (12D10) (Merck Millipore) and β-actin (Sigma-Aldrich) antibodies are used as primary antibodies. A HRP-conjugated secondary antibody (Rockland) is employed to detect immune-reactive bands using enhanced chemiluminescence (ECL Western Blotting Substrate, Pierce). Quantification of protein bands is done by densitometry using ImageJ software.

Gastrocnemius, quadriceps and tibialis anterior muscle weight from animals injected with CT26 tumor cells and treated with either vehicle or test compound are reported.

Percent of protein synthesis in gastrocnemius, quadriceps and tibialis anterior from animals injected with CT26 tumor cells and treated with either vehicle or test compound is also reported. The levels are normalized to β-actin expression and percentage is calculated as the percent relative to protein synthesis levels from muscle section of control mice which correspond to 100%.

### Example B11—Tumor Growth and Density Model

Wild type six-weeks-old male Balb/c mice obtained from the vivarium Fundación Ciencia & Vida Chile (Santiago, Chile) are used. Mice are housed in independent plastic cages in a room maintained at 25° C. with a 12-h: 12-h light:dark cycle.

1×10⁶ CT26 colon carcinoma cell line (ATCC # CRL-2638, ATCC Manassas, Va.) are injected subcutaneously in the right lower flank of each animal as described (Nat Commun. 2012 Jun. 12; 3:896). Non-injected animals are used as controls. At day 6 post tumor-cell injection, mean tumor volume is measured, animals are weighed and randomized into two groups and treated with 10 mg/kg of a selected compound formulated in 50% Polyethylene glycol (PEG-400) in distilled water, or with vehicle (50% PEG-400 in distilled water) by daily oral gavage for 13 days.

At the end of the study, tumors are measured with digital caliper and tumors volumes, expressed in mm³, are calculated with the following formula:

Tumor volume (mm³)=(a×b²)/2

where “a” is the largest perpendicular diameter and “b” is the smallest diameter. Animals are then sacrificed and tumors are extracted and weighed. The volumes and weight of the tumors of each animal are reported. Tumor density is calculated as weight/volume ratio for each animal and is reported. Statistical analyses are performed using GraphPad Prism software and significant difference is assessed by t test (*<0.05).

### Example B12—Protein Synthesis with a Cell-Free System

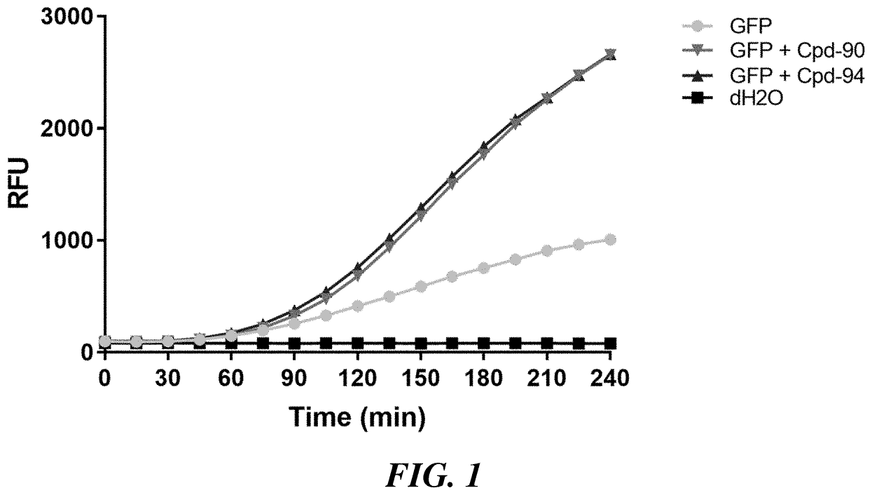

The expression of the green fluorescence protein (GFP) was evaluated using the 1-Step Human In vitro Protein Expression Kit based on HeLa cell lysates (ThermoFisher Scientific). HeLa lysate, accessory proteins, reaction mix and pCFE-GFP plasmid from the kit were thawed in ice. Reactions were prepared at room temperature in a 96-well optical plate by adding 12.5 μL of HeLa lysate, 2.5 μL accessory proteins, 5 μL reaction mix, 1 μg of pCFE-GFP plasmid and 1 μM of test compounds in 5 μL or 5 μL of distilled H₂O as a basal expression of GFP (vehicle). A well with dH₂O instead of pCFE-GFP plasmid was used as basal autofluorescence of the reaction. All reactions was made in duplicated. Fluorescence intensity was measured by a multi-mode microplate reader (Synergy-4; Biotek) during 4-hour treatments and capturing fluorescence at 15-minute intervals with 485/20 and 528/20 excitation and emission filters. Relative fluorescence intensity (RFU) of GFP treated with or without test compounds was shown in FIG. 1. The addition of tested compound to the kit's reaction mix increased the expression of GFP and hence its fluorescence compared to the expression obtained using the kit's reagents alone.

### Example B13—Protein Synthesis with a Yeast Cell-Based Assay

Two GS 115H Pichia pastoris yeast strains that stably express phospholipase C protein (PLC) under the control of a methanol-inducible promoter (pAOX-PLC) or a constitutive promoter (pGAP-PLC) are used to assess the secretion levels of PLC and its enzymatic activity. pAOX-PLC and pGAP-PLC yeast single colonies are inoculated in 2 ml of YPD (1% yeast extract, 2% peptone, 2% glucose) and grown at 30° C. in a deep 24-well microplate in a shaking incubator for 16-18 h at 250 rpm. These cultures are diluted to an OD600 of 1 in 2 ml of YPM (1% yeast extract, 2% peptone, 100 mM phosphate buffer pH 6 and 0.5% methanol) or 2 ml of YPM containing 10M of a test compound to induce gene expression and incubated at 30° C. in a shaking incubator at 250 rpm. Methanol is added every 24 h in order to maintain 0.5% methanol concentration. Condition without test compound is used as a control for basal secretion and subsequent activity assessment of PLC. After 72 h of induction, cells are harvested by centrifugation and the supernatant analyzed for protein expression by SDS-PAGE and PLC activity.

Gels were stained with 0.1% Coomassie Blue R250 in 10% acetic acid, 50% methanol, and 40% H₂O for 20 minutes. Stained gel is then washed twice for 2 hours with 10% acetic acid, 50% methanol and 40% dH₂O until the Coomassie Blue background was nearly clear. Photographs of the gels are taken in a gel imaging system.

PLC activity is measured in 96 well microplates using 1 mM O-(4-Nitrophenylphosphoryl)choline as a substrate. The assay is carried out at 50° C. in a 96 microwell plate by incubating 10 μL of culture supernatant, 10 μL 100 mM NPPC and 80 μL of 250 mM HEPES pH 7, 60% sorbitol, 0.1 mM ZnCl₂. Absorbance at 405 nm is monitored every 30 s for 1 h at 50° C. in a Synergy HT microplate reader (Biotek). 1 PLC unit is defined as the amount of enzyme releasing 1 nmol of p-nitrophenol per minute.

### Example B14—Protein Synthesis with a CHO Cell-Based Assay

CHO cells are maintained at 37° C. and 5% CO₂ in DMEM supplemented with 10% fetal bovine serum (FBS), 2 mM L-glutamine, 100 U/ml penicillin, and 100 μg/ml streptomycin. After reaching 80% of confluence, cells are detached and seeded on 6-well plates in complete media, allowed to recover for 48h. Cells are then washed three times with PBS and treated with test compounds at 1 μM, 5 μM or 10 μM in 1 mL of media without FBS for 24 h. Treatment with 0.1% DMSO is used as control (vehicle). After 24h treatment, supernatant (SN) which contains secreted proteins is extracted and protease and phosphatase inhibitors (Roche) are added to each sample. SN are centrifuged at 2,000 g for 10 min to discard any cellular debris and 900 μL SN are transferred to empty microtubes with 400 μL methanol by mixing well. 200 μL of chloroform is added to the mix and then samples are centrifuged at 14,000 g for 2 minutes. Top aqueous layer is discarded by pipetting off and 400 μL methanol is added to each sample by mixing well. Samples are then centrifuged at 17,000 g for 8 minutes and methanol is discarded by pipetting off without disturbing the protein pellet. Samples are left dry at room temperature and pellets are resuspended with SDS-PAGE sample buffer. Secreted proteins are analyzed by SDS-PAGE and Coomassie staining. Gels are stained with 0.1% Coomassie Blue R250 in 10% acetic acid, 50% methanol, and 40% H₂O for 20 minutes. Stained gel is then washed twice for 2 hours with 10% acetic acid, 50% methanol and 40% dH₂O until the Coomassie Blue background is nearly clear. Photographs of the gels are taken in a gel imaging system.

All references throughout, such as publications, patents, patent applications and published patent applications, are incorporated herein by reference in their entireties.

1. A compound of formula (I) 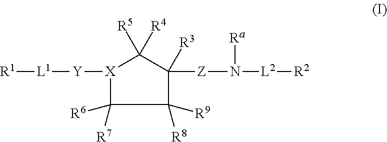
or a pharmaceutically acceptable salt thereof,
wherein:
X is N or CR¹²;
Y is a bond, NR^{a}, or NR^{a}NR^{a}; provided that:
(a) when X is N, then Y is a bond or NR^{a}; and
(b) when X is CR¹², then Y is NR^{a} or NR^{a}NR^{a};
Z is a bond, C(═O), CR¹¹R¹¹, or NR^{a};
L¹ is selected from the group consisting of *1-C(═O)-#1, *1-CH₂-#1, *1-CH₂CH₂-#1, *1-CH₂CH₂CH₂-#1, *1-OCH₂C(═O)-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1;
wherein *1 represents the attachment point to R¹ and #1 represents the attachment point to the remainder of the molecule;
L² is selected from the group consisting of #2-C(═O)-*2, #2-CH₂-*2, #2-CH₂CH₂-*2, #2-CH₂CH₂CH₂-*2, #2-C(═O)CH₂O—*2, #2-C(═O)CH₂CH₂O—*2, #2-C(═O)CH₂CH₂CH₂O-*2, #2-CH₂CH(OH)CH₂O—*2, #2-CH₂O-*2, #2-CH₂CH₂O-*2, and #2-CH₂CH₂CH₂O-*2;
wherein *2 represents the attachment point to R² and #2 represents the attachment point to the remainder of the molecule;
R¹ is selected from the group consisting of:
C₆-C₁₄ aryl substituted with one or more halo groups and optionally substituted with one or more R^{b}; and
5-14 membered heteroaryl substituted with one or more halo groups and optionally substituted with one or more R^{b};
R² is selected from the group consisting of:
C₆-C₁₄ aryl substituted with one or more halo groups and optionally substituted one or more R^{b}; and
5-14 membered heteroaryl substituted with one or more halo groups and optionally substituted one or more R^{b};
R³ is hydrogen, halogen, or C₁-C₆ alkyl; or R³ and R¹² are taken together to form a CR¹³R¹⁴ group;
R⁴, R⁵, R⁶, R⁷, R⁸, and R⁹, independently of each other, are selected from the group consisting of hydrogen, halogen, and C₁-C₆ alkyl;
R¹⁰ and R¹¹, independently of each other, are selected from the group consisting of hydrogen, halogen, and C₁-C₆ alkyl;
R¹² is hydrogen, halogen, or C₁-C₆ alkyl; or R³ and R¹² are taken together to form a CR¹³R¹⁴ group;
R¹³ and R¹⁴, independently of each other, are selected from the group consisting of hydrogen, halogen, and C₁-C₆ alkyl;
R^{a}, independently at each occurrence, is hydrogen or C₁-C₆ alkyl;
R^{b}, independently at each occurrence, is selected from the group consisting of NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, NR^{c}R^{d}, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, C(O)NR^{14-a}R^{14-b}, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, S(O)₂NR^{c}R^{d}, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl),
wherein R^{c} and R^{d} are taken together with the nitrogen atom to which they are attached to form a 3-10 membered heterocycle; and
provided that:
(i) when X is CR¹², Y is NR^{a}, Z is a bond, L¹ is *1-CH₂-#1, and L² is #2-CH₂-*2; then either:
(i-1) at least one of R³, R⁴, and R⁵ is hydrogen or halogen; or
(i-2) R³ and R¹² are taken together to form a CR¹³R¹⁴ group;
(ii) when X is CR¹², Y is NR^{a}, Z is a bond, and R³ and R¹² are taken together to form CR¹³R¹⁴; then either:
(ii-1) L¹ is selected from the group consisting of *1-C(═O)-#1, *1-CH₂-#1, *1-CH₂CH₂-#1, *1-CH₂CH₂CH₂-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1; or
(ii-2) L² is selected from the group consisting of #2-C(═O)-*2, #2-CH₂-*2, #2-CH₂CH₂-*2, #2-CH₂CH₂CH₂-*2, #2-C(═O)CH₂CH₂O—*2, #2-C(═O)CH₂CH₂CH₂O-*2, #2-CH₂CH(OH)CH₂O—*2, #2-CH₂O-*2, #2-CH₂CH₂O-*2, and #2-CH₂CH₂CH₂O-*2; and
(iii) when X is N and Y is a bond; then:
L¹ is selected from the group consisting of *1-OCH₂CH(OH)CH₂-#1, *1-OCH₂-#1, *1-OCH₂CH₂-#1, and *1-OCH₂CH₂CH₂-#1; and
further provided that, when Z is CR¹⁰R¹¹, then at least one of R¹ and R² is substituted by two or more halo groups.

2. The compound of claim 1, or a pharmaceutically acceptable salt thereof, wherein the compound of formula (I) is a compound of formula (II) 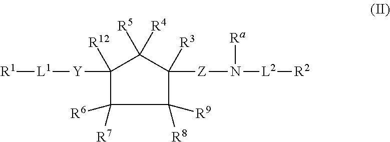
provided that:
(i) when Y is NR^{a}, Z is a bond, L¹ is *1-CH₂-#1, and L² is #2-CH₂-*2; then either:
(i-1) at least one of R³, R⁴, and R⁵ is hydrogen or halogen; or
(i-2) R³ and R¹² are taken together to form a CR¹³R¹⁴ group; and
(ii) when Y is NR^{a}, Z is a bond, and R³ and R¹² are taken together to form CR¹³R¹⁴; then either:
(ii-1) L¹ is selected from the group consisting of *1-C(═O)-#1, *1-CH₂-#1, *1-CH₂CH₂-#1, *1-CH₂CH₂CH₂-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1; or
(ii-2) L² is selected from the group consisting of #2-C(═O)-*2, #2-CH₂-*2, #2-CH₂CH₂-*2, #2-CH₂CH₂CH₂-*2, #2-C(═O)CH₂CH₂O—*2, #2-C(═O)CH₂CH₂CH₂O-*2, #2-CH₂CH(OH)CH₂O—*2, #2-CH₂O-*2, #2-CH₂CH₂O-*2, and #2-CH₂CH₂CH₂O-*2.

3. The compound of claim 2, or a pharmaceutically acceptable salt thereof, wherein the compound of formula (II) is a compound of formula (IV) 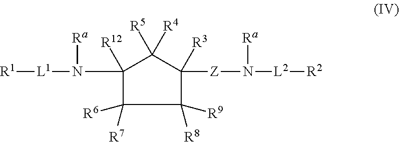
provided that:
(i) when Z is a bond, L¹ is *1-CH₂-#1, and L² is #2-CH₂-*2; then either:
(i-1) at least one of R³, R⁴, and R⁵ is hydrogen or halogen; or
(i-2) R³ and R¹² are taken together to form a CR¹³R¹⁴ group; and
(ii) when Z is a bond, and R³ and R¹² are taken together to form CR¹³R¹⁴; then either:
(ii-1) L¹ is selected from the group consisting of *1-C(═O)-#1, *1-CH₂-#1, *1-CH₂CH₂-#1, *1-CH₂CH₂CH₂-#1, *1-OCH₂CH₂C(═O)-#1, *1-OCH₂CH₂CH₂C(═O)-#1, *1-OCH₂CH(OH)CH₂-#1, *1-OCH₂-#1, *1-OCH₂CH₂-#1, and *1-OCH₂CH₂CH₂-#1; or
(ii-2) L² is selected from the group consisting of #2-C(═O)-*2, #2-CH₂-*2, #2-CH₂CH₂-*2, #2-CH₂CH₂CH₂-*2, #2-C(═O)CH₂CH₂O—*2, #2-C(═O)CH₂CH₂CH₂O-*2, #2-CH₂CH(OH)CH₂O—*2, #2-CH₂O-*2, #2-CH₂CH₂O-*2, and #2-CH₂CH₂CH₂O-*2.

4. The compound of claim 3, or a pharmaceutically acceptable salt thereof, wherein the compound of formula (IV) is a compound of formula (IV-a) 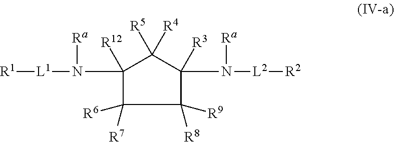
wherein:
R³ is hydrogen, halogen, or C₁-C₆ alkyl;
R¹² is hydrogen, halogen, or C₁-C₆ alkyl; and
provided that when L¹ is *1-CH₂-#1, and L² is #2-CH₂-*2; then at least one of R³, R⁴, and R⁵ is hydrogen or halogen.

5. The compound of claim 3, or a pharmaceutically acceptable salt thereof, wherein the compound of formula (IV) is a compound of formula (IV-e) 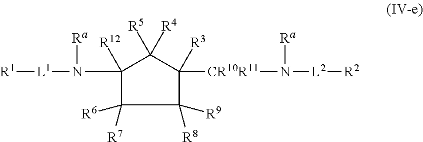
wherein:
R³ is hydrogen, halogen, or C₁-C₆ alkyl; and
R¹² is hydrogen, halogen, or C₁-C₆ alkyl.

6. The compound of claim 1, or a pharmaceutically acceptable salt thereof, wherein the compound of formula (I) is a compound of formula (III) 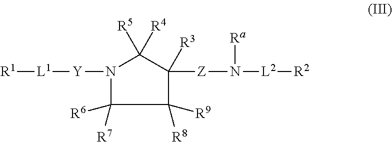
provided that when Y is a bond; then:
L¹ is selected from the group consisting of *1-OCH₂CH(OH)CH₂-#1, *1-OCH₂-#1, *1-OCH₂CH₂-#1, and *1-OCH₂CH₂CH₂-#1; and
further provided that, when Z is CR¹⁰R¹¹, then at least one of R¹ and R² is substituted by two or more halo groups.

7. The compound of claim 6, or a pharmaceutically acceptable salt thereof, wherein the compound of formula (III) is a compound of formula (VI) 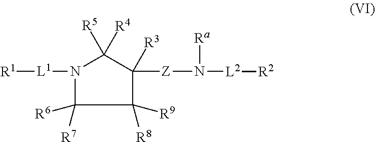
provided that L¹ is selected from the group consisting of *1-OCH₂CH(OH)CH₂-#1, *1-OCH₂-#1, *1-OCH₂CH₂-#1, and *1-OCH₂CH₂CH₂-#1; and
further provided that, when Z is CR¹⁰R¹¹, then at least one of R¹ and R² is substituted by two or more halo groups.

8. The compound of claim 7, or a pharmaceutically acceptable salt thereof, wherein the compound of formula (VI) is a compound of formula (VI-a) 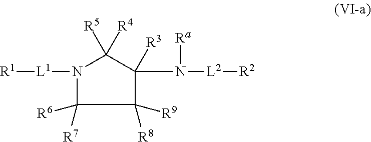
provided that L¹ is selected from the group consisting of *1-OCH₂CH(OH)CH₂-#1, *1-OCH₂-#1, *1-OCH₂CH₂-#1, and *1-OCH₂CH₂CH₂-#1.

9. The compound of claim 7, or a pharmaceutically acceptable salt thereof, wherein the compound of formula (VI) is a compound of formula (VI-c) 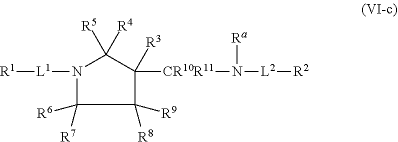
provided that L¹ is selected from the group consisting of *1-OCH₂CH(OH)CH₂-#1, *1-OCH₂-#1, *1-OCH₂CH₂-#1, and *1-OCH₂CH₂CH₂-#1; and at least one of R¹ and R² is substituted by two or more halo groups.

10. The compound of claim 6, or a pharmaceutically acceptable salt thereof, wherein the compound of formula (III) is a compound of formula (VII)

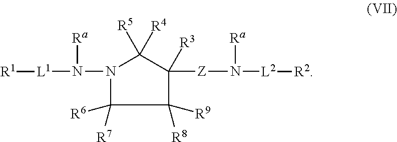

11. The compound of claim 10, or a pharmaceutically acceptable salt thereof, wherein the compound of formula (VII) is a compound of formula (VII-a)

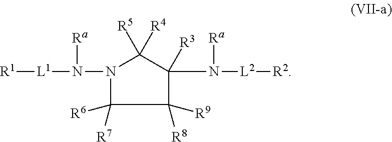

12. The compound of claim 10, or a pharmaceutically acceptable salt thereof, wherein the compound of formula (VII) is a compound of formula (VII-c)

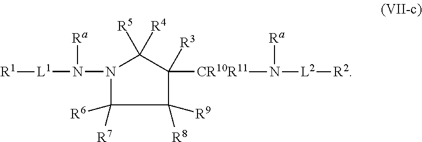

13. A compound selected from the group consisting of a compound of Table 1, or a pharmaceutically acceptable salt thereof.

14. A pharmaceutical composition comprising a compound of claim 1, or a pharmaceutically acceptable salt thereof, and a pharmaceutically acceptable carrier.

15. A method of treating a disease or disorder mediated by an integrated stress response (ISR) pathway in an individual in need thereof comprising administering to the individual a therapeutically effective amount of a compound of claim 1, or a pharmaceutically acceptable salt thereof.

16. The method of claim 15, wherein the disease or disorder is a neurodegenerative disease, an inflammatory disease, an autoimmune disease, a metabolic syndrome, a cancer, a vascular disease, an ocular disease, or a musculoskeletal disease.

17. The method of claim 15, wherein the disease is vanishing white matter disease, childhood ataxia with CNS hypomyelination, intellectual disability syndrome, Alzheimer's disease, prion disease, Creutzfeldt-Jakob disease, Parkinson's disease, amyotrophic lateral sclerosis (ALS) disease, cognitive impairment, frontotemporal dementia (FTD), traumatic brain injury, postoperative cognitive dysfunction (PCD), neuro-otological syndromes, hearing loss, Huntington's disease, stroke, chronic traumatic encephalopathy, spinal cord injury, dementias or cognitive impairment, arthritis, psoriatic arthritis, psoriasis, juvenile idiopathic arthritis, asthma, allergic asthma, bronchial asthma, tuberculosis, chronic airway disorder, cystic fibrosis, glomerulonephritis, membranous nephropathy, sarcoidosis, vasculitis, ichthyosis, transplant rejection, interstitial cystitis, atopic dermatitis or inflammatory bowel disease, Crohn's disease, ulcerative colitis, celiac disease, systemic lupus erythematosus, type 1 diabetes, multiple sclerosis, rheumatoid arthritis, alcoholic liver steatosis, obesity, glucose intolerance, insulin resistance, hyperglycemia, fatty liver, dyslipidemia, hyperlipidemia, type 2 diabetes, pancreatic cancer, breast cancer, kidney cancer, bladder cancer, prostate cancer, testicular cancer, urothelial cancer, endometrial cancer, ovarian cancer, cervical cancer, renal cancer, esophageal cancer, gastrointestinal stromal tumor (GIST), multiple myeloma, cancer of secretory cells, thyroid cancer, gastrointestinal carcinoma, chronic myeloid leukemia, hepatocellular carcinoma, colon cancer, melanoma, malignant glioma, glioblastoma, glioblastoma multiforme, astrocytoma, dysplastic gangliocytoma of the cerebellum, Ewing's sarcoma, rhabdomyosarcoma, ependymoma, medulloblastoma, ductal adenocarcinoma, adenosquamous carcinoma, nephroblastoma, acinar cell carcinoma, lung cancer, non-Hodgkin's lymphoma, Burkitt's lymphoma, chronic lymphocytic leukemia, monoclonal gammopathy of undetermined significance (MGUS), plasmocytoma, lymphoplasmacytic lymphoma, acute lymphoblastic leukemia, Pelizaeus-Merzbacher disease, atherosclerosis, abdominal aortic aneurism, carotid artery disease, deep vein thrombosis, Buerger's disease, chronic venous hypertension, vascular calcification, telangiectasia or lymphoedema, glaucoma, age-related macular degeneration, inflammatory retinal disease, retinal vascular disease, diabetic retinopathy, uveitis, rosacea, Sjogren's syndrome or neovascularization in proliferative retinopathy, hyperhomocysteinemia, skeletal muscle atrophy, myopathy, muscular dystrophy, muscular wasting, sarcopenia, Duchenne muscular dystrophy (DMD), Becker's disease, myotonic dystrophy, X-linked dilated cardiomyopathy, or spinal muscular atrophy (SMA).

18. A method of producing a protein, comprising contacting a eukaryotic cell comprising a nucleic acid encoding the protein with the compound or salt of claim 1.

19. A method of culturing a eukaryotic cell comprising a nucleic acid encoding a protein, comprising contacting the eukaryotic cell with an in vitro culture medium comprising a compound or salt of claim 1.

20. A method of producing a protein, comprising contacting a cell-free protein synthesis (CFPS) system comprising eukaryotic initiation factor 2 (eIF2) and a nucleic acid encoding a protein with the compound or salt of claim 1.

21. An in vitro cell culture medium, comprising the compound or salt of claim 1 and nutrients for cellular growth.

22. A cell-free protein synthesis (CFPS) system comprising eukaryotic initiation factor 2 (eIF2) and a nucleic acid encoding a protein with the compound or salt of claim 1.
